SEQUENCE LISTING (331 sequences)

SEQ ID NO: 1 - protein, 448 aa, Homo sapiens
  source 1..448: note = Human ILT3 amino acid sequence with predicted signal sequence
  SIGNAL 1..21
MIPTFTALLCLGLSLGPRTHMQAGPLPKPTLWAEPGSVISWGNSVTIWCQGTLEAREYRLDKEESPAPWDRQNPLEPKNKARFSIPSMTEDYAGRYRCYYRSPVGWSQPSDPLELVMTGAYSKPTLSALPSPLVTSGKSVTLLCQSRSPMDTFLLIKERAAHPLLHLRSEHGAQQHQAEFPMSPVTSVHGGTYRCFSSHGFSHYLLSHPSDPLELIVSGSLEDPRPSPTRSVSTAAGPEDQPLMPTGSVPHSGLRRHWEVLIGVLVVSILLLSLLLFLLLQHWRQGKHRTLAQRQADFQRPPGAAEPEPKDGGLQRRSSPAADVQGENFCAAVKNTQPEDGVEMDTRQSPHDEDPQAVTYAKVKHSRPRREMASPPSPLSGEFLDTKDRQAEEDRQMDTEAAASEAPQDVTYAQLHSFTLRQKATEPPPSQEGASPAEPSVYATLAIH

SEQ ID NO: 2 - protein, 427 aa, Homo sapiens
  source 1..427: note = Human ILT3 amino acid sequence without predicted signal sequence
QAGPLPKPTLWAEPGSVISWGNSVTIWCQGTLEAREYRLDKEESPAPWDRQNPLEPKNKARFSIPSMTEDYAGRYRCYYRSPVGWSQPSDPLELVMTGAYSKPTLSALPSPLVTSGKSVTLLCQSRSPMDTFLLIKERAAHPLLHLRSEHGAQQHQAEFPMSPVTSVHGGTYRCFSSHGFSHYLLSHPSDPLELIVSGSLEDPRPSPTRSVSTAAGPEDQPLMPTGSVPHSGLRRHWEVLIGVLVVSILLLSLLLFLLLQHWRQGKHRTLAQRQADFQRPPGAAEPEPKDGGLQRRSSPAADVQGENFCAAVKNTQPEDGVEMDTRQSPHDEDPQAVTYAKVKHSRPRREMASPPSPLSGEFLDTKDRQAEEDRQMDTEAAASEAPQDVTYAQLHSFTLRQKATEPPPSQEGASPAEPSVYATLAIH

SEQ ID NO: 3 - protein, 238 aa, Homo sapiens
  source 1..238: note = Human ILT3 Extracellular domain (aa 22-259)
QAGPLPKPTLWAEPGSVISWGNSVTIWCQGTLEAREYRLDKEESPAPWDRQNPLEPKNKARFSIPSMTEDYAGRYRCYYRSPVGWSQPSDPLELVMTGAYSKPTLSALPSPLVTSGKSVTLLCQSRSPMDTFLLIKERAAHPLLHLRSEHGAQQHQAEFPMSPVTSVHGGTYRCFSSHGFSHYLLSHPSDPLELIVSGSLEDPRPSPTRSVSTAAGPEDQPLMPTGSVPHSGLRRHWE

SEQ ID NO: 4 - protein, 92 aa, Homo sapiens
  source 1..92: note = Human ILT3 Ig-like C2-type Domain 1 amino acid sequence without N-terminal domain (aa 27-118)
PKPTLWAEPGSVISWGNSVTIWCQGTLEAREYRLDKEESPAPWDRQNPLEPKNKARFSIPSMTEDYAGRYRCYYRSPVGWSQPSDPLELVMT

SEQ ID NO: 5 - protein, 95 aa, Homo sapiens
  source 1..95: note = Human ILT3 Ig-like C2-type Domain 2 amino acid sequence (aa 124-218)
PTLSALPSPLVTSGKSVTLLCQSRSPMDTFLLIKERAAHPLLHLRSEHGAQQHQAEFPMSPVTSVHGGTYRCFSSHGFSHYLLSHPSDPLELIVS

SEQ ID NO: 6 - protein, 448 aa, Macaca fascicularis
  source 1..448: note = Cyno ILT3 amino acid sequence with predicted signal sequence
  SIGNAL 1..21
MTPPLTVLFCLGLSLGPRTCVQAGPLPKPTVWAEPGSVISWGSPVTIWCQGTLDAQEYHLDKEGSPAPWDTQNPLEPRNKAKFSIPSMTQHYAGRYRCYYHSHPDWSEDSDPLDLVMTGAYSKPILSVLPSPLVTSGESVTLLCQSQSPMDTFLLFKEGAAHPLPRLRSQHGAQLHWAEFPMGPVTSVHGGTYRCISSRSFSHYLLSRPSDPVELTVLGSLESPSPSPTRSISAAAGPEDQSLMPTGSDPQSGLRRHWEVLIGVLVVSILLLSLVFFLLLQHWRQGKHRTSAQRQADFQRPPGAAEPEPKDGGLQRRSRPAADVQGENPNAAMKDTQPEDGVELDSRQRPHDEDPQAVTYARVKHSGPRREMASPPSPLSEEFLDTKDTQAEEDRQMDTQAATSEAPQDVTYAQLQSLTLRREATEPPPPQKREPSAEPSVYATLAIH

SEQ ID NO: 7 - protein, 427 aa, Macaca fascicularis
  source 1..427: note = Cyno ILT3 amino acid sequence without predicted signal sequence
QAGPLPKPTVWAEPGSVISWGSPVTIWCQGTLDAQEYHLDKEGSPAPWDTQNPLEPRNKAKFSIPSMTQHYAGRYRCYYHSHPDWSEDSDPLDLVMTGAYSKPILSVLPSPLVTSGESVTLLCQSQSPMDTFLLFKEGAAHPLPRLRSQHGAQLHWAEFPMGPVTSVHGGTYRCISSRSFSHYLLSRPSDPVELTVLGSLESPSPSPTRSISAAAGPEDQSLMPTGSDPQSGLRRHWEVLIGVLVVSILLLSLVFFLLLQHWRQGKHRTSAQRQADFQRPPGAAEPEPKDGGLQRRSRPAADVQGENPNAAMKDTQPEDGVELDSRQRPHDEDPQAVTYARVKHSGPRREMASPPSPLSEEFLDTKDTQAEEDRQMDTQAATSEAPQDVTYAQLQSLTLRREATEPPPPQKREPSAEPSVYATLAIH

SEQ ID NO: 8 - protein, 238 aa, Macaca fascicularis
  source 1..238: note = Cyno ILT3 Extracellular domain (aa 22-259)
QAGPLPKPTVWAEPGSVISWGSPVTIWCQGTLDAQEYHLDKEGSPAPWDTQNPLEPRNKAKFSIPSMTQHYAGRYRCYYHSHPDWSEDSDPLDLVMTGAYSKPILSVLPSPLVTSGESVTLLCQSQSPMDTFLLFKEGAAHPLPRLRSQHGAQLHWAEFPMGPVTSVHGGTYRCISSRSFSHYLLSRPSDPVELTVLGSLESPSPSPTRSISAAAGPEDQSLMPTGSDPQSGLRRHWE

SEQ ID NO: 9 - protein, 92 aa, Macaca fascicularis
  source 1..92: note = Cyno ILT3 Ig-like C2-type Domain 1 amino acid sequence without N-terminal domain (aa 27-118)
PKPTVWAEPGSVISWGSPVTIWCQGTLDAQEYHLDKEGSPAPWDTQNPLEPRNKAKFSIPSMTQHYAGRYRCYYHSHPDWSEDSDPLDLVMT

SEQ ID NO: 10 - protein, 95 aa, Macaca fascicularis
  source 1..95: note = Cyno ILT3 Ig-like C2-type Domain 2 amino acid sequence without N-terminal domain (aa 124-218)
PILSVLPSPLVTSGESVTLLCQSQSPMDTFLLFKEGAAHPLPRLRSQHGAQLHWAEFPMGPVTSVHGGTYRCISSRSFSHYLLSRPSDPVELTVL

SEQ ID NO: 11 - protein, 10 aa, synthetic construct
  source 1..10: note = Antibody 3A3 VH CDR1 - Exemplary
GFSLTSYGVH

SEQ ID NO: 12 - protein, 16 aa, synthetic construct
  source 1..16: note = Antibody 3A3 VH CDR2 - Exemplary, Kabat
VIWPGGTINYNSALMS

SEQ ID NO: 13 - protein, 10 aa, synthetic construct
  source 1..10: note = Antibody 3A3 VH CDR3 - Exemplary, Chothia, AbM, Kabat
DKYDGGWFAY

SEQ ID NO: 14 - protein, 11 aa, synthetic construct
  source 1..11: note = Antibody 3A3 LH CDR1 - Exemplary, Chothia, AbM, Kabat
KASQNVRTAVA

SEQ ID NO: 15 - protein, 7 aa, synthetic construct
  source 1..7: note = Antibody 3A3 LH CDR2 - Exemplary, Chothia, AbM, Kabat
LASNRHT

SEQ ID NO: 16 - protein, 9 aa, synthetic construct
  source 1..9: note = Antibody 3A3 LH CDR3 - Exemplary, Chothia, AbM, Kabat LH
LQHLNYPLT

SEQ ID NO: 17 - protein, 7 aa, synthetic construct
  source 1..7: note = Antibody 3A3 VH CDR1 - Chothia
GFSLTSY

SEQ ID NO: 18 - protein, 5 aa, synthetic construct
  source 1..5: note = Antibody 3A3 VH CDR2 - Chothia
WPGGT

SEQ ID NO: 19 - protein, 9 aa, synthetic construct
  source 1..9: note = Antibody 3A3 VH CDR2 - AbM
VIWPGGTIN

SEQ ID NO: 20 - protein, 5 aa, synthetic construct
  source 1..5: note = Antibody 3A3 VH CDR1 - Kabat
SYGVH

SEQ ID NO: 21 - protein, 6 aa, synthetic construct
  source 1..6: note = Antibody 3A3 VH CDR1 - Contact
TSYGVH

SEQ ID NO: 22 - protein, 12 aa, synthetic construct
  source 1..12: note = Antibody 3A3 VH CDR2 - Contact
WLGVIWPGGTIN

SEQ ID NO: 23 - protein, 11 aa, synthetic construct
  source 1..11: note = Antibody 3A3 VH CDR3 - Contact
ASDKYDGGWFA

SEQ ID NO: 24 - protein, 7 aa, synthetic construct
  source 1..7: note = Antibody 3A3 LH CDR1 - Contact
RTAVAWY

SEQ ID NO: 25 - protein, 10 aa, synthetic construct
  source 1..10: note = Antibody 3A3 LH CDR2 - Contact
ALIYLASNRH

SEQ ID NO: 26 - protein, 8 aa, synthetic construct
  source 1..8: note = Antibody 3A3 LH CDR3 - Contact
LQHLNYPL

SEQ ID NO: 27 - protein, 10 aa, synthetic construct
  source 1..10: note = Antibody 5A7, 48A6, Hz5A7.v5 VH CDR1 - Exemplary, AbM
GFTFSSYGMS

SEQ ID NO: 28 - protein, 17 aa, synthetic construct
  source 1..17: note = Antibody 5A7, Hz5A7.v5 VH CDR2 - Exemplary, Kabat
TISGGGSYTNYPDSVKG

SEQ ID NO: 29 - protein, 12 aa, synthetic construct
  source 1..12: note = Antibody 5A7 VH CDR3 - Exemplary, Chothia, AbM, Kabat
REWRMTLYAMDY

SEQ ID NO: 30 - protein, 15 aa, synthetic construct
  source 1..15: note = Antibody 5A7 VH CDR1 - Exemplary, Chothia, AbM, Kabat
RASESVDSYGNSFMH

SEQ ID NO: 31 - protein, 7 aa, synthetic construct
  source 1..7: note = Antibody 5A7, Hz5A7.v5 LH CDR2 - Exemplary, Chothia, AbM, Kabat
LTSNLES

SEQ ID NO: 32 - protein, 9 aa, synthetic construct
  source 1..9: note = Antibody 5A7, Hz5A7.v5 LH CDR3 - Exemplary, Chothia LH, AbM, Kabat
QQNNEDPFT

SEQ ID NO: 33 - protein, 7 aa, synthetic construct
  source 1..7: note = Antibody 5A7, 48A6, Hz5A7.v5 VH CDR1 - Chothia
GFTFSSY

SEQ ID NO: 34 - protein, 6 aa, synthetic construct
  source 1..6: note = Antibody 5A7, Hz5A7.v5 VH CDR2 - Chothia
SGGGSY

SEQ ID NO: 35 - protein, 10 aa, synthetic construct
  source 1..10: note = Antibody 5A7, Hz5A7.v5 VH CDR2 - AbM
TISGGGSYTN

SEQ ID NO: 36 - protein, 5 aa, synthetic construct
  source 1..5: note = Antibody 5A7, 48A6, Hz5A7.v5 VH CDR1 - Kabat
SYGMS

SEQ ID NO: 37 - protein, 6 aa, synthetic construct
  source 1..6: note = Antibody 5A7, 48A6, Hz5A7.v5 VH CDR1 - Contact
SSYGMS

SEQ ID NO: 38 - protein, 13 aa, synthetic construct
  source 1..13: note = Antibody 5A7, Hz5A7.v5 VH CDR2 - Contact
WVATISGGGSYTN

SEQ ID NO: 39 - protein, 13 aa, synthetic construct
  source 1..13: note = Antibody 5A7 VH CDR3 - Contact
ARREWRMTLYAMD

SEQ ID NO: 40 - protein, 11 aa, synthetic construct
  source 1..11: note = Antibody 5A7 LH CDR1 - Contact
DSYGNSFMHWY

SEQ ID NO: 41 - protein, 10 aa, synthetic construct
  source 1..10: note = Antibody 5A7 LH CDR2 - Contact
LLIYLTSNLE

SEQ ID NO: 42 - protein, 8 aa, synthetic construct
  source 1..8: note = Antibody 5A7, Hz5A7.v5 LH CDR3 - Contact
QQNNEDPF

SEQ ID NO: 43 - protein, 10 aa, synthetic construct
  source 1..10: note = Antibody 12A12, 16C5 VH CDR1 - Exemplary, AbM
GYTFTDYNMD

SEQ ID NO: 44 - protein, 17 aa, synthetic construct
  source 1..17: note = Antibody 12A12 VH CDR2 - Exemplary, Kabat
YIYPNNGGTGYNQKFNS

SEQ ID NO: 45 - protein, 14 aa, synthetic construct
  source 1..14: note = Antibody 12A12 VH CDR3 - Exemplary, Chothia, AbM, Kabat
SPYYDYVGSYAMDY

SEQ ID NO: 46 - protein, 12 aa, synthetic construct
  source 1..12: note = Antibody 12A12 LH CDR1 - Exemplary, Chothia, AbM, Kabat
TASSSVSSSYLH

SEQ ID NO: 47 - protein, 7 aa, synthetic construct
  source 1..7: note = Antibody 12A12 LH CDR2 - Exemplary, Chothia, AbM, Kabat
STSNLAS

SEQ ID NO: 48 - protein, 9 aa, synthetic construct
  source 1..9: note = Antibody 12A12 LH CDR3 - Exemplary, Chothia, AbM, Kabat
HQYHRSPRT

SEQ ID NO: 49 - protein, 7 aa, synthetic construct
  source 1..7: note = Antibody 12A12, 16C5 VH CDR1 - Chothia
GYTFTDY

SEQ ID NO: 50 - protein, 6 aa, synthetic construct
  source 1..6: note = Antibody 12A12 VH CDR2 - Chothia
YPNNGG

SEQ ID NO: 51 - protein, 10 aa, synthetic construct
  source 1..10: note = Antibody 12A12 VH CDR2 - AbM
YIYPNNGGTG

SEQ ID NO: 52 - protein, 5 aa, synthetic construct
  source 1..5: note = Antibody 12A12, 16C5 VH CDR1 - Kabat
DYNMD

SEQ ID NO: 53 - protein, 6 aa, synthetic construct
  source 1..6: note = Antibody 12A12, 16C5 VH CDR1 - Contact
TDYNMD

SEQ ID NO: 54 - protein, 13 aa, synthetic construct
  source 1..13: note = Antibody 12A12 VH CDR2 - Contact
WIGYIYPNNGGTG

SEQ ID NO: 55 - protein, 15 aa, synthetic construct
  source 1..15: note = Antibody 12A12 VH CDR3 - Contact
ASSPYYDYVGSYAMD

SEQ ID NO: 56 - protein, 8 aa, synthetic construct
  source 1..8: note = Antibody 12A12 LH CDR1 - Contact
SSSYLHWY

SEQ ID NO: 57 - protein, 10 aa, synthetic construct
  source 1..10: note = Antibody 12A12 LH CDR2 - Contact
LWIYSTSNLA

SEQ ID NO: 58 - protein, 8 aa, synthetic construct
  source 1..8: note = Antibody 12A12 LH CDR3 - Contact
HQYHRSPR

SEQ ID NO: 59 - protein, 17 aa, synthetic construct
  source 1..17: note = Antibody 16C5 VH CDR2 - Exemplary, Kabat
YIYPSNGGTGYNQKFKS

SEQ ID NO: 60 - protein, 14 aa, synthetic construct
  source 1..14: note = Antibody 16C5 VH CDR3 - Exemplary, Chothia, AbM, Kabat
VPYYDYLYYYAMDY

SEQ ID NO: 61 - protein, 10 aa, synthetic construct
  source 1..10: note = Antibody 16C5 VH CDR1 - Exemplary, Chothia, AbM, Kabat
RASSSVSFMH

SEQ ID NO: 62 - protein, 7 aa, synthetic construct
  source 1..7: note = Antibody 16C5 VH CDR2 - Exemplary, Chothia, AbM, Kabat
ATSNLAS

SEQ ID NO: 63 - protein, 11 aa, synthetic construct
  source 1..11: note = Antibody 16C5 LH CDR3 - Exemplary, Chothia, AbM, Kabat
QQWSTNPYMYT

SEQ ID NO: 64 - protein, 6 aa, synthetic construct
  source 1..6: note = Antibody 16C5 VH CDR2 - Chothia
YPSNGG

SEQ ID NO: 65 - protein, 10 aa, synthetic construct
  source 1..10: note = Antibody 16C5 VH CDR2 - AbM
YIYPSNGGTG

SEQ ID NO: 66 - protein, 13 aa, synthetic construct
  source 1..13: note = Antibody 16C5 VH CDR2 - Contact
WIGYIYPSNGGTG

SEQ ID NO: 67 - protein, 15 aa, synthetic construct
  source 1..15: note = Antibody 16C5 VH CDR3 - Contact
ARVPYYDYLYYYAMD

SEQ ID NO: 68 - protein, 6 aa, synthetic construct
  source 1..6: note = Antibody 16C5 LH CDR1 - Contact
SFMHWY

SEQ ID NO: 69 - protein, 10 aa, synthetic construct
  source 1..10: note = Antibody 16C5 LH CDR2 - Contact
PWIYATSNLA

SEQ ID NO: 70 - protein, 10 aa, synthetic construct
  source 1..10: note = Antibody 16C5 LH CDR3 - Contact
QQWSTNPYMY

SEQ ID NO: 71 - protein, 10 aa, synthetic construct
  source 1..10: note = Antibody 45G10, 53F10 VH CDR1 - Exemplary, AbM
GFTFSDYGMH

SEQ ID NO: 72 - protein, 17 aa, synthetic construct
  source 1..17: note = Antibody 45G10 VH CDR2 - Exemplary, Kabat
YIFSGSSTIYYADTVKG

SEQ ID NO: 73 - protein, 9 aa, synthetic construct
  source 1..9: note = Antibody 45G10, 53F10 VH CDR3 - Exemplary, Chothia, AbM, Kabat
ADGRGAMDY

SEQ ID NO: 74 - protein, 11 aa, synthetic construct
  source 1..11: note = Antibody 45G10 LH CDR1 - Exemplary, Chothia, AbM, Kabat
RASQDISKFLN

SEQ ID NO: 75 - protein, 7 aa, synthetic construct
  source 1..7: note = Antibody 45G10, 53F10 LH CDR2 - Exemplary, Chothia, AbM, Kabat
YTSRLHS

SEQ ID NO: 76 - protein, 9 aa, synthetic construct
  source 1..9: note = Antibody 45G10, 53F10 LH CDR3 - Exemplary, Chothia, AbM, Kabat
QQGNTLPWT

SEQ ID NO: 77 - protein, 7 aa, synthetic construct
  source 1..7: note = Antibody 45G10, 53F10 VH CDR1 - Chothia
GFTFSDY

SEQ ID NO: 78 - protein, 6 aa, synthetic construct
  source 1..6: note = Antibody 45G10 VH CDR2 - Chothia
FSGSST

SEQ ID NO: 79 - protein, 10 aa, synthetic construct
  source 1..10: note = Antibody 45G10 VH CDR2 - AbM
YIFSGSSTIY

SEQ ID NO: 80 - protein, 5 aa, synthetic construct
  source 1..5: note = Antibody 45G10, 53F10 VH CDR1 - Kabat
DYGMH

SEQ ID NO: 81 - protein, 6 aa, synthetic construct
  source 1..6: note = Antibody 45G10, 53F10 VH CDR1 - Contact
SDYGMH

SEQ ID NO: 82 - protein, 13 aa, synthetic construct
  source 1..13: note = Antibody 45G10 VH CDR2 - Contact
WVAYIFSGSSTIY

SEQ ID NO: 83 - protein, 10 aa, synthetic construct
  source 1..10: note = Antibody 45G10, 53F10 VH CDR3 - Contact
ARADGRGAMD

SEQ ID NO: 84 - protein, 7 aa, synthetic construct
  source 1..7: note = Antibody 45G10 LH CDR1 - Contact
SKFLNWY

SEQ ID NO: 85 - protein, 10 aa, synthetic construct
  source 1..10: note = Antibody 45G10, 53F10 LH CDR2 - Contact
LLIYYTSRLH

SEQ ID NO: 86 - protein, 8 aa, synthetic construct
  source 1..8: note = Antibody 45G10, 53F10 LH CDR3 - Contact
QQGNTLPW

SEQ ID NO: 87 - protein, 17 aa, synthetic construct
  source 1..17: note = Antibody 48A6 VH CDR2 - Exemplary, Kabat
TISSGGTYTFYPDSVKG

SEQ ID NO: 88 - protein, 12 aa, synthetic construct
  source 1..12: note = Antibody 48A6 VH CDR3 - Exemplary, Chothia, AbM, Kabat
RGWLLHYYAMDY

SEQ ID NO: 89 - protein, 15 aa, synthetic construct
  source 1..15: note = Antibody 48A6 VH CDR1 - Exemplary, Chothia, AbM, Kabat
RPSESVDSFGNSFMH

SEQ ID NO: 90 - protein, 7 aa, synthetic construct
  source 1..7: note = Antibody 48A6 LH CDR2 - Exemplary, Chothia, AbM, Kabat
LSSKLES

SEQ ID NO: 91 - protein, 9 aa, synthetic construct
  source 1..9: note = Antibody 48A6 LH CDR3 - Exemplary, Chothia, AbM, Kabat
QQHNEDPFT

SEQ ID NO: 92 - protein, 6 aa, synthetic construct
  source 1..6: note = Antibody 48A6 VH CDR2 - Chothia
SSGGTY

SEQ ID NO: 93 - protein, 10 aa, synthetic construct
  source 1..10: note = Antibody 48A6 VH CDR2 - AbM
TISSGGTYTF

SEQ ID NO: 94 - protein, 13 aa, synthetic construct
  source 1..13: note = Antibody 48A6 VH CDR2 - Contact
WVATISSGGTYTF

SEQ ID NO: 95 - protein, 13 aa, synthetic construct
  source 1..13: note = Antibody 48A6 VH CDR3 - Contact
ARRGWLLHYYAMD

SEQ ID NO: 96 - protein, 11 aa, synthetic construct
  source 1..11: note = Antibody 48A6 LH CDR1 - Contact
DSFGNSFMHWF

SEQ ID NO: 97 - protein, 10 aa, synthetic construct
  source 1..10: note = Antibody 48A6 LH CDR2 - Contact
LLIYLSSKLE

SEQ ID NO: 98 - protein, 8 aa, synthetic construct
  source 1..8: note = Antibody 48A6 LH CDR3 - Contact
QQHNEDPF

SEQ ID NO: 99 - protein, 17 aa, synthetic construct
  source 1..17: note = Antibody 53F10 VH CDR2 - Exemplary, Kabat
YISTGIITVYYADTVKG

SEQ ID NO: 100 - protein, 11 aa, synthetic construct
  source 1..11: note = Antibody 53F10 LH CDR1 - Exemplary, Chothia, AbM, Kabat
RASQDISNFLN

SEQ ID NO: 101 - protein, 6 aa, synthetic construct
  source 1..6: note = Antibody 53F10 VH CDR2 - Chothia
STGIIT

SEQ ID NO: 102 - protein, 10 aa, synthetic construct
  source 1..10: note = Antibody 53F10 VH CDR2 - AbM
YISTGIITVY

SEQ ID NO: 103 - protein, 13 aa, synthetic construct
  source 1..13: note = Antibody 53F10 VH CDR2 - Contact
WVAYISTGIITVY

SEQ ID NO: 104 - protein, 7 aa, synthetic construct
  source 1..7: note = Antibody 53F10 LH CDR1 - Contact
SNFLNWY

SEQ ID NO: 105 - protein, 12 aa, synthetic construct
  source 1..12: note = Antibody Hz5A7.v5 VH CDR3 - Exemplary, Chothia, AbM, Kabat
REWRYTLYAMDY

SEQ ID NO: 106 - protein, 15 aa, synthetic construct
  source 1..15: note = Antibody Hz5A7.v5 LH CDR1 - Exemplary, Chothia, AbM, Kabat
RASESVESYGSSFMH

SEQ ID NO: 107 - protein, 13 aa, synthetic construct
  source 1..13: note = Antibody Hz5A7.v5 VH CDR3 - Contact
ARREWRYTLYAMD

SEQ ID NO: 108 - protein, 11 aa, synthetic construct
  source 1..11: note = Antibody Hz5A7.v5 LH CDR1 - Contact
ESYGSSFMHWY

SEQ ID NO: 109 - protein, 118 aa, synthetic construct
  source 1..118: note = 3A3 VH amino acid sequence
QVQLKESGPGLVAPSQSLSITCTVSGFSLTSYGVHWVRQPPGKGLEWLGVIWPGGTINYNSALMSRLSISKDNSKSQVFLKLNSLQTDDTAMYYCASDKYDGGWFAYWGQGTLVTVSA

SEQ ID NO: 110 - protein, 107 aa, synthetic construct
  source 1..107: note = 3A3 LH amino acid sequence
DIVMTQSQKFMSTSVGDRVSITCKASQNVRTAVAWYQQKPGQSPEALIYLASNRHTGVPDRFTGSGSGTDFSLSISNVQSEDLADYFCLQHLNYPLTFGSGTKLEIK

SEQ ID NO: 111 - protein, 121 aa, synthetic construct
  source 1..121: note = 5A7 VH amino acid sequence
EVKLVESGGGLVKPGGSLKLSCAASGFTFSSYGMSWVRQTPEKRLEWVATISGGGSYTNYPDSVKGRLTISRDNAKKNLYLEMSSLRSEDTALYYCARREWRMTLYAMDYWGQGTSVTVSS

SEQ ID NO: 112 - protein, 111 aa, synthetic construct
  source 1..111: note = 5A7 LH amino acid sequence
NIVLTQSPASLAVSLGQRATISCRASESVDSYGNSFMHWYQQKPGQAPKLLIYLTSNLESGVPARFSGSGSRTDFTLTIDPVEADDAATYYCQQNNEDPFTFGSGTKLEIK

SEQ ID NO: 113 - protein, 123 aa, synthetic construct
  source 1..123: note = 12A12 VH amino acid sequence
EVQLQQSGPELVKPGASVKISCKASGYTFTDYNMDWVKQSHGKSLEWIGYIYPNNGGTGYNQKFNSKATLTVDKSSSTAYMELHSLTSEDSAVYYCASSPYYDYVGSYAMDYWGQGTSVTVSS

SEQ ID NO: 114 - protein, 108 aa, synthetic construct
  source 1..108: note = 12A12 LH amino acid sequence
QIVLTQSPAIMSASLGERVTMTCTASSSVSSSYLHWYQQKPGSSPKLWIYSTSNLASGVPARFSGSGSGTSYSLTISSMEAEDAATYYCHQYHRSPRTFGGGTKLEIK

SEQ ID NO: 115 - protein, 123 aa, synthetic construct
  source 1..123: note = 16C5 VH amino acid sequence
EVQLQQSGPELVKPGASVKISCKASGYTFTDYNMDWVKQSHGKSLEWIGYIYPSNGGTGYNQKFKSKATLTVDKSSNTAYMELHSLTSEDSAVYYCARVPYYDYLYYYAMDYWGQGTSVTVSS

SEQ ID NO: 116 - protein, 108 aa, synthetic construct
  source 1..108: note = 16C5 LH amino acid sequence
QIVLSQSPAILSASPGEKVTMACRASSSVSFMHWYQQKPGSSPQPWIYATSNLASGVPARFSGSGSGTSYSLTISRVEAEDAATYYCQQWSTNPYMYTFGGGTKLEIK

SEQ ID NO: 117 - protein, 118 aa, synthetic construct
  source 1..118: note = 45G10 VH amino acid sequence
EVQLVESGGGLVKPGGSLKLSCAASGFTFSDYGMHWVRQAPEKGLEWVAYIFSGSSTIYYADTVKGRFTISRDNAKNTLFLQMTSLRSEDTAMYYCARADGRGAMDYWGQGTSVTVSS

SEQ ID NO: 118 - protein, 107 aa, synthetic construct
  source 1..107: note = 45G10 LH amino acid sequence
DVQMTQTTSSLSASLGDRVTISCRASQDISKFLNWYQQKPDGTVTLLIYYTSRLHSGVPSRFSGSGSGTDYSLTISNLDQEDIATYFCQQGNTLPWTFGGGTKLEIK

SEQ ID NO: 119 - protein, 121 aa, synthetic construct
  source 1..121: note = 48A6 VH amino acid sequence
EVQLVESGGDLMKPGGSLKLSCAASGFTFSSYGMSWVRQTPDKRLEWVATISSGGTYTFYPDSVKGRFTISRDNAKNTLYLQMSSLKSEDTAMYYCARRGWLLHYYAMDYWGQGTSVTVSS

SEQ ID NO: 120 - protein, 111 aa, synthetic construct
  source 1..111: note = 48A6 LH amino acid sequence
NIVLTQSPASLAVSLGQRATISCRPSESVDSFGNSFMHWFQQKPGQPPKLLIYLSSKLESGVPARFSGSGSRTDFTLTIDPVEADDAATYYCQQHNEDPFTFGSGTKLEIK

SEQ ID NO: 121 - protein, 118 aa, synthetic construct
  source 1..118: note = 53F10 VH amino acid sequence
EVQVVESGGGLVKPGGSLKLSCAASGFTFSDYGMHWVRQAPEKGLEWVAYISTGIITVYYADTVKGRFTMSRDNAKNTLFLQMTSLRSEDTAIYYCARADGRGAMDYWGQGTSVIVSS

SEQ ID NO: 122 - protein, 107 aa, synthetic construct
  source 1..107: note = 53F10 LH amino acid sequence
DIQMTQTTSSLSASLGDRVTISCRASQDISNFLNWYQQKPDGTVTLLIYYTSRLHSGVPSRFSGSGSGTDYSLTISNLEQEDFATYFCQQGNTLPWTFGGGTKLEIK

SEQ ID NO: 123 - protein, 121 aa, synthetic construct
  source 1..121: note = Hz5A7.v5 VH amino acid sequence
EVQLVESGGGLVQPGGSLRLSCAASGFTFSSYGMSWVRQAPGKGLEWVATISGGGSYTNYPDSVKGRFTISRDNAKNSLYLQMNSLRAEDTAVYYCARREWRYTLYAMDYWGQGTTVTVSS

SEQ ID NO: 124 - protein, 111 aa, synthetic construct
  source 1..111: note = Hz5A7.v5 LH amino acid sequence
DIQLTQSPSFLSASVGDRVTITCRASESVESYGSSFMHWYQQKPGKAPKLLIYLTSNLESGVPSRFSGSGSGTEFTLTISSLQPEDFATYYCQQNNEDPFTFGQGTKLEIK

SEQ ID NO: 125 - protein, 473 aa, synthetic construct
  source 1..473: note = Hz5A7.v5 VH amino acid sequence with signal sequence
  SIGNAL 1..22
MDMRVPAQLLGLLLLWLRGARCEVQLVESGGGLVQPGGSLRLSCAASGFTFSSYGMSWVRQAPGKGLEWVATISGGGSYTNYPDSVKGRFTISRDNAKNSLYLQMNSLRAEDTAVYYCARREWRYTLYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 126 - protein, 451 aa, synthetic construct
  source 1..451: note = Hz5A7.v5 VH amino acid sequence without signal sequence
EVQLVESGGGLVQPGGSLRLSCAASGFTFSSYGMSWVRQAPGKGLEWVATISGGGSYTNYPDSVKGRFTISRDNAKNSLYLQMNSLRAEDTAVYYCARREWRYTLYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 127 - protein, 240 aa, synthetic construct
  source 1..240: note = Hz5A7.v5 LH amino acid sequence with signal sequence
  SIGNAL 1..22
MDMRVPAQLLGLLLLWLRGARCDIQLTQSPSFLSASVGDRVTITCRASESVESYGSSFMHWYQQKPGKAPKLLIYLTSNLESGVPSRFSGSGSGTEFTLTISSLQPEDFATYYCQQNNEDPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 128 - protein, 218 aa, synthetic construct
  source 1..218: note = Hz5A7.v5 LH amino acid sequence without signal sequence
DIQLTQSPSFLSASVGDRVTITCRASESVESYGSSFMHWYQQKPGKAPKLLIYLTSNLESGVPSRFSGSGSGTEFTLTISSLQPEDFATYYCQQNNEDPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 129 - protein, 330 aa, Homo sapiens
  source 1..330: note = Human IgG1 constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 130 - protein, 330 aa, Homo sapiens
  source 1..330: note = Human IgG1 constant region E233A/L235A
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPALAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 131 - protein, 330 aa, Homo sapiens
  source 1..330: note = Human IgG1 constant region L234A/L235A
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 132 - protein, 330 aa, Homo sapiens
  source 1..330: note = Human IgG1 constant region L234A/L235A/P329G
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 133 - protein, 330 aa, Homo sapiens
  source 1..330: note = Human IgG1 constant region N297G
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 134 - protein, 330 aa, Homo sapiens
  source 1..330: note = Human IgG1 constant region N297G/H310A
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLAQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 135 - protein, 330 aa, Homo sapiens
  source 1..330: note = Human IgG1 constant region L234F/L235E/P331G
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAGIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 136 - protein, 330 aa, Homo sapiens
  source 1..330: note = Human IgG1 constant region L234A/L235A/P331G
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAGIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 137 - protein, 107 aa, Homo sapiens [sequence omitted]

SEQ ID NO: 138 - protein, 106 aa, Homo sapiens
  source 1..106: note = Human Lambda LH constant region
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 139 - protein, 197 aa, Homo sapiens
  source 1..197: note = Human ILT3 Ig-like C2-type D1 and D2 amino acid sequence without N-terminal domain (aa 27-218)
QAGPLPKPTLWAEPGSVISWGNSVTIWCQGTLEAREYRLDKEESPAPWDRQNPLEPKNKARFSIPSMTEDYAGRYRCYYRSPVGWSQPSDPLELVMTGAYSKPTLSALPSPLVTSGKSVTLLCQSRSPMDTFLLIKERAAHPLLHLRSEHGAQQHQAEFPMSPVTSVHGGTYRCFSSHGFSHYLLSHPSDPLELIVS

SEQ ID NO: 140 - protein, 2477 aa, Homo sapiens
  source 1..2477: note = Human Fibronectin with predicted signal sequenc
  SIGNAL 1..31
MLRGPGPGLLLLAVQCLGTAVPSTGASKSKRQAQQMVQPQSPVAVSQSKPGCYDNGKHYQINQQWERTYLGNALVCTCYGGSRGFNCESKPEAEETCFDKYTGNTYRVGDTYERPKDSMIWDCTCIGAGRGRISCTIANRCHEGGQSYKIGDTWRRPHETGGYMLECVCLGNGKGEWTCKPIAEKCFDHAAGTSYVVGETWEKPYQGWMMVDCTCLGEGSGRITCTSRNRCNDQDTRTSYRIGDTWSKKDNRGNLLQCICTGNGRGEWKCERHTSVQTTSSGSGPFTDVRAAVYQPQPHPQPPPYGHCVTDSGVVYSVGMQWLKTQGNKQMLCTCLGNGVSCQETAVTQTYGGNSNGEPCVLPFTYNGRTFYSCTTEGRQDGHLWCSTTSNYEQDQKYSFCTDHTVLVQTRGGNSNGALCHFPFLYNNHNYTDCTSEGRRDNMKWCGTTQNYDADQKFGFCPMAAHEEICTTNEGVMYRIGDQWDKQHDMGHMMRCTCVGNGRGEWTCIAYSQLRDQCIVDDITYNVNDTFHKRHEEGHMLNCTCFGQGRGRWKCDPVDQCQDSETGTFYQIGDSWEKYVHGVRYQCYCYGRGIGEWHCQPLQTYPSSSGPVEVFITETPSQPNSHPIQWNAPQPSHISKYILRWRPKNSVGRWKEATIPGHLNSYTIKGLKPGVVYEGQLISIQQYGHQEVTRFDFTTTSTSTPVTSNTVTGETTPFSPLVATSESVTEITASSFVVSWVSASDTVSGFRVEYELSEEGDEPQYLDLPSTATSVNIPDLLPGRKYIVNVYQISEDGEQSLILSTSQTTAPDAPPDTTVDQVDDTSIVVRWSRPQAPITGYRIVYSPSVEGSSTELNLPETANSVTLSDLQPGVQYNITIYAVEENQESTPVVIQQETTGTPRSDTVPSPRDLQFVEVTDVKVTIMWTPPESAVTGYRVDVIPVNLPGEHGQRLPISRNTFAEVTGLSPGVTYYFKVFAVSHGRESKPLTAQQTTKLDAPTNLQFVNETDSTVLVRWTPPRAQITGYRLTVGLTRRGQPRQYNVGPSVSKYPLRNLQPASEYTVSLVAIKGNQESPKATGVFTTLQPGSSIPPYNTEVTETTIVITWTPAPRIGFKLGVRPSQGGEAPREVTSDSGSIVVSGLTPGVEYVYTIQVLRDGQERDAPIVNKVVTPLSPPTNLHLEANPDTGVLTVSWERSTTPDITGYRITTTPTNGQQGNSLEEVVHADQSSCTFDNLSPGLEYNVSVYTVKDDKESVPISDTIIPEVPQLTDLSFVDITDSSIGLRWTPLNSSTIIGYRITVVAAGEGIPIFEDFVDSSVGYYTVTGLEPGIDYDISVITLINGGESAPTTLTQQTAVPPPTDLRFTNIGPDTMRVTWAPPPSIDLTNFLVRYSPVKNEEDVAELSISPSDNAVVLTNLLPGTEYVVSVSSVYEQHESTPLRGRQKTGLDSPTGIDFSDITANSFTVHWIAPRATITGYRIRHHPEHFSGRPREDRVPHSRNSITLTNLTPGTEYVVSIVALNGREESPLLIGQQSTVSDVPRDLEVVAATPTSLLISWDAPAVTVRYYRITYGETGGNSPVQEFTVPGSKSTATISGLKPGVDYTITVYAVTGRGDSPASSKPISINYRTEIDKPSQMQVTDVQDNSISVKWLPSSSPVTGYRVTTTPKNGPGPTKTKTAGPDQTEMTIEGLQPTVEYVVSVYAQNPSGESQPLVQTAVTNIDRPKGLAFTDVDVDSIKIAWESPQGQVSRYRVTYSSPEDGIHELFPAPDGEEDTAELQGLRPGSEYTVSVVALHDDMESQPLIGTQSTAIPAPTDLKFTQVTPTSLSAQWTPPNVQLTGYRVRVTPKEKTGPMKEINLAPDSSSVVVSGLMVATKYEVSVYALKDTLTSRPAQGVVTTLENVSPPRRARVTDATETTITISWRTKTETITGFQVDAVPANGQTPIQRTIKPDVRSYTITGLQPGTDYKIYLYTLNDNARSSPVVIDASTAIDAPSNLRFLATTPNSLLVSWQPPRARITGYIIKYEKPGSPPREVVPRPRPGVTEATITGLEPGTEYTIYVIALKNNQKSEPLIGRKKTDELPQLVTLPHPNLHGPEILDVPSTVQKTPFVTHPGYDTGNGIQLPGTSGQQPSVGQQMIFEEHGFRRTTPPTTATPIRHRPRPYPPNVGEEIQIGHIPREDVDYHLYPHGPGLNPNASTGQEALSQTTISWAPFQDTSEYIISCHPVGTDEEPLQFRVPGTSTSATLTGLTRGATYNVIVEALKDQQRHKVREEVVTVGNSVNEGLNQPTDDSCFDPYTVSHYAVGDEWERMSESGFKLLCQCLGFGSGHFRCDSSRWCHDNGVNYKIGEKWDRQGENGQMMSCTCLGNGKGEFKCDPHEATCYDDGKTYHVGEQWQKEYLGAICSCTCFGGQRGWRCDNCRRPGGEPSPEGTTGQSYNQYSQRYHQRTNTNVNCPIECFMPLDVQADREDSRE

SEQ ID NO: 141 - protein, 2446 aa, Homo sapiens
  source 1..2446: note = Human Fibronectin without predicted signal sequence
QAQQMVQPQSPVAVSQSKPGCYDNGKHYQINQQWERTYLGNALVCTCYGGSRGFNCESKPEAEETCFDKYTGNTYRVGDTYERPKDSMIWDCTCIGAGRGRISCTIANRCHEGGQSYKIGDTWRRPHETGGYMLECVCLGNGKGEWTCKPIAEKCFDHAAGTSYVVGETWEKPYQGWMMVDCTCLGEGSGRITCTSRNRCNDQDTRTSYRIGDTWSKKDNRGNLLQCICTGNGRGEWKCERHTSVQTTSSGSGPFTDVRAAVYQPQPHPQPPPYGHCVTDSGVVYSVGMQWLKTQGNKQMLCTCLGNGVSCQETAVTQTYGGNSNGEPCVLPFTYNGRTFYSCTTEGRQDGHLWCSTTSNYEQDQKYSFCTDHTVLVQTRGGNSNGALCHFPFLYNNHNYTDCTSEGRRDNMKWCGTTQNYDADQKFGFCPMAAHEEICTTNEGVMYRIGDQWDKQHDMGHMMRCTCVGNGRGEWTCIAYSQLRDQCIVDDITYNVNDTFHKRHEEGHMLNCTCFGQGRGRWKCDPVDQCQDSETGTFYQIGDSWEKYVHGVRYQCYCYGRGIGEWHCQPLQTYPSSSGPVEVFITETPSQPNSHPIQWNAPQPSHISKYILRWRPKNSVGRWKEATIPGHLNSYTIKGLKPGVVYEGQLISIQQYGHQEVTRFDFTTTSTSTPVTSNTVTGETTPFSPLVATSESVTEITASSFVVSWVSASDTVSGFRVEYELSEEGDEPQYLDLPSTATSVNIPDLLPGRKYIVNVYQISEDGEQSLILSTSQTTAPDAPPDTTVDQVDDTSIVVRWSRPQAPITGYRIVYSPSVEGSSTELNLPETANSVTLSDLQPGVQYNITIYAVEENQESTPVVIQQETTGTPRSDTVPSPRDLQFVEVTDVKVTIMWTPPESAVTGYRVDVIPVNLPGEHGQRLPISRNTFAEVTGLSPGVTYYFKVFAVSHGRESKPLTAQQTTKLDAPTNLQFVNETDSTVLVRWTPPRAQITGYRLTVGLTRRGQPRQYNVGPSVSKYPLRNLQPASEYTVSLVAIKGNQESPKATGVFTTLQPGSSIPPYNTEVTETTIVITWTPAPRIGFKLGVRPSQGGEAPREVTSDSGSIVVSGLTPGVEYVYTIQVLRDGQERDAPIVNKVVTPLSPPTNLHLEANPDTGVLTVSWERSTTPDITGYRITTTPTNGQQGNSLEEVVHADQSSCTFDNLSPGLEYNVSVYTVKDDKESVPISDTIIPEVPQLTDLSFVDITDSSIGLRWTPLNSSTIIGYRITVVAAGEGIPIFEDFVDSSVGYYTVTGLEPGIDYDISVITLINGGESAPTTLTQQTAVPPPTDLRFTNIGPDTMRVTWAPPPSIDLTNFLVRYSPVKNEEDVAELSISPSDNAVVLTNLLPGTEYVVSVSSVYEQHESTPLRGRQKTGLDSPTGIDFSDITANSFTVHWIAPRATITGYRIRHHPEHFSGRPREDRVPHSRNSITLTNLTPGTEYVVSIVALNGREESPLLIGQQSTVSDVPRDLEVVAATPTSLLISWDAPAVTVRYYRITYGETGGNSPVQEFTVPGSKSTATISGLKPGVDYTITVYAVTGRGDSPASSKPISINYRTEIDKPSQMQVTDVQDNSISVKWLPSSSPVTGYRVTTTPKNGPGPTKTKTAGPDQTEMTIEGLQPTVEYVVSVYAQNPSGESQPLVQTAVTNIDRPKGLAFTDVDVDSIKIAWESPQGQVSRYRVTYSSPEDGIHELFPAPDGEEDTAELQGLRPGSEYTVSVVALHDDMESQPLIGTQSTAIPAPTDLKFTQVTPTSLSAQWTPPNVQLTGYRVRVTPKEKTGPMKEINLAPDSSSVVVSGLMVATKYEVSVYALKDTLTSRPAQGVVTTLENVSPPRRARVTDATETTITISWRTKTETITGFQVDAVPANGQTPIQRTIKPDVRSYTITGLQPGTDYKIYLYTLNDNARSSPVVIDASTAIDAPSNLRFLATTPNSLLVSWQPPRARITGYIIKYEKPGSPPREVVPRPRPGVTEATITGLEPGTEYTIYVIALKNNQKSEPLIGRKKTDELPQLVTLPHPNLHGPEILDVPSTVQKTPFVTHPGYDTGNGIQLPGTSGQQPSVGQQMIFEEHGFRRTTPPTTATPIRHRPRPYPPNVGEEIQIGHIPREDVDYHLYPHGPGLNPNASTGQEALSQTTISWAPFQDTSEYIISCHPVGTDEEPLQFRVPGTSTSATLTGLTRGATYNVIVEALKDQQRHKVREEVVTVGNSVNEGLNQPTDDSCFDPYTVSHYAVGDEWERMSESGFKLLCQCLGFGSGHFRCDSSRWCHDNGVNYKIGEKWDRQGENGQMMSCTCLGNGKGEFKCDPHEATCYDDGKTYHVGEQWQKEYLGAICSCTCFGGQRGWRCDNCRRPGGEPSPEGTTGQSYNQYSQRYHQRTNTNVNCPIECFMPLDVQADREDSRE

SEQ ID NO: 142 - protein, 577 aa, Homo sapiens
  source 1..577: note = Human Fibronectin fragment containing heparin-binding and collagen-binding domains (˜70 kDa fragment)
QAQQMVQPQSPVAVSQSKPGCYDNGKHYQINQQWERTYLGNALVCTCYGGSRGFNCESKPEAEETCFDKYTGNTYRVGDTYERPKDSMIWDCTCIGAGRGRISCTIANRCHEGGQSYKIGDTWRRPHETGGYMLECVCLGNGKGEWTCKPIAEKCFDHAAGTSYVVGETWEKPYQGWMMVDCTCLGEGSGRITCTSRNRCNDQDTRTSYRIGDTWSKKDNRGNLLQCICTGNGRGEWKCERHTSVQTTSSGSGPFTDVRAAVYQPQPHPQPPPYGHCVTDSGVVYSVGMQWLKTQGNKQMLCTCLGNGVSCQETAVTQTYGGNSNGEPCVLPFTYNGRTFYSCTTEGRQDGHLWCSTTSNYEQDQKYSFCTDHTVLVQTRGGNSNGALCHFPFLYNNHNYTDCTSEGRRDNMKWCGTTQNYDADQKFGFCPMAAHEEICTTNEGVMYRIGDQWDKQHDMGHMMRCTCVGNGRGEWTCIAYSQLRDQCIVDDITYNVNDTFHKRHEEGHMLNCTCFGQGRGRWKCDPVDQCQDSETGTFYQIGDSWEKYVHGVRYQCYCYGRGIGEWHCQPLQTYPSS

SEQ ID NO: 143 - protein, 241 aa, Homo sapiens
  source 1..241: note = Human Fibronectin fragment containing heparin-binding domain (˜30 kDa fragment)
QAQQMVQPQSPVAVSQSKPGCYDNGKHYQINQQWERTYLGNALVCTCYGGSRGFNCESKPEAEETCFDKYTGNTYRVGDTYERPKDSMIWDCTCIGAGRGRISCTIANRCHEGGQSYKIGDTWRRPHETGGYMLECVCLGNGKGEWTCKPIAEKCFDHAAGTSYVVGETWEKPYQGWMMVDCTCLGEGSGRITCTSRNRCNDQDTRTSYRIGDTWSKKDNRGNLLQCICTGNGRGEWKCER

SEQ ID NO: 144 - protein, 336 aa, Homo sapiens
  source 1..336: note = Human Fibronectin fragment containing collagen-binding domain (˜45 kDa fragment)
HTSVQTTSSGSGPFTDVRAAVYQPQPHPQPPPYGHCVTDSGVVYSVGMQWLKTQGNKQMLCTCLGNGVSCQETAVTQTYGGNSNGEPCVLPFTYNGRTFYSCTTEGRQDGHLWCSTTSNYEQDQKYSFCTDHTVLVQTRGGNSNGALCHFPFLYNNHNYTDCTSEGRRDNMKWCGTTQNYDADQKFGFCPMAAHEEICTTNEGVMYRIGDQWDKQHDMGHMMRCTCVGNGRGEWTCIAYSQLRDQCIVDDITYNVNDTFHKRHEEGHMLNCTCFGQGRGRWKCDPVDQCQDSETGTFYQIGDSWEKYVHGVRYQCYCYGRGIGEWHCQPLQTYPSS

SEQ ID NO: 145 - protein, 44 aa, Homo sapiens
  source 1..44: note = Human Fibronectin fragment containing type I repeat 1
PGCYDNGKHYQINQQWERTYLGNALVCTCYGGSRGFNCESKPEA

SEQ ID NO: 146 - protein, 44 aa, Homo sapiens
  source 1..44: note = Human Fibronectin fragment containing type I repeat 2
ETCFDKYTGNTYRVGDTYERPKDSMIWDCTCIGAGRGRISCTIA

SEQ ID NO: 147 - protein, 44 aa, Homo sapiens
  source 1..44: note = Human Fibronectin fragment containing type I repeat 3
NRCHEGGQSYKIGDTWRRPHETGGYMLECVCLGNGKGEWTCKPI

SEQ ID NO: 148 - protein, 46 aa, Homo sapiens
  source 1..46: note = Human Fibronectin fragment containing type I repeat 4
AEKCFDHAAGTSYVVGETWEKPYQGWMMVDCTCLGEGSGRITCTSR

SEQ ID NO: 149 - protein, 45 aa, Homo sapiens
  source 1..45: note = Human Fibronectin fragment containing type I repeat 5
NRCNDQDTRTSYRIGDTWSKKDNRGNLLQCICTGNGRGEWKCERH

SEQ ID NO: 150 - protein, 372 aa, Homo sapiens
  source 1..372: note = Human CNTFR-alpha with predicted signal sequence
  SIGNAL 1..22
MAAPVPWACCAVLAAAAAVVYAQRHSPQEAPHVQYERLGSDVTLPCGTANWDAAVTWRVNGTDLAPDLLNGSQLVLHGLELGHSGLYACFHRDSWHLRHQVLLHVGLPPREPVLSCRSNTYPKGFYCSWHLPTPTYIPNTFNVTVLHGSKIMVCEKDPALKNRCHIRYMHLFSTIKYKVSISVSNALGHNATAITFDEFTIVKPDPPENVVARPVPSNPRRLEVTWQTPSTWPDPESFPLKFFLRYRPLILDQWQHVELSDGTAHTITDAYAGKEYIIQVAAKDNEIGTWSDWSVAAHATPWTEEPRHLTTEAQAAETTTSTTSSLAPPPTTKICDPGELGSGGGPSAPFLVSVPITLALAAAAATASSLLI

SEQ ID NO: 151 - protein, 350 aa, Homo sapiens
  source 1..350: note = Human CNTFR-alpha without predicted signal sequence
QRHSPQEAPHVQYERLGSDVTLPCGTANWDAAVTWRVNGTDLAPDLLNGSQLVLHGLELGHSGLYACFHRDSWHLRHQVLLHVGLPPREPVLSCRSNTYPKGFYCSWHLPTPTYIPNTFNVTVLHGSKIMVCEKDPALKNRCHIRYMHLFSTIKYKVSISVSNALGHNATAITFDEFTIVKPDPPENVVARPVPSNPRRLEVTWQTPSTWPDPESFPLKFFLRYRPLILDQWQHVELSDGTAHTITDAYAGKEYIIQVAAKDNEIGTWSDWSVAAHATPWTEEPRHLTTEAQAAETTTSTTSSLAPPPTTKICDPGELGSGGGPSAPFLVSVPITLALAAAAATASSLLI

SEQ ID NO: 152 - protein, 320 aa, Homo sapiens
  source 1..320: note = Human CNTFR-alpha - mature form
QRHSPQEAPHVQYERLGSDVTLPCGTANWDAAVTWRVNGTDLAPDLLNGSQLVLHGLELGHSGLYACFHRDSWHLRHQVLLHVGLPPREPVLSCRSNTYPKGFYCSWHLPTPTYIPNTFNVTVLHGSKIMVCEKDPALKNRCHIRYMHLFSTIKYKVSISVSNALGHNATAITFDEFTIVKPDPPENVVARPVPSNPRRLEVTWQTPSTWPDPESFPLKFFLRYRPLILDQWQHVELSDGTAHTITDAYAGKEYIIQVAAKDNEIGTWSDWSVAAHATPWTEEPRHLTTEAQAAETTTSTTSSLAPPPTTKICDPGELGS

SEQ ID NO: 153 - protein, 82 aa, Homo sapiens
  source 1..82: note = Human CNTFR-alpha fragment containing Ig-like C2 type domain
QRHSPQEAPHVQYERLGSDVTLPCGTANWDAAVTWRVNGTDLAPDLLNGSQLVLHGLELGHSGLYACFHRDSWHLRHQVLLH

SEQ ID NO: 154 - protein, 118 aa, Homo sapiens
  source 1..118: note = Human CNTFR-alpha fragment containing FN type III domain 1
PPREPVLSCRSNTYPKGFYCSWHLPTPTYIPNTFNVTVLHGSKIMVCEKDPALKNRCHIRYMHLFSTIKYKVSISVSNALGHNATAITFDEFTIVKPDPPENVVARPVPSNPRRLEVT

SEQ ID NO: 155 - protein, 81 aa, Homo sapiens
  source 1..81: note = Human CNTFR-alpha fragment containing FN type III domain 2
WQTPSTWPDPESFPLKFFLRYRPLILDQWQHVELSDGTAHTITDAYAGKEYIIQVAAKDNEIGTWSDWSVAAHATPWTEEP

SEQ ID NO: 156 - protein, 6 aa, synthetic construct
  source 1..6: note = Hexahistidine peptide tag
HHHHHH

SEQ ID NO: 157 - protein, 136 aa, Homo sapiens
  source 1..136: note = Human ILT3 Domain 2 with stem amino acid sequence (aa 124-259)
PTLSALPSPLVTSGKSVTLLCQSRSPMDTFLLIKERAAHPLLHLRSEHGAQQHQAEFPMSPVTSVHGGTYRCFSSHGFSHYLLSHPSDPLELIVSGSLEDPRPSPTRSVSTAAGPEDQPLMPTGSVPHSGLRRHWE

SEQ ID NO: 158 - protein, 121 aa, synthetic construct
  source 1..121: note = Hz48A6 VH amino acid sequence - HC-B0
EVQLVESGGGLVQPGGSLRLSCAASGFTFSSYGMSWVRQAPGKGLEWVATISSGGTYTFYPDSVKGRFTISRDNAKNSLYLQMNSLRAEDTAVYYCARRGWLLHYYAMDYWGQGTSVTVSS

SEQ ID NO: 159 - protein, 121 aa, synthetic construct
  source 1..121: note = Hz48A6 VH amino acid sequence - HC-B1
EVQLVESGGGLMQPGGSLRLSCAASGFTFSSYGMSWVRQAPGKGLEWVATISSGGTYTFYPDSVKGRFTISRDNAKNSLYLQMNSLRAEDTAVYYCARRGWLLHYYAMDYWGQGTSVTVSS

SEQ ID NO: 160 - protein, 121 aa, synthetic construct
  source 1..121: note = Hz48A6 VH amino acid sequence - HC-C0
EVQLVESGGGLVQPGGSLRLSCAASGFTFSSYGMSWVRQAPGKGLEWVGTISSGGTYTFYPDSVKGRFTISRDNSKNTLYLQMNSLRAEDTAVYYCARRGWLLHYYAMDYWGQGTLVTVSS

SEQ ID NO: 161 - protein, 121 aa, synthetic construct
  source 1..121: note = Hz48A6 VH amino acid sequence - HC-C1
EVQLVESGGGLVQPGGSLRLSCAASGFTFSSYGMSWVRQAPGKGLEWVATISSGGTYTFYPDSVKGRFTISRDNSKNTLYLQMNSLRAEDTAVYYCARRGWLLHYYAMDYWGQGTLVTVSS

SEQ ID NO: 162 - protein, 121 aa, synthetic construct
  source 1..121: note = Hz48A6 VH amino acid sequence - HC-C2
EVQLVESGGGLMQPGGSLRLSCAASGFTFSSYGMSWVRQAPGKGLEWVATISSGGTYTFYPDSVKGRFTISRDNSKNTLYLQMNSLRAEDTAVYYCARRGWLLHYYAMDYWGQGTLVTVSS

SEQ ID NO: 163 - protein, 111 aa, synthetic construct
  source 1..111: note = Hz48A6 LH amino acid sequence - LC-C2
DIQLTQSPSFLSASVGDRVTITCRPSESVDSFGNSFMHWFQQKPGKAPKLLIYLSSKLESGVPSRFSGSGSGTEFTLTISSLQPEDFATYYCQQHNEDPFTFGQGTKVEIK

SEQ ID NO: 164 - protein, 118 aa, synthetic construct
  source 1..118: note = Hz45G10 VH amino acid sequence - HC-A2
EVQLVESGGGLVQPGGSLRLSCAASGFTFSDYGMHWVRQAPGKGLEWVAYIFSGSSTIYYADTVKGRFTISRDNAKNSLYLQMNSLRAEDTAVYYCARADGRGAMDYWGQGTTVTVSS

SEQ ID NO: 165 - protein, 118 aa, synthetic construct
  source 1..118: note = Hz45G10 VH amino acid sequence - HC-B1
EVQLVESGGGLVKPGGSLRLSCAASGFTFSDYGMHWVRQAPGKGLEWVSYIFSGSSTIYYADTVKGRFTISRDNAKNSLYLQMNSLRAEDTAVYYCARADGRGAMDYWGQGTLVTVSS

SEQ ID NO: 166 - protein, 107 aa, synthetic construct
  source 1..107: note = Hz45G10 LH amino acid sequence - LC-A4
DIQMTQSPSSLSASVGDRVTITCRASQDISKFLNWYQQKPGKAPKLLIYYTSRLHSGVPSRFSGSGSGTDFTFTISSLQPEDIATYFCQQGNTLPWTFGGGTKLEIK

SEQ ID NO: 167 - protein, 287 aa, Homo sapiens
  source 1..287: note = Human LAIR-1 amino acid sequence with predicted signal sequence
  SIGNAL 1..21
MSPHPTALLGLVLCLAQTIHTQEEDLPRPSISAEPGTVIPLGSHVTFVCRGPVGVQTFRLERESRSTYNDTEDVSQASPSESEARFRIDSVSEGNAGPYRCIYYKPPKWSEQSDYLELLVKETSGGPDSPDTEPGSSAGPTQRPSDNSHNEHAPASQGLKAEHLYILIGVSVVFLFCLLLLVLFCLHRQNQIKQGPPRSKDEEQKPQQRPDLAVDVLERTADKATVNGLPEKDRETDTSALAAGSSQEVTYAQLDHWALTQRTARAVSPQSTKPMAESITYAAVARH

SEQ ID NO: 168 - protein, 266 aa, Homo sapiens
  source 1..266: note = Human LAIR-1 amino acid sequence without predicted signal sequence
QEEDLPRPSISAEPGTVIPLGSHVTFVCRGPVGVQTFRLERESRSTYNDTEDVSQASPSESEARFRIDSVSEGNAGPYRCIYYKPPKWSEQSDYLELLVKETSGGPDSPDTEPGSSAGPTQRPSDNSHNEHAPASQGLKAEHLYILIGVSVVFLFCLLLLVLFCLHRQNQIKQGPPRSKDEEQKPQQRPDLAVDVLERTADKATVNGLPEKDRETDTSALAAGSSQEVTYAQLDHWALTQRTARAVSPQSTKPMAESITYAAVARH

SEQ ID NO: 169 - protein, 144 aa, Homo sapiens
  source 1..144: note = Human LAIR-1 extracellular domain (aa 22-165)
QEEDLPRPSISAEPGTVIPLGSHVTFVCRGPVGVQTFRLERESRSTYNDTEDVSQASPSESEARFRIDSVSEGNAGPYRCIYYKPPKWSEQSDYLELLVKETSGGPDSPDTEPGSSAGPTQRPSDNSHNEHAPASQGLKAEHLY

SEQ ID NO: 170 - protein, 89 aa, Homo sapiens
  source 1..89: note = Human LAIR-1 Ig-like C2-type domain amino acid sequence (aa 29-117)
PSISAEPGTVIPLGSHVTFVCRGPVGVQTFRLERESRSTYNDTEDVSQASPSESEARFRIDSVSEGNAGPYRCIYYKPPKWSEQSDYLE

SEQ ID NO: 171 - protein, 287 aa, Macaca fascicularis
  source 1..287: note = Cyno LAIR-1 amino acid sequence with predicted signal sequence
  SIGNAL 1..21
MSPHPTALLGLVLCLAQTIHAQEGPLPRPSISAEPGTVIPPGRPVTIVCRGPVGVDQFRLEREDRSKFNDTKDVSQASPSESEARFRIDSVSEGNAGHYRCLYVKSTRWSEHSDYLDLVVKETSGDTDSPVTEPDSSAGPTQRPSDNSHNEHAPASQGLSAEHLYILIGVSVVFLFCLLLLVLFFLHRQNQMKQGPPRSKDEEQKLQQRPDLAVDVLERTADKATVNGLPEKDRETDTSAPAAGSSQEVTYAQLDHWALTWRTAQAVSPQSTEPMAESSTYAAVARH

SEQ ID NO: 172 - protein, 266 aa, Macaca fascicularis
  source 1..266: note = Cyno LAIR-1 amino acid sequence without predicted signal sequence
QEGPLPRPSISAEPGTVIPPGRPVTIVCRGPVGVDQFRLEREDRSKFNDTKDVSQASPSESEARFRIDSVSEGNAGHYRCLYVKSTRWSEHSDYLDLVVKETSGDTDSPVTEPDSSAGPTQRPSDNSHNEHAPASQGLSAEHLYILIGVSVVFLFCLLLLVLFFLHRQNQMKQGPPRSKDEEQKLQQRPDLAVDVLERTADKATVNGLPEKDRETDTSAPAAGSSQEVTYAQLDHWALTWRTAQAVSPQSTEPMAESSTYAAVARH

SEQ ID NO: 173 - protein, 142 aa, Macaca fascicularis
  source 1..142: note = Cyno LAIR-1 extracellular domain (aa 22-165)
QEGPLPRPSISAEPGTVIPPGRPVTIVCRGPVGVDQFRLEREDRSKFNDTKDVSQASPSESEARFRIDSVSEGNAGHYRCLYVKSTRWSEHSDYLDLVVKETSGDTDSPVTEPDSSAGPTQRPSDNSHNEHAPASQGLSAEH

SEQ ID NO: 174 - protein, 88 aa, Macaca fascicularis
  source 1..88: note = Cyno LAIR-1 Ig-like C2-type domain amino acid sequence (aa 29-117)
EPGTVIPPGRPVTIVCRGPVGVDQFRLEREDRSKFNDTKDVSQASPSESEARFRIDSVSEGNAGHYRCLYVKSTRWSEHSDYLDLVVK

SEQ ID NO: 175 - protein, 121 aa, synthetic construct
  source 1..121: note = 47A1 VH amino acid sequence
EVQLVESGGGLVQPKGSLKLSCAASGFTFNTYAIHWVRQAPGKGLEWVARIRSKSTNYATYYADSVKDRFTISRDDSQSMVFLQMNNLKTEDTAMYYCVRENWYYYALDYWGQGTSVTVSS

SEQ ID NO: 176 - protein, 107 aa, synthetic construct
  source 1..107: note = 47A1 LH amino acid sequence
DIQMTQSPASLSASVGETVTITCRASGNIHNYLTWYQQKQGKSPQVLVYNAKTLEDGVPSRFSGSESGTQYSLKINSLQPEDFGSYYCQHFWSTPFTFGSGTKLEIK

SEQ ID NO: 177 - protein, 119 aa, synthetic construct
  source 1..119: note = 47H1 VH amino acid sequence
EVQLVETGGGLVQPKGSLKLSCAASGFTFNINAMNWVRQAPGKGLEWVARIRTKNNNYATFYADSVKDRFTISRDDSQSMLYLQMNNLKTDDTAMYYCVRDRAGFFAYWGQGTPVTVSA

SEQ ID NO: 178 - protein, 107 aa, synthetic construct
  source 1..107: note = 47H1 LH amino acid sequence
DIQMTQSPASQSASLGESVTITCLASQTIGTWLGWYRQKPGKSPQLLIYAATSLADGVPSRFSGSGSGTKFSFKISSLQAEDFVIYYCQQLYSTPLTFGSGTKLEIK

SEQ ID NO: 179 - protein, 119 aa, synthetic construct
  source 1..119: note = Hz47H1.v4 VH amino acid sequence
EVQLVESGGGLVQPGGSLRLSCAASGFTFNINAMNWVRQAPGKGLEWVARIRTKNYNYATFYADSVKDRFTISRDDSKNSLYLQMNSLKTEDTAVYYCVRDRAGFFAYWGQGTTVTVSS

SEQ ID NO: 180 - protein, 107 aa, synthetic construct
  source 1..107: note = Hz47H1.v4 LH amino acid sequence
DIQMTQSPSSLSASVGDRVTITCLASQTIGTWLGWYQQKPGKAPKLLIYAATSLAEGVPSRFSGSGSGTDFTLTISSLQPEDFATYYCQQLYSTPLTFGGGTKVEIK

SEQ ID NO: 181 - protein, 118 aa, synthetic construct
  source 1..118: note = 57D12 VH amino acid sequence
QVQLQQSGAELARPGASVNLSCRASGYSFTSFGISWVKQRTGQGLEWIGEIYPRSDNTFYNEKFKGKATLTADKSSSTAYMELRSLTSEDSAVYFCARHFGSSSFDYWGQGTTLTVSS

SEQ ID NO: 182 - protein, 108 aa, synthetic construct
  source 1..108: note = 57D12 LH amino acid sequence
ENVLTQSPPIMAASLGQKVTMTCSASSSVSSIYFHWYQQKSGTSPKPLIHRASNLASGVPARFSGSGSGTSYSLTISSVEAEDDATYYCQQWSGYPLTFGGGTKLEIK

SEQ ID NO: 183 - protein, 122 aa, synthetic construct
  source 1..122: note = 61H4 VH amino acid sequence
EVQLQQSGPEVLKPGASVKISCKASGYTFTDYYYMNWVKQSHGKSLEWIGYIYPNNGATSYNQKFKGKATLTVDKSSSTAYMELRSLTSEDSAVYYCARDGYSSNYYTMDYWGQGTSVTVSS

SEQ ID NO: 184 - protein, 107 aa, synthetic construct
  source 1..107: note = 61H4 LH amino acid sequence
DVQMIQSPSSLSASLGDIVTMTCQASQGTSINLNWFQQKPGKAPKLLIYGASNLEDGVPSRFSGSRYGTDFTLTISSLEDEDMATYFCLQHTYLPYTFGGGTKLEIK

SEQ ID NO: 185 - protein, 117 aa, synthetic construct
  source 1..117: note = 62G10 VH amino acid sequence
EVQLVETGGGLVQPKGSLKLSCATSGFTFNINAMNWVRQAPGKGLEWVARIRTKNNNFATYYADSVKDRFTISRDDSQSMLYLQMNNLKTEDTAMYYCVRGPYFDYWGQGTTLTVSS

SEQ ID NO: 186 - protein, 107 aa, synthetic construct
  source 1..107: note = 62G10 LH amino acid sequence
DIQMTQSPASQSASLGESVTITCLASQTIGTWLAWYQQKPGKSPQLLIYAATSLADGVPSRFSGSGSGTKFSFKISNLQAEDFVTYYCQQLYSTPYTFGGGTKLEIK

SEQ ID NO: 187 - protein, 117 aa, synthetic construct
  source 1..117: note = Hz62G10.v1 VH amino acid sequence
EVQLVESGGGLVKPGGSLRLSCAASGFTFNINAMNWVRQAPGKGLEWVARIRTKNNNFATYYADSVKDRFTISRDDSKNTLYLQMNSLKTEDTAVYYCVRGPYFDYWGQGTLVTVSS

SEQ ID NO: 188 - protein, 107 aa, synthetic construct
  source 1..107: note = Hz62G10.v1 LH amino acid sequence
DIQMTQSPSSLSASVGDRVTITCLASQTIGTWLAWYQQKPGKAPKLLIYAATSLADGVPSRFSGSGSGTDFTLTISSLQPEDFATYYCQQLYSTPYTFGGGTKVEIK

SEQ ID NO: 189 - protein, 120 aa, synthetic construct
  source 1..120: note = 108D10 VH amino acid sequence
EVQLVETGGGLVQPKGSLKLSCAASGFTFNINAMNWVRQAPGKGLEWVARIRTKNNNYATYYADSVKDRFTISRDDSESMLYLQMNNLKTEDTAMYYCVRDRYGGAMAYWGQGTSVTVSS

SEQ ID NO: 190 - protein, 107 aa, synthetic construct
  source 1..107: note = 108D10 LH amino acid sequence
DIQMTQSSSSFSVSLGDRVTITCKASEDIYNRLAWYQQKPGNVPRLLISSATSLETGVPSRFSGSGSGKDYTLSLTSLQSEDVATYYCQQYWTIPYTFGGGTKLEIK

SEQ ID NO: 191 - protein, 118 aa, synthetic construct
  source 1..118: note = 43H2 VH amino acid sequence
EVQLVESGGGLVQPGRSLKVSCAASGFTFSNYGIHWIRQAPTKGLEWVASISPSGRSTYFRDSVKGRFTISRDNAKNTLYLQLDSLRSEDTATYYCATGINYSSFDYWGQGVMVTVSS

SEQ ID NO: 192 - protein, 107 aa, synthetic construct
  source 1..107: note = 43H2 LH amino acid sequence
DIVMTQSPTSMSISVGDRVTMNCKASQNVGSHVDWYQQKTGQSPKLLISTASNRYTGVPDRFTGSGSGTDFTFTINNMQTEDLAVYYCMQSNSYPPTFGGGTKLELK

SEQ ID NO: 193 - protein, 471 aa, synthetic construct
  source 1..471: note = Hz47H1.v4 VH amino acid sequence with signal sequence
  SIGNAL 1..22
MDMRVPAQLLGLLLLWLRGARCEVQLVESGGGLVQPGGSLRLSCAASGFTFNINAMNWVRQAPGKGLEWVARIRTKNYNYATFYADSVKDRFTISRDDSKNSLYLQMNSLKTEDTAVYYCVRDRAGFFAYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 194 - protein, 449 aa, synthetic construct
  source 1..449: note = Hz47H1.v4 VH amino acid sequence without signal sequence
EVQLVESGGGLVQPGGSLRLSCAASGFTFNINAMNWVRQAPGKGLEWVARIRTKNYNYATFYADSVKDRFTISRDDSKNSLYLQMNSLKTEDTAVYYCVRDRAGFFAYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 195 - protein, 236 aa, synthetic construct
  source 1..236: note = Hz47H1.v4 LH amino acid sequence with signal sequence
  SIGNAL 1..22
MDMRVPAQLLGLLLLWLRGARCDIQMTQSPSSLSASVGDRVTITCLASQTIGTWLGWYQQKPGKAPKLLIYAATSLAEGVPSRFSGSGSGTDFTLTISSLQPEDFATYYCQQLYSTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 196 - protein, 214 aa, synthetic construct
  source 1..214: note = Hz47H1.v4 LH amino acid sequence without signal sequence
DIQMTQSPSSLSASVGDRVTITCLASQTIGTWLGWYQQKPGKAPKLLIYAATSLAEGVPSRFSGSGSGTDFTLTISSLQPEDFATYYCQQLYSTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 197 - protein, 469 aa, synthetic construct
  source 1..469: note = Hz62G10.v1 VH amino acid sequence with signal sequence underlined
  SIGNAL 1..22
MDMRVPAQLLGLLLLWLRGARCEVQLVESGGGLVKPGGSLRLSCAASGFTFNINAMNWVRQAPGKGLEWVARIRTKNNNFATYYADSVKDRFTISRDDSKNTLYLQMNSLKTEDTAVYYCVRGPYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 198 - protein, 447 aa, synthetic construct
  source 1..447: note = Hz62G10.v1 VH amino acid sequence without signal sequence
EVQLVESGGGLVKPGGSLRLSCAASGFTFNINAMNWVRQAPGKGLEWVARIRTKNNNFATYYADSVKDRFTISRDDSKNTLYLQMNSLKTEDTAVYYCVRGPYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 199 - protein, 236 aa, synthetic construct
  source 1..236: note = Hz62G10.v1 LH amino acid sequence with signal sequence
  SIGNAL 1..22
MDMRVPAQLLGLLLLWLRGARCDIQMTQSPSSLSASVGDRVTITCLASQTIGTWLAWYQQKPGKAPKLLIYAATSLADGVPSRFSGSGSGTDFTLTISSLQPEDFATYYCQQLYSTPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 200 - protein, 214 aa, synthetic construct
  source 1..214: note = Hz62G10.v1 LH amino acid sequence without signal sequence
DIQMTQSPSSLSASVGDRVTITCLASQTIGTWLAWYQQKPGKAPKLLIYAATSLADGVPSRFSGSGSGTDFTLTISSLQPEDFATYYCQQLYSTPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 201 - protein, 263 aa, Mus musculus
  source 1..263: note = Mouse LAIR-1 amino acid sequence with predicted signal sequence
  SIGNAL 1..21
MSLHPVILLVLVLCLGWKINTQEGSLPDITIFPNSSLMISQGTFVTVVCSYSDKHDLYNMVRLEKDGSTFMEKSTEPYKTEDEFEIGPVNETITGHYSCIYSKGITWSERSKTLELKVIKENVIQTPAPGPTSDTSWLKTYSIYIFTVVSVIFLLCLSALLFCFLRHRQKKQGLPNNKRQQQRPEERLNLATNGLEMTPDIVADDRLPEDRWTETWTPVAGDLQEVTYIQLDHHSLTQRAVGAVTSQSTDMAESSTYAAIIRH

SEQ ID NO: 202 - protein, 242 aa, Mus musculus
  source 1..242: note = Mouse LAIR-1 amino acid sequence without predicted signal sequence
QEGSLPDITIFPNSSLMISQGTFVTVVCSYSDKHDLYNMVRLEKDGSTFMEKSTEPYKTEDEFEIGPVNETITGHYSCIYSKGITWSERSKTLELKVIKENVIQTPAPGPTSDTSWLKTYSIYIFTVVSVIFLLCLSALLFCFLRHRQKKQGLPNNKRQQQRPEERLNLATNGLEMTPDIVADDRLPEDRWTETWTPVAGDLQEVTYIQLDHHSLTQRAVGAVTSQSTDMAESSTYAAIIRH

SEQ ID NO: 203 - protein, 123 aa, Mus musculus
  source 1..123: note = Mouse LAIR-1 extracellular domain (aa 22-144)
QEGSLPDITIFPNSSLMISQGTFVTVVCSYSDKHDLYNMVRLEKDGSTFMEKSTEPYKTEDEFEIGPVNETITGHYSCIYSKGITWSERSKTLELKVIKENVIQTPAPGPTSDTSWLKTYSIY

SEQ ID NO: 204 - protein, 88 aa, Mus musculus
  source 1..88: note = Mouse LAIR-1 Ig-like C2-type domain amino acid sequence (aa 27-114)
PDITIFPNSSLMISQGTFVTVVCSYSDKHDLYNMVRLEKDGSTFMEKSTEPYKTEDEFEIGPVNETITGHYSCIYSKGITWSERSKTL

SEQ ID NO: 205 - protein, 117 aa, synthetic construct
  source 1..117: note = 6D8 VH amino acid sequence
QVQLKESGPGLVAPSQSLSIACTVSGFSLTNYAISWVRQPPGKGLEWLGVIWTGGGTNYNSTLKSRLSISKDNSKSQVFLKMNSLQTDDTARYNCARNSGDWYFDVWGPGTTVTVSS

SEQ ID NO: 206 - protein, 113 aa, synthetic construct
  source 1..113: note = 6D8 LH amino acid sequence
DIVMSQSPSSLAVSVGEKVTMSCKSSQSLLYSSNQKNYLAWYQQKPGQSPHLLIYWASTRESGVPDRFIGSGSGTDFTLTISSVKAEDLAVYYCQQYYDYPPTFGSGTKLEIK

SEQ ID NO: 207 - protein, 119 aa, synthetic construct
  source 1..119: note = 10G4 VH amino acid sequence
QVQLKESGPGLVAPSQSLSITCTVSGFSLSSYPISWVRQPPGKGLEWLGIIWTGGGTNYNSALKSRLSISKDNSKSQVFLKMNSLQTDDTARYYCASQNWDVNSALDYWGQGTSVTVSS

SEQ ID NO: 208 - protein, 107 aa, synthetic construct
  source 1..107: note = 10G4 LH amino acid sequence
DIQMTQSPASLSVSVGETVTITCRASENIYSNLAWYQQKQGKSPQLLVYTATNLADGVPSRFSGSGSGTQYSLRINRLQSEDFGNYYCQHFWNAPWTFGGGTKLEIK

SEQ ID NO: 209 - protein, 125 aa, synthetic construct
  source 1..125: note = 15A3 VH amino acid sequence
EIQLQQSGPELVKPGASVKVSCKASGYSFTDYNMYWVKQSHGKSLEWIGYIDPYNGGTSYNQKFKGKATLTVDKSSSTAFMHLNSLTSEDSAVYYCARLRSPYDYDRGDYVMDYWGQGTSVTVSS

SEQ ID NO: 210 - protein, 111 aa, synthetic construct
  source 1..111: note = 15A3 LH amino acid sequence
DIVLTQSPASLAVSLGQRATISCRASKSVSTSGYTYMHWYQQKPGQPPKLLIYLASNLESGVPARFSGSGSGTDFTLNIHPVEEEDAATYYCQHSRELPLTFGAGTKLELK

SEQ ID NO: 211 - protein, 469 aa, synthetic construct
  source 1..469: note = 6D8 VH amino acid sequence with signal sequence
  SIGNAL 1..22
MDMRVPAQLLGLLLLWLRGARCQVQLKESGPGLVAPSQSLSIACTVSGFSLTNYAISWVRQPPGKGLEWLGVIWTGGGTNYNSTLKSRLSISKDNSKSQVFLKMNSLQTDDTARYNCARNSGDWYFDVWGPGTTVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 212 - protein, 447 aa, synthetic construct
  source 1..447: note = 6D8 VH amino acid sequence without signal sequence
QVQLKESGPGLVAPSQSLSIACTVSGFSLTNYAISWVRQPPGKGLEWLGVIWTGGGTNYNSTLKSRLSISKDNSKSQVFLKMNSLQTDDTARYNCARNSGDWYFDVWGPGTTVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 213 - protein, 242 aa, synthetic construct
  source 1..242: note = 6D8 LH amino acid sequence with signal sequence
  SIGNAL 1..22
MDMRVPAQLLGLLLLWLRGARCDIVMSQSPSSLAVSVGEKVTMSCKSSQSLLYSSNQKNYLAWYQQKPGQSPHLLIYWASTRESGVPDRFIGSGSGTDFTLTISSVKAEDLAVYYCQQYYDYPPTFGSGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 214 - protein, 220 aa, synthetic construct
  source 1..220: note = 6D8 LH amino acid sequence without signal sequence
DIVMSQSPSSLAVSVGEKVTMSCKSSQSLLYSSNQKNYLAWYQQKPGQSPHLLIYWASTRESGVPDRFIGSGSGTDFTLTISSVKAEDLAVYYCQQYYDYPPTFGSGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 215 - protein, 471 aa, synthetic construct
  source 1..471: note = 10G4 VH amino acid sequence with signal sequence
  SIGNAL 1..22
MDMRVPAQLLGLLLLWLRGARCQVQLKESGPGLVAPSQSLSITCTVSGFSLSSYPISWVRQPPGKGLEWLGIIWTGGGTNYNSALKSRLSISKDNSKSQVFLKMNSLQTDDTARYYCASQNWDVNSALDYWGQGTSVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 216 - protein, 449 aa, synthetic construct
  source 1..449: note = 10G4 VH amino acid sequence without signal sequence
QVQLKESGPGLVAPSQSLSITCTVSGFSLSSYPISWVRQPPGKGLEWLGIIWTGGGTNYNSALKSRLSISKDNSKSQVFLKMNSLQTDDTARYYCASQNWDVNSALDYWGQGTSVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 217 - protein, 236 aa, synthetic construct
  source 1..236: note = 10G4 LH amino acid sequence with signal sequence
  SIGNAL 1..22
MDMRVPAQLLGLLLLWLRGARCDIQMTQSPASLSVSVGETVTITCRASENIYSNLAWYQQKQGKSPQLLVYTATNLADGVPSRFSGSGSGTQYSLRINRLQSEDFGNYYCQHFWNAPWTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 218 - protein, 214 aa, synthetic construct
  source 1..214: note = 10G4 LH amino acid sequence without signal sequence
DIQMTQSPASLSVSVGETVTITCRASENIYSNLAWYQQKQGKSPQLLVYTATNLADGVPSRFSGSGSGTQYSLRINRLQSEDFGNYYCQHFWNAPWTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 219 - protein, 477 aa, synthetic construct
  source 1..477: note = 15A3 VH amino acid sequence with signal sequence
  SIGNAL 1..22
MDMRVPAQLLGLLLLWLRGARCEIQLQQSGPELVKPGASVKVSCKASGYSFTDYNMYWVKQSHGKSLEWIGYIDPYNGGTSYNQKFKGKATLTVDKSSSTAFMHLNSLTSEDSAVYYCARLRSPYDYDRGDYVMDYWGQGTSVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 220 - protein, 455 aa, synthetic construct
  source 1..455: note = 15A3 VH amino acid sequence without signal sequence
EIQLQQSGPELVKPGASVKVSCKASGYSFTDYNMYWVKQSHGKSLEWIGYIDPYNGGTSYNQKFKGKATLTVDKSSSTAFMHLNSLTSEDSAVYYCARLRSPYDYDRGDYVMDYWGQGTSVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 221 - protein, 240 aa, synthetic construct
  source 1..240: note = 15A3 LH amino acid sequence with signal sequence
  SIGNAL 1..22
MDMRVPAQLLGLLLLWLRGARCDIVLTQSPASLAVSLGQRATISCRASKSVSTSGYTYMHWYQQKPGQPPKLLIYLASNLESGVPARFSGSGSGTDFTLNIHPVEEEDAATYYCQHSRELPLTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 222 - protein, 218 aa, synthetic construct
  source 1..218: note = 15A3 LH amino acid sequence without signal sequence
DIVLTQSPASLAVSLGQRATISCRASKSVSTSGYTYMHWYQQKPGQPPKLLIYLASNLESGVPARFSGSGSGTDFTLNIHPVEEEDAATYYCQHSRELPLTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 223 - protein, 152 aa, Homo sapiens
  source 1..152: note = Human LAIR-2 amino acid sequence with predicted signal sequence
  SIGNAL 1..21
MSPHLTALLGLVLCLAQTIHTQEGALPRPSISAEPGTVISPGSHVTFMCRGPVGVQTFRLEREDRAKYKDSYNVFRLGPSESEARFHIDSVSEGNAGLYRCLYYKPPGWSEHSDFLELLVKESSGGPDSPDTEPGSSAGTVPGTEASGFDAP

SEQ ID NO: 224 - protein, 131 aa, Homo sapiens
  source 1..131: note = Human LAIR-2 amino acid sequence without predicted signal sequence
QEGALPRPSISAEPGTVISPGSHVTFMCRGPVGVQTFRLEREDRAKYKDSYNVFRLGPSESEARFHIDSVSEGNAGLYRCLYYKPPGWSEHSDFLELLVKESSGGPDSPDTEPGSSAGTVPGTEASGFDAP

SEQ ID NO: 225 - protein, 89 aa, Homo sapiens
  source 1..89: note = Human LAIR-2 amino acid sequence Ig-like C2 type domain (aa 29-177)
PSISAEPGTVISPGSHVTFMCRGPVGVQTFRLEREDRAKYKDSYNVFRLGPSESEARFHIDSVSEGNAGLYRCLYYKPPGWSEHSDFLE

SEQ ID NO: 226 - protein, 10 aa, synthetic construct
  source 1..10: note = Antibody 47A1 VH CDR1 - Exemplary, AbM
GFTFNTYAIH

SEQ ID NO: 227 - protein, 19 aa, synthetic construct
  source 1..19: note = Antibody 47A1 VH CDR2 - Exemplary, Kabat
RIRSKSTNYATYYADSVKD

SEQ ID NO: 228 - protein, 10 aa, synthetic construct
  source 1..10: note = Antibody 47A1 VH CDR3 - Exemplary, Chothia, AbM, Kabat
ENWYYYALDY

SEQ ID NO: 229 - protein, 11 aa, synthetic construct
  source 1..11: note = Antibody 47A1 VL CDR1 - Exemplary, Chothia, AbM, Kabat
RASGNIHNYLT

SEQ ID NO: 230 - protein, 7 aa, synthetic construct
  source 1..7: note = Antibody 47A1 VL CDR2 - Exemplary, Chothia, AbM, Kabat
NAKTLED

SEQ ID NO: 231 - protein, 9 aa, synthetic construct
  source 1..9: note = Antibody 47A1 VL CDR3 - Exemplary, Chothia, AbM, Kabat
QHFWSTPFT

SEQ ID NO: 232 - protein, 7 aa, synthetic construct
  source 1..7: note = Antibody 47A1 VH CDR1 - Chothia
GFTFNTY

SEQ ID NO: 233 - protein, 8 aa, synthetic construct
  source 1..8: note = Antibody 47A1 VH CDR2 - Chothia
RSKSTNYA

SEQ ID NO: 234 - protein, 12 aa, synthetic construct
  source 1..12: note = Antibody 47A1 VH CDR2 - AbM
RIRSKSTNYATY

SEQ ID NO: 235 - protein, 5 aa, synthetic construct
  source 1..5: note = Antibody 47A1 VH CDR1 - Kabat
TYAIH

SEQ ID NO: 236 - protein, 6 aa, synthetic construct
  source 1..6: note = Antibody 47A1 VH CDR1 - Contact
NTYAIH

SEQ ID NO: 237 - protein, 15 aa, synthetic construct
  source 1..15: note = Antibody 47A1 VH CDR2 - Contact
WVARIRSKSTNYATY

SEQ ID NO: 238 - protein, 11 aa, synthetic construct
  source 1..11: note = Antibody 47A1 VH CDR3 - Contact
VRENWYYYALD

SEQ ID NO: 239 - protein, 7 aa, synthetic construct
  source 1..7: note = Antibody 47A1 VL CDR1 - Contact
HNYLTWY

SEQ ID NO: 240 - protein, 10 aa, synthetic construct
  source 1..10: note = Antibody 47A1 VL CDR2 - Contact
VLVYNAKTLE

SEQ ID NO: 241 - protein, 8 aa, synthetic construct
  source 1..8: note = Antibody 47A1 VL CDR3 - Contact
QHFWSTPF

SEQ ID NO: 242 - protein, 10 aa, synthetic construct
  source 1..10: note = Antibody 47H1, Hz47H1.v4, 62G10, Hz62G10.v1, 108D10 VH CDR1 - Exemplary, AbM
GFTFNINAMN

SEQ ID NO: 243 - protein, 19 aa, synthetic construct
  source 1..19: note = Antibody 47H1 VH CDR2 - Exemplary, Kabat
RIRTKNNNYATFYADSVKD

SEQ ID NO: 244 - protein, 8 aa, synthetic construct
  source 1..8: note = Antibody 47H1, Hz47H1.v4 VH CDR3 - Exemplary, Chothia, AbM, Kabat
DRAGFFAY

SEQ ID NO: 245 - protein, 11 aa, synthetic construct
  source 1..11: note = Antibody 47H1, Hz47H1.v4 VL CDR1 - Exemplary, Chothia, AbM, Kabat
LASQTIGTWLG

SEQ ID NO: 246 - protein, 7 aa, synthetic construct
  source 1..7: note = Antibody 47H1, 62G10 and Hz62G10.v1 VL CDR2 - Exemplary, Chothia, AbM, Kabat
AATSLAD

SEQ ID NO: 247 - protein, 9 aa, synthetic construct
  source 1..9: note = Antibody 47H1, Hz47H1.v4 VL CDR3 - Exemplary, Chothia, AbM, Kabat
QQLYSTPLT

SEQ ID NO: 248 - protein, 7 aa, synthetic construct
  source 1..7: note = Antibody 47H1, Hz47H1.v4, 62G10 and Hz62G10.v1, 108D10 VH CDR1 - Chothia
GFTFNIN

SEQ ID NO: 249 - protein, 12 aa, synthetic construct
  source 1..12: note = Antibody 47H1 VH CDR2 - AbM
RIRTKNNNYATF

SEQ ID NO: 250 - protein, 5 aa, synthetic construct
  source 1..5: note = Antibody 47H1, HzAntibody 47H1.v4, 62G10 and Hz62G10.v1, 108D10 VH CDR1 - Kabat
INAMN

SEQ ID NO: 251 - protein, 6 aa, synthetic construct
  source 1..6: note = Antibody 47H1, HzAntibody 47H1.v4, 62G10 and Hz62G10.v1, 108D10 VH CDR1 - Contact
NINAMN

SEQ ID NO: 252 - protein, 15 aa, synthetic construct
  source 1..15: note = Antibody 47H1 VH CDR2 - Contact
WVARIRTKNNNYATF

SEQ ID NO: 253 - protein, 9 aa, synthetic construct
  source 1..9: note = Antibody 47H1, Hz47H1.v4 VH CDR3 - Contact
VRDRAGFFA

SEQ ID NO: 254 - protein, 7 aa, synthetic construct
  source 1..7: note = Antibody 47H1, Hz47H1.v4 VL CDR1 - Contact
GTWLGWY

SEQ ID NO: 255 - protein, 10 aa, synthetic construct
  source 1..10: note = Antibody 47H1, Hz47H1.v4, 62G10 and Hz62G10.v1 VL CDR2 - Contact
LLIYAATSLA

SEQ ID NO: 256 - protein, 8 aa, synthetic construct
  source 1..8: note = Antibody 47H1, Hz47H1.v4 VL CDR3 - Contact
QQLYSTPL

SEQ ID NO: 257 - protein, 19 aa, synthetic construct
  source 1..19: note = Antibody Hz47H1.v4 VH CDR2 - Exemplary, Kabat
RIRTKNYNYATFYADSVKD

SEQ ID NO: 258 - protein, 7 aa, synthetic construct
  source 1..7: note = Antibody Hz47H1.v4 VL CDR2 - Exemplary, Chothia, AbM, Kabat
AATSLAE

SEQ ID NO: 259 - protein, 8 aa, synthetic construct
  source 1..8: note = Antibody Hz47H1.v4 VH CDR2 - Chothia
RTKNYNYA

SEQ ID NO: 260 - protein, 12 aa, synthetic construct
  source 1..12: note = Antibody Hz47H1.v4 VH CDR2 - AbM
RIRTKNYNYATF

SEQ ID NO: 261 - protein, 15 aa, synthetic construct
  source 1..15: note = Antibody Hz47H1.v4 VH CDR2 - Contact
WVARIRTKNYNYATF

SEQ ID NO: 262 - protein, 10 aa, synthetic construct
  source 1..10: note = Antibody 57D12 VH CDR1 - Exemplary, AbM
GYSFTSFGIS

SEQ ID NO: 263 - protein, 17 aa, synthetic construct
  source 1..17: note = Antibody 57D12 VH CDR2 - Exemplary, Kabat
EIYPRSDNTFYNEKFKG

SEQ ID NO: 264 - protein, 9 aa, synthetic construct
  source 1..9: note = Antibody 57D12 VH CDR3 - Exemplary, Chothia, AbM, Kabat
HFGSSSFDY

SEQ ID NO: 265 - protein, 12 aa, synthetic construct
  source 1..12: note = Antibody 57D12 VL CDR1 - Exemplary, Chothia, AbM, Kabat
SASSSVSSIYFH

SEQ ID NO: 266 - protein, 7 aa, synthetic construct
  source 1..7: note = Antibody 57D12 VL CDR2 - Exemplary, Chothia, AbM, Kabat
RASNLAS

SEQ ID NO: 267 - protein, 9 aa, synthetic construct
  source 1..9: note = Antibody 57D12 VL CDR3 - Exemplary, Chothia, AbM, Kabat
QQWSGYPLT

SEQ ID NO: 268 - protein, 7 aa, synthetic construct
  source 1..7: note = Antibody 57D12 VH CDR1 - Chothia
GYSFTSF

SEQ ID NO: 269 - protein, 6 aa, synthetic construct
  source 1..6: note = Antibody 57D12 VH CDR2 - Chothia
YPRSDN

SEQ ID NO: 270 - protein, 10 aa, synthetic construct
  source 1..10: note = Antibody 57D12 VH CDR2 - AbM
EIYPRSDNTF

SEQ ID NO: 271 - protein, 5 aa, synthetic construct
  source 1..5: note = Antibody 57D12 VH CDR1 - Kabat
SFGIS

SEQ ID NO: 272 - protein, 6 aa, synthetic construct
  source 1..6: note = Antibody 57D12 VH CDR1 - Contact
TSFGIS

SEQ ID NO: 273 - protein, 13 aa, synthetic construct
  source 1..13: note = Antibody 57D12 VH CDR2 - Contact
WIGEIYPRSDNTF

SEQ ID NO: 274 - protein, 10 aa, synthetic construct
  source 1..10: note = Antibody 57D12 VH CDR3 - Contact
ARHFGSSSFD

SEQ ID NO: 275 - protein, 8 aa, synthetic construct
  source 1..8: note = Antibody 57D12 VL CDR1 - Contact
SSIYFHWY

SEQ ID NO: 276 - protein, 10 aa, synthetic construct
  source 1..10: note = Antibody 57D12 VL CDR2 - Contact
PLIHRASNLA

SEQ ID NO: 277 - protein, 8 aa, synthetic construct
  source 1..8: note = Antibody 57D12 VL CDR3 - Contact
QQWSGYPL

SEQ ID NO: 278 - protein, 11 aa, synthetic construct
  source 1..11: note = Antibody 61H4 VH CDR1 - Exemplary, AbM
GYTFTDYYYMN

SEQ ID NO: 279 - protein, 17 aa, synthetic construct
  source 1..17: note = Antibody 61H4 VH CDR2 - Exemplary, Kabat
YIYPNNGATSYNQKFKG

SEQ ID NO: 280 - protein, 12 aa, synthetic construct
  source 1..12: note = Antibody 61H4 VH CDR3 - Exemplary, Chothia, AbM, Kabat
DGYSSNYYTMDY

SEQ ID NO: 281 - protein, 11 aa, synthetic construct
  source 1..11: note = Antibody 61H4 VL CDR1 - Exemplary, Chothia, AbM, Kabat
QASQGTSINLN

SEQ ID NO: 282 - protein, 7 aa, synthetic construct
  source 1..7: note = Antibody 61H4 VL CDR2 - Exemplary, Chothia, AbM, Kabat
GASNLED

SEQ ID NO: 283 - protein, 9 aa, synthetic construct
  source 1..9: note = Antibody 61H4 VL CDR3 - Exemplary, Chothia, AbM, Kabat
LQHTYLPYT

SEQ ID NO: 284 - protein, 8 aa, synthetic construct
  source 1..8: note = Antibody 61H4 VH CDR1 - Chothia
GYTFTDYY

SEQ ID NO: 285 - protein, 6 aa, synthetic construct
  source 1..6: note = Antibody 61H4 VH CDR2 - Chothia
YPNNGA

SEQ ID NO: 286 - protein, 10 aa, synthetic construct
  source 1..10: note = Antibody 61H4 VH CDR2 - AbM
YIYPNNGATS

SEQ ID NO: 287 - protein, 6 aa, synthetic construct
  source 1..6: note = Antibody 61H4 VH CDR1 - Kabat
DYYYMN

SEQ ID NO: 288 - protein, 7 aa, synthetic construct
  source 1..7: note = Antibody 61H4 VH CDR1 - Contact
TDYYYMN

SEQ ID NO: 289 - protein, 13 aa, synthetic construct
  source 1..13: note = Antibody 61H4 VH CDR2 - Contact
WIGYIYPNNGATS

SEQ ID NO: 290 - protein, 13 aa, synthetic construct
  source 1..13: note = Antibody 61H4 VH CDR3 - Contact
ARDGYSSNYYTMD

SEQ ID NO: 291 - protein, 7 aa, synthetic construct
  source 1..7: note = Antibody 61H4 VL CDR1 - Contact
SINLNWF

SEQ ID NO: 292 - protein, 10 aa, synthetic construct
  source 1..10: note = Antibody 61H4 VL CDR2 - Contact
LLIYGASNLE

SEQ ID NO: 293 - protein, 8 aa, synthetic construct
  source 1..8: note = Antibody 61H4 VL CDR3 - Contact
LQHTYLPY

SEQ ID NO: 294 - protein, 19 aa, synthetic construct
  source 1..19: note = Antibody 62G10 and Hz62G10.v1 VH CDR2 - Exemplary, Kabat
RIRTKNNNFATYYADSVKD

SEQ ID NO: 295 - protein, 6 aa, synthetic construct
  source 1..6: note = Antibody 62G10 and Hz62G10.v1 VH CDR3 - Exemplary, Chothia, AbM, Kabat
GPYFDY

SEQ ID NO: 296 - protein, 11 aa, synthetic construct
  source 1..11: note = Antibody 62G10 and Hz62G10.v1 VL CDR1 - Exemplary, Chothia, AbM, Kabat
LASQTIGTWLA

SEQ ID NO: 297 - protein, 9 aa, synthetic construct
  source 1..9: note = Antibody 62G10 and Hz62G10.v1 VL CDR3 - Exemplary, Chothia, AbM, Kabat
QQLYSTPYT

SEQ ID NO: 298 - protein, 8 aa, synthetic construct
  source 1..8: note = Antibody 62G10 and Hz62G10.v1 VH CDR2 - Chothia
RTKNNNFA

SEQ ID NO: 299 - protein, 12 aa, synthetic construct
  source 1..12: note = Antibody 62G10 and Hz62G10.v1 VH CDR2 - AbM
RIRTKNNNFATY

SEQ ID NO: 300 - protein, 15 aa, synthetic construct
  source 1..15: note = Antibody 62G10 and Hz62G10.v1 VH CDR2 - Contact
WVARIRTKNNNFATY

SEQ ID NO: 301 - protein, 7 aa, synthetic construct
  source 1..7: note = Antibody 62G10 and Hz62G10.v1 VH CDR3 - Contact
VRGPYFD

SEQ ID NO: 302 - protein, 7 aa, synthetic construct
  source 1..7: note = Antibody 62G10 and Hz62G10.v1 VL CDR1 - Contact
GTWLAWY

SEQ ID NO: 303 - protein, 8 aa, synthetic construct
  source 1..8: note = Antibody 62G10 and Hz62G10.v1 VL CDR3 - Contact
QQLYSTPY

SEQ ID NO: 304 - protein, 19 aa, synthetic construct
  source 1..19: note = Antibody 108D10 VH CDR2 - Exemplary, Kabat
RIRTKNNNYATYYADSVKD

SEQ ID NO: 305 - protein, 9 aa, synthetic construct
  source 1..9: note = Antibody 108D10 VH CDR3 - Exemplary, Chothia, AbM, Kabat
DRYGGAMAY

SEQ ID NO: 306 - protein, 11 aa, synthetic construct
  source 1..11: note = Antibody 108D10 VL CDR1 - Exemplary, Chothia, AbM, Kabat
KASEDIYNRLA

SEQ ID NO: 307 - protein, 7 aa, synthetic construct
  source 1..7: note = Antibody 108D10 VL CDR2 - Exemplary, Chothia, AbM, Kabat
SATSLET

SEQ ID NO: 308 - protein, 9 aa, synthetic construct
  source 1..9: note = Antibody 108D10 VL CDR3 - Exemplary, Chothia, AbM, Kabat
QQYWTIPYT

SEQ ID NO: 309 - protein, 12 aa, synthetic construct
  source 1..12: note = Antibody 108D10 VH CDR2 - AbM
RIRTKNNNYATY

SEQ ID NO: 310 - protein, 15 aa, synthetic construct
  source 1..15: note = Antibody 108D10 VH CDR2 - Contact
WVARIRTKNNNYATY

SEQ ID NO: 311 - protein, 10 aa, synthetic construct
  source 1..10: note = Antibody 108D10 VH CDR3 - Contact
VRDRYGGAMA

SEQ ID NO: 312 - protein, 7 aa, synthetic construct
  source 1..7: note = Antibody 108D10 VL CDR1 - Contact
YNRLAWY

SEQ ID NO: 313 - protein, 10 aa, synthetic construct
  source 1..10: note = Antibody 108D10 VL CDR2 - Contact
LLISSATSLE

SEQ ID NO: 314 - protein, 8 aa, synthetic construct
  source 1..8: note = Antibody 108D10 VL CDR3 - Contact
QQYWTIPY

SEQ ID NO: 315 - protein, 10 aa, synthetic construct
  source 1..10: note = Antibody 43H2 VH CDR1 - Exemplary, AbM
GFTFSNYGIH

SEQ ID NO: 316 - protein, 17 aa, synthetic construct
  source 1..17: note = Antibody 43H2 VH CDR2 - Exemplary, Kabat
SISPSGRSTYFRDSVKG

SEQ ID NO: 317 - protein, 9 aa, synthetic construct
  source 1..9: note = Antibody 43H2 VH CDR3 - Exemplary, Chothia, AbM, Kabat
GINYSSFDY

SEQ ID NO: 318 - protein, 11 aa, synthetic construct
  source 1..11: note = Antibody 43H2 VL CDR1 - Exemplary, Chothia, AbM, Kabat
KASQNVGSHVD

SEQ ID NO: 319 - protein, 7 aa, synthetic construct
  source 1..7: note = Antibody 43H2 VL CDR2 - Exemplary, Chothia, AbM, Kabat
TASNRYT

SEQ ID NO: 320 - protein, 9 aa, synthetic construct
  source 1..9: note = Antibody 43H2 VL CDR3 - Exemplary, Chothia, AbM, Kabat
MQSNSYPPT

SEQ ID NO: 321 - protein, 7 aa, synthetic construct
  source 1..7: note = Antibody 43H2 VH CDR1 - Chothia
GFTFSNY

SEQ ID NO: 322 - protein, 6 aa, synthetic construct
  source 1..6: note = Antibody 43H2 VH CDR2 - Chothia
SPSGRS

SEQ ID NO: 323 - protein, 10 aa, synthetic construct
  source 1..10: note = Antibody 43H2 VH CDR2 - AbM
SISPSGRSTY

SEQ ID NO: 324 - protein, 5 aa, synthetic construct
  source 1..5: note = Antibody 43H2 VH CDR1 - Kabat
NYGIH

SEQ ID NO: 325 - protein, 6 aa, synthetic construct
  source 1..6: note = Antibody 43H2 VH CDR1 - Contact
SNYGIH

SEQ ID NO: 326 - protein, 13 aa, synthetic construct
  source 1..13: note = Antibody 43H2 VH CDR2 - Contact
WVASISPSGRSTY

SEQ ID NO: 327 - protein, 10 aa, synthetic construct
  source 1..10: note = Antibody 43H2 VH CDR3 - Contact
ATGINYSSFD

SEQ ID NO: 328 - protein, 7 aa, synthetic construct
  source 1..7: note = Antibody 43H2 VL CDR1 - Contact
GSHVDWY

SEQ ID NO: 329 - protein, 10 aa, synthetic construct
  source 1..10: note = Antibody 43H2 VL CDR2 - Contact
LLISTASNRY

SEQ ID NO: 330 - protein, 8 aa, synthetic construct
  source 1..8: note = Antibody 43H2 VL CDR3 - Contact
MQSNSYPP

SEQ ID NO: 331 - protein, 8 aa, synthetic construct
  source 1..8: note = Antibody 47H1, 108D10 VH CDR2 - Chothia
RTKNNNYA

(19) United States
(12) Patent Application Publication
(10) Pub. No.: US 2023/0220076 A1
(43) Pub. Date: Jul. 13, 2023

(54) COMBINATIONAL USE OF AN ANTI-ILT3 ANTIBODY AND AN ANTI-LAIR-1 ANTIBODY

(71) Applicant: NGM BIOPHARMACEUTICALS, INC., South San Francisco, CA (US)

(72) Inventors: Varun Kapoor, Alameda, CA (US); Peirong Chen, San Mateo, CA (US); Julie Michelle Roda, Pacifica, CA (US)

(21) Appl. No.: 18/069,173

(22) Filed: Dec. 20, 2022

Related U.S. Application Data
(60) Provisional application No. 63/265,824, filed on Dec. 21, 2021.

(51) Int. Cl. C07K 16/28; A61P 35/00; A61P 37/04; A61K 45/06; A61K 39/395

(52) U.S. Cl. CPC C07K 16/2803; A61P 35/00; A61P 37/04; A61K 45/06; A61K 39/3955; A61K 2039/507

(57) ABSTRACT

The present disclosure provides combination therapies comprising the use of an antibody that binds human ILT3 and an antibody that binds human LAIR-1.

### CROSS-REFERENCE TO RELATED APPLICATION

This application claims the benefit of priority to U.S. Provisional Application No. 63/265,824 filed Dec. 21, 2021, the content of which is incorporated by reference in its entirety.

### SEQUENCE LISTING

This application contains a computer readable Sequence Listing which has been submitted in XML file format with this application, the entire content of which is incorporated by reference herein in its entirety. The Sequence Listing XML file submitted with this application is entitled “13370-157-999_SequenceListing.xml”, was created on Dec. 12, 2022 and is 387,510 bytes in size.

### 1. FIELD

The present disclosure generally relates to combination therapies comprising the use of an antibody that binds human ILT3 and an antibody that binds human LAIR-1.

### 2. BACKGROUND

The basis for immunotherapy is the manipulation and/or modulation of the immune system, including both innate immune responses and adaptive immune responses. The general aim of immunotherapy is to treat diseases by controlling the immune response to a “foreign agent”, for example, a pathogen or a tumor cell. However, in some instances, immunotherapy is used to treat autoimmune diseases, which may arise from an abnormal immune response against proteins, molecules, and/or tissues normally present in the body. Thus, immunotherapy may include methods of inducing or enhancing specific immune responses, or inhibiting or reducing specific immune responses.

Immune system is a highly complex system made up of a great number of cell types, including but not limited to, T-cells, B-cells, natural killer cells, antigen-presenting cells, dendritic cells, monocytes, and macrophages. These cells possess complex and subtle systems for controlling their interactions and responses. The cells utilize both activating and inhibitory mechanisms and feedback loops to keep responses in check and avoid negative consequences of an uncontrolled immune response (e.g., autoimmune diseases or a cytokine storm).

Some of the inhibitory mechanisms of the immune system rely on signaling proteins that contain ITIMs (immunoreceptor tyrosine-based inhibitory motifs). These proteins are generally cell-surface receptors comprising the ITIMs in their cytoplasmic tails. The majority of cells in the immune system express at least one, and often many, inhibitory receptors. Inhibitory receptors (i) use specific intracellular effector pathways that affect a variety of activation signals, (ii) recognize distinct ligands across a range of locations in cells and tissues, and (iii) are differentially expressed between cell types and during differentiation and activation of cells. This allows these receptors to have an important part in a myriad of immune responses throughout the body. Many of the receptors are members of the Ig superfamily and include leukocyte immunoglobulin-like receptor subfamily B members (e.g., LILRB1, LILRB2, LILRB3, LILRB4, and LILRB5) and leukocyte-associated immunoglobulin-like receptor-1 (LAIR-1; also known as CD305) and LAIR-2. (See, for example, Meyaard et al., 1997, Immunity, 7:283-290; Meyaard, 2008, J. Leukocyte Biol., 83:799-803).

The concept of cancer immunosurveillance is based on the theory that the immune system can recognize tumor cells, mount an immune response, and suppress the development and/or growth of a tumor. However, many cancerous cells have developed mechanisms and/or hijacked normal inhibitory mechanisms to evade the immune system, which can allow for uninhibited growth of tumors. Cancer/tumor immunotherapy (immuno-oncology) focuses on the development of new and novel agents that can activate and/or boost the immune system to achieve a more effective attack against cancer/tumor cells, resulting in increased killing of cancer/tumor cells and/or inhibition of cancer/tumor growth. Agents and methods for boosting the immune response to uncontrolled cell proliferation, i.e., tumor growth or cancer, are needed.

### 3. SUMMARY OF THE INVENTION

The present disclosure generally relates to combination therapies comprising an antibody that binds human ILT3 and an antibody that binds human LAIR-1.

In one aspect, provided herein is a method of activating an immune cell associated with a tumor or a tumor microenvironment, wherein the method comprises contacting the immune cell with an effective amount of an anti-ILT3 antibody and an effective amount of an anti-LAIR-1 antibody. In another aspect, provided herein is a use of an anti-ILT3 antibody and an anti-LAIR-1 antibody for activating an immune cell associated with a tumor or a tumor microenvironment, wherein the use comprises contacting the immune cell with an effective amount of the anti-ILT3 antibody and an effective amount of the anti-LAIR-1 antibody. In certain embodiments, the immune cell is a myeloid cell. In certain embodiments, the myeloid cell is a monocyte, a macrophage, a dendritic cell, or an APC. In certain embodiments, the immune cell is a T cell. In certain embodiments, the T cell is a cytotoxic T-cell (CTL). In certain embodiments, the immune cell is a natural killer cell.

In another aspect, provided herein is a method of reducing or inhibiting immune suppressive activity of an immune cell associated with a tumor or a tumor microenvironment, wherein the method comprises contacting the immune cell with an effective amount of an anti-ILT3 antibody and an effective amount of an anti-LAIR-1 antibody. In another aspect, provided herein is a use of an anti-ILT3 antibody and an anti-LAIR-1 antibody for reducing or inhibiting immune suppressive activity of an immune cell associated with a tumor or a tumor microenvironment, wherein the use comprises contacting the immune cell with an effective amount of the anti-ILT3 antibody and an effective amount of the anti-LAIR-1 antibody. In certain embodiments, the immune cell is a regulatory T-cell (Treg). In certain embodiments, the immune cell is a tolerogenic dendritic cell. In certain embodiments, the immune cell is a myeloid-derived suppressor cell (MDSC).

In certain embodiments, the method disclosed herein further comprises contacting the immune cell with an additional therapeutic agent. In certain embodiments, the additional therapeutic agent is an immune-checkpoint inhibitor. In certain embodiments, the immune-checkpoint inhibitor is a PD-1 inhibitor or a PD-L1 inhibitor.

In another aspect, provided herein is a method of activating an immune cell in a subject diagnosed with cancer, wherein the method comprises administering to the subject a therapeutically effective amount of an anti-ILT3 antibody and a therapeutically effective amount of an anti-LAIR-1 antibody. In another aspect, provided herein is a use of an anti-ILT3 antibody and an anti-LAIR-1 antibody for activating an immune cell in a subject diagnosed with cancer, wherein the use comprises administering to the subject a therapeutically effective amount of the anti-ILT3 antibody and a therapeutically effective amount of the anti-LAIR-1 antibody. In certain embodiments, the immune cell is a myeloid cell. In certain embodiments, the myeloid cell is a monocyte, a macrophage, a dendritic cell, or an APC. In certain embodiments, the immune cell is a T cell. In certain embodiments, the T cell is a cytotoxic T-cell (CTL). In certain embodiments, the immune cell is a natural killer cell.

In another aspect, provided herein is a method of reducing or inhibiting immune suppressive activity of in an immune cell in a subject diagnosed with cancer, wherein the method comprises administering to the subject a therapeutically effective amount of an anti-ILT3 antibody and a therapeutically effective amount of an anti-LAIR-1 antibody. In another aspect, provided is a use of an anti-ILT3 antibody and an anti-LAIR-1 antibody for reducing or inhibiting immune suppressive activity of an immune cell in a subject diagnosed with cancer, wherein the use comprises administering to the subject a therapeutically effective amount of the anti-ILT3 antibody and a therapeutically effective amount of the anti-LAIR-1 antibody. In certain embodiments, the immune cell is a regulatory T-cell (Treg). In certain embodiments, the immune cell is a tolerogenic dendritic cell. In certain embodiments, the immune cell is a myeloid-derived suppressor cell (MDSC).

In another aspect, provided herein is a method of reversing stromal-mediated immunosuppression in a subject diagnosed with cancer, wherein the method comprises administering to the subject a therapeutically effective amount of an anti-ILT3 antibody and a therapeutically effective amount of an anti-LAIR-1 antibody. In another aspect, provided herein is a method of treating cancer in a subject, wherein the method comprises administering to the subject a therapeutically effective amount of an anti-ILT3 antibody and a therapeutically effective amount of an anti-LAIR-1 antibody. In another aspect, provided herein is a method of enhancing an immune response to cancer in a subject diagnosed with the cancer, wherein the method comprises administering to the subject a therapeutically effective amount of an anti-ILT3 antibody and a therapeutically effective amount of an anti-LAIR-1 antibody. In another aspect, provided herein is a use of an anti-ILT3 antibody and an anti-LAIR-1 antibody for reversing stromal-mediated immunosuppression in a subject diagnosed with cancer, wherein the use comprises administering to the subject a therapeutically effective amount of the anti-ILT3 antibody and a therapeutically effective amount of the anti-LAIR-1 antibody. In another aspect, provided herein is a use of an anti-ILT3 antibody and an anti-LAIR-1 antibody for treating cancer in a subject, wherein the use comprises administering to the subject a therapeutically effective amount of the anti-ILT3 antibody and a therapeutically effective amount of the anti-LAIR-1 antibody. In another aspect, provided herein is a use of an anti-ILT3 antibody and an anti-LAIR-1 antibody for enhancing an immune response to cancer in a subject diagnosed with the cancer, wherein the use comprises administering to the subject a therapeutically effective amount of the anti-ILT3 antibody and a therapeutically effective amount of the anti-LAIR-1 antibody.

In certain embodiments of any of the methods or uses disclosed herein, the cancer is a hematologic cancer. In certain embodiments, the cancer is a solid tumor. In certain embodiments, the cancer is a pancreatic cancer, a breast cancer, a mesothelioma, a gastric cancer, an NSCLC, a cervical cancer, an endocervical cancer, a biliary duct cancer, a SCCHN, a bladder cancer, a urothelial cancer, a CRC, an esophageal cancer, an ovarian cancer, an RCC, a prostate cancer, or a melanoma. In certain embodiments, the pancreatic cancer is pancreatic ductal adenocarcinoma. In certain embodiments, the cancer is a tumor mutational burden-high (TMB-H) cancer. In certain embodiments, the cancer is a microsatellite instability-high (MSI-H) cancer.

In certain embodiments of any of the methods or uses described herein, further comprises administering to the subject an additional therapeutic agent. In certain embodiments, the additional therapeutic agent is an immune-checkpoint inhibitor. In certain embodiments, the immune-checkpoint inhibitor is a PD-1 inhibitor or a PD-L1 inhibitor. In certain embodiments, the subject is a human.

In certain embodiments of any of the methods or uses described herein, the anti-ILT3 antibody and the anti-LAIR-1 antibody are formulated into two separate pharmaceutical compositions, where a first pharmaceutical composition comprises the anti-ILT3 antibody and a pharmaceutically acceptable carrier; and a second pharmaceutical composition comprises the anti-LAIR-1 antibody and a pharmaceutically acceptable carrier.

In certain embodiments of any of the methods or uses described above, the anti-ILT3 antibody and the anti-LAIR-1 antibody are formulated into a pharmaceutical composition, wherein the pharmaceutical composition comprises the anti-ILT3 antibody, the anti-LAIR-1 antibody, and a pharmaceutically acceptable carrier.

Also provided herein is a kit which comprises a first container, a second container and a package insert, wherein the first container comprises at least one dose of a medicament comprising an anti-ILT3 antibody, the second container comprises at least one dose of a medicament comprising an anti-LAIR-1 antibody, and the package insert comprises instructions for treating a subject diagnosed with cancer using the medicaments. In certain embodiments, the subject is a human.

Also provided in an embodiment is a combination comprising an anti-LAIR-1 antibody and an anti-ILT3 antibody. In certain embodiments, the anti-LAIR-1 antibody and the anti-ILT3 antibody are formulated separately. In certain embodiments, the anti-LAIR-1 antibody and the anti-ILT3 antibody are co-formulated. In certain embodiments, the anti-LAIR-1 antibody and the anti-ILT3 antibody are co-formulated as a pharmaceutical composition. In certain embodiments, the anti-LAIR-1 antibody and the anti-ILT3 antibody are each separately formulated as a pharmaceutical composition. In certain embodiments, the anti-LAIR-1 antibody and the anti-ILT3 antibody synergistically overcomes stromal-mediated immunosuppression in a tumor microenvironment

In certain embodiments of any of the methods, uses, kits, or combinations described above, the anti-ILT3 antibody inhibits binding of ILT3 to fibronectin. In certain embodiments of any of the methods, uses, kits, or combinations described above, the anti-ILT3 antibody inhibits binding of ILT3 to APOE. In certain embodiments of any of the methods, uses, kits, or combinations described above, the anti-ILT3 antibody inhibits binding of ILT3 to CNTFR. In certain embodiments of any of the methods, uses, kits, or combinations described above, the anti-ILT3 antibody inhibits ILT3 activity. In certain embodiments of any of the methods, uses, kits, or combinations described above, the anti-LAIR-1 antibody inhibits LAIR-1 activity. In certain embodiments of any of the methods, uses, kits, or combinations described above, the anti-LAIR-1 antibody inhibits binding of LAIR-1 to collagen. In certain embodiments of any of the methods, uses, kits, or combinations described above, the anti-LAIR-1 antibody inhibits binding of LAIR-1 to MARCO. In certain embodiments of any of the methods, uses, kits, or combinations described above, the anti-LAIR-1 antibody inhibits binding of LAIR-1 to COLEC12.

In certain embodiments of any of the methods, uses, kits, or combinations described above, the anti-ILT3 antibody comprises: (a) a heavy chain variable region (VH) comprising a VH-complementarity determining region (CDR)1, a VH-CDR2, and a VH-CDR3 from the amino acid sequence of SEQ ID NO:111; and a light chain variable region (VL) comprising a VL-CDR1, a VL-CDR2, and a VL-CDR3 from the amino acid sequence of SEQ ID NO:112; or (b) a VH comprising a VH-CDR1, a VH-CDR2, and a VH-CDR3 from the amino acid sequence of SEQ ID NO:123; and a VL comprising a VL-CDR1, a VL-CDR2, and a VL-CDR3 from the amino acid sequence of SEQ ID NO:124.

In certain embodiments of any of the methods, uses, kits, or combinations described above, (a) the VH comprises a VH-CDR1 comprising the amino acid sequence GFTFSSYGMS (SEQ ID NO:27), a VH-CDR2 comprising the amino acid sequence TISGGGSYTNYPDSVKG (SEQ ID NO:28), and a VH-CDR3 comprising the amino acid sequence REWRYTLYAMDY (SEQ ID NO:105); and the VL comprises a VL-CDR1 comprising the amino acid sequence RASESVESYGSSFMH (SEQ ID NO:106), a VL-CDR2 comprising the amino acid sequence LTSNLES (SEQ ID NO:31), and a VL-CDR3 comprising the amino acid sequence QQNNEDPFT (SEQ ID NO:32); (b) the VH comprises a VH-CDR1 comprising the amino acid sequence GFTFSSY (SEQ ID NO:33), a VH-CDR2 comprising the amino acid sequence SGGGSY (SEQ ID NO:34), and a VH-CDR3 comprising the amino acid sequence REWRYTLYAMDY (SEQ ID NO:105); and the VL comprises a VL-CDR1 comprising the amino acid sequence RASESVESYGSSFMH (SEQ ID NO:106), a VL-CDR2 comprising the amino acid sequence LTSNLES (SEQ ID NO:31), and a VL-CDR3 comprising the amino acid sequence QQNNEDPFT (SEQ ID NO:32); (c) the VH comprises a VH-CDR1 comprising the amino acid sequence GFTFSSYGMS (SEQ ID NO:27), a VH-CDR2 comprising the amino acid sequence TISGGGSYTN (SEQ ID NO:35), and a VH-CDR3 comprising the amino acid sequence REWRYTLYAMDY (SEQ ID NO:105); and the VL comprises a VL-CDR1 comprising the amino acid sequence RASESVESYGSSFMH (SEQ ID NO:106), a VL-CDR2 comprising the amino acid sequence LTSNLES (SEQ ID NO:31), and a VL-CDR3 comprising the amino acid sequence QQNNEDPFT (SEQ ID NO:32); (d) the VH comprises a VH-CDR1 comprising the amino acid sequence SYGMS (SEQ ID NO:36), a VH-CDR2 comprising the amino acid sequence TISGGGSYTNYPDSVKG (SEQ ID NO:28), and a VH-CDR3 comprising the amino acid sequence REWRYTLYAMDY (SEQ ID NO:105); and the VL comprises a VL-CDR1 comprising the amino acid sequence RASESVESYGSSFMH (SEQ ID NO:106), a VL-CDR2 comprising the amino acid sequence LTSNLES (SEQ ID NO:31), and a VL-CDR3 comprising the amino acid sequence QQNNEDPFT (SEQ ID NO:32); or (e) the VH comprises a VH-CDR1 comprising the amino acid sequence SSYGMS (SEQ ID NO:37), a VH-CDR2 comprising the amino acid sequence WVATISGGGSYTN (SEQ ID NO:38), and a VH-CDR3 comprising the amino acid sequence ARREWRYTLYAMD (SEQ ID NO:107), and the VL comprises a VL-CDR1 comprising the amino acid sequence ESYGSSFMHWY (SEQ ID NO:108), a VL-CDR2 comprising the amino acid sequence LLIYLTSNLE (SEQ ID NO:41), and a VL-CDR3 comprising the amino acid sequence QQNNEDPF (SEQ ID NO:42).

In certain embodiments of any of the methods, uses, kits, or combinations described above, (a) the VH comprises an amino acid sequence having at least 80% sequence identity to the amino acid sequence of SEQ ID NO:123; (b) the VL comprises an amino acid sequence having at least 80% sequence identity to the amino acid sequence of SEQ ID NO:124; or (c) the VH comprises an amino acid sequence having at least 80% sequence identity to the amino acid sequence of SEQ ID NO:123 and the VL comprises an amino acid sequence having at least 80% sequence identity to the amino acid sequence of SEQ ID NO:124.

In certain embodiments of any of the methods, uses, kits, or combinations described above, the VH comprises the amino acid sequence of SEQ ID NO:123 and the VL comprises the amino acid sequence of SEQ ID NO:124.

In certain embodiments of any of the methods, uses, kits, or combinations described above, the anti-ILT3 antibody comprises: (a) a heavy chain comprising an amino acid sequence having at least 80% sequence identity to the amino acid sequence of SEQ ID NO:126; (b) a light chain comprising an amino acid sequence having at least 80% sequence identity to the amino acid sequence of SEQ ID NO:128; or (c) a heavy chain comprising an amino acid sequence having at least 80% sequence identity to the amino acid sequence of SEQ ID NO:126, and a light chain comprising an amino acid sequence having at least 80% sequence identity to the amino acid sequence of SEQ ID NO:128.

In certain embodiments of any of the methods, uses, kits, or combinations described above, the heavy chain comprises the amino acid sequence of SEQ ID NO:126 and the light chain comprises the amino acid sequence of SEQ ID NO:128.

In certain embodiments of any of the methods, uses, kits, or combinations described above, the anti-LAIR-1 antibody comprises: a heavy chain variable region (VH) comprising a VH-complementarity determining region (CDR)1, a VH-CDR2 and a VH-CDR3 from SEQ ID NO:179, and a light chain variable region (VL) comprising a VL-CDR1, a VL-CDR2 and a VL-CDR3 from SEQ ID NO:180.

In certain embodiments of any of the methods, uses, kits, or combinations described above, (1) the VH comprises the VH-CDR1 comprising the amino acid sequence of SEQ ID NO:242, the VH-CDR2 comprising the amino acid sequence of SEQ ID NO:257, and the VH-CDR3 comprising the amino acid sequence of SEQ ID NO:244; and the VL comprises the VL-CDR1 comprising the amino acid sequence of SEQ ID NO:245, the VL-CDR2 comprising the amino acid sequence of SEQ ID NO:258, and the VL-CDR3 comprising the amino acid sequence of SEQ ID NO:247; (2) the VH comprises the VH-CDR1 comprising the amino acid sequence of SEQ ID NO:248, the VH-CDR2 comprising the amino acid sequence of SEQ ID NO:259, and the VH-CDR3 comprising the amino acid sequence of SEQ ID NO:244; and the VL comprises the VL-CDR1 comprising the amino acid sequence of SEQ ID NO:245, the VL-CDR2 comprising the amino acid sequence of SEQ ID NO:258, and the VL-CDR3 comprising the amino acid sequence of SEQ ID NO:247; (3) the VH comprises the VH-CDR1 comprising the amino acid sequence of SEQ ID NO:242, the VH-CDR2 comprising the amino acid sequence of SEQ ID NO:260, and the VH-CDR3 comprising the amino acid sequence of SEQ ID NO:244; and the VL comprises the VL-CDR1 comprising the amino acid sequence of SEQ ID NO:245, the VL-CDR2 comprising the amino acid sequence of SEQ ID NO:258, and the VL-CDR3 comprising the amino acid sequence of SEQ ID NO:247; (4) the VH comprises the VH-CDR1 comprising the amino acid sequence of SEQ ID NO:250, the VH-CDR2 comprising the amino acid sequence of SEQ ID NO:257, and the VH-CDR3 comprising the amino acid sequence of SEQ ID NO:244; and the VL comprises the VL-CDR1 comprising the amino acid sequence of SEQ ID NO:245, the VL-CDR2 comprising the amino acid sequence of SEQ ID NO:258, and the VL-CDR3 comprising the amino acid sequence of SEQ ID NO:247; or (5) the VH comprises the VH-CDR1 comprising the amino acid sequence of SEQ ID NO:251, the VH-CDR2 comprising the amino acid sequence of SEQ ID NO:261, and the VH-CDR3 comprising the amino acid sequence of SEQ ID NO:253; and the VL comprises the VL-CDR1 comprising the amino acid sequence of SEQ ID NO:254, the VL-CDR2 comprising the amino acid sequence of SEQ ID NO:255, and the VL-CDR3 comprising the amino acid sequence of SEQ ID NO:256.

In certain embodiments of any of the methods, uses, kits, or combinations described above, the VH has at least 80% sequence identity to the amino acid sequence of SEQ ID NO:179 and/or the VL has at least 80% sequence identity to the amino acid sequence of SEQ ID NO:180.

In certain embodiments of any of the methods, uses, kits, or combinations described above, the VH comprises the amino acid sequence of SEQ ID NO:179 and/or the VL comprises the amino acid sequence of SEQ ID NO:180.

In certain embodiments of any of the methods, uses, kits, or combinations described above, the anti-LAIR-1 antibody comprises: a heavy chain comprising an amino acid sequence with 80% identity to the amino acid sequence of SEQ ID NO:194, and/or a light chain comprising an amino acid sequence with 80% identity to the amino acid sequence of SEQ ID NO:196.

In certain embodiments of any of the methods, uses, kits, or combinations described above, the anti-LAIR-1 antibody comprises: the heavy chain comprises the amino acid sequence of SEQ ID NO:194, and/or the light chain comprises the amino acid sequence of SEQ ID NO:196.

### 4. BRIEF DESCRIPTION OF DRAWINGS

FIG. 1 shows TNF-α secretion by monocyte-derived DCs (moDCs) in the presence of an anti-ILT3 antibody (hz5A7.v5), an anti-LAIR-1 antibody (hz47H1.v4), or a combination of both. “No COL/FN” indicates the experimental condition where no collagen and fibronectin was present. Anti-KLH=asterisk (*); hz5A7=circle (●); hz47H1=square (▪); hz5A7+hz47H1.v4=triangle (▴); No COL/FN=gray x (“X”).

FIG. 2 shows the level of MIP-1α secretion by monocyte-derived DCs (moDCs) in the presence of an anti-ILT3 antibody (hz5A7.v5), an anti-LAIR-1 antibody (hz47H1.v4), or a combination of both. The collagen:fibronectin (COL:FN) ratios used to coat the wells are indicated. Both collagen and fibronectin were given in μg/ml. * indicates p<0.001. In each of the conditions, the first bar is COL:FN 4:1.25, the second or middle bar is COL:FN 1.25:1.25, and the third bar is COL:FN 1.25:4.

FIG. 3 shows the level of MIP-1α secretion by tolerogenic DCs in the presence of an anti-ILT3 antibody (hz5A7.v5), an anti-LAIR-1 antibody (hz47H1.v4), or a combination of both. Antibody concentration started at 10 μg/mL, and 2-fold dilutions were performed. Anti-KLH=gray asterisk (*); hz5A7=circle (●); hz47H1=square (▪); hz5A7+hz47H1=triangle (▴).

FIG. 4 shows the level of chemokine secretion by tolerogenic DCs in the presence of an anti-ILT3 antibody (hz5A7.v5), an anti-LAIR-1 antibody (hz47H1.v4), or a combination of both. In each of the conditions, the first bar is anti-KLH (circles ●), the second bar is hz5A7 (squares ▪), the third bar is hz47H1 (diamonds ♦), and the last bar is hz5A7+hz47H1 (triangles ▴).

FIG. 5 shows the level of chemokine secretion by M2a macrophages in the presence of an anti-ILT3 antibody (hz5A7.v5), an anti-LAIR-1 antibody (hz47H1.v4), or a combination of both. In each of the conditions, the first bar is anti-KLH (circles ●), the second bar is hz5A7 (squares ▪), the third bar is hz47H1 (diamonds ♦), and the last bar is hz5A7+hz47H1 (triangles ▴).

FIG. 6 shows mRNA downregulation of scavenger receptors in tolerogenic DCs in the presence of an anti-ILT3 antibody (hz5A7.v5), an anti-LAIR-1 antibody (hz47H1.v4), or a combination of both. In each of the conditions, the first bar is anti-KLH (circles ●), the second bar is hz47H1 (squares ▪), the third bar is hz5A7 (triangles ▴), and the last bar is hz5A7+hz47H1 (diamonds ♦).

FIG. 7 shows mRNA downregulation of myeloid cell inhibitory receptors in tolerogenic DCs in the presence of an anti-ILT3 antibody (hz5A7.v5), an anti-LAIR-1 antibody (hz47H1.v4), or a combination of both. In each of the conditions, the first bar is anti-KLH (circles ●), the second bar is hz47H1 (squares ▪), the third bar is hz5A7 (triangles ▴), and the last bar is hz5A7+hz47H1 (diamonds ♦).

FIG. 8 shows mRNA downregulation of markers of dendritic cell tolerization and immaturity in tolerogenic DCs in the presence of an anti-ILT3 antibody (hz5A7.v5), an anti-LAIR-1 antibody (hz47H1.v4), or a combination of both. In each of the conditions, the first bar is anti-KLH (circles ●), the second bar is hz47H1 (squares ▪), the third bar is hz5A7 (triangles ▴), and the last bar is hz5A7+hz47H1 (diamonds ♦).

FIG. 9 shows mRNA upregulation of genes involved in antigen presentation of tolerogenic DCs in the presence of an anti-ILT3 antibody (hz5A7.v5), an anti-LAIR-1 antibody (hz47H1.v4), or a combination of both. In each of the conditions, the first bar is anti-KLH (circles ●), the second bar is hz47H1 (squares ▪), the third bar is hz5A7 (triangles ▴), and the last bar is hz5A7+hz47H1 (diamonds ♦).

FIG. 10 shows mRNA upregulation of genes involved in T cell stimulation of tolerogenic DCs in the presence of an anti-ILT3 antibody (hz5A7.v5), an anti-LAIR-1 antibody (hz47H1.v4), or a combination of both. In each of the conditions, the first bar is anti-KLH (circles ●), the second bar is hz47H1 (squares ▪), the third bar is hz5A7 (triangles ▴), and the last bar is hz5A7+hz47H1 (diamonds ♦).

FIG. 11 shows mRNA upregulation and downregulation of cytokines and chemokines secreted by tolerogenic DCs in the presence of an anti-ILT3 antibody (hz5A7.v5), an anti-LAIR-1 antibody (hz47H1.v4), or a combination of both. In each of the conditions, the first bar is anti-KLH (circles ●), the second bar is hz47H1 (squares ▪), the third bar is hz5A7 (triangles ▴), and the last bar is hz5A7+hz47H1 (diamonds ♦).

FIG. 12A shows differentially regulated genes (>1.5 fold) in tolerogenic DCs by an anti-ILT3 antibody (hz5A7.v5), an anti-LAIR-1 antibody (hz47H1.v4), or the combination of both. FIG. 12B shows differentially regulated pathways in tolerogenic DCs by an anti-ILT3 antibody (hz5A7.v5), an anti-LAIR-1 antibody (hz47H1.v4), or the combination of both.

FIG. 13 shows the number of upregulated and downregulated genes and their fold expression changes in tolerogenic DCs by an anti-ILT3 antibody (hz5A7.v5), an anti-LAIR-1 antibody (hz47H1.v4) or the combination of both.

FIG. 14 shows the levels of cell surface receptor expression after the treatment of an anti-ILT3 antibody (hz5A7.v5), an anti-LAIR-1 antibody (hz47H1.v4), or the combination of both in tolerogenic DCs isolated from two donors.

FIG. 15A shows the percentage of LAIR-1 positive cells after treating humanized mice with an anti-ILT3 antibody. FIG. 15B shows the LAIR-1 expression level on tumor-associated myeloid APCs after treating the humanized mice with an anti-ILT3 antibody. Anti-KLH antibody was used as a control. *p=0.05, **p<0.01 vs. anti-KLH control.

FIG. 16 shows migration of dendritic cells and macrophages after the treatment with the combination of the anti-ILT3 and anti-LAIR-1 antibodies. Each data point represents the mean of experimental replicates from an individual donor. Cells from 4 donors were assayed.

### 5. DETAILED DESCRIPTION OF THE INVENTION

Unless explained otherwise, all technical and scientific terms used herein have the same meaning as commonly understood to one of ordinary skill in the art to which this disclosure belongs. It is understood that the term “including” or “include(s),” is used interchangeably with “comprising” or “comprise(s)” to describe embodiments herein.

### 5.1. Antibodies

### 5.1.1. Antibodies in General

The term “antibody” is used in the broadest sense and comprises, for example, an intact immunoglobulin, and an antibody fragment containing an antigen binding portion. Traditional antibody structural units typically include a tetramer. Each tetramer is typically composed of two identical pairs of polypeptide chains, where each pair has one “light” chain (typically having a molecular weight of about 25 kDa) and one “heavy” chain (typically having a molecular weight of about 50-70 kDa). Human light chains are classified as kappa and lambda light chains. The present disclosure is directed to antibodies that generally are based on the IgG class, which has several subclasses, including, but not limited to IgG1, IgG2, IgG3, and IgG4. In general, IgG1, IgG2 and IgG4 are used more frequently than IgG3. It should be noted that IgGs have different allotypes. All IgGs allotypes can be used for the presently disclosed subject matter. For example, IgG1 has polymorphisms at 356 (D or E) and 358 (L or M). The sequences depicted herein use the 356E/358M allotype, however, the other allotypes are included herein. As will be appreciated by those in the art, the exact numbering and placement of the Complementary Determining Regions (CDRs) can be different among different numbering systems. However, it should be understood that the present disclosure of a variable heavy and/or variable light region sequence comprises the present disclosure of the associated (inherent) CDRs. Accordingly, the present disclosure of each variable heavy region (VH) is a disclosure of the VH-CDRs (e.g., VH-CDR1, VH-CDR2, and VH-CDR3) and the present disclosure of each variable light region (VL) is a disclosure of the VL-CDRs (e.g., VL-CDR1, VL-CDR2, and VL-CDR3).

CDRs of an antibody can be defined using a variety of methods/systems by those skilled in the art. These systems and/or definitions have been developed and refined over a number of years and include Kabat, Chothia, IMGT, AbM, and Contact. The Kabat definition is based on sequence variability and is commonly used. The Chothia definition is based on the location of the structural loop regions. The IMGT system is based on sequence variability and location within the structure of the variable region. The AbM definition is a compromise between Kabat and Chothia. The Contact definition is based on analyses of the available antibody crystal structures. An Exemplary system disclosed herein is a combination of Kabat and Chothia. Software programs (e.g., abYsis) are available and known to those of skill in the art for analysis of antibody sequences and determination of CDRs. It will be understood that reference in this disclosure to a VH-CDR or VH-CDRs and/or a VL-CDR or VL-CDRs of a specific antibody, including a specific VH amino acid sequence and/or a VL amino acid sequence, will encompass all CDR definitions known to those of skill in the art.

Various methods for generating humanized antibodies are known in the art. In certain embodiments, a humanized antibody comprises one or more amino acid residues that have been introduced into it from a source that is non-human. In certain embodiments, humanization is performed by substituting one or more non-human CDR sequences for the corresponding CDR sequences of a human antibody. In certain embodiments, the humanized antibodies are constructed by substituting all six CDRs of a non-human antibody (e.g., a mouse antibody) for the corresponding CDRs of a human antibody.

The choice of which human heavy chain variable region and/or light chain variable region is used for generating humanized antibodies can be made based on a variety of factors and by a variety of methods known in the art. In certain embodiments, the “best-fit” method is used where the sequence of the variable region of a non-human (e.g., rodent) antibody is screened against the entire library of known human variable region sequences. The human sequence that is most similar to that of the non-human (e.g., rodent) sequence is selected as the human variable region framework for the humanized antibody. In certain embodiments, a particular variable region framework derived from a consensus sequence of all human antibodies of a particular subgroup of light or heavy chains is selected as the variable region framework. In certain embodiments, the variable region framework sequence is derived from the consensus sequences of the most abundant human subclasses. In certain embodiments, human germline genes are used as the source of the variable region framework sequences.

Other methods for humanization include, but are not limited to, a method called “superhumanization” which is described as the direct transfer of CDRs to a human germline framework, a method termed Human String Content (HSC) which is based on a metric of “antibody humanness”, methods based on generation of large libraries of humanized variants (including phage, ribosomal, and yeast display libraries), and methods based on framework region shuffling.

### 5.1.2. Anti-ILT3 Antibodies

ILT3 (also known as LILRB4, CD85K, ILT-3, ILT3, LIR-5, LIRS, leukocyte immunoglobulin like receptor B4, and B4) is a single pass type I transmembrane protein with a predicted molecular weight of approximately 47 kDa. ILT3 is predominantly expressed on myeloid antigen presenting cells, such as normal monocytes, macrophages, and dendritic cells. ILT3 has an extracellular domain comprising two Ig-like C2 type domains, a transmembrane domain, and a long cytoplasmic domain containing 3 ITIM domains (see, e.g., Cella et al., 1997, J Exp. Med., 185:1743-1751). The two Ig-like C2-type domains is referred to herein as Domain 1 (D1) and Domain 2 (D2). D1 is situated at the N-terminal portion of the protein and D2 is situated closest to the transmembrane region. As characterized by UniProtKB, human ILT3 is a protein of 448 amino acids (aa) long, where the signal sequence is aa 1-21, the extracellular domain is aa 22-259, the transmembrane region is aa 260-280, and the cytoplasmic domain is aa 281-448. Within the extracellular domain, D1 is aa 27-188, D2 is aa 124-218, and the “stem region” is aa 219-259. With the cytoplasmic domain, ITIMs are aa 358-363, 410-415, and 440-445.

The amino acid (aa) sequence for human ILT3 (UniProtKB No. Q8NHJ6) is shown below and includes the predicted signal sequence (underlined residues):

(SEQ ID NO: 1)
MIPTFTALLCLGLSLGPRTHMQAGPLPKPTLWAEPGSVISWGNSVTIWCQ
GTLEAREYRLDKEESPAPWDRQNPLEPKNKARFSIPSMTEDYAGRYRCYY
RSPVGWSQPSDPLELVMTGAYSKPTLSALPSPLVTSGKSVTLLCQSRSPM
DTFLLIKERAAHPLLHLRSEHGAQQHQAEFPMSPVTSVHGGTYRCFSSHG
FSHYLLSHPSDPLELIVSGSLEDPRPSPTRSVSTAAGPEDQPLMPTGSVP
HSGLRRHWEVLIGVLVVSILLLSLLLFLLLQHWRQGKHRTLAQRQADFQR
PPGAAEPEPKDGGLQRRSSPAADVQGENFCAAVKNTQPEDGVEMDTRQSP
HDEDPQAVTYAKVKHSRPRREMASPPSPLSGEFLDTKDRQAEEDRQMDTE
AAASEAPQDVTYAQLHSFTLRQKATEPPPSQEGASPAEPSVYATLAI

The amino acid (aa) sequence for cynomolgus monkey (“cyno”) ILT3 (NCBI Ref No. XP_015297198) is shown below and includes the predicted signal sequence (underlined residues):

(SEQ ID NO: 6)
MTPPLTVLFCLGLSLGPRTCVQAGPLPKPTVWAEPGSVISWGSPVTIWCQ
GTLDAQEYHLDKEGSPAPWDTQNPLEPRNKAKFSIPSMTQHYAGRYRCYY
HSHPDWSEDSDPLDLVMTGAYSKPILSVLPSPLVTSGESVTLLCQSQSPM
DTFLLFKEGAAHPLPRLRSQHGAQLHWAEFPMGPVTSVHGGTYRCISSRS
FSHYLLSRPSDPVELTVLGSLESPSPSPTRSISAAAGPEDQSLMPTGSDP
QSGLRRHWEVLIGVLVVSILLLSLVFFLLLQHWRQGKHRTSAQRQADFQR
PPGAAEPEPKDGGLQRRSRPAADVQGENPNAAMKDTQPEDGVELDSRQRP
HDEDPQAVTYARVKHSGPRREMASPPSPLSEEFLDTKDTQAEEDRQMDTQ
AATSEAPQDVTYAQLQSLTLRREATEPPPPQKREPSAEPSVYATLAIH

As used herein, reference to amino acid positions of ILT3 refer to the numbering of amino acid sequences comprising the signal sequences.

The present disclosure provides agents (e.g., antibodies) that bind ILT3. In certain embodiments, the anti-ILT3 antibody binds a fragment of ILT3. In certain embodiments, the anti-ILT3 antibody binds within a specific region of ILT3. In certain embodiments, the anti-ILT3 antibody binds within the extracellular domain of ILT3. In certain embodiments, the anti-ILT3 antibody binds within the D1 domain of ILT3. In certain embodiments, the anti-ILT3 antibody binds within the D2 domain of ILT3. In certain embodiments, the anti-ILT3 antibody binds within the D2-stem region of ILT3. In certain embodiments, the anti-ILT3 antibody binds within the junction between D1 and D2 domains of ILT3. In certain embodiments, the anti-ILT3 antibody binds an epitope of ILT3. In certain embodiments, the anti-ILT3 antibody binds a conformational epitope of ILT3. In certain embodiments, the anti-ILT3 antibody does not bind other human LILRB proteins (e.g., ILT2, ILT4, ILT5, or LILRB5). In certain embodiments, the anti-ILT3 antibody does not bind human LILRA proteins (e.g., LILRA1, LILRA2, LILRA4, LILRA5, or LILRA6).

In certain embodiments, the anti-ILT3 antibody binds human ILT3. In certain embodiments, the anti-ILT3 antibody binds cyno ILT3. In certain embodiments, the anti-ILT3 antibody binds human ILT3 and cyno ILT3.

In certain embodiments, the anti-ILT3 antibody binds human ILT3 and has at least one or more of the following properties: (i) binds cyno ILT3; (ii) binds human and cyno ILT3; (iii) does not bind ILT2, ILT4, ILT5, and LILRB5; (iv) does not bind LILRA1, LILRA2, LILRA4, LILRA5, and LILRA6; (v) is an ILT3 antagonist; (vi) inhibits ILT3 activity; (vii) inhibits ILT3 signaling in cells that express ILT3; (viii) inhibits the binding of ILT3 to APOE; (ix) inhibits the binding of ILT3 to fibronectin; (x) inhibits the binding of ILT3 to CNTFR; (xi) inhibits ILT3-induced suppression of myeloid cells; (xii) inhibits ILT3-induced suppression of myeloid cell activity; (xiii) restores FcR activity in myeloid cells that express ILT3; and (xiv) restores the ability of myeloid cells that express ILT3 to respond to chemokines.

In certain embodiments, the anti-ILT3 antibody comprises a VH comprising a VH-CDR1, a VH-CDR2, and a VH-CDR3, and a VL comprising a VL-CDR1, a VL-CDR2, and a VL-CDR3, wherein the VH-CDR1, VH-CDR2, VH-CDR3, VL-CDR1, VL-CDR2 and VL-CDR3 are from any one of the VH and VL sequences of the anti-ILT3 antibodies described herein (e.g., 3A3, 5A7, 12A12, 16C5, 45G10, 48A6, 53F10, or Hz5A7.v5), such as the amino acid sequences depicted in Tables 1-8. In certain embodiments, the anti-ILT3 antibody is a humanized version of an antibody described herein, (e.g., 3A3, 5A7, 12A12, 16C5, 45G10, 48A6, 53F10).

The antibody designated 3A3 comprises a VH sequence that is set forth in SEQ ID NO:109 and a VL sequence that is set forth in SEQ ID NO:110 (see Table 1). In certain embodiments, a humanized 3A3 antibody comprises a VH comprising a VH-CDR1, a VH-CDR2, and a VH-CDR3, and a VL comprising a VL-CDR1, a VL-CDR2, and a VL-CDR3, wherein the VH-CDR1, VH-CDR2, and VH-CDR3 are from the amino acid sequence of SEQ ID NO:109, and the VL-CDR1, VL-CDR2 and VL-CDR3 are from the amino acid sequence of SEQ ID NO:110. The antibody designated 5A7 comprises a VH sequence that is set forth in SEQ ID NO:111 and a VL sequence that is set forth in SEQ ID NO:112 (see Table 2). In certain embodiments, a humanized 5A7 antibody comprises a VH comprising a VH-CDR1, a VH-CDR2, a VH-CDR3, and a VL comprising a VL-CDR1, a VL-CDR2, and a VL-CDR3, wherein the VH-CDR1, VH-CDR2, and VH-CDR3 are from the amino acid sequence of SEQ ID NO:111, and the VL-CDR1, VL-CDR2 and VL-CDR3 are from the amino acid sequence of SEQ ID NO:112. The antibody designated 12A12 comprises a VH sequence that is set forth in SEQ ID NO:113 and a VL sequence that is set forth in SEQ ID NO:114 (see Table 3). In certain embodiments, a humanized 12A12 comprises a VH comprising a VH-CDR1, a VH-CDR2, a VH-CDR3, and a VL comprising a VL-CDR1, a VL-CDR2, and a VL-CDR3, wherein the VH-CDR1, VH-CDR2, and VH-CDR3 are from the amino acid sequence of SEQ ID NO:113, and the VL-CDR1, VL-CDR2 and VL-CDR3 are from the amino acid sequence of SEQ ID NO:114. The antibody designated 16C5 comprises a VH sequence that is set forth in SEQ ID NO:115 and a VL sequence that is set forth in SEQ ID NO:116 (see Table 4). In certain embodiments, a humanized 16C5 antibody comprises a VH comprising a VH-CDR1, a VH-CDR2, a VH-CDR3, and a VL comprising a VL-CDR1, a VL-CDR2, and a VL-CDR3, wherein the VH-CDR1, VH-CDR2, and VH-CDR3 are from the amino acid sequence of SEQ ID NO:115, and the VL-CDR1, VL-CDR2 and VL-CDR3 are from the amino acid sequence of SEQ ID NO:116. The antibody designated 45G10 comprises a VH sequence that is set forth in SEQ ID NO:117 and a VL sequence that is set forth in SEQ ID NO:118 (see Table 5). In certain embodiments, a humanized 45G10 antibody comprises a VH comprising a VH-CDR1, a VH-CDR2, a VH-CDR3, and a VL comprising a VL-CDR1, a VL-CDR2, and a VL-CDR3, wherein the VH-CDR1, VH-CDR2, and VH-CDR3 are from the amino acid sequence of SEQ ID NO:117, and the VL-CDR1, VL-CDR2 and VL-CDR3 are from the amino acid sequence of SEQ ID NO:118. The antibody designated 48A6 comprises a VH sequence that is set forth in SEQ ID NO:119 and a VL sequence that is set forth in SEQ ID NO:120 (see Table 6). In certain embodiments, a humanized 48A6 antibody comprises a VH comprising a VH-CDR1, a VH-CDR2, a VH-CDR3, and a VL comprising a VL-CDR1, a VL-CDR2, and a VL-CDR3, wherein the VH-CDR1, VH-CDR2, and VH-CDR3 are from the amino acid sequence of SEQ ID NO:119, and the VL-CDR1, VL-CDR2 and VL-CDR3 are from the amino acid sequence of SEQ ID NO:120. The antibody designated 53F10 comprises a VH sequence that is set forth in SEQ ID NO:121 and a VL sequence that is set forth in SEQ ID NO:122 (see Table 7). In certain embodiments, a humanized 53F10 antibody comprises a VH comprising a VH-CDR1, a VH-CDR2, a VH-CDR3, and a VL comprising a VL-CDR1, a VL-CDR2, and a VL-CDR3, wherein the VH-CDR1, VH-CDR2, and VH-CDR3 are from the amino acid sequence of SEQ ID NO:121, and the VL-CDR1, VL-CDR2 and VL-CDR3 are from the amino acid sequence of SEQ ID NO:122. The antibody designated Hz5A7.v5 comprises a VH set forth in SEQ ID NO:123 and a VL sequence that is set forth in SEQ ID NO:124 (see Table 8).

TABLE 1
Antibody 3A3 Sequences

| | Exemplary | Chothia | AbM | Kabat | Contact |
|---|---|---|---|---|---|
| VH- | GFSLTSY | GFSLTSY | GFSLTSY | SYGVH | TSYGVH |
| CDR1 | GVH | (SEQ ID | GVH | (SEQ ID | (SEQ ID |
| | (SEQ ID | NO: 17) | (SEQ ID | NO: 20) | NO: 21) |
| | NO: 11) | | NO: 11) | | |
| VH- | VIWPGGT | WPGGT | VIWPGGT | VIWPGGT | WLGVIWP |
| CDR2 | INYNSAL | (SEQ ID | IN | INY | GGTIN |
| | MS | NO: 18) | (SEQ ID | NSALMS | (SEQ ID |
| | (SEQ ID | | NO: 19) | (SEQ ID | NO: 22) |
| | NO: 12) | | | NO: 12) | |
| VH- | DKYDGGW | DKYDGGW | DKYDGGW | DKYDGGW | ASDKYDG |
| CDR3 | FAY | FAY | FAY | FAY | GWFA |
| | (SEQ ID | (SEQ ID | (SEQ ID | (SEQ ID | (SEQ ID |
| | NO: 13) | NO: 13) | NO: 13) | NO: 13) | NO: 23) |
| VL- | KASQNVR | KASQNVR | KASQNVR | KASQNVR | RTAVAWY |
| CDR1 | TAVA | TAVA | TAVA | TAVA | (SEQ ID |
| | (SEQ ID | (SEQ ID | (SEQ ID | (SEQ ID | NO: 24) |
| | NO: 14) | NO: 14) | NO: 14) | NO: 14) | |
| VL- | LASNRHT | LASNRHT | LASNRHT | LASNRHT | ALIYLAS |
| CDR2 | (SEQ ID | (SEQ ID | (SEQ ID | (SEQ ID | NRH |
| | NO: 15) | NO: 15) | NO: 15) | NO: 15) | (SEQ ID |
| | | | | | NO: 25) |
| VL- | LQHLNYP | LQHLNYP | LQHLNYP | LQHLNYP | LQHLNYPL |
| CDR3 | LT | LT | LT | LT | (SEQ ID |
| | (SEQ ID | (SEQ ID | (SEQ ID | (SEQ ID | NO: 26) |
| | NO: 16) | NO: 16) | NO: 16) | NO: 16) | |

3A3 Heavy chain variable region (SEQ ID NO: 109)
QVQLKESGPGLVAPSQSLSITCTVSGISLTSYGVHWVRQPPGKGLEWLG
VIWPGGTINYNSALMSRLSISKDNSKSQVFLKLNSLQTDDTAMYYCASD
KYDGGWFAYWGQGTLVTVSA
3A3 Light chain variable region (SEQ ID NO: 110)
DIVMTQSQKFMSTSVGDRVSITCKASQNVRTAVAWYQQKPGQSPEALIY
LASNRHTGVPDRFTGSGSGTDFSLSISNVQSEDLADYFCLQHLNYPLTF
GSGTKLEIK

TABLE 2
Antibody 5A7 Sequences

| | Exemplary | Chothia | AbM | Kabat | Contact |
|---|---|---|---|---|---|
| VH- | GFTFSSY | GFTFSSY | GFTFSSY | SYGMS | SSYGMS |
| CDR1 | GMS | (SEQ ID | GMS | (SEQ ID | (SEQ ID |
| | (SEQ ID | NO: 33) | (SEQ ID | NO: 36) | NO: 37) |
| | NO: 27) | | NO: 27) | | |
| VH- | TISGGGS | SGGGSY | TISGGGS | TISGGGS | WVATISG |
| CDR2 | YTNYPDS | (SEQ ID | YTN | YTNYPDS | GGSYTN |
| | VKG | NO: 34) | (SEQ ID | VKG | (SEQ ID |
| | (SEQ ID | | NO: 35) | (SEQ ID | NO: 38) |
| | NO: 28) | | | NO: 28) | |
| VH- | REWRMTL | REWRMTL | REWRMTL | REWRMTL | ARREWRM |
| CDR3 | YAMDY | YAMDY | YAMDY | YAMDY | TLYAMD |
| | (SEQ ID | (SEQ ID | (SEQ ID | (SEQ ID | (SEQ ID |
| | NO: 29) | NO: 29) | NO: 29) | NO: 29) | NO: 39) |
| VL- | RASESVD | RASESVD | RASESVD | RASESVD | DSYGNSF |
| CDR1 | SYGNSFM | SYGNSFM | SYGNSFM | SYGNSFM | MHWY |
| | H | H | H | H | (SEQ ID |
| | (SEQ ID | (SEQ ID | (SEQ ID | (SEQ ID | NO: 40) |
| | NO: 30) | NO: 30) | NO: 30) | NO: 30) | |
| VL- | LTSNLES | LTSNLES | LTSNLES | LTSNLES | LLIYLTS |
| CDR2 | (SEQ ID | (SEQ ID | (SEQ ID | (SEQ ID | NLE |
| | NO: 31) | NO: 31) | NO: 31) | NO: 31) | (SEQ ID |
| | | | | | NO: 41) |
| VL- | QQNNEDP | QQNNEDP | QQNNEDP | QQNNEDP | QQNNED |
| CDR3 | FT | FT | FT | FT | PF |
| | (SEQ ID | (SEQ ID | (SEQ ID | (SEQ ID | (SEQ ID |
| | NO: 32) | NO: 32) | NO: 32) | NO: 32) | NO: 42) |

5A7 Heavy chain variable region (SEQ ID NO: 111)
EVKLVESGGGLVKPGGSLKLSCAASGFTFSSYGMSWVRQTPEKRLEWVAT
ISGGGSYTNYPDSVKGRLTISRDNAKKNLYLEMSSLRSEDTALYYCARRE
WRMTLYAMDYWGQGTSVTVSS
5A7 Light chain variable region (SEQ ID NO: 112)
NIVLTQSPASLAVSLGQRATISCRASESVDSYGNSFMHWYQQKPGQAPKL
LIYLTSNLESGVPARFSGSGSRTDFTLTIDPVEADDAATYYCQQNNEDPF
TFGSGTKLEIK

TABLE 3
Antibody 12A12 Sequences

| | Exemplary | Chothia | AbM | Kabat | Contact |
|---|---|---|---|---|---|
| VH- | GYTFTDY | GYTFTDY | GYTFTDY | DYNMD | TDYNMD |
| CDR1 | NMD | | NMD | (SEQ ID | (SEQ ID |
| | (SEQ ID | (SEQ ID | (SEQ ID | NO: 52) | NO: 53) |
| | NO: 43) | NO: 49) | NO: 43) | | |
| VH- | YIYPNNG | YPNNGG | YIYPNNG | YIYPNNG | WIGYIYP |
| CDR2 | GTGYNQK | (SEQ ID | GTG | GTGYNQK | NNGGTG |
| | FNS | NO: 50) | (SEQ ID | FN | (SEQ ID |
| | (SEQ ID | | NO: 51) | (SEQ ID | NO: 54) |
| | NO: 44) | | | NO: 44) | |
| VH- | SPYYDYV | SPYYDYV | SPYYDYV | SPYYDYVG | ASSPYYD |
| CDR3 | GSYAMDY | GSYAMDY | GSYAMDY | SYAMDY | YVGSYAM |
| | (SEQ ID | (SEQ ID | (SEQ ID | (SEQ ID | D |
| | NO: 45) | NO: 45) | NO: 45) | NO: 45) | (SEQ ID |
| | | | | | NO: 55) |
| VL- | TASSSVS | TASSSVS | TASSSVS | TASSSVS | SSSYLHW |
| CDR1 | SSYLH | SSYLH | SSYLH | SSYLH | Y |
| | (SEQ ID | (SEQ ID | (SEQ ID | (SEQ ID | (SEQ ID |
| | NO: 46) | NO: 46) | NO: 46) | NO: 46) | NO: 56) |
| VL- | STSNLAS | STSNLAS | STSNLAS | STSNLAS | LWIYSTS |
| CDR2 | (SEQ ID | (SEQ ID | (SEQ ID | (SEQ ID | NLA |
| | NO: 47) | NO: 47) | NO: 47) | NO: 47) | (SEQ ID |
| | | | | | NO: 57) |
| VL- | HQYHRSP | HQYHRSP | HQYHRSP | HQYHRSP | HQYHRSP |
| CDR3 | RT | RT | RT | RT | R |
| | (SEQ ID | (SEQ ID | (SEQ ID | (SEQ ID | (SEQ ID |
| | NO: 48) | NO: 48) | NO: 48) | NO: 48) | NO: 58) |

12A12 Heavy chain variable region (SEQ ID NO: 113)
EVQLQQSGPELVKPGASVKISCKASGYTFTDYNMDWVKQSHGKSLEWIGY
IYPNNGGTGYNQKFNSKATLTVDKSSSTAYMELHSLTSEDSAVYYCASSP
YYDYVGSYAMDYWGQGTSVTVSS
12A12 Light chain variable region (SEQ ID NO: 114)
QIVLTQSPAIMSASLGERVTMTCTASSSVSSSYLHWYQQKPGSSPKLWIY
STSNLASGVPARFSGSGSGTSYSLTISSMEAEDAATYYCHQYHRSPRTFG
GGTKLEIK

TABLE 4
Antibody 16C5 Sequences

| | Exemplary | Chothia | AbM | Kabat | Contact |
|---|---|---|---|---|---|
| VH- | GYTFTDY | GYTFTDY | GYTFTDY | DYNMD | TDYNMD |
| CDR1 | NMD | (SEQ ID | NMD | (SEQ ID | (SEQ ID |
| | (SEQ ID | NO: 49) | (SEQ ID | NO: 52) | NO: 53) |
| | NO: 43) | | NO: 43) | | |
| VH- | YIYPSNG | YPSNGG | YIYPSNG | YIYPSNG | WIGYIYPS |
| CDR2 | GTGYNQK | (SEQ ID | GTG | GTGYNQK | NGGTG |
| | FK | NO: 64) | (SEQ ID | FKS | (SEQ ID |
| | (SEQ ID | | NO: 65) | (SEQ ID | NO: 66) |
| | NO: 59) | | | NO: 59) | |
| VH- | VPYYDYL | VPYYDYL | VPYYDYL | VPYYDYL | ARVPYYD |
| CDR3 | YYYAMDY | YYYAMDY | YYYAMD | YYYAMDY | YLYYYAM |
| | (SEQ ID | (SEQ ID | (SEQ ID | (SEQ ID | D |
| | NO: 60) | NO: 60) | NO: 60) | NO: 60) | (SEQ ID |
| | | | | | NO: 67) |
| VL- | RASSSVS | RASSSVS | RASSSVS | RASSSVS | SFMHWY |
| CDR1 | FMH | FMH | FMH | FMH | (SEQ ID |
| | (SEQ ID | (SEQ ID | (SEQ ID | (SEQ ID | NO: 68) |
| | NO: 61) | NO: 61) | NO: 61) | NO: 61) | |
| VL- | ATSNLAS | ATSNLAS | ATSNLAS | ATSNLAS | PWIYATS |
| CDR2 | (SEQ ID | (SEQ ID | (SEQ ID | (SEQ ID | NLA |
| | NO: 62) | NO: 62) | NO: 62) | NO: 62) | (SEQ ID |
| | | | | | NO: 69) |
| VL- | QQWSTNP | QQWSTNP | QQWSTNP | QQWSTNP | QQWSTNP |
| CDR3 | YMYT | YMYT | YMYT | YMYT | YMY |
| | (SEQ ID | (SEQ ID | (SEQ ID | (SEQ ID | (SEQ ID |
| | NO: 63) | NO: 63) | NO: 63) | NO: 63) | NO: 70) |

16C5 Heavy chain variable region (SEQ ID NO: 115)
EVQLQQSGPELVKPGASVKISCKASGYTFTDYNMDWVKQSHGKSLEWIGY
IYPSNGGTGYNQKFKSKATLTVDKSSNTAYMELHSLTSEDSAVYYCARVP
YYDYLYYYAMDYWGQGTSVTVSS
16C5 Light chain variable region (SEQ ID NO: 116)
QIVLSQSPAILSASPGEKVTMACRASSSVSFMHWYQQKPGSSPQPWIYAT
SNLASGVPARFSGSGSGTSYSLTISRVEAEDAATYYCQQWSTNPYMYTFG
GGTKLEIK

TABLE 5
Antibody 45G10 Sequences

| | Exemplary | Chothia | AbM | Kabat | Contact |
|---|---|---|---|---|---|
| VH- | GFTFSDY | GFTFSDY | GFTFSDY | DYGMH | SDYGMH |
| CDR1 | GMH | (SEQ ID | GMH | (SEQ ID | (SEQ ID |
| | (SEQ ID | NO: 77) | (SEQ ID | NO: 80) | NO: 81) |
| | NO: 71) | | NO: 71) | | |
| VH- | YIFSGSS | FSGSST | YIFSGSS | YIFSGSS | WVAYIFS |
| CDR2 | TIYYADT | (SEQ ID | TIY | TIYYADT | GSSTIY |
| | VKG | NO: 78) | (SEQ ID | VKG | (SEQ ID |
| | (SEQ ID | | NO: 79) | (SEQ ID | NO: 82) |
| | NO: 72) | | | NO: 72) | |
| VH- | ADGRGAM | ADGRGAM | ADGRGAM | ADGRGAM | ARADGRG |
| CDR3 | DY | DY | DY | DY | AMD |
| | (SEQ ID | (SEQ ID | (SEQ ID | (SEQ ID | (SEQ ID |
| | NO: 73) | NO: 73) | NO: 73) | NO: 73) | NO: 83) |
| VL- | RASQDIS | RASQDIS | RASQDIS | RASQDIS | SKFLNWY |
| CDR1 | KFLN | KFLN | KFLN | KFLN | (SEQ ID |
| | (SEQ ID | (SEQ ID | (SEQ ID | (SEQ ID | NO: 84) |
| | NO: 74) | NO: 74) | NO: 74) | NO: 74) | |
| VL- | YTSRLHS | YTSRLHS | YTSRLHS | YTSRLHS | LLIYYTS |
| CDR2 | (SEQ ID | (SEQ ID | (SEQ ID | (SEQ ID | RLH |
| | NO: 75) | NO: 75) | NO: 75) | NO: 75) | (SEQ ID |
| | | | | | NO: 85) |
| VL- | QQGNTLP | QQGNTLP | QQGNTLP | QQGNTLP | QQGNTLP |
| CDR3 | WT | WT | WT | WT | W |
| | (SEQ ID | (SEQ ID | (SEQ ID | (SEQ ID | (SEQ ID |
| | NO: 76) | NO: 76) | NO: 76) | NO: 76) | NO: 86) |

45G10 Heavy chain variable region (SEQ ID NO: 117)
EVQLVESGGGLVKPGGSLKLSCAASGFTFSDYGMHWVRQAPEKGLEWVAY
IFSGSSTIYYADTVKGRFTISRDNAKNTLFLQMTSLRSEDTAMYYCARAD
GRGAMDYWGQGTSVTVSS
45G10 Light chain variable region (SEQ ID NO: 118)
DVQMTQTTSSLSASLGDRVTISCRASQDISKFLNWYQQKPDGTVTLLIYY
TSRLHSGVPSRFSGSGSGTDYSLTISNLDQEDIATYFCQQGNTLPWTFGG
GTKLEIK

TABLE 6
Antibody 48A6 Sequences

| | Exemplary | Chothia | AbM | Kabat | Contact |
|---|---|---|---|---|---|
| VH- | GFTFSSY | GFTFSSY | GFTFSSY | SYGMS | SSYGMS |
| CDR1 | GMS | (SEQ ID | GMS | (SEQ ID | (SEQ ID |
| | (SEQ ID | NO: 33) | (SEQ ID | NO: 36) | NO: 37) |
| | NO: 27) | | NO: 27) | | |
| VH- | TISSGGT | SSG | TISSG | TISSGGT | WVATISS |
| CDR2 | YTFYPDS | GTY | GTYT | YTFYPDS | GGTYTF |
| | VKG | (SEQ ID | F | VKG | (SEQ ID |
| | (SEQ ID | NO: 2) | (SEQ ID | (SEQ ID | NO: 4) |
| | NO: 87) | | NO: 3) | NO: 87) | |
| VH- | RGWLLHY | RGWLLHY | RGWLLHY | RGWLLHY | ARRGWLL |
| CDR3 | YAMDY | YAMDY | YAMDY | YAMDY | HYYAMD |
| | (SEQ ID | (SEQ ID | (SEQ ID | (SEQ ID | (SEQ ID |
| | NO: 88) | NO: 88) | NO: 88) | NO: 88) | NO: 5) |
| VL- | RPSESVD | RPSESVD | RPSESVD | RPSESVD | DSFGNSF |
| CDR1 | SFGNSFM | SFGNSFM | SFGNSFM | SFGNSFM | MHWF |
| | H | H | H | H | (SEQ ID |
| | (SEQ ID | (SEQ ID | (SEQ ID | (SEQ ID | NO: 6) |
| | NO: 89) | NO: 89) | NO: 89) | NO: 89) | |
| VL- | LSSKL | LSSKL | LSSK | LSSKL | LLIYLSS |
| CDR2 | ES | ES | LES | ES | KLE |
| | (SEQ ID | (SEQ ID | (SEQ ID | (SEQ ID | (SEQ ID |
| | NO: 90) | NO: 90) | NO: 90) | NO: 90) | NO: 7) |
| VL- | QQHNEDP | QQHNEDP | QQHNEDP | QQHNEDP | QQHNEDP |
| CDR3 | FT | FT | FT | FT | F |
| | (SEQ ID | (SEQ ID | (SEQ ID | (SEQ ID | (SEQ ID |
| | NO: 91) | NO: 91) | NO: 91) | NO: 91) | NO: 8) |

48A6 Heavy chain variable region
(SEQ ID NO: 119)
EVQLVESGGDLMKPGGSLKLSCAASGFTFSSYGMSWVRQTPDKRLEWVA
TISSGGTYTFYPDSVKGRFTISRDNAKNTLYLQMSSLKSEDTAMYYCAR
RGWLLHYYAMDYWGQGTSVTVSS
48A6 Light chain variable region
(SEQ ID NO: 120)
NIVLTQSPASLAVSLGQRATISCRPSESVDSFGNSFMHWFQQKPGQP
PKLLIYLSSKLESGVPARFSGSGSRTDFTLTIDPVEADDAATYYCQQ
HNEDPFTFGSGTKLEIK

TABLE 7
Antibody 53F10 Sequences

| | Exemplary | Chothia | AbM | Kabat | Contact |
|---|---|---|---|---|---|
| VH- | GFTFSDY | GFTFS | GFTFSDY | DYGM | SDYGM |
| CDR1 | GMH | DY | GMH | H | H |
| | (SEQ ID | (SEQ ID | (SEQ ID | (SEQ ID | (SEQ ID |
| | NO: 71) | NO: 77) | NO: 71) | NO: 80) | NO: 81) |
| VH- | YISTGII | STGIIT | YISTGII | YISTGII | WVAYIST |
| CDR2 | TVYYADT | (SEQ ID | TVY | TVYYADT | GIITVY |
| | VKG | NO: 101) | (SEQ ID | VKG | (SEQ ID |
| | (SEQ ID | | NO: 102) | (SEQ ID | NO: 103) |
| | NO: 99) | | | NO: 99) | |
| VH- | ADGRGAM | ADGRGAM | ADGRGAM | ADGRGAM | ARADGRG |
| CDR3 | DY | DY | DY | DY | AMD |
| | (SEQ ID | (SEQ ID | (SEQ ID | (SEQ ID | (SEQ ID |
| | NO: 73) | NO: 73) | NO: 73) | NO: 73) | NO: 83) |
| VL- | RASQDIS | RASQDIS | RASQDIS | RASQDIS | SNFLNWY |
| CDR1 | NFLN | NFLN | NFLN | NFLN | (SEQ ID |
| | (SEQ ID | (SEQ ID | (SEQ ID | (SEQ ID | NO: 104) |
| | NO: 100) | NO: 100) | NO: 100) | NO: 100) | |
| VL- | YTSRL | YTSRL | YTSRL | YTSRL | LLIYYTS |
| CDR2 | HS | HS | HS | HS | RLH |
| | (SEQ ID | (SEQ ID | (SEQ ID | (SEQ ID | (SEQ ID |
| | NO: 75) | NO: 75) | NO: 75) | NO: 75) | NO: 85) |
| VL- | QQGNTLP | QQGNTLP | QQGNTLP | QQGNTLP | QQGNTLP |
| CDR3 | WT | WT | WT | WT | W |
| | (SEQ ID | (SEQ ID | (SEQ ID | (SEQ ID | (SEQ ID |
| | NO: 76) | NO: 76) | NO: 76) | NO: 76) | NO: 86) |

53F10 Heavy chain variable region
(SEQ ID NO: 121)
EVQVVESGG GLVKPGGSLKLSCAASGFTFSDYGMHWVRQAPEKGLEWVAY
ISTGIITVYYADTVKGRFTMSRDNAKNTLFLQMTSLRSEDTAIYYCARADG
RGAMDYWGQGTSVIVSS
53F10 Light chain variable region
(SEQ ID NO: 122)
DIQMTQTTSSLSASLGDRVTISCRASQDISNFLNWYQQKPDGTVTLLIYY
TSRLHSGVPSRFSGSGSGTDYSLTISNLEQEDFATYFCQQGNTLPWTFGG
GTKLEIK

TABLE 8
Antibody Hz5A7.v5 Sequences

| | Exemplary | Chothia | AbM | Kabat | Contact |
|---|---|---|---|---|---|
| VH- | GFTFSSY | GFTFSSY | GFTFSSY | SYGMS | SSYGMS |
| CDR1 | GMS | (SEQ ID | GMS | (SEQ ID | (SEQ ID |
| | (SEQ ID | NO: | (SEQ ID | NO: | NO: |
| | NO: | 33) | NO: | 36) | 37) |
| | 27) | | 27) | | |
| VH- | TISGGGS | SGGGSY | TISGGGS | TISGGGS | WVATISG |
| CDR2 | YTNYPDS | (SEQ ID | YTN | YTNYPDS | GGSYTN |
| | VKG | NO: | (SEQ ID | VK | (SEQ ID |
| | (SEQ ID | 34) | NO: | (SEQ ID | NO: |
| | NO: | | 35) | NO: | 38) |
| | 28) | | | 28) | |
| VH- | REWRYTL | REWRYTL | REWRYTL | REWRYTL | ARREWRY |
| CDR3 | YAMDY | YAMDY | YAMDY | YAMDY | TLYAMD |
| | (SEQ ID | (SEQ ID | (SEQ ID | (SEQ ID | (SEQ ID |
| | NO: | NO: | NO: | NO: | NO: |
| | 105) | 105) | 105) | 105) | 107) |
| VL- | RASESVE | RASESVE | RASESVE | RASESVE | ESYGSSF |
| CDR1 | SYGSSFM | SYGSSFM | SYGSSFM | SYGSSFM | MHWY |
| | H | H | H | H | (SEQ ID |
| | (SEQ ID | (SEQ ID | (SEQ ID | (SEQ ID | NO: |
| | NO: | NO: | NO: | NO: | 108) |
| | 106) | 106) | 106) | 106) | |
| VL- | LTSNLES | LTSNLES | LTSNLES | LTSNLES | LLIYLTS |
| CDR2 | (SEQ ID | (SEQ ID | (SEQ ID | (SEQ ID | NLE |
| | NO: | NO: | NO: | NO: | (SEQ ID |
| | 31) | 31) | 31) | 31) | NO: |
| | | | | | 41) |
| VL- | QQNNEDP | QQNNEDP | QQNNEDP | QQNNEDP | QQNNEDP |
| CDR3 | FT | FT | FT | FT | F |
| | (SEQ ID | (SEQ ID | (SEQ ID | (SEQ ID | (SEQ ID |
| | NO: | NO: | NO: | NO: | NO: |
| | 32) | 32) | 32) | 32) | 42) |

Hz5A7.v5 Heavy chain variable region
(SEQ ID NO: 123)
EVQLVESGGGLVQPGGSLRLSCAASGFTFSSYGMSWVRQAPGKGLEWVAT
ISGGGSYTNYPDSVKGRFTISRDNAKNSLYLQMNSLRAEDTAVYYCARRE
WRYTLYAMDYWGQGTTVTVSS
Hz5A7.v5 Light chain variable region
(SEQ ID NO: 124)
DIQLTQSPSFLSASVGDRVTITCRASESVESYGSSFMHWYQQKPGKAPKL
LIYLTSNLESGVPSRFSGSGSGTEFTLTCISSLQPEDFATYYQQNNEDPF
TFGQGTKLEIK

In certain embodiments, the anti-ILT3 antibody comprises (i) a VH comprising a VH-CDR1, a VH-CDR2, and a VH-CDR3 defined by any one of the CDR definitions from Table 1; and (ii) a VL comprising a VL-CDR1, a VL-CDR2, and a VL-CDR3 defined by any one of the CDR definitions from Table 1.

In certain embodiments, the anti-ILT3 antibody comprises (i) a VH comprising a VH-CDR1, a VH-CDR2, and a VH-CDR3 defined by any one of the CDR definitions from Table 2; and (ii) a VL comprising a VL-CDR1, a VL-CDR2, and a VL-CDR3 defined by any one of the CDR definitions from Table 2.

In certain embodiments, the anti-ILT3 antibody comprises (i) a VH comprising a VH-CDR1, a VH-CDR2, and a VH-CDR3 defined by any one of the CDR definitions from Table 3; and (ii) a VL comprising a VL-CDR1, a VL-CDR2, and a VL-CDR3 defined by any one of the CDR definitions from Table 3.

In certain embodiments, the anti-ILT3 antibody comprises (i) a VH comprising a VH-CDR1, a VH-CDR2, and a VH-CDR3 defined by any one of the CDR definitions from Table 4; and (ii) a VL comprising a VL-CDR1, a VL-CDR2, and a VL-CDR3 defined by any one of the CDR definitions from Table 4.

In certain embodiments, the anti-ILT3 antibody comprises (i) a VH comprising a VH-CDR1, a VH-CDR2, and a VH-CDR3 defined by any one of the CDR definitions from Table 5; and (ii) a VL comprising a VL-CDR1, a VL-CDR2, and a VL-CDR3 defined by any one of the CDR definitions from Table 5.

In certain embodiments, the anti-ILT3 antibody comprises (i) a VH comprising a VH-CDR1, a VH-CDR2, and a VH-CDR3 defined by any one of the CDR definitions from Table 6; and (ii) a VL comprising a VL-CDR1, a VL-CDR2, and a VL-CDR3 defined by any one of the CDR definitions from Table 6.

In certain embodiments, the anti-ILT3 antibody comprises (i) a VH comprising a VH-CDR1, a VH-CDR2, and a VH-CDR3 defined by any one of the CDR definitions from Table 7; and (ii) a VL comprising a VL-CDR1, a VL-CDR2, and a VL-CDR3 defined by any one of the CDR definitions from Table 7.

In certain embodiments, the anti-ILT3 antibody comprises (i) a VH comprising a VH-CDR1, a VH-CDR2, and a VH-CDR3 defined by any one of the CDR definitions from Table 8; and (ii) a VL comprising a VL-CDR1, a VL-CDR2, and a VL-CDR3 defined by any one of the CDR definitions from Table 8.

In certain embodiments, the anti-ILT3 antibody comprises (i) a VH comprising the VH-CDR1, the VH-CDR2, and the VH-CDR3 from the amino acid sequence set forth in SEQ ID NO:109; and (ii) a VL comprising the VL-CDR1, the VL-CDR2, and the VL-CDR3 from the amino acid sequence set forth in SEQ ID NO:110 (i.e., the six CDRs of antibody 3A3 of Table 1).

In certain embodiments, the anti-ILT3 antibody comprises (i) a VH comprising the VH-CDR1, the VH-CDR2, and the VH-CDR3 from the amino acid sequence set forth in SEQ ID NO:111; and (ii) a VL comprising the VL-CDR1, the VL-CDR2, and the VL-CDR3 from the amino acid sequence set forth in SEQ ID NO:112 (i.e., the six CDRs of antibody 5A7 of Table 2).

In certain embodiments, the anti-ILT3 antibody comprises (i) a VH comprising the VH-CDR1, the VH-CDR2, and the VH-CDR3 from the amino acid sequence set forth in SEQ ID NO:113; and (ii) a VL comprising the VL-CDR1, the VL-CDR2, and the VL-CDR3 from the amino acid sequence set forth in SEQ ID NO:114 (i.e., the six CDRs of antibody 12A12 of Table 3).

In certain embodiments, the anti-ILT3 antibody comprises (i) a VH comprising the VH-CDR1, the VH-CDR2, and the VH-CDR3 from the amino acid sequence set forth in SEQ ID NO:115; and (ii) a VL comprising the VL-CDR1, the VL-CDR2, and the VL-CDR3 from the amino acid sequence set forth in SEQ ID NO:116 (i.e., the six CDRs of antibody 16C5 of Table 4).

In certain embodiments, the anti-ILT3 antibody comprises (i) a VH comprising the VH-CDR1, the VH-CDR2, and the VH-CDR3 from the amino acid sequence set forth in SEQ ID NO:117; and (ii) a VL comprising the VL-CDR1, the VL-CDR2, and the VL-CDR3 from the amino acid sequence set forth in SEQ ID NO:118 (i.e., the six CDRs of antibody 45G10 of Table 5).

In certain embodiments, the anti-ILT3 antibody comprises (i) a VH comprising the VH-CDR1, the VH-CDR2, and the VH-CDR3 from the amino acid sequence set forth in SEQ ID NO:119; and (ii) a VL comprising the VL-CDR1, the VL-CDR2, and the VL-CDR3 from the amino acid sequence set forth in SEQ ID NO:120 (i.e., the six CDRs of antibody 48A6 of Table 6).

In certain embodiments, the anti-ILT3 antibody comprises (i) a VH comprising the VH-CDR1, the VH-CDR2, and the VH-CDR3 from the amino acid sequence set forth in SEQ ID NO:121; and (ii) a VL comprising the VL-CDR1, the VL-CDR2, and the VL-CDR3 from the amino acid sequence set forth in SEQ ID NO:122 (i.e., the six CDRs of antibody 53F10 of Table 7).

In certain embodiments, the anti-ILT3 antibody comprises (i) a VH comprising the VH-CDR1, the VH-CDR2, and the VH-CDR3 from the amino acid sequence set forth in SEQ ID NO:123; and (ii) a VL comprising the VL-CDR1, the VL-CDR2, and the VL-CDR3 from the amino acid sequence set forth in SEQ ID NO:124 (i.e., the six CDRs of antibody Hz5A7.v5 of Table 8).

In certain embodiments, the CDRs of the anti-ILT3 antibody are defined according to the Exemplary designation. In certain embodiments, the anti-ILT3 antibody comprises: (i) a VH comprising a VH-CDR1 comprising the amino acid sequence set forth in SEQ ID NO:11, a VH-CDR2 comprising the amino acid sequence set forth in SEQ ID NO:12, and a VH-CDR3 comprising the amino acid sequence set forth in SEQ ID NO:13; and (ii) a VL comprising a VL-CDR1 comprising the amino acid sequence set forth in SEQ ID NO:14, a VL-CDR2 comprising the amino acid sequence set forth in SEQ ID NO:15, and a VL-CDR3 comprising the amino acid sequence set forth in SEQ ID NO:16. In certain embodiments, the anti-ILT3 antibody comprises: (i) a VH comprising a VH-CDR1 comprising the amino acid sequence set forth in SEQ ID NO:27, a VH-CDR2 comprising the amino acid sequence set forth in SEQ ID NO:28, and a VH-CDR3 comprising the amino acid sequence set forth in SEQ ID NO:29; and (ii) a VL comprising a VL-CDR1 comprising the amino acid sequence set forth in SEQ ID NO:30, a VL-CDR2 comprising the amino acid sequence set forth in SEQ ID NO:31, and a VL-CDR3 comprising the amino acid sequence set forth in SEQ ID NO:32. In certain embodiments, the anti-ILT3 antibody comprises: (i) a VH comprising a VH-CDR1 comprising the amino acid sequence set forth in SEQ ID NO:43, a VH-CDR2 comprising the amino acid sequence set forth in SEQ ID NO:44, and a VH-CDR3 comprising the amino acid sequence set forth in SEQ ID NO:45; and (ii) a VL comprising a VL-CDR1 comprising the amino acid sequence set forth in SEQ ID NO:46, a VL-CDR2 comprising the amino acid sequence set forth in SEQ ID NO:47, and a VL-CDR3 comprising the amino acid sequence set forth in SEQ ID NO:48. In certain embodiments, the anti-ILT3 antibody comprises: (i) a VH comprising a VH-CDR1 comprising the amino acid sequence set forth in SEQ ID NO:43, a VH-CDR2 comprising the amino acid sequence set forth in SEQ ID NO:59, and a VH-CDR3 comprising the amino acid sequence set forth in SEQ ID NO:60; and (ii) a VL comprising a VL-CDR1 comprising the amino acid sequence set forth in SEQ ID NO:61, a VL-CDR2 comprising the amino acid sequence set forth in SEQ ID NO:62, and a VL-CDR3 comprising the amino acid sequence set forth in SEQ ID NO:63. In certain embodiments, the anti-ILT3 antibody comprises: (i) a VH comprising a VH-CDR1 comprising the amino acid sequence set forth in SEQ ID NO:71, a VH-CDR2 comprising the amino acid sequence set forth in SEQ ID NO:72, and a VH-CDR3 comprising the amino acid sequence set forth in SEQ ID NO:73; and (ii) a VL comprising a VL-CDR1 comprising the amino acid sequence set forth in SEQ ID NO:74, a VL-CDR2 comprising the amino acid sequence set forth in SEQ ID NO:75, and a VL-CDR3 comprising the amino acid sequence set forth in SEQ ID NO:76. In certain embodiments, the anti-ILT3 antibody comprises: (i) a VH comprising a VH-CDR1 comprising the amino acid sequence set forth in SEQ ID NO:27, a VH-CDR2 comprising the amino acid sequence set forth in SEQ ID NO:87, and a VH-CDR3 comprising the amino acid sequence set forth in SEQ ID NO:88; and (ii) a VL comprising a VL-CDR1 comprising the amino acid sequence set forth in SEQ ID NO:89, a VL-CDR2 comprising the amino acid sequence set forth in SEQ ID NO:90, and a VL-CDR3 comprising the amino acid sequence set forth in SEQ ID NO:91. In certain embodiments, the anti-ILT3 antibody comprises: (i) a VH comprising a VH-CDR1 comprising the amino acid sequence set forth in SEQ ID NO:71, a VH-CDR2 comprising the amino acid sequence set forth in SEQ ID NO:99, and a VH-CDR3 comprising the amino acid sequence set forth in SEQ ID NO:73; and (ii) a VL comprising a VL-CDR1 comprising the amino acid sequence set forth in SEQ ID NO:100, a VL-CDR2 comprising the amino acid sequence set forth in SEQ ID NO:75, and a VL-CDR3 comprising the amino acid sequence set forth in SEQ ID NO:76. In certain embodiments, the anti-ILT3 antibody comprises: (i) a VH comprising a VH-CDR1 comprising the amino acid sequence set forth in SEQ ID NO:27, a VH-CDR2 comprising the amino acid sequence set forth in SEQ ID NO:28, and a VH-CDR3 comprising the amino acid sequence set forth in SEQ ID NO:105; and (ii) a VL comprising a VL-CDR1 comprising the amino acid sequence set forth in SEQ ID NO:106, a VL-CDR2 comprising the amino acid sequence set forth in SEQ ID NO:31, and a VL-CDR3 comprising the amino acid sequence set forth in SEQ ID NO:32.

In certain embodiments, the CDRs of the anti-ILT3 antibody are defined according to the Chothia designation. In certain embodiments, the anti-ILT3 antibody comprises: (i) a VH comprising a VH-CDR1 comprising the amino acid sequence set forth in SEQ ID NO:17, a VH-CDR2 comprising the amino acid sequence set forth in SEQ ID NO:18, and a VH-CDR3 comprising the amino acid sequence set forth in SEQ ID NO:13; and (ii) a VL comprising a VL-CDR1 comprising the amino acid sequence set forth in SEQ ID NO:14, a VL-CDR2 comprising the amino acid sequence set forth in SEQ ID NO:15, and a VL-CDR3 comprising the amino acid sequence set forth in SEQ ID NO:16. In certain embodiments, the anti-ILT3 antibody comprises: (i) a VH comprising a VH-CDR1 comprising the amino acid sequence set forth in SEQ ID NO:33, a VH-CDR2 comprising the amino acid sequence set forth in SEQ ID NO:34, and a VH-CDR3 comprising the amino acid sequence set forth in SEQ ID NO:29; and (ii) a VL comprising a VL-CDR1 comprising the amino acid sequence set forth in SEQ ID NO:30, a VL-CDR2 comprising the amino acid sequence set forth in SEQ ID NO:31, and a VL-CDR3 comprising the amino acid sequence set forth in SEQ ID NO:32. In certain embodiments, the anti-ILT3 antibody comprises: (i) a VH comprising a VH-CDR1 comprising the amino acid sequence set forth in SEQ ID NO:49, a VH-CDR2 comprising the amino acid sequence set forth in SEQ ID NO:50, and a VH-CDR3 comprising the amino acid sequence set forth in SEQ ID NO:45; and (ii) a VL comprising a VL-CDR1 comprising the amino acid sequence set forth in SEQ ID NO:46, a VL-CDR2 comprising the amino acid sequence set forth in SEQ ID NO:47, and a VL-CDR3 comprising the amino acid sequence set forth in SEQ ID NO:48. In certain embodiments, the anti-ILT3 antibody comprises: (i) a VH comprising a VH-CDR1 comprising the amino acid sequence set forth in SEQ ID NO:49, a VH-CDR2 comprising the amino acid sequence set forth in SEQ ID NO:64, and a VH-CDR3 comprising the amino acid sequence set forth in SEQ ID NO:60; and (ii) a VL comprising a VL-CDR1 comprising the amino acid sequence set forth in SEQ ID NO:61, a VL-CDR2 comprising the amino acid sequence set forth in SEQ ID NO:62, and a VL-CDR3 comprising the amino acid sequence set forth in SEQ ID NO:63. In certain embodiments, the anti-ILT3 antibody comprises: (i) a VH comprising a VH-CDR1 comprising the amino acid sequence set forth in SEQ ID NO:77, a VH-CDR2 comprising the amino acid sequence set forth in SEQ ID NO:78, and a VH-CDR3 comprising the amino acid sequence set forth in SEQ ID NO:73; and (ii) a VL comprising a VL-CDR1 comprising the amino acid sequence set forth in SEQ ID NO:74, a VL-CDR2 comprising the amino acid sequence set forth in SEQ ID NO:75, and a VL-CDR3 comprising the amino acid sequence set forth in SEQ ID NO:76. In certain embodiments, the anti-ILT3 antibody comprises: (i) a VH comprising a VH-CDR1 comprising the amino acid sequence set forth in SEQ ID NO:33, a VH-CDR2 comprising the amino acid sequence set forth in SEQ ID NO:92, and a VH-CDR3 comprising the amino acid sequence set forth in SEQ ID NO:88; and (ii) a VL comprising a VL-CDR1 comprising the amino acid sequence set forth in SEQ ID NO:89, a VL-CDR2 comprising the amino acid sequence set forth in SEQ ID NO:90, and a VL-CDR3 comprising the amino acid sequence set forth in SEQ ID NO:91. In certain embodiments, the anti-ILT3 antibody comprises: (i) a VH comprising a VH-CDR1 comprising the amino acid sequence set forth in SEQ ID NO:77, a VH-CDR2 comprising the amino acid sequence set forth in SEQ ID NO:101, and a VH-CDR3 comprising the amino acid sequence set forth in SEQ ID NO:73; and (ii) a VL comprising a VL-CDR1 comprising the amino acid sequence set forth in SEQ ID NO:100, a VL-CDR2 comprising the amino acid sequence set forth in SEQ ID NO:75, and a VL-CDR3 comprising the amino acid sequence set forth in SEQ ID NO:76. In certain embodiments, the anti-ILT3 antibody comprises: (i) a VH comprising a VH-CDR1 comprising the amino acid sequence set forth in SEQ ID NO:33, a VH-CDR2 comprising the amino acid sequence set forth in SEQ ID NO:34, and a VH-CDR3 comprising the amino acid sequence set forth in SEQ ID NO:105; and (ii) a VL comprising a VL-CDR1 comprising the amino acid sequence set forth in SEQ ID NO:106, a VL-CDR2 comprising the amino acid sequence set forth in SEQ ID NO:31, and a VL-CDR3 comprising the amino acid sequence set forth in SEQ ID NO:32.

In certain embodiments, the CDRs of the anti-ILT3 antibody are defined according to the AbM designation. In certain embodiments, the anti-ILT3 antibody comprises: (i) a VH comprising a VH-CDR1 comprising the amino acid sequence set forth in SEQ ID NO:11, a VH-CDR2 comprising the amino acid sequence set forth in SEQ ID NO:19, and a VH-CDR3 comprising the amino acid sequence set forth in SEQ ID NO:13; and (ii) a VL comprising a VL-CDR1 comprising the amino acid sequence set forth in SEQ ID NO:14, a VL-CDR2 comprising the amino acid sequence set forth in SEQ ID NO:15, and a VL-CDR3 comprising the amino acid sequence set forth in SEQ ID NO:16. In certain embodiments, the anti-ILT3 antibody comprises: (i) a VH comprising a VH-CDR1 comprising the amino acid sequence set forth in SEQ ID NO:27, a VH-CDR2 comprising the amino acid sequence set forth in SEQ ID NO:35, and a VH-CDR3 comprising the amino acid sequence set forth in SEQ ID NO:29; and (ii) a VL comprising a VL-CDR1 comprising the amino acid sequence set forth in SEQ ID NO:30, a VL-CDR2 comprising the amino acid sequence set forth in SEQ ID NO:31, and a VL-CDR3 comprising the amino acid sequence set forth in SEQ ID NO:32. In certain embodiments, the anti-ILT3 antibody comprises: (i) a VH comprising a VH-CDR1 comprising the amino acid sequence set forth in SEQ ID NO:43, a VH-CDR2 comprising the amino acid sequence set forth in SEQ ID NO:51, and a VH-CDR3 comprising the amino acid sequence set forth in SEQ ID NO:45; and (ii) a VL comprising a VL-CDR1 comprising the amino acid sequence set forth in SEQ ID NO:46, a VL-CDR2 comprising the amino acid sequence set forth in SEQ ID NO:47, and a VL-CDR3 comprising the amino acid sequence set forth in SEQ ID NO:48. In certain embodiments, the anti-ILT3 antibody comprises: (i) a VH comprising a VH-CDR1 comprising the amino acid sequence set forth in SEQ ID NO:43, a VH-CDR2 comprising the amino acid sequence set forth in SEQ ID NO:65, and a VH-CDR3 comprising the amino acid sequence set forth in SEQ ID NO:60; and (ii) a VL comprising a VL-CDR1 comprising the amino acid sequence set forth in SEQ ID NO:61, a VL-CDR2 comprising the amino acid sequence set forth in SEQ ID NO:62, and a VL-CDR3 comprising the amino acid sequence set forth in SEQ ID NO:63. In certain embodiments, the anti-ILT3 antibody comprises: (i) a VH comprising a VH-CDR1 comprising the amino acid sequence set forth in SEQ ID NO:71, a VH-CDR2 comprising the amino acid sequence set forth in SEQ ID NO:79, and a VH-CDR3 comprising the amino acid sequence set forth in SEQ ID NO:73; and (ii) a VL comprising a VL-CDR1 comprising the amino acid sequence set forth in SEQ ID NO:74, a VL-CDR2 comprising the amino acid sequence set forth in SEQ ID NO:75, and a VL-CDR3 comprising the amino acid sequence set forth in SEQ ID NO:76. In certain embodiments, the anti-ILT3 antibody comprises: (i) a VH comprising a VH-CDR1 comprising the amino acid sequence set forth in SEQ ID NO:27, a VH-CDR2 comprising the amino acid sequence set forth in SEQ ID NO:93, and a VH-CDR3 comprising the amino acid sequence set forth in SEQ ID NO:88; and (ii) a VL comprising a VL-CDR1 comprising the amino acid sequence set forth in SEQ ID NO:89, a VL-CDR2 comprising the amino acid sequence set forth in SEQ ID NO:90, and a VL-CDR3 comprising the amino acid sequence set forth in SEQ ID NO:91. In certain embodiments, the anti-ILT3 antibody comprises: (i) a VH comprising a VH-CDR1 comprising the amino acid sequence set forth in SEQ ID NO:71, a VH-CDR2 comprising the amino acid sequence set forth in SEQ ID NO:102, and a VH-CDR3 comprising the amino acid sequence set forth in SEQ ID NO:73; and (ii) a VL comprising a VL-CDR1 comprising the amino acid sequence set forth in SEQ ID NO:100, a VL-CDR2 comprising the amino acid sequence set forth in SEQ ID NO:75, and a VL-CDR3 comprising the amino acid sequence set forth in SEQ ID NO:76. In certain embodiments, the anti-ILT3 antibody comprises: (i) a VH comprising a VH-CDR1 comprising the amino acid sequence set forth in SEQ ID NO:27, a VH-CDR2 comprising the amino acid sequence set forth in SEQ ID NO:35, and a VH-CDR3 comprising the amino acid sequence set forth in SEQ ID NO:105; and (ii) a VL comprising a VL-CDR1 comprising the amino acid sequence set forth in SEQ ID NO:106, a VL-CDR2 comprising the amino acid sequence set forth in SEQ ID NO:31, and a VL-CDR3 comprising the amino acid sequence set forth in SEQ ID NO:32.

In certain embodiments, the CDRs of the anti-ILT3 antibody are defined according to the Kabat designation. In certain embodiments, the anti-ILT3 antibody comprises: (i) a VH comprising a VH-CDR1 comprising the amino acid sequence set forth in SEQ ID NO:20, a VH-CDR2 comprising the amino acid sequence set forth in SEQ ID NO:12, and a VH-CDR3 comprising the amino acid sequence set forth in SEQ ID NO:13; and (ii) a VL comprising a VL-CDR1 comprising the amino acid sequence set forth in SEQ ID NO:14, a VL-CDR2 comprising the amino acid sequence set forth in SEQ ID NO:15, and a VL-CDR3 comprising the amino acid sequence set forth in SEQ ID NO:16. In certain embodiments, the anti-ILT3 antibody comprises: (i) a VH comprising a VH-CDR1 comprising the amino acid sequence set forth in SEQ ID NO:36, a VH-CDR2 comprising the amino acid sequence set forth in SEQ ID NO:28, and a VH-CDR3 comprising the amino acid sequence set forth in SEQ ID NO:29; and (ii) a VL comprising a VL-CDR1 comprising the amino acid sequence set forth in SEQ ID NO:30, a VL-CDR2 comprising the amino acid sequence set forth in SEQ ID NO:31, and a VL-CDR3 comprising the amino acid sequence set forth in SEQ ID NO:32. In certain embodiments, the anti-ILT3 antibody comprises: (i) a VH comprising a VH-CDR1 comprising the amino acid sequence set forth in SEQ ID NO:52, a VH-CDR2 comprising the amino acid sequence set forth in SEQ ID NO:44, and a VH-CDR3 comprising the amino acid sequence set forth in SEQ ID NO:45; and (ii) a VL comprising a VL-CDR1 comprising the amino acid sequence set forth in SEQ ID NO:46, a VL-CDR2 comprising the amino acid sequence set forth in SEQ ID NO:47, and a VL-CDR3 comprising the amino acid sequence set forth in SEQ ID NO:48. In certain embodiments, the anti-ILT3 antibody comprises: (i) a VH comprising a VH-CDR1 comprising the amino acid sequence set forth in SEQ ID NO:52, a VH-CDR2 comprising the amino acid sequence set forth in SEQ ID NO:59, and a VH-CDR3 comprising the amino acid sequence set forth in SEQ ID NO:60; and (ii) a VL comprising a VL-CDR1 comprising the amino acid sequence set forth in SEQ ID NO:61, a VL-CDR2 comprising the amino acid sequence set forth in SEQ ID NO:62, and a VL-CDR3 comprising the amino acid sequence set forth in SEQ ID NO:63. In certain embodiments, the anti-ILT3 antibody comprises: (i) a VH comprising a VH-CDR1 comprising the amino acid sequence set forth in SEQ ID NO:80, a VH-CDR2 comprising the amino acid sequence set forth in SEQ ID NO:72, and a VH-CDR3 comprising the amino acid sequence set forth in SEQ ID NO:73; and (ii) a VL comprising a VL-CDR1 comprising the amino acid sequence set forth in SEQ ID NO:74, a VL-CDR2 comprising the amino acid sequence set forth in SEQ ID NO:75, and a VL-CDR3 comprising the amino acid sequence set forth in SEQ ID NO:76. In certain embodiments, the anti-ILT3 antibody comprises: (i) a VH comprising a VH-CDR1 comprising the amino acid sequence set forth in SEQ ID NO:36, a VH-CDR2 comprising the amino acid sequence set forth in SEQ ID NO:87, and a VH-CDR3 comprising the amino acid sequence set forth in SEQ ID NO:88; and (ii) a VL comprising a VL-CDR1 comprising the amino acid sequence set forth in SEQ ID NO:89, a VL-CDR2 comprising the amino acid sequence set forth in SEQ ID NO:90, and a VL-CDR3 comprising the amino acid sequence set forth in SEQ ID NO:91. In certain embodiments, the anti-ILT3 antibody comprises: (i) a VH comprising a VH-CDR1 comprising the amino acid sequence set forth in SEQ ID NO:80, a VH-CDR2 comprising the amino acid sequence set forth in SEQ ID NO:99, and a VH-CDR3 comprising the amino acid sequence set forth in SEQ ID NO:73; and (ii) a VL comprising a VL-CDR1 comprising the amino acid sequence set forth in SEQ ID NO:100, a VL-CDR2 comprising the amino acid sequence set forth in SEQ ID NO:75, and a VL-CDR3 comprising the amino acid sequence set forth in SEQ ID NO:76. In certain embodiments, the anti-ILT3 antibody comprises: (i) a VH comprising a VH-CDR1 comprising the amino acid sequence set forth in SEQ ID NO:36, a VH-CDR2 comprising the amino acid sequence set forth in SEQ ID NO:28, and a VH-CDR3 comprising the amino acid sequence set forth in SEQ ID NO:105; and (ii) a VL comprising a VL-CDR1 comprising the amino acid sequence set forth in SEQ ID NO:106, a VL-CDR2 comprising the amino acid sequence set forth in SEQ ID NO:31, and a VL-CDR3 comprising the amino acid sequence set forth in SEQ ID NO:32.

In certain embodiments, the CDRs of the anti-ILT3 antibody are defined according to the Contact designation. In certain embodiments, the anti-ILT3 antibody comprises: (i) a VH comprising a VH-CDR1 comprising the amino acid sequence set forth in SEQ ID NO:21, a VH-CDR2 comprising the amino acid sequence set forth in SEQ ID NO:22, and a VH-CDR3 comprising the amino acid sequence set forth in SEQ ID NO:23; and (ii) a VL comprising a VL-CDR1 comprising the amino acid sequence set forth in SEQ ID NO:24, a VL-CDR2 comprising the amino acid sequence set forth in SEQ ID NO:25, and a VL-CDR3 comprising the amino acid sequence set forth in SEQ ID NO:26. In certain embodiments, the anti-ILT3 antibody comprises: (i) a VH comprising a VH-CDR1 comprising the amino acid sequence set forth in SEQ ID NO:37, a VH-CDR2 comprising the amino acid sequence set forth in SEQ ID NO:38, and a VH-CDR3 comprising the amino acid sequence set forth in SEQ ID NO:39; and (ii) a VL comprising a VL-CDR1 comprising the amino acid sequence set forth in SEQ ID NO:40, a VL-CDR2 comprising the amino acid sequence set forth in SEQ ID NO:41, and a VL-CDR3 comprising the amino acid sequence set forth in SEQ ID NO:42. In certain embodiments, the anti-ILT3 antibody comprises: (i) a VH comprising a VH-CDR1 comprising the amino acid sequence set forth in SEQ ID NO:53, a VH-CDR2 comprising the amino acid sequence set forth in SEQ ID NO:54, and a VH-CDR3 comprising the amino acid sequence set forth in SEQ ID NO:55; and (ii) a VL comprising a VL-CDR1 comprising the amino acid sequence set forth in SEQ ID NO:56, a VL-CDR2 comprising the amino acid sequence set forth in SEQ ID NO:57, and a VL-CDR3 comprising the amino acid sequence set forth in SEQ ID NO:58. In certain embodiments, the anti-ILT3 antibody comprises: (i) a VH comprising a VH-CDR1 comprising the amino acid sequence set forth in SEQ ID NO:53, a VH-CDR2 comprising the amino acid sequence set forth in SEQ ID NO:66, and a VH-CDR3 comprising the amino acid sequence set forth in SEQ ID NO:67; and (ii) a VL comprising a VL-CDR1 comprising the amino acid sequence set forth in SEQ ID NO:68, a VL-CDR2 comprising the amino acid sequence set forth in SEQ ID NO:69, and a VL-CDR3 comprising the amino acid sequence set forth in SEQ ID NO:70. In certain embodiments, the anti-ILT3 antibody comprises: (i) a VH comprising a VH-CDR1 comprising the amino acid sequence set forth in SEQ ID NO:81, a VH-CDR2 comprising the amino acid sequence set forth in SEQ ID NO:82, and a VH-CDR3 comprising the amino acid sequence set forth in SEQ ID NO:83; and (ii) a VL comprising a VL-CDR1 comprising the amino acid sequence set forth in SEQ ID NO:84, a VL-CDR2 comprising the amino acid sequence set forth in SEQ ID NO:85, and a VL-CDR3 comprising the amino acid sequence set forth in SEQ ID NO:86. In certain embodiments, the anti-ILT3 antibody comprises: (i) a VH comprising a VH-CDR1 comprising the amino acid sequence set forth in SEQ ID NO:37, a VH-CDR2 comprising the amino acid sequence set forth in SEQ ID NO:94, and a VH-CDR3 comprising the amino acid sequence set forth in SEQ ID NO:95; and (ii) a VL comprising a VL-CDR1 comprising the amino acid sequence set forth in SEQ ID NO:96, a VL-CDR2 comprising the amino acid sequence set forth in SEQ ID NO:97, and a VL-CDR3 comprising the amino acid sequence set forth in SEQ ID NO:98. In certain embodiments, the anti-ILT3 antibody comprises: (i) a VH comprising a VH-CDR1 comprising the amino acid sequence set forth in SEQ ID NO:81, a VH-CDR2 comprising the amino acid sequence set forth in SEQ ID NO:103, and a VH-CDR3 comprising the amino acid sequence set forth in SEQ ID NO:83; and (ii) a VL comprising a VL-CDR1 comprising the amino acid sequence set forth in SEQ ID NO:104, a VL-CDR2 comprising the amino acid sequence set forth in SEQ ID NO:85, and a VL-CDR3 comprising the amino acid sequence set forth in SEQ ID NO:86. In certain embodiments, the anti-ILT3 antibody comprises: (i) a VH comprising a VH-CDR1 comprising the amino acid sequence set forth in SEQ ID NO:37, a VH-CDR2 comprising the amino acid sequence set forth in SEQ ID NO:38, and a VH-CDR3 comprising the amino acid sequence set forth in SEQ ID NO:107; and (ii) a VL comprising a VL-CDR1 comprising the amino acid sequence set forth in SEQ ID NO:108, a VL-CDR2 comprising the amino acid sequence set forth in SEQ ID NO:41, and a VL-CDR3 comprising the amino acid sequence set forth in SEQ ID NO:42.

In certain embodiments, the anti-ILT3 antibody comprises a humanized framework region (FR) sequence, e.g., as described herein.

In certain embodiments, the anti-ILT3 antibody comprises a VH comprising the VH-CDR1, the VH-CDR2, and the VH-CDR3 of antibody 3A3 (or a humanized version thereof), and wherein the VH comprises an amino acid sequence having at least 75%, at least 80%, at least 85%, at least 90%, at least 95%, at least 96%, at least 97%, at least 98%, or at least 99% identity to the amino acid sequence set forth in SEQ ID NO:109.

In certain embodiments, the anti-ILT3 antibody comprises a VL comprising the VL-CDR1, the VL-CDR2, and the VL-CDR3 of antibody 3A3 (or a humanized version thereof), and wherein the VL comprises an amino acid sequence having at least 75%, at least 80%, at least 85%, at least 90%, at least 95%, at least 96%, at least 97%, at least 98%, or at least 99% identity to the amino acid sequence set forth in SEQ ID NO:110.

In certain embodiments, the anti-ILT3 antibody comprises a VH comprising the VH-CDR1, the VH-CDR2, and the VH-CDR3 of antibody 5A7 (or a humanized version thereof), and wherein the VH comprises an amino acid sequence having at least 75%, at least 80%, at least 85%, at least 90%, at least 95%, at least 96%, at least 97%, at least 98%, or at least 99% identity to the amino acid sequence set forth in SEQ ID NO:111.

In certain embodiments, the anti-ILT3 antibody comprises a VL comprising the VL-CDR1, the VL-CDR2, and the VL-CDR3 of antibody 5A7 (or a humanized version thereof), and wherein the VL comprises an amino acid sequence having at least 75%, at least 80%, at least 85%, at least 90%, at least 95%, at least 96%, at least 97%, at least 98%, or at least 99% identity to the amino acid sequence set forth in SEQ ID NO:112.

In certain embodiments, the anti-ILT3 antibody comprises a VH comprising the VH-CDR1, the VH-CDR2, and the VH-CDR3 of antibody 12A12 (or a humanized version thereof), and wherein the VH comprises an amino acid sequence having at least 75%, at least 80%, at least 85%, at least 90%, at least 95%, at least 96%, at least 97%, at least 98%, or at least 99% identity to the amino acid sequence set forth in SEQ ID NO:113.

In certain embodiments, the anti-ILT3 antibody comprises a VL comprising the VL-CDR1, the VL-CDR2, and the VL-CDR3 of antibody 12A12 (or a humanized version thereof), and wherein the VL comprises an amino acid sequence having at least 75%, at least 80%, at least 85%, at least 90%, at least 95%, at least 96%, at least 97%, at least 98%, or at least 99% identity to the amino acid sequence set forth in SEQ ID NO:114.

In certain embodiments, the anti-ILT3 antibody comprises a VH comprising the VH-CDR1, the VH-CDR2, and the VH-CDR3 of antibody 16C5 (or a humanized version thereof), and wherein the VH comprises an amino acid sequence having at least 75%, at least 80%, at least 85%, at least 90%, at least 95%, at least 96%, at least 97%, at least 98%, or at least 99% identity to the amino acid sequence set forth in SEQ ID NO:115.

In certain embodiments, the anti-ILT3 antibody comprises a VL comprising the VL-CDR1, the VL-CDR2, and the VL-CDR3 of antibody 16C5 (or a humanized version thereof), and wherein the VL comprises an amino acid sequence having at least 75%, at least 80%, at least 85%, at least 90%, at least 95%, at least 96%, at least 97%, at least 98%, or at least 99% identity to the amino acid sequence set forth in SEQ ID NO:116.

In certain embodiments, the anti-ILT3 antibody comprises a VH comprising the VH-CDR1, the VH-CDR2, and the VH-CDR3 of antibody 45G10 (or a humanized version thereof), and wherein the VH comprises an amino acid sequence having at least 75%, at least 80%, at least 85%, at least 90%, at least 95%, at least 96%, at least 97%, at least 98%, or at least 99% identity to the amino acid sequence set forth in SEQ ID NO:117.

In certain embodiments, the anti-ILT3 antibody comprises a VL comprising the VL-CDR1, the VL-CDR2, and the VL-CDR3 of antibody 45G10 (or a humanized version thereof), and wherein the VL comprises an amino acid sequence having at least 75%, at least 80%, at least 85%, at least 90%, at least 95%, at least 96%, at least 97%, at least 98%, or at least 99% identity to the amino acid sequence set forth in SEQ ID NO:118.

In certain embodiments, the anti-ILT3 antibody comprises a VH comprising the VH-CDR1, the VH-CDR2, and the VH-CDR3 of antibody 48A6 (or a humanized version thereof), and wherein the VH comprises an amino acid sequence having at least 75%, at least 80%, at least 85%, at least 90%, at least 95%, at least 96%, at least 97%, at least 98%, or at least 99% identity to the amino acid sequence set forth in SEQ ID NO:119.

In certain embodiments, the anti-ILT3 antibody comprises a VL comprising the VL-CDR1, the VL-CDR2, and the VL-CDR3 of antibody 48A6 (or a humanized version thereof), and wherein the VL comprises an amino acid sequence having at least 75%, at least 80%, at least 85%, at least 90%, at least 95%, at least 96%, at least 97%, at least 98%, or at least 99% identity to the amino acid sequence set forth in SEQ ID NO:120.

In certain embodiments, the anti-ILT3 antibody comprises a VH comprising the VH-CDR1, the VH-CDR2, and the VH-CDR3 of antibody 53F10 (or a humanized version thereof), and wherein the VH comprises an amino acid sequence having at least 75%, at least 80%, at least 85%, at least 90%, at least 95%, at least 96%, at least 97%, at least 98%, or at least 99% identity to the amino acid sequence set forth in SEQ ID NO:121.

In certain embodiments, the anti-ILT3 antibody comprises a VL comprising the VL-CDR1, the VL-CDR2, and the VL-CDR3 of antibody 53F10 (or a humanized version thereof), and wherein the VL comprises an amino acid sequence having at least 75%, at least 80%, at least 85%, at least 90%, at least 95%, at least 96%, at least 97%, at least 98%, or at least 99% identity to the amino acid sequence set forth in SEQ ID NO:122.

In certain embodiments, the anti-ILT3 antibody comprises a VH comprising the VH-CDR1, the VH-CDR2, and the VH-CDR3 of antibody Hz5A7.v5, and wherein the VH comprises an amino acid sequence having at least 75%, at least 80%, at least 85%, at least 90%, at least 95%, at least 96%, at least 97%, at least 98%, or at least 99% identity to the amino acid sequence set forth in SEQ ID NO:123.

In certain embodiments, the anti-ILT3 antibody comprises a VL comprising the VL-CDR1, the VL-CDR2, and the VL-CDR3 of antibody Hz5A7.v5, and wherein the VL comprises an amino acid sequence having at least 75%, at least 80%, at least 85%, at least 90%, at least 95%, at least 96%, at least 97%, at least 98%, or at least 99% identity to the amino acid sequence set forth in SEQ ID NO:124.

In certain embodiments, the anti-ILT3 antibody comprises: (i) a VH comprising the VH-CDR1, the VH-CDR2, and the VH-CDR3 of antibody 3A3 (or a humanized version thereof), and wherein the VH comprises an amino acid sequence having at least 75%, at least 80%, at least 85%, at least 90%, at least 95%, at least 96%, at least 97%, at least 98%, or at least 99% identity to the amino acid sequence set forth in SEQ ID NO:109; and a VL comprising the VL-CDR1, the VL-CDR2, and the VL-CDR3 of antibody 3A3 (or a humanized version thereof), and wherein the VL comprises an amino acid sequence having at least 75%, at least 80%, at least 85%, at least 90%, at least 95%, at least 96%, at least 97%, at least 98%, or at least 99% identity to the amino acid sequence set forth in SEQ ID NO:110.

In certain embodiments, the anti-ILT3 antibody comprises: (i) a VH comprising the VH-CDR1, the VH-CDR2, and the VH-CDR3 of antibody 5A7 (or a humanized version thereof), and wherein the VH comprises an amino acid sequence having at least 75%, at least 80%, at least 85%, at least 90%, at least 95%, at least 96%, at least 97%, at least 98%, or at least 99% identity to the amino acid sequence set forth in SEQ ID NO:111; and (ii) a VL comprising the VL-CDR1, the VL-CDR2, and the VL-CDR3 of antibody 5A7 (or a humanized version thereof), and wherein the VL comprises an amino acid sequence having at least 75%, at least 80%, at least 85%, at least 90%, at least 95%, at least 96%, at least 97%, at least 98%, or at least 99% identity to the amino acid sequence set forth in SEQ ID NO:112.

In certain embodiments, the anti-ILT3 antibody comprises: (i) a VH comprising the VH-CDR1, the VH-CDR2, and the VH-CDR3 of antibody 12A12 (or a humanized version thereof), and wherein the VH comprises an amino acid sequence having at least 75%, at least 80%, at least 85%, at least 90%, at least 95%, at least 96%, at least 97%, at least 98%, or at least 99% identity to the amino acid sequence set forth in SEQ ID NO:113; and (ii) a VL comprising the VL-CDR1, the VL-CDR2, and the VL-CDR3 of antibody 12A12 (or a humanized version thereof), and wherein the VL comprises an amino acid sequence having at least 75%, at least 80%, at least 85%, at least 90%, at least 95%, at least 96%, at least 97%, at least 98%, or at least 99% identity to the amino acid sequence set forth in SEQ ID NO:114. In certain embodiments, the anti-ILT3 antibody comprises: (i) a VH comprising the VH-CDR1, the VH-CDR2, and the VH-CDR3 of antibody 16C5 (or a humanized version thereof), and wherein the VH comprises an amino acid sequence having at least 75%, at least 80%, at least 85%, at least 90%, at least 95%, at least 96%, at least 97%, at least 98%, or at least 99% identity to the amino acid sequence set forth in SEQ ID NO:115; and (ii) a VL comprising the VL-CDR1, the VL-CDR2, and the VL-CDR3 of antibody 16C5 (or a humanized version thereof), and wherein the VL comprises an amino acid sequence having at least 75%, at least 80%, at least 85%, at least 90%, at least 95%, at least 96%, at least 97%, at least 98%, or at least 99% identity to the amino acid sequence set forth in SEQ ID NO:116.

In certain embodiments, the anti-ILT3 antibody comprises: (i) a VH comprising the VH-CDR1, the VH-CDR2, and the VH-CDR3 of antibody 45G10 (or a humanized version thereof), and wherein the VH comprises an amino acid sequence having at least 75%, at least 80%, at least 85%, at least 90%, at least 95%, at least 96%, at least 97%, at least 98%, or at least 99% identity to the amino acid sequence set forth in SEQ ID NO:117; and (ii) a VL comprising the VL-CDR1, the VL-CDR2, and the VL-CDR3 of antibody 45G10 (or a humanized version thereof), and wherein the VL comprises an amino acid sequence having at least 75%, at least 80%, at least 85%, at least 90%, at least 95%, at least 96%, at least 97%, at least 98%, or at least 99% identity to the amino acid sequence set forth in SEQ ID NO:118.

In certain embodiments, the anti-ILT3 antibody comprises: (i) a VH comprising the VH-CDR1, the VH-CDR2, and the VH-CDR3 of antibody 48A6 (or a humanized version thereof), and wherein the VH comprises an amino acid sequence having at least 75%, at least 80%, at least 85%, at least 90%, at least 95%, at least 96%, at least 97%, at least 98%, or at least 99% identity to the amino acid sequence set forth in SEQ ID NO:119; and (ii) a VL comprising the VL-CDR1, the VL-CDR2, and the VL-CDR3 of antibody 48A6 (or a humanized version thereof), and wherein the VL comprises an amino acid sequence having at least 75%, at least 80%, at least 85%, at least 90%, at least 95%, at least 96%, at least 97%, at least 98%, or at least 99% identity to the amino acid sequence set forth in SEQ ID NO:120.

In certain embodiments, the anti-ILT3 antibody comprises: (i) a VH comprising the VH-CDR1, the VH-CDR2, and the VH-CDR3 of antibody 53F10 (or a humanized version thereof), and wherein the VH comprises an amino acid sequence having at least 75%, at least 80%, at least 85%, at least 90%, at least 95%, at least 96%, at least 97%, at least 98%, or at least 99% identity to the amino acid sequence set forth in SEQ ID NO:121; and (ii) a VL comprising the VL-CDR1, the VL-CDR2, and the VL-CDR3 of antibody 53F10 (or a humanized version thereof), and wherein the VL comprises an amino acid sequence having at least 75%, at least 80%, at least 85%, at least 90%, at least 95%, at least 96%, at least 97%, at least 98%, or at least 99% identity to the amino acid sequence set forth in SEQ ID NO:122.

In certain embodiments, the anti-ILT3 antibody comprises: (i) a VH comprising the VH-CDR1, the VH-CDR2, and the VH-CDR3 of antibody Hz5A7.v5, and wherein the VH comprises an amino acid sequence having at least 75%, at least 80%, at least 85%, at least 90%, at least 95%, at least 96%, at least 97%, at least 98%, or at least 99% identity to the amino acid sequence set forth in SEQ ID NO:123; and (ii) a VL comprising the VL-CDR1, the VL-CDR2, and the VL-CDR3 of antibody Hz5A7.v5, and wherein the VL comprises an amino acid sequence having at least 75%, at least 80%, at least 85%, at least 90%, at least 95%, at least 96%, at least 97%, at least 98%, or at least 99% identity to the amino acid sequence set forth in SEQ ID NO:124.

In certain embodiments, the anti-ILT3 antibody comprises a VH comprising the amino acid sequence set forth in SEQ ID NO:109 (or a humanized version thereof).

In certain embodiments, the anti-ILT3 antibody comprises a VH comprising the amino acid sequence set forth in SEQ ID NO:111 (or a humanized version thereof).

In certain embodiments, the anti-ILT3 antibody comprises a VH comprising the amino acid sequence set forth in SEQ ID NO:113 (or a humanized version thereof).

In certain embodiments, the anti-ILT3 antibody comprises a VH comprising the amino acid sequence set forth in SEQ ID NO:115 (or a humanized version thereof).

In certain embodiments, the anti-ILT3 antibody comprises a VH comprising the amino acid sequence set forth in SEQ ID NO:117 (or a humanized version thereof).

In certain embodiments, the anti-ILT3 antibody comprises a VH comprising the amino acid sequence set forth in SEQ ID NO:119 (or a humanized version thereof).

In certain embodiments, the anti-ILT3 antibody comprises a VH comprising the amino acid sequence set forth in SEQ ID NO:121 (or a humanized version thereof).

In certain embodiments, the anti-ILT3 antibody comprises a VH comprising the amino acid sequence set forth in SEQ ID NO:123.

In certain embodiments, the anti-ILT3 antibody comprises a VL comprising the amino acid sequence set forth in SEQ ID NO:110 (or a humanized version thereof).

In certain embodiments, the anti-ILT3 antibody comprises a VL comprising the amino acid sequence set forth in SEQ ID NO:112 (or a humanized version thereof).

In certain embodiments, the anti-ILT3 antibody comprises a VL comprising the amino acid sequence set forth in SEQ ID NO:114 (or a humanized version thereof).

In certain embodiments, the anti-ILT3 antibody comprises a VL comprising the amino acid sequence set forth in SEQ ID NO:116 (or a humanized version thereof).

In certain embodiments, the anti-ILT3 antibody comprises a VL comprising the amino acid sequence set forth in SEQ ID NO:118 (or a humanized version thereof).

In certain embodiments, the anti-ILT3 antibody comprises a VL comprising the amino acid sequence set forth in SEQ ID NO:120 (or a humanized version thereof).

In certain embodiments, the anti-ILT3 antibody comprises a VL comprising the amino acid sequence set forth in SEQ ID NO:122 (or a humanized version thereof).

In certain embodiments, the anti-ILT3 antibody comprises a VL comprising the amino acid sequence set forth in SEQ ID NO:124.

In certain embodiments, the anti-ILT3 antibody comprises: (i) a VH comprising the amino acid sequence set forth in SEQ ID NO:109 (or a humanized version thereof); and (ii) a VL comprising the amino acid sequence set forth in SEQ ID NO:110 (or a humanized version thereof).

In certain embodiments, the anti-ILT3 antibody comprises: (i) a VH comprising the amino acid sequence set forth in SEQ ID NO:111 (or a humanized version thereof); and (ii) a VL comprising the amino acid sequence set forth in SEQ ID NO:112 (or a humanized version thereof).

In certain embodiments, the anti-ILT3 antibody comprises: (i) a VH comprising the amino acid sequence set forth in SEQ ID NO:113 (or a humanized version thereof); and (ii) a VL comprising the amino acid sequence set forth in SEQ ID NO:114 (or a humanized version thereof).

In certain embodiments, the anti-ILT3 antibody comprises: (i) a VH comprising the amino acid sequence set forth in SEQ ID NO:115 (or a humanized version thereof); and (ii) a VL comprising the amino acid sequence set forth in SEQ ID NO:116 (or a humanized version thereof).

In certain embodiments, the anti-ILT3 antibody comprises: (i) a VH comprising the amino acid sequence set forth in SEQ ID NO:117 (or a humanized version thereof); and (ii) a VL comprising the amino acid sequence set forth in SEQ ID NO:118 (or a humanized version thereof).

In certain embodiments, the anti-ILT3 antibody comprises: (i) a VH comprising the amino acid sequence set forth in SEQ ID NO:119 (or a humanized version thereof); and (ii) a VL comprising the amino acid sequence set forth in SEQ ID NO:120 (or a humanized version thereof).

In certain embodiments, the anti-ILT3 antibody comprises: (i) a VH comprising the amino acid sequence set forth in SEQ ID NO:121 (or a humanized version thereof); and (ii) a VL comprising the amino acid sequence set forth in SEQ ID NO:122 (or a humanized version thereof).

In certain embodiments, the anti-ILT3 antibody comprises: (i) a VH comprising the amino acid sequence set forth in SEQ ID NO:123; and (ii) a VL comprising the amino acid sequence set forth in SEQ ID NO:124.

In certain embodiments, the anti-ILT3 antibody comprises a heavy chain comprising a VH comprising the VH-CDR1, the VH-CDR2, and the VH-CDR3 from the amino acid sequence set forth in SEQ ID NO:123, and wherein the heavy chain comprises an amino acid sequence having at least 75%, at least 80%, at least 85%, at least 90%, at least 95%, at least 96%, at least 97%, at least 98%, at least 99%, or 100% identity to the amino acid sequence set forth in SEQ ID NO:126.

In certain embodiments, the anti-ILT3 antibody comprises a light chain comprising a VL comprising the VL-CDR1, the VL-CDR2, and the VL-CDR3 from the amino acid sequence set forth in SEQ ID NO:124, and wherein the light chain comprises an amino acid sequence having at least 75%, at least 80%, at least 85%, at least 90%, at least 95%, at least 96%, at least 97%, at least 98%, at least 99%, or 100% identity to the amino acid sequence set forth in SEQ ID NO:128.

In certain embodiments, the anti-ILT3 antibody comprises: (i) a heavy chain comprising a VH comprising the VH-CDR1, the VH-CDR2, and the VH-CDR3 from the amino acid sequence set forth in SEQ ID NO:123, and wherein the heavy chain comprises an amino acid sequence having at least 75%, at least 80%, at least 85%, at least 90%, at least 95%, at least 96%, at least 97%, at least 98%, at least 99%, or 100% identity to the amino acid sequence set forth in SEQ ID NO:126; and (ii) a light chain comprising a VL comprising the VL-CDR1, the VL-CDR2, and the VL-CDR3 from the amino acid sequence set forth in SEQ ID NO:124, and wherein the light chain comprises an amino acid sequence having at least 75%, at least 80%, at least 85%, at least 90%, at least 95%, at least 96%, at least 97%, at least 98%, at least 99%, or 100% identity to the amino acid sequence set forth in SEQ ID NO:128.

In certain embodiments, the anti-ILT3 antibody comprises a heavy chain comprising the amino acid sequence set forth in SEQ ID NO:126. In certain embodiments, the anti-ILT3 antibody comprises a heavy chain consisting of the amino acid sequence set forth in SEQ ID NO:126.

In certain embodiments, the anti-ILT3 antibody comprises a light chain comprising the amino acid sequence set forth in SEQ ID NO:128. In certain embodiments, the anti-ILT3 antibody comprises a light chain consisting of the amino acid sequence set forth in SEQ ID NO:128.

In certain embodiments, the anti-ILT3 antibody comprises: (i) a heavy chain comprising the amino acid sequence set forth in SEQ ID NO:126; and (ii) a light chain comprising the amino acid sequence set forth in SEQ ID NO:128. In certain embodiments, the anti-ILT3 antibody comprises: (i) a heavy chain consisting of the amino acid sequence set forth in SEQ ID NO:126; and (ii) a light chain consisting of the amino acid sequence set forth in SEQ ID NO:128.

In certain embodiments, the anti-ILT3 antibody is an anti-ILT3 antibody described in any of the following publications: US20190153093A1, WO2020056077A1, WO2021183839A2, US20200031926A1, US20210221887A1, US20150110714A1, US20200031926A1, US20190241655A1, WO2020180789A1, and WO2020056077A1, which are all incorporated herein by reference in their entireties.

In certain embodiments, the anti-ILT3 antibody is an antibody described in International Patent Application Publication No. WO 2021127200A1, which is incorporated by reference herein in its entirety.

In certain embodiments, the anti-ILT3 antibody is a humanized version of an antibody described in International Patent Application Publication No. WO 2021127200A1.

In certain embodiments, the anti-ILT3 antibody binds to the same epitope as an antibody having the VH and VL of any one of Tables 1-8. In certain embodiments, the anti-ILT3 antibody binds to the same epitope as antibody Hz5A7.v5 (see Table 8).

In certain embodiments, the anti-ILT3 antibody binds ILT3 or a fragment of ILT3. In certain embodiments, the fragment of ILT3 comprises the extracellular domain of ILT3. In certain embodiments, the fragment of ILT3 comprises one of the Ig-like C2 type domains (e.g., D1 or D2). In certain embodiments, the fragment of ILT3 comprises both of the Ig-like C2 type domains (e.g., D1-D2). In certain embodiments, the fragment of ILT3 comprises both of the Ig-like C2 type domains and the stem region (e.g., D1-D2-stem). In certain embodiments, the fragment of ILT3 comprises one of the Ig-like C2 type domains and the stem region (e.g., D1-stem or D2-stem). In certain embodiments, the anti-ILT3 antibody binds human ILT3 or a fragment of human ILT3. In certain embodiments, the fragment of human ILT3 comprises the extracellular domain of human ILT3. In certain embodiments, the extracellular domain of human ILT3 comprises amino acids 22-259 of SEQ ID NO:1. In certain embodiments, D1 domain of human ILT3 comprises amino acids 27-118 of SEQ ID NO:1. In certain embodiments, D2 domain of human ILT3 comprises amino acids 124-218 of SEQ ID NO:1. In certain embodiments, D1-D2 of human ILT3 comprises amino acids 27-218 of SEQ ID NO:1. In certain embodiments, D1-D2-stem of human ILT3 comprises amino acids 27-259 of SEQ ID NO:1. In certain embodiments, D2-stem of human ILT3 comprises amino acids 124-259 of SEQ ID NO:1. In certain embodiments, the fragment of human ILT3 comprises the amino acid sequence of SEQ ID NO:3. In certain embodiments, the fragment of human ILT3 comprises the amino acid sequence of SEQ ID NO:4. In certain embodiments, the fragment of human ILT3 comprises the amino acid sequence of SEQ ID NO:5. In certain embodiments, the fragment of human ILT3 comprises the amino acid sequence of SEQ ID NO:4 and SEQ ID NO:5. In certain embodiments, the anti-ILT3 antibody binds cyno ILT3 or a fragment of cyno ILT3. In certain embodiments, the fragment of cyno ILT3 comprises the extracellular domain of cyno ILT3. In certain embodiments, the extracellular domain of cyno ILT3 comprises amino acids 22-259 of SEQ ID NO:6. In certain embodiments, D1 of cyno ILT3 comprises amino acids 27-118 of SEQ ID NO:6. In certain embodiments, D2 of cyno ILT3 comprises amino acids 124-218 of SEQ ID NO:6. In certain embodiments, D1-D2 of cyno ILT3 comprises amino acids 27-218 of SEQ ID NO:6. In certain embodiments, D1-D2-stem of cyno ILT3 comprises amino acids 27-259 of SEQ ID NO:6. In certain embodiments, D2-stem of cyno ILT3 comprises amino acids 124-259 of SEQ ID NO:6. In certain embodiments, a fragment of cyno ILT3 comprises the amino acid sequence of SEQ ID NO:8. In certain embodiments, the fragment of cyno ILT3 comprises the amino acid sequence of SEQ ID NO:9. In certain embodiments, the fragment of cyno ILT3 comprises the amino acid sequence of SEQ ID NO:10. In certain embodiments, the fragment of cyno ILT3 comprises the amino acid sequence of SEQ ID NO:9 and SEQ ID NO:10. It is understood that the domains of ILT3 (e.g., human ILT3 or cyno ILT3) may be defined differently by those of skill in the art, therefore the N-terminal amino acids and the C-terminal amino acids of any ILT3 domain or region may vary by 1, 2, 3, 4, 5, or more amino acid residues.

In certain embodiments, the anti-ILT3 antibody binds human ILT3. In certain embodiments, the anti-ILT3 antibody binds cyno ILT3. In certain embodiments, the anti-ILT3 antibody binds human ILT3 and cyno ILT3. In certain embodiments, the anti-ILT3 antibody binds SEQ ID NO:1. In certain embodiments, the anti-ILT3 antibody binds SEQ ID NO:2. In certain embodiments, the anti-ILT3 antibody binds within amino acids 22-259 of SEQ ID NO:1. In certain embodiments, the anti-ILT3 antibody binds within amino acids 27-118 of SEQ ID NO:1. In certain embodiments, the anti-ILT3 antibody binds within amino acids 124-218 of SEQ ID NO:1. In certain embodiments, the anti-ILT3 antibody binds within amino acids 124-259 of SEQ ID NO:1. In certain embodiments, the anti-ILT3 antibody binds within amino acids 27-218 of SEQ ID NO:1. In certain embodiments, the anti-ILT3 antibody binds SEQ ID NO:3. In certain embodiments, the anti-ILT3 antibody binds SEQ ID NO:4. In certain embodiments, the anti-ILT3 antibody binds SEQ ID NO:5. In certain embodiments, the anti-ILT3 antibody binds a fragment of ILT3 that comprises SEQ ID NO:4 and SEQ ID NO:5. In certain embodiments, the anti-ILT3 antibody binds SEQ ID NO:6. In certain embodiments, the anti-ILT3 antibody binds SEQ ID NO:7. In certain embodiments, the anti-ILT3 antibody binds within amino acids 22-259 of SEQ ID NO:6. In certain embodiments, the anti-ILT3 antibody binds within amino acids 27-118 of SEQ ID NO:6. In certain embodiments, the anti-ILT3 antibody binds within amino acids 124-218 of SEQ ID NO:6. In certain embodiments, the anti-ILT3 antibody binds within amino acids 27-218 of SEQ ID NO:6. In certain embodiments, the anti-ILT3 antibody binds SEQ ID NO:8. In certain embodiments, the anti-ILT3 antibody binds SEQ ID NO:9. In certain embodiments, the anti-ILT3 antibody binds SEQ ID NO:10. In certain embodiments, the anti-ILT3 antibody binds a fragment of ILT3 that comprises SEQ ID NO:9 and SEQ ID NO:10.

In certain embodiments, the anti-ILT3 antibody binds a polypeptide comprising the amino acid sequence of SEQ ID NO:2. In certain embodiments, the anti-ILT3 antibody binds a polypeptide comprising the amino acid sequence of SEQ ID NO:3. In certain embodiments, the anti-ILT3 antibody binds a polypeptide comprising the amino acid sequence of SEQ ID NO:4. In certain embodiments, the anti-ILT3 antibody binds a polypeptide comprising the amino acid sequence of SEQ ID NO:5. In certain embodiments, the anti-ILT3 antibody binds a polypeptide comprising the amino acid sequence of SEQ ID NO:4 and the amino acid sequence of SEQ ID NO:5. In certain embodiments, the anti-ILT3 antibody binds a polypeptide comprising the amino acid sequence of SEQ ID NO:7. In certain embodiments, the anti-ILT3 antibody binds a polypeptide comprising the amino acid sequence of SEQ ID NO:8. In certain embodiments, the anti-ILT3 antibody binds a polypeptide comprising the amino acid sequence of SEQ ID NO:9. In certain embodiments, the anti-ILT3 antibody binds a polypeptide comprising the amino acid sequence of SEQ ID NO:10. In certain embodiments, the anti-ILT3 antibody binds a polypeptide comprising the amino acid sequence of SEQ ID NO:9 and the amino acid sequence of SEQ ID NO:10.

In certain embodiments, the anti-ILT3 antibody binds an epitope comprising amino acids within SEQ ID NO:2. In certain embodiments, the anti-ILT3 antibody binds an epitope comprising amino acids within SEQ ID NO:3. In certain embodiments, the anti-ILT3 antibody binds an epitope comprising amino acids within SEQ ID NO:4. In certain embodiments, the anti-ILT3 antibody binds an epitope comprising amino acids within SEQ ID NO:5. In certain embodiments, then anti-ILT3 antibody binds an epitope comprising amino acids within SEQ ID NO:4 and SEQ ID NO:5. In certain embodiments, the anti-ILT3 antibody binds an epitope comprising amino acids within SEQ ID NO:7. In certain embodiments, the anti-ILT3 antibody binds an epitope comprising amino acids within SEQ ID NO:8. In certain embodiments, the anti-ILT3 antibody binds an epitope comprising amino acids within SEQ ID NO:9. In certain embodiments, the anti-ILT3 antibody binds an epitope comprising amino acids within SEQ ID NO:10. In certain embodiments, the anti-ILT3 antibody binds an epitope comprising amino acids within SEQ ID NO:9 and SEQ ID NO:10.

In certain embodiments, the anti-ILT3 antibody binds an ILT3 epitope within the extracellular domain of human ILT3. In certain embodiments, the anti-ILT3 antibody binds an ILT3 epitope within the extracellular domain of cyno ILT3. In certain embodiments, the anti-ILT3 antibody binds an epitope comprising at least one amino acid within amino acids 22-259 of SEQ ID NO:1. In certain embodiments, the anti-ILT3 antibody binds an epitope comprising at least one amino acid within amino acids 22-120 of SEQ ID NO:1. In certain embodiments, the anti-ILT3 antibody binds an epitope comprising at least one amino acid within amino acids 27-118 of SEQ ID NO:1. In certain embodiments, the anti-ILT3 antibody binds an epitope comprising at least one amino acid within amino acids 121-259 of SEQ ID NO:1. In certain embodiments, the anti-ILT3 antibody binds an epitope comprising at least one amino acid within amino acids 124-218 of SEQ ID NO:1. In certain embodiments, the anti-ILT3 antibody binds an epitope comprising at least one amino acid within amino acids 124-259 of SEQ ID NO:1. In certain embodiments, the anti-ILT3 antibody binds an epitope comprising amino acids within SEQ ID NO:3. In certain embodiments, the anti-ILT3 antibody binds an epitope comprising amino acids within SEQ ID NO:4. In certain embodiments, the anti-ILT3 antibody binds an epitope comprising amino acids within SEQ ID NO:5. In certain embodiments, the anti-ILT3 antibody binds an epitope comprising amino acids within SEQ ID NO:4 and SEQ ID NO:5. In certain embodiments, the epitope is a conformational epitope. In certain embodiments, the epitope is a linear epitope.

In certain embodiments, the anti-ILT3 antibody competes with a second agent for binding within the extracellular domain of human ILT3. In certain embodiments, the anti-ILT3 antibody competes with a second agent for binding within the extracellular domain of cyno ILT3. In certain embodiments, the anti-ILT3 antibody competes with a second agent for binding within amino acids 22-259 of SEQ ID NO:1. In certain embodiments, the anti-ILT3 antibody competes with a second agent for binding within amino acids 22-120 of SEQ ID NO:1. In certain embodiments, the anti-ILT3 antibody competes with a second agent for binding within amino acids 27-118 of SEQ ID NO:1. In certain embodiments, the anti-ILT3 antibody competes with a second agent for binding within amino acids 121-259 of SEQ ID NO:1. In certain embodiments, the anti-ILT3 antibody competes with a second agent for binding within amino acids 124-218 of SEQ ID NO:1. In certain embodiments, the anti-ILT3 antibody competes with a second agent for binding within amino acids 124-259 of SEQ ID NO:1. In certain embodiments, the anti-ILT3 antibody competes with a second agent for binding within amino acid sequence SEQ ID NO:3. In certain embodiments, the anti-ILT3 antibody competes with a second agent for binding within amino acid sequence SEQ ID NO:4. In certain embodiments, the anti-ILT3 antibody competes with a second agent for binding within amino acid sequence SEQ ID NO:5. In certain embodiments, the anti-ILT3 antibody competes with a second agent for binding within amino acid sequences SEQ ID NO:4 and SEQ ID NO:5. In certain embodiments, the second agent is an anti-ILT3-antibody described in any of the following publications: US20190153093A1, WO2020056077A1, WO2021183839A2, US20200031926A1, US20210221887A1, US20150110714A1, US20200031926A1, US20190241655A1, WO2020180789A1, and WO2020056077A1, which are all incorporated herein by reference in their entireties. In certain embodiments, the second agent is any one of the anti-ILT3-antibodies described WO 2021127200A1.

In certain embodiments, the anti-ILT3 antibody is a humanized antibody.

In certain embodiments, the anti-ILT3 antibody is a human IgG1 antibody. In certain embodiments, the anti-ILT3 antibody is a human IgG2 antibody. In certain embodiments, the anti-ILT3 antibody is a human IgG3 antibody. In certain embodiments, the anti-ILT3 antibody is a human IgG4 antibody. In certain embodiments, the anti-ILT3 antibody comprises a human kappa light chain constant region. In certain embodiments, the anti-ILT3 antibody comprises a human lambda light chain constant region. In certain embodiments, the anti-ILT3 antibody comprises a partial constant region sequence of a human IgG1 antibody. In certain embodiments, the anti-ILT3 antibody comprises a partial constant region sequence of a human IgG2 antibody. In certain embodiments, the anti-ILT3 antibody comprises a partial constant region sequence of a human IgG3 antibody. In certain embodiments, the anti-ILT3 antibody comprises a partial constant region sequence of a human IgG4 antibody.

In certain embodiments, the anti-ILT3 antibody is an antibody fragment comprising at least one antigen-binding site. In certain embodiments, the anti-ILT3 antibody is a Fab, a Fab′, a F(ab′)₂, a Fv, an scFv, an (scFv)₂, a single chain antibody, a dual variable region antibody, a single variable region antibody, a linear antibody, a diabody, a nanobody, or a V region antibody.

The anti-ILT3 antibodies described herein can be produced by any suitable methods known in the art. Such methods range from direct protein synthesis methods to constructing a DNA sequence encoding polypeptide sequences and expressing those sequences in a suitable host. See, e.g., International Patent Application Publication No. WO 2021127200A1, which is incorporated by reference herein in its entirety, for a description of various methods for producing antibodies.

### 5.1.3. Anti-LAIR-1 Antibodies

LAIR-1 (also known as LAIR-1, CD305, LAIR-1, leukocyte associated immunoglobulin like receptor 1) is a single pass type I transmembrane protein with a predicted molecular weight of approximately 32 kDa. As characterized within UniProtKB, human LAIR-1 is a protein of 287 amino acids (aa) long—the signal sequence is aa 1-21, the extracellular domain is aa 22-165, the transmembrane region is aa 166-186, and the cytoplasmic domain is aa 187-287. Within the extracellular domain, the Ig-like C2-type domain is aa 29-117 and the “stem region” is aa 118-165. Within the cytoplasmic domain, ITIMs are positioned at aa 249-254 and 279-284. LAIR-1 is expressed on almost all immune cells, including NK cells, T-cells, B-cells, monocytes, dendritic cells, eosinophils, basophils, and mast cells. LAIR-1 is characterized by an extracellular domain including one Ig-like C2 type domain, a transmembrane domain, and a cytoplasmic domain containing 2 ITIM domains (see, e.g., Meyaard et al., 1997, Immunity, 7:283-290; Meyaard et al., 2008, J Leuk. Biol., 83:799-803). LAIR-1 is known to bind to multiple transmembrane and extracellular matrix collagens. As described herein, MARCO and COLEC12 were identified as new and novel ligands for LAIR-1.

Cyno LAIR-1 has an amino acid sequence identity to human LAIR-1 of 88%. As characterized within UniProtKB, cyno LAIR-1 is a protein of 287 amino acids long and it is believed that the structural characteristics of cyno LAIR-1 are similar to human LAIR-1. Thus, for cyno LAIR-1 the signal sequence is predicted to be aa 1-21, the extracellular domain is predicted to be aa 22-165, the transmembrane region is predicted to be aa 166-186, and the cytoplasmic domain is predicted to be aa 187-287. Within the extracellular domain, the Ig-like C2-type domain is predicted to be aa 29-117 and the “stem region” is predicted to be aa 118-165. Within the cytoplasmic domain, ITIMs are positioned at aa 249-254 and 279-284.

Mouse LAIR-1 has an amino acid sequence identity to human LAIR-1 of 42%. As characterized within UniProtKB, mouse LAIR-1 is a protein of 263 amino acids long and has structural characteristics similar to human LAIR-1. Thus, for mouse LAIR-1 the signal sequence is aa 1-21, the extracellular domain is aa 22-144, the transmembrane region is aa 145-165, and the cytoplasmic domain is aa 166-263. Within the extracellular domain, the Ig-like C2-type domain is aa 27-114 and the “stem region” is aa 115-144. Within the cytoplasmic domain, ITIMs are positioned at aa 226-231 and 255-260.

The amino acid (aa) sequence for human LAIR-1 (UniProtKB No. Q6GTX8) is shown below and includes the predicted signal sequence (underlined residues):

(SEQ ID NO: 167)
MSPHPTALLGLVLCLAQTIHTQEEDLPRPSISAEP
GTVIPLGSHVTFVCRGPVGVOTFRLERESRSTYND
TEDVSQASPSESEARFRIDSVSEGNAGPYRCIYYK
PPKWSEQSDYLELLVKETSGGPDSPDTEPGSSAGP
TQRPSDNSHNEHAPASQGLKAEHLYILIGVSVVFL
FCLLLLVLFCLHRQNQIKQGPPRSKDEEQKPQQRP
DLAVDVLERTADKATVNGLPEKDRETDTSALAAGS
SQEVTYAQLDHWALTQRTARAVSPQSTKPMAESIT
YAAVARH

The amino acid (aa) sequence for cynomolgus monkey (“cyno”) LAIR-1 (UniProtKB No. A0A2K5TN26) is shown below and includes the predicted signal sequence (underlined residues):

(SEQ ID NO: 171)
MSPHPTALLGLVLCLAQTIHAQEGPLPRPSISAEP
GTVIPPGRPVTIVCRGPVGVDQFRLEREDRSKFND
TKDVSQASPSESEARFRIDSVSEGNAGHYRCLYVK
STRWSEHSDYLDLVVKETSGDTDSPVTEPDSSAGP
TQRPSDNSHNEHAPASQGLSAEHLYILIGVSVVFL
FCLLLLVLFFLHRQNQMKQGPPRSKDEEQKLQQRP
DLAVDVLERTADKATVNGLPEKDRETDTSAPAAGS
SQEVTYAQLDHWALTWRTAQAVSPQSTEPMAESST
YAAVARH

The amino acid (aa) sequence for mouse LAIR-1 (UniProtKB No. Q8BG84) is shown below and includes the predicted signal sequence (underlined residues):

SEQ ID NO: 201)
MSLHPVILLVLVLCLGWKINTQEGSLPDITIFPNS
SLMISQGTFVTVVCSYSDKHDLYNMVRLEKDGSTF
MEKSTEPYKTEDEFEIGPVNETITGHYSCIYSKGI
TWSERSKTLELKVIKENVIQTPAPGPTSDTSWLKT
YSIYIFTVVSVIFLLCLSALLFCFLRHRQKKQGLP
NNKRQQQRPEERLNLATNGLEMTPDIVADDRLPED
RWTETWTPVAGDLQEVTYIQLDHHSLTQRAVGAVT
SQSTDMAESSTYAAIIRH

As used herein, reference to amino acid positions of LAIR-1 refer to the numbering of amino acid sequences including the signal sequences.

It is understood that the regions and/or domains of LAIR-1 (e.g., human LAIR-1, cyno LAIR-1, or mouse LAIR-1) may be defined differently by those of skill in the art, therefore the N-terminal amino acids and the C-terminal amino acids of any LAIR-1 domain or region may vary by 1, 2, 3, 4, 5, or more amino acid residues.

The present disclosure provides agents (e.g., antibodies) that bind LAIR-1. In certain embodiments, the anti-LAIR-1 antibody binds a fragment of LAIR-1. In certain embodiments, the anti-LAIR-1 antibody binds within a specific region of LAIR-1. In certain embodiments, the anti-LAIR-1 antibody binds within the extracellular domain of LAIR-1. In certain embodiments, the anti-LAIR-1 antibody binds within the D1 domain of LAIR-1. In certain embodiments, the anti-LAIR-1 antibody binds within the D1-stem domain of LAIR-1. In certain embodiments, the anti-LAIR-1 antibody binds an epitope on LAIR-1. In certain embodiments, the anti-LAIR-1 antibody binds a conformational epitope on LAIR-1.

In certain embodiments, the anti-LAIR-1 antibody has at least one or more of the following properties: (i) binds human LAIR-1; (ii) binds cyno LAIR-1; (iii) does not bind mouse LAIR-1; (iv) does not bind human LAIR-2; (v) is a LAIR-1 antagonist; (vi) inhibits LAIR-1 activity; (vii) inhibits LAIR-1 signaling in cells that express LAIR-1; (viii) inhibits binding of LAIR-1 to collagen; (ix) inhibits binding of LAIR-1 to MARCO; (x) inhibits binding of LAIR-1 to COLEC12; (xi) inhibits LAIR-1-induced suppression of myeloid cells; (xii) inhibits LAIR-1-induced suppression of myeloid cell activity; (xiii) restores FcR activation in myeloid cells; (xiv) restores cytokine and/or chemokine production in myeloid cells; (xv) inhibits LAIR-1-induced suppression of NK cells; (xvi) inhibits LAIR-1-induced suppression of NK activity; (xvii) inhibits LAIR-1-induced suppression of T-cell activity; and/or (xviii) inhibits MDSC activity. In certain embodiments, the myeloid cells are monocytes. In certain embodiments, the myeloid cells are macrophages. In certain embodiments, the myeloid cells are dendritic cells. In certain embodiments, the myeloid cells are antigen-presenting cells (APCs).

In certain embodiments, the anti-LAIR-1 antibody comprises a VH comprising a VH-CDR1, a VH-CDR2, and a VH-CDR3, and a VL comprising a VL-CDR1, a VL-CDR2, and a VL-CDR3, wherein the VH-CDR1, VH-CDR2, VH-CDR3, VL-CDR1, VL-CDR2, and VL-CDR3 are from any one of the VH and VL sequences of the antibodies described herein (e.g., 47A1, 47H1, Hz47H1.v4, 57D12, 61H4, 62G10, Hz62G10.v1, 108D10, or 43H2), such as the amino acid sequences depicted in Tables 9-15. In certain embodiments, the anti-LAIR-1 antibody is a humanized version of an antibody described herein, (e.g., 47A1, 47H1, 57D12, 61H4, 62G10, 108D10, or 43H2).

The antibody designated 47A1 comprises a VH sequence that is set forth in SEQ ID NO:175 and a VL sequence that is set forth in SEQ ID NO:176 (see Table 1). In certain embodiments, a humanized 47A1 antibody comprises a VH comprising a VH-CDR1, a VH-CDR2, and a VH-CDR3, and a VL comprising a VL-CDR1, a VL-CDR2, and a VL-CDR3, wherein the VH-CDR1, VH-CDR2, and VH-CDR3 are from the amino acid sequence of SEQ ID NO:175, and the VL-CDR1, VL-CDR2 and VL-CDR3 are from the amino acid sequence of SEQ ID NO:176. The antibody designated 47H1 comprises a VH sequence that is set forth in SEQ ID NO:177 and a VL sequence that is set forth in SEQ ID NO:178 (see Table 10A). In certain embodiments, a humanized 47H1 antibody comprises a VH comprising a VH-CDR1, a VH-CDR2, and a VH-CDR3, and a VL comprising a VL-CDR1, a VL-CDR2, and a VL-CDR3, wherein the VH-CDR1, VH-CDR2, and VH-CDR3 are from the amino acid sequence of SEQ ID NO:177, and the VL-CDR1, VL-CDR2, and VL-CDR3 are from the amino acid sequence of SEQ ID NO:178. In certain embodiments, a humanized 47H1 antibody comprises a VH comprising a VH-CDR1, a VH-CDR2, and a VH-CDR3, and a VL comprising a VL-CDR1, a VL-CDR2, and a VL-CDR3, wherein the VH-CDR1, VH-CDR2, and VH-CDR3 are from the amino acid sequence of SEQ ID NO:179, and the VL-CDR1, VL-CDR2 and VL-CDR3 are from the amino acid sequence of SEQ ID NO:180 (see Table 10B). The antibody designated 57D12 comprises a VH sequence that is set forth in SEQ ID NO:181 and a VL sequence that is set forth in SEQ ID NO:182 (see Table 11). In certain embodiments, a humanized 57D12 comprises a VH comprising a VH-CDR1, a VH-CDR2, and a VH-CDR3, and a VL comprising a VL-CDR1, a VL-CDR2, and a VL-CDR3, wherein the VH-CDR1, VH-CDR2, and VH-CDR3 are from the amino acid sequence of SEQ ID NO:181, and the VL-CDR1, VL-CDR2, and VL-CDR3 are from the amino acid sequence of SEQ ID NO:182. The antibody designated 61H4 comprises a VH sequence that is set forth in SEQ ID NO:183 and a VL sequence that is set forth in SEQ ID NO:184 (see Table 12). In certain embodiments, a humanized 61H4 antibody comprises a VH comprising a VH-CDR1, a VH-CDR2, and a VH-CDR3, and a VL comprising a VL-CDR1, a VL-CDR2, and a VL-CDR3, wherein the VH-CDR1, VH-CDR2, and VH-CDR3 are from the amino acid sequence of SEQ ID NO:183, and the VL-CDR1, VL-CDR2 and VL-CDR3 are from the amino acid sequence of SEQ ID NO:184. The antibody designated 62G10 comprises a VH sequence that is set forth in SEQ ID NO:185 and a VL sequence that is set forth in SEQ ID NO:186 (see Table 13). In certain embodiments, a humanized 62G10 antibody comprises a VH comprising a VH-CDR1, a VH-CDR2, and a VH-CDR3, and a VL comprising a VL-CDR1, a VL-CDR2, and a VL-CDR3, wherein the VH-CDR1, VH-CDR2, and VH-CDR3 are from the amino acid sequence of SEQ ID NO:185, and the VL-CDR1, VL-CDR2 and VL-CDR3 are from the amino acid sequence of SEQ ID NO:186. In certain embodiments, a humanized 62G10 antibody comprises a VH comprising a VH-CDR1, a VH-CDR2, and a VH-CDR3, and a VL comprising a VL-CDR1, a VL-CDR2, and a VL-CDR3, wherein the VH-CDR1, VH-CDR2, and VH-CDR3 are from the amino acid sequence of SEQ ID NO:187, and the VL-CDR1, VL-CDR2 and VL-CDR3 are from the amino acid sequence of SEQ ID NO:188. The antibody designated 108D10 comprises a VH sequence that is set forth in SEQ ID NO:189 and a VL sequence that is set forth in SEQ ID NO:190 (see Table 14). In certain embodiments, a humanized 108D10 antibody comprises a VH comprising a VH-CDR1, a VH-CDR2, and a VH-CDR3, and a VL comprising a VL-CDR1, a VL-CDR2, and a VL-CDR3, wherein the VH-CDR1, VH-CDR2, and VH-CDR3 are from the amino acid sequence of SEQ ID NO:189, and the VL-CDR1, VL-CDR2, and VL-CDR3 are from the amino acid sequence of SEQ ID NO:190. The antibody designated 43H2 comprises a VH sequence that is set forth in SEQ ID NO:191 and a VL sequence that is set forth in SEQ ID NO:192 (see Table 15). In certain embodiments, a humanized 43H2 antibody comprises a VH comprising a VH-CDR1, a VH-CDR2, a VH-CDR3, and a VL comprising a VL-CDR1, a VL-CDR2, and a VL-CDR3, wherein the VH-CDR1, VH-CDR2, and VH-CDR3 are from the amino acid sequence of SEQ ID NO:191, and the VL-CDR1, VL-CDR2, and VL-CDR3 are from the amino acid sequence of SEQ ID NO:192.

TABLE 9
Antibody 47A1 Sequences

| | Exemplary | Chothia | AbM | Kabat | Contact |
|---|---|---|---|---|---|
| VH- | GFTFNTY | GFTF | GFTFNTY | TYAIH | NTYAIH |
| CDR1 | AIH | NTY | AIH | (SEQ ID | (SEQ ID |
| | (SEQ ID | (SEQ ID | (SEQ ID | NO: 235) | NO: 236) |
| | NO: 226) | NO: 232) | NO: 226) | | |
| VH- | RIRSKST | RSKSTNY | RIRSKST | RIRSKST | WVARIRS |
| CDR2 | NYATYYA | A | NYATY | NYATYYA | KSTNYA |
| | DSV | (SEQ ID | (SEQ ID | D | TY |
| | KD | NO: 233) | NO: 234) | SVKD | (SEQ ID |
| | (SEQ ID | | | (SEQ ID | NO: 237) |
| | NO: 227) | | | NO: 227) | |
| VH- | ENWYYYA | ENWYYYA | ENWYYYA | ENWYYYA | VRENWYY |
| CDR3 | LDY | LDY | LDY | LDY | YALD |
| | (SEQ ID | (SEQ ID | (SEQ ID | (SEQ ID | (SEQ ID |
| | NO: 228) | NO: 228) | NO: 228) | NO: 228) | NO: 238) |
| VL- | RASGNIH | RASGNIH | RASGNIH | RASGNIH | HNYLTWY |
| CDR1 | NYLT | NYLT | NYLT | NYLT | |
| | (SEQ ID | (SEQ ID | (SEQ ID | (SEQ ID | (SEQ ID |
| | NO: 229) | NO: 229) | NO: 229) | NO: 229) | NO: 239) |
| VL- | NAKT | NAKT | NAKT | NAKT | VLVYNAK |
| CDR2 | LED | LED | LED | LED | TLE |
| | (SEQ ID | (SEQ ID | (SEQ ID | (SEQ ID | (SEQ ID |
| | NO: 230) | NO: 230) | NO: 230) | NO: 230) | NO: 240) |
| VL- | QHFWSTP | QHFWSTP | QHFWSTP | QHFWSTP | QHFWSTP |
| CDR3 | FT | FT | FT | FT | F |
| | (SEQ ID | (SEQ ID | (SEQ ID | (SEQ ID | (SEQ ID |
| | NO: 231) | NO: 231) | NO: 231) | NO: 231) | NO: 241) |

47A1 Heavy chain variable region
(SEQ ID NO: 175)
EVQLVESGGGLVQPKGSLKLSCAASGFTFNTYAIHWVRQAPGKGLEWVAR
IRSKSTNYATYYADSVKDRFTISRDDSQSMVFLQMNNLKTEDTAMYYCVR
ENWYYYALDYWGQGTSVTVSS
47A1 Light chain variable region
(SEQ ID NO: 176)
DIQMTQSPASLSASVGETVTITCRASGNIHNYLTWYQQKQGKSPQVLVYN
AKTLEDGVPSRFSGSESGTQYSLKINSLQPEDFGSYYCQHRVSTPFTFGS
GTKLEIK

TABLE 10A
Antibody 47H1 Sequences

| | Exemplary | Chothia | AbM | Kabat | Contact |
|---|---|---|---|---|---|
| VH- | GFTFNIN | GFTFNIN | GFTFNIN | INAMN | NINAMN |
| CDR1 | AMN | (SEQ ID | AMN | (SEQ ID | (SEQ ID |
| | (SEQ ID | NO: 248) | (SEQ ID | NO: 250) | NO: 251) |
| | NO: 242) | | NO: 242) | | |
| VH- | RIRTKNN | RTKNNNY | RIRTKNN | RIRTKNN | WVARIRT |
| CDR2 | NYATFYA | A | NYATF | NYATFYA | KNNN |
| | DSVRD | (SEQ ID | (SEQ ID | DSVKD | YATF |
| | (SEQ ID | NO: 331) | NO: 249) | (SEQ ID | (SEQ ID |
| | NO: 243) | | | NO: 243) | NO: 252) |
| VH- | DRAGFFA | DRAGFFA | DRAGFFA | DRAGFFA | VRDRAGF |
| CDR3 | Y | Y | Y | Y | FA |
| | (SEQ ID | (SEQ ID | (SEQ ID | (SEQ ID | (SEQ ID |
| | NO: 244) | NO: 244) | NO: 244) | NO: 244) | NO: 253) |
| VL- | LASQTIG | LASQTIG | LASQTIG | LASQTIG | GTW |
| CDR1 | TWLG | TWLG | TWLG | TWLG | LGWY |
| | (SEQ ID | (SEQ ID | (SEQ ID | (SEQ ID | (SEQ ID |
| | NO: 245) | NO: 245) | NO: 245) | NO: 245) | NO: 254) |
| VL- | AATSLAD | AATSLAD | AATSLAD | AATSLAD | LLIYAAT |
| CDR2 | (SEQ ID | (SEQ ID | (SEQ ID | (SEQ ID | SLA |
| | NO: 246) | NO: 246) | NO: 246) | NO: 246) | (SEQ ID |
| | | | | | NO: 255) |
| VL- | QQLYSTP | QQLYSTP | QQLYSTP | QQLYSTP | QQLYSTP |
| CDR3 | LT | LT | LT | LT | L |
| | (SEQ ID | (SEQ ID | (SEQ ID | (SEQ ID | (SEQ ID |
| | NO: 247) | NO: 247) | NO: 247) | NO: 247) | NO: 256) |

47H1 Heavy chain variable region
(SEQ ID NO: 177)
EVQLVETGGGLVQPKGSLKLSCAASGFTFNINAMNWVRQAPGKGLEWVA
RIRTKNNNYATFYADSVKDRFTISRDDSQSMLYLQMNNLKTDDTAMYYC
VRDRAGFFAYWGQGTPVTVSA
47H1 Light chain variable region
(SEQ ID NO: 178)
DIQMTQSPASQSASLGESVTITCLASQTIGTWLGWYRQKPGKSPQLLIY
AATSLADGVPSRFSGSGSGTKFSFKISSLQAEDFVIYYCQQLYSTPLTF
GSGTKLIEK

TABLE 10B
Antibody Hz47H1.v4 Sequences

| | Exemplary | Chothia | AbM | Kabat | Contact |
|---|---|---|---|---|---|
| VH- | GFTFNIN | GFTFNIN | GFTFNIN | INAMN | NINAMN |
| CDR1 | AMN | (SEQ ID | AMN | (SEQ ID | (SEQ ID |
| | (SEQ ID | NO: 248 | (SEQ ID | NO: 250) | NO: 251) |
| | NO: 242) | | NO: 242) | | |
| VH- | RIRTKNY | RTKNYNY | RIRTKNY | RIRTKNY | WVARIRT |
| CDR2 | NYATFYA | A | NYATF | NYATFYA | KNYN |
| | DSV | (SEQ ID | (SEQ ID | DSV | YATF |
| | RD | NO: 259) | NO: 260) | KD | (SEQ ID |
| | (SEQ ID | | | (SEQ ID | NO: 261) |
| | NO: 257) | | | NO: 257) | |
| VH- | DRAGFFA | DRAGFFA | DRAGFFA | DRAGFFA | VRDRAGF |
| CDR3 | Y | Y | Y | Y | FA |
| | (SEQ ID | (SEQ ID | (SEQ ID | (SEQ ID | (SEQ ID |
| | NO: 244) | NO: 244) | NO: 244) | NO: 244) | NO: 253) |
| VL- | LASQTIG | LASQTIG | LASQTIG | LASQTIG | GTWLGWY |
| CDR1 | TWLG | TWLG | TWLG | TWLG | (SEQ ID |
| | (SEQ ID | (SEQ ID | (SEQ ID | (SEQ ID | NO: 254) |
| | NO: 245) | NO: 245) | NO: 245) | NO: 245) | |
| VL- | AATSLAE | AATSLAE | AATSLAE | AATSLAE | LLIYAAT |
| CDR2 | (SEQ ID | (SEQ ID | (SEQ ID | (SEQ ID | SLA |
| | NO: 258) | NO: 258) | NO: 258) | NO: 258) | (SEQ ID |
| | | | | | NO: 255) |
| VL- | QQLYSTP | QQLYSTP | QQLYSTP | QQLYSTP | QQLYSTP |
| CDR3 | LT | LT | LT | LT | L |
| | (SEQ ID | (SEQ ID | (SEQ ID | (SEQ ID | (SEQ ID |
| | NO: 247) | NO: 247) | NO: 247) | NO: 247) | NO: 256) |

Hz47H1.v4 Heavy chain variable region
(SEQ ID NO: 179)
EVQLVESGGGLVQPGGSLRLSCAASGFTFNINAMNWVRQAPGKGLEWVA
RIRTKNYNYATFYADSVKDRFTISRDDSKNSLYLQMNSLKTEDTAVYYC
VRDRAGFFAYWGQGTTVTVSS
Hz47H1.v4 Light chain variable region
(SEQ ID NO: 180)
DIQMTQSPSSLSASVGDRVTITCLASQTIGTWLGWYQQKPGKAPKLLIY
AATSLAEGVPSRFSGSGSGTDFTLTISSLQPEDFATYYCQQLYSTPLTF
GGGTKVEIK

TABLE 11
Antibody 57D12 Sequences

| | Exemplary | Chothia | AbM | Kabat | Contact |
|---|---|---|---|---|---|
| VH | GYSFTSF | GYSFTSF | GYSFTSF | SFGIS | TSFGIS |
| CDR1 | GIS | (SEQ ID | GIS | (SEQ ID | (SEQ ID |
| | (SEQ ID | NO: 268) | (SEQ ID | NO: 271) | NO: 272) |
| | NO: 262) | | NO: 262) | | |
| VH | EIYPRSD | YPRSDN | EIYPRSD | EIYPRSD | WIGEIYP |
| CDR2 | NTFYNEK | (SEQ ID | NTF | NTFYNEK | RSDNTF |
| | FKG | NO: 269) | (SEQ ID | FKG | (SEQ ID |
| | (SEQ ID | | NO: 270) | (SEQ ID | NO: 273) |
| | NO: 263) | | | NO: 263) | |
| VH | HFGSSSF | HFGSSSF | HFGSSSF | HFGSSSF | ARHFGSS |
| CDR3 | DY | DY | DY | DY | SFD |
| | (SEQ ID | (SEQ ID | (SEQ ID | (SEQ ID | (SEQ ID |
| | NO: 264) | NO: 264) | NO: 264) | NO: 264) | NO: 274) |
| VL- | SASSSVS | SASSSVS | SASSSVS | SASSSVS | SSIYFHW |
| CDR1 | SIYFH | SIYFH | SIYFH | SIYFH | Y |
| | (SEQ ID | (SEQ ID | (SEQ ID | (SEQ ID | (SEQ ID |
| | NO: 265) | NO: 265) | NO: 265) | NO: 265) | NO: 275) |
| VL- | RASNLAS | RASNLAS | RASNLAS | RASNLAS | PLIHRAS |
| CDR2 | (SEQ ID | (SEQ ID | (SEQ ID | (SEQ ID | NLA |
| | NO: 266) | NO: 266) | NO: 266) | NO: 266) | (SEQ ID |
| | | | | | NO: 276) |
| VL- | QQWSGYP | QQWSGYP | QQWSGYP | QQWSGYP | QQWSGYP |
| CDR3 | LT | LT | LT | LT | L |
| | (SEQ ID | (SEQ ID | (SEQ ID | (SEQ ID | (SEQ ID |
| | NO: 267) | NO: 267) | NO: 267) | NO: 267) | NO: 277) |

57D12 Heavy chain variable region
(SEQ ID NO: 181)
QVQLQQSGAELARPGASVNLSCRASGYSFTSFGISWVKQRTGQGLEWIGEI
YPRSDNTFYNEKFKGKATLTADKSSSTAYMELRSLTSEDSAVYFCARHFGS
SSFDYWGQGTTLTVSS
57D12 Light chain variable region
(SEQ ID NO: 182)
ENVLTQSPPIMAASLGQKVTMTCSASSSVSSIYFHWYQQKSGTSPKPLIHR
GASNLASVPARFSGSGSGTSYSLTISSVEAEDDATYYCQQWSGYPLTFGGG
TKLEIK

TABLE 12
Antibody 61H4 Sequences

| | Exemplary | Chothia | AbM | Kabat | Contact |
|---|---|---|---|---|---|
| VH- | GYTFTDY | GYTFTDY | GYTFTDY | DYYYMN | TDYYYMN |
| CDR1 | YYMN | Y | YYMN | (SEQ ID | (SEQ ID |
| | (SEQ ID | (SEQ ID | (SEQ ID | NO: 287) | NO: 288) |
| | NO: 278) | NO: 284) | NO: 278) | | |
| VH- | YIYPNNG | YPNNGA | YIYPNNG | YIYPNNG | WIGYIYP |
| CDR2 | ATSYNQK | (SEQ ID | ATS | ATSYNQK | NNGATS |
| | FKG | NO: 285) | (SEQ ID | FKG | (SEQ ID |
| | (SEQ ID | | NO: 286) | (SEQ ID | NO: 289) |
| | NO: 279) | | | NO: 279) | |
| VH- | DGYSSNY | DGYSSNY | DGYSSNY | DGYSSNY | ARDGYSS |
| CDR3 | YTMDY | YTMDY | YTMDY | YTMDY | NYYTMD |
| | (SEQ ID | (SEQ ID | (SEQ ID | (SEQ ID | (SEQ ID |
| | NO: 280) | NO: 280) | NO: 280) | NO: 280) | NO: 290) |
| VL- | QASQGTS | QASQGTS | QASQGTS | QASQGTS | SINL |
| CDR1 | INLN | INLN | INLN | INLN | NWF |
| | (SEQ ID | (SEQ ID | (SEQ ID | (SEQ ID | (SEQ ID |
| | NO: 281) | NO: 281) | NO: 281) | NO: 281) | NO: 291) |
| VL- | GASNLED | GASNLED | GASNLED | GASNLED | LLIYGAS |
| CDR2 | (SEQ ID | (SEQ ID | (SEQ ID | (SEQ ID | NLE |
| | NO: 282) | NO: 282) | NO: 282) | NO: 282) | (SEQ ID |
| | | | | | NO: 292) |
| VL- | LQHTYLP | LQHTYLP | LQHTYLP | LQHTYLP | LQHTYLP |
| CDR3 | YT | YT | YT | YT | Y |
| | (SEQ ID | (SEQ ID | (SEQ ID | (SEQ ID | (SEQ ID |
| | NO: 283) | NO: 283) | NO: 283) | NO: 283) | NO: 293) |

61H4 Heavy chain variable region
(SEQ ID NO: 183)
EVQLQQSGPEVLKPGASVKISCKASGYTFTDYYYMNWVKQSHGKSLEWIGY
IYPNNGATSYNQKFKGKATLTVDKSSSTAYMELRSLTSEDSAVYYCARDGY
SSNYYTMDYWGQGTSVTVSS
61H4 Light chain variable region
(SEQ ID NO: 184)
DVQMIQSPSSLSASLGDIVTMTCQASQGTSINLNWFQQKPGKAPKLLIYGA
SNLEDGVPSRFSGSRYGTDFTLTISSLEDEDMATYFCLQHTYLPYTFGGGT
KLEIK

TABLE 13
Antibody 62G10 and Hz62G10.v1 Sequences

| | Exemplary | Chothia | AbM | Kabat | Contact |
|---|---|---|---|---|---|
| VH- | GFTFNIN | GFTFNIN | GFTFNIN | INAMN | NINAMN |
| CDR1 | AMN | (SEQ ID | AMN | (SEQ ID | (SEQ ID |
| | (SEQ ID | NO: 248 | (SEQ ID | NO: 250) | NO: 251) |
| | NO: 242) | | NO: 242) | | |
| VH- | RIRTKNN | RTKNNNF | RIRTKNN | RIRTKNN | WVARIR |
| CDR2 | NFATYYA | A | NFATY | NFATYYA | TKNN |
| | DSVRD | (SEQ ID | (SEQ ID | DSVKD | NFATY |
| | (SEQ ID | NO: 298) | NO: 299) | (SEQ ID | (SEQ ID |
| | NO: 294) | | | NO: 294) | NO: 300) |
| VH- | GPYFDY | GPYFDY | GPYFDY | GPYFDY | VRGPYFD |
| CDR3 | (SEQ ID | (SEQ ID | (SEQ ID | (SEQ ID | (SEQ ID |
| | NO: 295) | NO: 295) | NO: 295) | NO: 295) | NO: 301) |
| VL- | LASQTIG | LASQTIG | LASQTIG | LASQTIG | GTWLAWY |
| CDR1 | TWLA | TWLA | TWLA | TWLA | (SEQ ID |
| | (SEQ ID | (SEQ ID | (SEQ ID | (SEQ ID | NO: 302) |
| | NO: 296) | NO: 296) | NO: 296) | NO: 296) | |
| VL- | AATSLAD | AATSLAD | AATSLAD | AATSLAD | LLIYAAT |
| CDR2 | (SEQ ID | (SEQ ID | (SEQ ID | (SEQ ID | SLA |
| | NO: 246) | NO: 246) | NO: 246) | NO: 246) | (SEQ ID |
| | | | | | NO: 255) |
| VL- | QQLYSTP | QQLYSTP | QQLYSTP | QQLYSTP | QQLYSTP |
| CDR3 | YT | YT | YT | YT | Y |
| | (SEQ ID | (SEQ ID | (SEQ ID | (SEQ ID | (SEQ ID |
| | NO: 297) | NO: 297) | NO: 297) | NO: 297) | NO: 303) |

62G10 Heavy chain variable region
(SEQ ID NO: 185)
EVQLVETGGGLVQPKGSLKLSCATSGFTFNINAMNWVRQAPGKGLEWVARI
RTKNNNFATYYADSVKDRFTISRDDSQSMLYLQMNNLKTEDTAMYYCVRGP
YFDYWGQGTTLTVSS
62G10 Light chain variable region
(SEQ ID NO: 186)
DIQMTQSPASQSASLGESVTITCLASQTIGTWLAWYQQKPGKSPQLLIYAA
TSLADGVPSRFSGSGSGTKFSFKISNLQAEDFVTYYCQQLYSTPYTFGGGT
KLEIK
Hz62G10.v1 Heavy chain variable region
(SEQ ID NO: 187)
EVQLVESGGGLVKPGGSLRLSCAASGFTFNINAMNWVRQAPGKGLEWVAR
IRTKNNNFATYYADSVKDRFTISRDDSKNTLYLQMNSLKTEDTAVYYCVR
GPYFDYWGQGTLVTVSS
Hz62G10.v1 Light chain variable region
(SEQ ID NO: 188)
DIQMTQSPSSLSASVGDRVTITCLASQTIGTWLAWYQQKPGKAPKLLIYA
ATSLADGVPSRFSGSGSGTDFTLTISSLQPEDFATYYCQQLYSTPYTFGG
GTKVEIK

TABLE 14
Antibody 108D10 Sequences

| | Exemplary | Chothia | AbM | Kabat | Contact |
|---|---|---|---|---|---|
| VH- | GFTFNIN | GFTFNIN | GFTFNIN | INAMN | NINAMN |
| CDR1 | AMN | (SEQ ID | AMN | (SEQ ID | (SEQ ID |
| | (SEQ ID | NO: 248 | (SEQ ID | NO: 250) | NO: 251) |
| | NO: 242) | | NO: 242) | | |
| VH- | RIRTKNN | RTKNNNY | RIRTKNN | RIRTKNN | WVARIRT |
| CDR2 | NYATYYA | A | NYATY | NYATYYA | KNNNYA |
| | DSVK | (SEQ ID | (SEQ ID | DS | TY |
| | D | NO: 331) | NO: 309) | VKD | (SEQ ID |
| | (SEQ ID | | | (SEQ ID | NO: 310) |
| | NO: 304) | | | NO: 304) | |
| VH- | DRYGGAM | DRYGGAM | DRYGGAM | DRYGGAM | VRDRYGG |
| CDR3 | AY | AY | AY | AY | AMA |
| | (SEQ ID | (SEQ ID | (SEQ ID | (SEQ ID | (SEQ ID |
| | NO: 305) | NO: 305) | NO: 305) | NO: 305) | NO: 311) |
| VL- | KASEDIY | KASEDIY | KASEDIY | KASEDIY | YNRLAWY |
| CDR1 | NRLA | NRLA | NRLA | NRLA | (SEQ ID |
| | (SEQ ID | (SEQ ID | (SEQ ID | (SEQ ID | NO: 312) |
| | NO: 306) | NO: 306) | NO: 306) | NO: 306) | |
| VL- | SATSLET | SATSLET | SATSLET | SATSLET | LLISSAT |
| CDR2 | (SEQ ID | (SEQ ID | (SEQ ID | (SEQ ID | SLE |
| | NO: 307) | NO: 307) | NO: 307) | NO: 307) | (SEQ ID |
| | | | | | NO: 313) |
| VL- | QQYWTIP | QQYWTIP | QQYWTIP | QQYWTIP | QQYWTIP |
| CDR3 | YT | YT | YT | YT | Y |
| | (SEQ ID | (SEQ ID | (SEQ ID | (SEQ ID | (SEQ ID |
| | NO: 308) | NO: 308) | NO: 308) | NO: 308) | NO: 314) |

108D10 Heavy chain variable region
(SEQ ID NO: 189)
EVQLVETGGGLVQPKGSLKLSCAASGFTFNINAMNWVRQAPGKGLEWVARI
RTKNNNYATYYADSVKDRFTISRDDSESMLYLQMNNLKTEDTAMYYCVRD
RYGGAMAYWGQGTSVTVSS
108D10 Light chain variable region
(SEQ ID NO: 190)
DIQMTQSSSSFSVSLGDRVTITCKASEDIYNRLAWYQQKPGNVPRLLISSA
TSLETGVPSRFSGSGSGKDYTLSLTSLQSEDVATYYCQQYWTIPYTFGGG
TKLEIK

TABLE 15
Antibody 43H2 Sequences

| | Exemplary | Chothia | AbM | Kabat | Contact |
|---|---|---|---|---|---|
| VH- | GFTFSNY | GFTFSNY | GFTFSNY | NYGIH | SNYGIH |
| CDR1 | GIH | (SEQ ID | GIH | (SEQ ID | (SEQ ID |
| | (SEQ ID | NO: 321) | (SEQ ID | NO: 324) | NO: 325) |
| | NO: 315) | | NO: 315) | | |
| VH- | SISPSGR | SPSGRS | SISPSGR | SISPSGR | WVASISP |
| CDR2 | STYFRDS | (SEQ ID | STY | STYFRDS | SGRSTY |
| | VKG | NO: 322) | (SEQ ID | VKG | (SEQ ID |
| | (SEQ ID | | NO: 323) | (SEQ ID | NO: 326) |
| | NO: 316) | | | NO: 316) | |
| VH- | GINYSSF | GINYSSF | GINYSSF | GINYSSF | ATGINYS |
| CDR3 | DY | DY | DY | DY | SFD |
| | (SEQ ID | (SEQ ID | (SEQ ID | (SEQ ID | (SEQ ID |
| | NO: 317) | NO: 317) | NO: 317) | NO: 317) | NO: 327) |
| VL- | KASQNVG | KASQNVG | KASQNVG | KASQNVG | GSHVDWY |
| CDR1 | SHVD | SHVD | SHVD | SHVD | (SEQ ID |
| | (SEQ ID | (SEQ ID | (SEQ ID | (SEQ ID | NO: 328) |
| | NO: 318) | NO: 318) | NO: 318) | NO: 318) | |
| VL- | TASNRYT | TASNRYT | TASNRYT | TASNRYT | LLISTAS |
| CDR2 | (SEQ ID | (SEQ ID | (SEQ ID | (SEQ ID | NRY |
| | NO: 319) | NO: 319) | NO: 319) | NO: 319) | (SEQ ID |
| | | | | | NO: 329) |
| VL- | MQSNSYP | MQSNSYP | MQSNSYP | MQSNSYP | MQSNSYP |
| CDR3 | PT | PT | PT | PT | P |
| | (SEQ ID | (SEQ ID | (SEQ ID | (SEQ ID | (SEQ ID |
| | NO: 320) | NO: 320) | NO: 320) | NO: 320) | NO: 330) |

43H2 Heavy chain variable region
(SEQ ID NO: 191)
EVQLVESGGGLVQPGRSLKVSCAASGFTFSNYGIHWIRQAPTKGLEWVASI
SPSGRSTYFRDSVKGRFTISRDNAKNTLYLQLDSLRSEDTATYYCATGIN
YSSFDYWGQGVMVTVSS
43H2 Light chain variable region
(SEQ ID NO: 192)
DIVMTQSPTSMSISVGDRVTMNCKASQNVGSHVDWYQQKTGQSPKLLISTA
SNRYTGVPDRFTGSGSGTDFTFTINNMQTEDLAVYYCMQSNSYPPTFGGGT
KLELK

In certain embodiments, the anti-LAIR-1 antibody comprises (i) a VH comprising a VH-CDR1, a VH-CDR2, and a VH-CDR3 defined by any one of the CDR definitions from Table 9; and (ii) a VL comprising a VL-CDR1, a VL-CDR2, and a VL-CDR3 defined by any one of the CDR definitions from Table 9.

In certain embodiments, the anti-LAIR-1 antibody comprises (i) a VH comprising a VH-CDR1, a VH-CDR2, and a VH-CDR3 defined by any one of the CDR definitions from Table 10; and (ii) a VL comprising a VL-CDR1, a VL-CDR2, and a VL-CDR3 defined by any one of the CDR definitions from Table 10A.

In certain embodiments, the anti-LAIR-1 antibody comprises (i) a VH comprising a VH-CDR1, a VH-CDR2, and a VH-CDR3 defined by any one of the CDR definitions from Table 10A; and (ii) a VL comprising a VL-CDR1, a VL-CDR2, and a VL-CDR3 defined by any one of the CDR definitions from Table 10B.

In certain embodiments, the anti-LAIR-1 antibody comprises (i) a VH comprising a VH-CDR1, a VH-CDR2, and a VH-CDR3 defined by any one of the CDR definitions from Table 11; and (ii) a VL comprising a VL-CDR1, a VL-CDR2, and a VL-CDR3 defined by any one of the CDR definitions from Table 11.

In certain embodiments, the anti-LAIR-1 antibody comprises (i) a VH comprising a VH-CDR1, a VH-CDR2, and a VH-CDR3 defined by any one of the CDR definitions from Table 12; and (ii) a VL comprising a VL-CDR1, a VL-CDR2, and a VL-CDR3 defined by any one of the CDR definitions from Table 12.

In certain embodiments, the anti-LAIR-1 antibody comprises (i) a VH comprising a VH-CDR1, a VH-CDR2, and a VH-CDR3 defined by any one of the CDR definitions from Table 13; and (ii) a VL comprising a VL-CDR1, a VL-CDR2, and a VL-CDR3 defined by any one of the CDR definitions from Table 13.

In certain embodiments, the anti-LAIR-1 antibody comprises (i) a VH comprising a VH-CDR1, a VH-CDR2, and a VH-CDR3 defined by any one of the CDR definitions from Table 14; and (ii) a VL comprising a VL-CDR1, a VL-CDR2, and a VL-CDR3 defined by any one of the CDR definitions from Table 15.

In certain embodiments, the anti-LAIR-1 antibody comprises (i) a VH comprising a VH-CDR1, a VH-CDR2, and a VH-CDR3 defined by any one of the CDR definitions from Table 15; and (ii) a VL comprising a VL-CDR1, a VL-CDR2, and a VL-CDR3 defined by any one of the CDR definitions from Table 15.

In certain embodiments, the anti-LAIR-1 antibody comprises (i) a VH comprising the VH-CDR1, the VH-CDR2, and the VH-CDR3 from the amino acid sequence set forth in SEQ ID NO:175; and (ii) a VL comprising the VL-CDR1, the VL-CDR2, and the VL-CDR3 from the amino acid sequence set forth in SEQ ID NO:176 (i.e., the six CDRs of antibody 47A1 of Table 9).

In certain embodiments, the anti-LAIR-1 antibody comprises (i) a VH comprising the VH-CDR1, the VH-CDR2, and the VH-CDR3 from the amino acid sequence set forth in SEQ ID NO:177; and (ii) a VL comprising the VL-CDR1, the VL-CDR2, and the VL-CDR3 from the amino acid sequence set forth in SEQ ID NO:178 (i.e., the six CDRs of antibody 47H1 of Table 10A).

In certain embodiments, the anti-LAIR-1 antibody comprises (i) a VH comprising the VH-CDR1, the VH-CDR2, and the VH-CDR3 from the amino acid sequence set forth in SEQ ID NO:179; and (ii) a VL comprising the VL-CDR1, the VL-CDR2, and the VL-CDR3 from the amino acid sequence set forth in SEQ ID NO:180 (i.e., the six CDRs of antibody Hz47H1.v4 of Table 10B).

In certain embodiments, the anti-LAIR-1 antibody comprises (i) a VH comprising the VH-CDR1, the VH-CDR2, and the VH-CDR3 from the amino acid sequence set forth in SEQ ID NO:181; and (ii) a VL comprising the VL-CDR1, the VL-CDR2, and the VL-CDR3 from the amino acid sequence set forth in SEQ ID NO:182 (i.e., the six CDRs of antibody 57D12 of Table 11).

In certain embodiments, the anti-LAIR-1 antibody comprises (i) a VH comprising the VH-CDR1, the VH-CDR2, and the VH-CDR3 from the amino acid sequence set forth in SEQ ID NO:183; and (ii) a VL comprising the VL-CDR1, the VL-CDR2, and the VL-CDR3 from the amino acid sequence set forth in SEQ ID NO:184 (i.e., the six CDRs of antibody 61H4 of Table 12).

In certain embodiments, the anti-LAIR-1 antibody comprises (i) a VH comprising the VH-CDR1, the VH-CDR2, and the VH-CDR3 from the amino acid sequence set forth in SEQ ID NO:185; and (ii) a VL comprising the VL-CDR1, the VL-CDR2, and the VL-CDR3 from the amino acid sequence set forth in SEQ ID NO:186; or (i) a VH comprising the VH-CDR1, the VH-CDR2, and the VH-CDR3 from the amino acid sequence set forth in SEQ ID NO:187; and (ii) a VL comprising the VL-CDR1, the VL-CDR2, and the VL-CDR3 from the amino acid sequence set forth in SEQ ID NO:188 (i.e., the six CDRs of antibodies 62G10 and Hz62G10.v1 of Table 13).

In certain embodiments, the anti-LAIR-1 antibody comprises (i) a VH comprising the VH-CDR1, the VH-CDR2, and the VH-CDR3 from the amino acid sequence set forth in SEQ ID NO:189; and (ii) a VL comprising the VL-CDR1, the VL-CDR2, and the VL-CDR3 from the amino acid sequence set forth in SEQ ID NO:190 (i.e., the six CDRs of antibody 108D10 of Table 14).

In certain embodiments, the anti-LAIR-1 antibody comprises (i) a VH comprising the VH-CDR1, the VH-CDR2, and the VH-CDR3 from the amino acid sequence set forth in SEQ ID NO:191; and (ii) a VL comprising the VL-CDR1, the VL-CDR2, and the VL-CDR3 from the amino acid sequence set forth in SEQ ID NO:192 (i.e., the six CDRs of antibody 43H2 of Table 15).

In certain embodiments, the CDRs of the anti-LAIR-1 antibody are according to the Exemplary designation. In certain embodiments, the anti-LAIR-1 antibody comprises: (i) a VH comprising a VH-CDR1 comprising the amino acid sequence set forth in SEQ ID NO:226, a VH-CDR2 comprising the amino acid sequence set forth in SEQ ID NO:227, and a VH-CDR3 comprising the amino acid sequence set forth in SEQ ID NO:228; and (ii) a VL comprising a VL-CDR1 comprising the amino acid sequence set forth in SEQ ID NO:229, a VL-CDR2 comprising the amino acid sequence set forth in SEQ ID NO:230, and a VL-CDR3 comprising the amino acid sequence set forth in SEQ ID NO:231. In certain embodiments, the anti-LAIR-1 antibody comprises: (i) a VH comprising a VH-CDR1 comprising the amino acid sequence set forth in SEQ ID NO:242, a VH-CDR2 comprising the amino acid sequence set forth in SEQ ID NO:243, and a VH-CDR3 comprising the amino acid sequence set forth in SEQ ID NO:244; and (ii) a VL comprising a VL-CDR1 comprising the amino acid sequence set forth in SEQ ID NO:245, a VL-CDR2 comprising the amino acid sequence set forth in SEQ ID NO:246, and a VL-CDR3 comprising the amino acid sequence set forth in SEQ ID NO:247. In certain embodiments, the anti-LAIR-1 antibody comprises: (i) a VH comprising a VH-CDR1 comprising the amino acid sequence set forth in SEQ ID NO:242, a VH-CDR2 comprising the amino acid sequence set forth in SEQ ID NO:257, and a VH-CDR3 comprising the amino acid sequence set forth in SEQ ID NO:244; and (ii) a VL comprising a VL-CDR1 comprising the amino acid sequence set forth in SEQ ID NO:245, a VL-CDR2 comprising the amino acid sequence set forth in SEQ ID NO:258, and a VL-CDR3 comprising the amino acid sequence set forth in SEQ ID NO:247. In certain embodiments, the anti-LAIR-1 antibody comprises: (i) a VH comprising a VH-CDR1 comprising the amino acid sequence set forth in SEQ ID NO:262, a VH-CDR2 comprising the amino acid sequence set forth in SEQ ID NO:263, and a VH-CDR3 comprising the amino acid sequence set forth in SEQ ID NO:264; and (ii) a VL comprising a VL-CDR1 comprising the amino acid sequence set forth in SEQ ID NO:265, a VL-CDR2 comprising the amino acid sequence set forth in SEQ ID NO:266, and a VL-CDR3 comprising the amino acid sequence set forth in SEQ ID NO:267. In certain embodiments, the anti-LAIR-1 antibody comprises: (i) a VH comprising a VH-CDR1 comprising the amino acid sequence set forth in SEQ ID NO:278, a VH-CDR2 comprising the amino acid sequence set forth in SEQ ID NO:279, and a VH-CDR3 comprising the amino acid sequence set forth in SEQ ID NO:280; and (ii) a VL comprising a VL-CDR1 comprising the amino acid sequence set forth in SEQ ID NO:281, a VL-CDR2 comprising the amino acid sequence set forth in SEQ ID NO:282, and a VL-CDR3 comprising the amino acid sequence set forth in SEQ ID NO:283. In certain embodiments, the anti-LAIR-1 antibody comprises: (i) a VH comprising a VH-CDR1 comprising the amino acid sequence set forth in SEQ ID NO:242, a VH-CDR2 comprising the amino acid sequence set forth in SEQ ID NO:294, and a VH-CDR3 comprising the amino acid sequence set forth in SEQ ID NO:295; and (ii) a VL comprising a VL-CDR1 comprising the amino acid sequence set forth in SEQ ID NO:296, a VL-CDR2 comprising the amino acid sequence set forth in SEQ ID NO:246, and a VL-CDR3 comprising the amino acid sequence set forth in SEQ ID NO:297. In certain embodiments, the anti-LAIR-1 antibody comprises: (i) a VH comprising a VH-CDR1 comprising the amino acid sequence set forth in SEQ ID NO:242, a VH-CDR2 comprising the amino acid sequence set forth in SEQ ID NO:304, and a VH-CDR3 comprising the amino acid sequence set forth in SEQ ID NO:305; and (ii) a VL comprising a VL-CDR1 comprising the amino acid sequence set forth in SEQ ID NO:306, a VL-CDR2 comprising the amino acid sequence set forth in SEQ ID NO:307, and a VL-CDR3 comprising the amino acid sequence set forth in SEQ ID NO:308. In certain embodiments, the anti-LAIR-1 antibody comprises: (i) a VH comprising a VH-CDR1 comprising the amino acid sequence set forth in SEQ ID NO:315, a VH-CDR2 comprising the amino acid sequence set forth in SEQ ID NO:316, and a VH-CDR3 comprising the amino acid sequence set forth in SEQ ID NO:317; and (ii) a VL comprising a VL-CDR1 comprising the amino acid sequence set forth in SEQ ID NO:318, a VL-CDR2 comprising the amino acid sequence set forth in SEQ ID NO:319, and a VL-CDR3 comprising the amino acid sequence set forth in SEQ ID NO:320.

In certain embodiments, the CDRs of the anti-LAIR-1 antibody are according to the Chothia designation. In certain embodiments, the anti-LAIR-1 antibody comprises: (i) a VH comprising a VH-CDR1 comprising the amino acid sequence set forth in SEQ ID NO:232, a VH-CDR2 comprising the amino acid sequence set forth in SEQ ID NO:233, and a VH-CDR3 comprising the amino acid sequence set forth in SEQ ID NO:228; and (ii) a VL comprising a VL-CDR1 comprising the amino acid sequence set forth in SEQ ID NO:229, a VL-CDR2 comprising the amino acid sequence set forth in SEQ ID NO:230, and a VL-CDR3 comprising the amino acid sequence set forth in SEQ ID NO:231. In certain embodiments, the anti-LAIR-1 antibody comprises: (i) a VH comprising a VH-CDR1 comprising the amino acid sequence set forth in SEQ ID NO:248, a VH-CDR2 comprising the amino acid sequence set forth in SEQ ID NO:331, and a VH-CDR3 comprising the amino acid sequence set forth in SEQ ID NO:244; and (ii) a VL comprising a VL-CDR1 comprising the amino acid sequence set forth in SEQ ID NO:245, a VL-CDR2 comprising the amino acid sequence set forth in SEQ ID NO:246, and a VL-CDR3 comprising the amino acid sequence set forth in SEQ ID NO:247. In certain embodiments, the anti-LAIR-1 antibody comprises: (i) a VH comprising a VH-CDR1 comprising the amino acid sequence set forth in SEQ ID NO:248, a VH-CDR2 comprising the amino acid sequence set forth in SEQ ID NO:259, and a VH-CDR3 comprising the amino acid sequence set forth in SEQ ID NO:244; and (ii) a VL comprising a VL-CDR1 comprising the amino acid sequence set forth in SEQ ID NO:245, a VL-CDR2 comprising the amino acid sequence set forth in SEQ ID NO:258, and a VL-CDR3 comprising the amino acid sequence set forth in SEQ ID NO:247. In certain embodiments, the anti-LAIR-1 antibody comprises: (i) a VH comprising a VH-CDR1 comprising the amino acid sequence set forth in SEQ ID NO:268, a VH-CDR2 comprising the amino acid sequence set forth in SEQ ID NO:269, and a VH-CDR3 comprising the amino acid sequence set forth in SEQ ID NO:264; and (ii) a VL comprising a VL-CDR1 comprising the amino acid sequence set forth in SEQ ID NO:265, a VL-CDR2 comprising the amino acid sequence set forth in SEQ ID NO:266, and a VL-CDR3 comprising the amino acid sequence set forth in SEQ ID NO:267. In certain embodiments, the anti-LAIR-1 antibody comprises: (i) a VH comprising a VH-CDR1 comprising the amino acid sequence set forth in SEQ ID NO:284, a VH-CDR2 comprising the amino acid sequence set forth in SEQ ID NO:285, and a VH-CDR3 comprising the amino acid sequence set forth in SEQ ID NO:280; and (ii) a VL comprising a VL-CDR1 comprising the amino acid sequence set forth in SEQ ID NO:281, a VL-CDR2 comprising the amino acid sequence set forth in SEQ ID NO:282, and a VL-CDR3 comprising the amino acid sequence set forth in SEQ ID NO:283. In certain embodiments, the anti-LAIR-1 antibody comprises: (i) a VH comprising a VH-CDR1 comprising the amino acid sequence set forth in SEQ ID NO:248, a VH-CDR2 comprising the amino acid sequence set forth in SEQ ID NO:298, and a VH-CDR3 comprising the amino acid sequence set forth in SEQ ID NO:295; and (ii) a VL comprising a VL-CDR1 comprising the amino acid sequence set forth in SEQ ID NO:296, a VL-CDR2 comprising the amino acid sequence set forth in SEQ ID NO:246, and a VL-CDR3 comprising the amino acid sequence set forth in SEQ ID NO:297. In certain embodiments, the anti-LAIR-1 antibody comprises: (i) a VH comprising a VH-CDR1 comprising the amino acid sequence set forth in SEQ ID NO:248, a VH-CDR2 comprising the amino acid sequence set forth in SEQ ID NO:331, and a VH-CDR3 comprising the amino acid sequence set forth in SEQ ID NO:305; and (ii) a VL comprising a VL-CDR1 comprising the amino acid sequence set forth in SEQ ID NO:306, a VL-CDR2 comprising the amino acid sequence set forth in SEQ ID NO:307, and a VL-CDR3 comprising the amino acid sequence set forth in SEQ ID NO:308. In certain embodiments, the anti-LAIR-1 antibody comprises: (i) a VH comprising a VH-CDR1 comprising the amino acid sequence set forth in SEQ ID NO:321, a VH-CDR2 comprising the amino acid sequence set forth in SEQ ID NO:322, and a VH-CDR3 comprising the amino acid sequence set forth in SEQ ID NO:317; and (ii) a VL comprising a VL-CDR1 comprising the amino acid sequence set forth in SEQ ID NO:318, a VL-CDR2 comprising the amino acid sequence set forth in SEQ ID NO:319, and a VL-CDR3 comprising the amino acid sequence set forth in SEQ ID NO:320.

In certain embodiments, the CDRs of the anti-LAIR-1 antibody are according to the AbM designation. In certain embodiments, the anti-LAIR-1 antibody comprises: (i) a VH comprising a VH-CDR1 comprising the amino acid sequence set forth in SEQ ID NO:226, a VH-CDR2 comprising the amino acid sequence set forth in SEQ ID NO:234, and a VH-CDR3 comprising the amino acid sequence set forth in SEQ ID NO:228; and (ii) a VL comprising a VL-CDR1 comprising the amino acid sequence set forth in SEQ ID NO:229, a VL-CDR2 comprising the amino acid sequence set forth in SEQ ID NO:230, and a VL-CDR3 comprising the amino acid sequence set forth in SEQ ID NO:231. In certain embodiments, the anti-LAIR-1 antibody comprises: (i) a VH comprising a VH-CDR1 comprising the amino acid sequence set forth in SEQ ID NO:242, a VH-CDR2 comprising the amino acid sequence set forth in SEQ ID NO:249, and a VH-CDR3 comprising the amino acid sequence set forth in SEQ ID NO:244; and (ii) a VL comprising a VL-CDR1 comprising the amino acid sequence set forth in SEQ ID NO:245, a VL-CDR2 comprising the amino acid sequence set forth in SEQ ID NO:246, and a VL-CDR3 comprising the amino acid sequence set forth in SEQ ID NO:247. In certain embodiments, the anti-LAIR-1 antibody comprises: (i) a VH comprising a VH-CDR1 comprising the amino acid sequence set forth in SEQ ID NO:242, a VH-CDR2 comprising the amino acid sequence set forth in SEQ ID NO:260, and a VH-CDR3 comprising the amino acid sequence set forth in SEQ ID NO:244; and (ii) a VL comprising a VL-CDR1 comprising the amino acid sequence set forth in SEQ ID NO:245, a VL-CDR2 comprising the amino acid sequence set forth in SEQ ID NO:258, and a VL-CDR3 comprising the amino acid sequence set forth in SEQ ID NO:247. In certain embodiments, the anti-LAIR-1 antibody comprises: (i) a VH comprising a VH-CDR1 comprising the amino acid sequence set forth in SEQ ID NO:262, a VH-CDR2 comprising the amino acid sequence set forth in SEQ ID NO:270, and a VH-CDR3 comprising the amino acid sequence set forth in SEQ ID NO:264; and (ii) a VL comprising a VL-CDR1 comprising the amino acid sequence set forth in SEQ ID NO:265, a VL-CDR2 comprising the amino acid sequence set forth in SEQ ID NO:266, and a VL-CDR3 comprising the amino acid sequence set forth in SEQ ID NO:267. In certain embodiments, the anti-LAIR-1 antibody comprises: (i) a VH comprising a VH-CDR1 comprising the amino acid sequence set forth in SEQ ID NO:278, a VH-CDR2 comprising the amino acid sequence set forth in SEQ ID NO:286, and a VH-CDR3 comprising the amino acid sequence set forth in SEQ ID NO:280; and (ii) a VL comprising a VL-CDR1 comprising the amino acid sequence set forth in SEQ ID NO:281, a VL-CDR2 comprising the amino acid sequence set forth in SEQ ID NO:282, and a VL-CDR3 comprising the amino acid sequence set forth in SEQ ID NO:283. In certain embodiments, the anti-LAIR-1 antibody comprises: (i) a VH comprising a VH-CDR1 comprising the amino acid sequence set forth in SEQ ID NO:242, a VH-CDR2 comprising the amino acid sequence set forth in SEQ ID NO:299, and a VH-CDR3 comprising the amino acid sequence set forth in SEQ ID NO:295; and (ii) a VL comprising a VL-CDR1 comprising the amino acid sequence set forth in SEQ ID NO:296, a VL-CDR2 comprising the amino acid sequence set forth in SEQ ID NO:246, and a VL-CDR3 comprising the amino acid sequence set forth in SEQ ID NO:297. In certain embodiments, the anti-LAIR-1 antibody comprises: (i) a VH comprising a VH-CDR1 comprising the amino acid sequence set forth in SEQ ID NO:242, a VH-CDR2 comprising the amino acid sequence set forth in SEQ ID NO:309, and a VH-CDR3 comprising the amino acid sequence set forth in SEQ ID NO:305; and (ii) a VL comprising a VL-CDR1 comprising the amino acid sequence set forth in SEQ ID NO:306, a VL-CDR2 comprising the amino acid sequence set forth in SEQ ID NO:307, and a VL-CDR3 comprising the amino acid sequence set forth in SEQ ID NO:308. In certain embodiments, the anti-LAIR-1 antibody comprises: (i) a VH comprising a VH-CDR1 comprising the amino acid sequence set forth in SEQ ID NO:315, a VH-CDR2 comprising the amino acid sequence set forth in SEQ ID NO:323, and a VH-CDR3 comprising the amino acid sequence set forth in SEQ ID NO:317; and (ii) a VL comprising a VL-CDR1 comprising the amino acid sequence set forth in SEQ ID NO:318, a VL-CDR2 comprising the amino acid sequence set forth in SEQ ID NO:319, and a VL-CDR3 comprising the amino acid sequence set forth in SEQ ID NO:320.

In certain embodiments, the CDRs of the anti-LAIR-1 antibody are according to the Kabat designation. In certain embodiments, the anti-LAIR-1 antibody comprises: (i) a VH comprising a VH-CDR1 comprising the amino acid sequence set forth in SEQ ID NO:235, a VH-CDR2 comprising the amino acid sequence set forth in SEQ ID NO:227, and a VH-CDR3 comprising the amino acid sequence set forth in SEQ ID NO:228; and (ii) a VL comprising a VL-CDR1 comprising the amino acid sequence set forth in SEQ ID NO:229, a VL-CDR2 comprising the amino acid sequence set forth in SEQ ID NO:230, and a VL-CDR3 comprising the amino acid sequence set forth in SEQ ID NO:231. In certain embodiments, the anti-LAIR-1 antibody comprises: (i) a VH comprising a VH-CDR1 comprising the amino acid sequence set forth in SEQ ID NO:250, a VH-CDR2 comprising the amino acid sequence set forth in SEQ ID NO:243, and a VH-CDR3 comprising the amino acid sequence set forth in SEQ ID NO:244; and (ii) a VL comprising a VL-CDR1 comprising the amino acid sequence set forth in SEQ ID NO:245, a VL-CDR2 comprising the amino acid sequence set forth in SEQ ID NO:246, and a VL-CDR3 comprising the amino acid sequence set forth in SEQ ID NO:247. In certain embodiments, the anti-LAIR-1 antibody comprises: (i) a VH comprising a VH-CDR1 comprising the amino acid sequence set forth in SEQ ID NO:250, a VH-CDR2 comprising the amino acid sequence set forth in SEQ ID NO:257, and a VH-CDR3 comprising the amino acid sequence set forth in SEQ ID NO:244; and (ii) a VL comprising a VL-CDR1 comprising the amino acid sequence set forth in SEQ ID NO:245, a VL-CDR2 comprising the amino acid sequence set forth in SEQ ID NO:258, and a VL-CDR3 comprising the amino acid sequence set forth in SEQ ID NO:247. In certain embodiments, the anti-LAIR-1 antibody comprises: (i) a VH comprising a VH-CDR1 comprising the amino acid sequence set forth in SEQ ID NO:271, a VH-CDR2 comprising the amino acid sequence set forth in SEQ ID NO:263, and a VH-CDR3 comprising the amino acid sequence set forth in SEQ ID NO:264; and (ii) a VL comprising a VL-CDR1 comprising the amino acid sequence set forth in SEQ ID NO:265, a VL-CDR2 comprising the amino acid sequence set forth in SEQ ID NO:266, and a VL-CDR3 comprising the amino acid sequence set forth in SEQ ID NO:267. In certain embodiments, the anti-LAIR-1 antibody comprises: (i) a VH comprising a VH-CDR1 comprising the amino acid sequence set forth in SEQ ID NO:287, a VH-CDR2 comprising the amino acid sequence set forth in SEQ ID NO:279, and a VH-CDR3 comprising the amino acid sequence set forth in SEQ ID NO:280; and (ii) a VL comprising a VL-CDR1 comprising the amino acid sequence set forth in SEQ ID NO:281, a VL-CDR2 comprising the amino acid sequence set forth in SEQ ID NO:282, and a VL-CDR3 comprising the amino acid sequence set forth in SEQ ID NO:283. In certain embodiments, the anti-LAIR-1 antibody comprises: (i) a VH comprising a VH-CDR1 comprising the amino acid sequence set forth in SEQ ID NO:250, a VH-CDR2 comprising the amino acid sequence set forth in SEQ ID NO:294, and a VH-CDR3 comprising the amino acid sequence set forth in SEQ ID NO:295; and (ii) a VL comprising a VL-CDR1 comprising the amino acid sequence set forth in SEQ ID NO:296, a VL-CDR2 comprising the amino acid sequence set forth in SEQ ID NO:246, and a VL-CDR3 comprising the amino acid sequence set forth in SEQ ID NO:297. In certain embodiments, the anti-LAIR-1 antibody comprises: (i) a VH comprising a VH-CDR1 comprising the amino acid sequence set forth in SEQ ID NO:250, a VH-CDR2 comprising the amino acid sequence set forth in SEQ ID NO:304, and a VH-CDR3 comprising the amino acid sequence set forth in SEQ ID NO:305; and (ii) a VL comprising a VL-CDR1 comprising the amino acid sequence set forth in SEQ ID NO:306, a VL-CDR2 comprising the amino acid sequence set forth in SEQ ID NO:307, and a VL-CDR3 comprising the amino acid sequence set forth in SEQ ID NO:308. In certain embodiments, the anti-LAIR-1 antibody comprises: (i) a VH comprising a VH-CDR1 comprising the amino acid sequence set forth in SEQ ID NO:324, a VH-CDR2 comprising the amino acid sequence set forth in SEQ ID NO:316, and a VH-CDR3 comprising the amino acid sequence set forth in SEQ ID NO:317; and (ii) a VL comprising a VL-CDR1 comprising the amino acid sequence set forth in SEQ ID NO:318, a VL-CDR2 comprising the amino acid sequence set forth in SEQ ID NO:319, and a VL-CDR3 comprising the amino acid sequence set forth in SEQ ID NO:320.

In certain embodiments, the CDRs of the anti-LAIR-1 antibody are according to the Contact designation. In certain embodiments, the anti-LAIR-1 antibody comprises: (i) a VH comprising a VH-CDR1 comprising the amino acid sequence set forth in SEQ ID NO:236, a VH-CDR2 comprising the amino acid sequence set forth in SEQ ID NO:237, and a VH-CDR3 comprising the amino acid sequence set forth in SEQ ID NO:238; and (ii) a VL comprising a VL-CDR1 comprising the amino acid sequence set forth in SEQ ID NO:239, a VL-CDR2 comprising the amino acid sequence set forth in SEQ ID NO:240, and a VL-CDR3 comprising the amino acid sequence set forth in SEQ ID NO:241. In certain embodiments, the anti-LAIR-1 antibody comprises: (i) a VH comprising a VH-CDR1 comprising the amino acid sequence set forth in SEQ ID NO:251, a VH-CDR2 comprising the amino acid sequence set forth in SEQ ID NO:252, and a VH-CDR3 comprising the amino acid sequence set forth in SEQ ID NO:253; and (ii) a VL comprising a VL-CDR1 comprising the amino acid sequence set forth in SEQ ID NO:254, a VL-CDR2 comprising the amino acid sequence set forth in SEQ ID NO:255, and a VL-CDR3 comprising the amino acid sequence set forth in SEQ ID NO:256. In certain embodiments, the anti-LAIR-1 antibody comprises: (i) a VH comprising a VH-CDR1 comprising the amino acid sequence set forth in SEQ ID NO:251, a VH-CDR2 comprising the amino acid sequence set forth in SEQ ID NO:261, and a VH-CDR3 comprising the amino acid sequence set forth in SEQ ID NO:253; and (ii) a VL comprising a VL-CDR1 comprising the amino acid sequence set forth in SEQ ID NO:254, a VL-CDR2 comprising the amino acid sequence set forth in SEQ ID NO:255, and a VL-CDR3 comprising the amino acid sequence set forth in SEQ ID NO:256. In certain embodiments, the anti-LAIR-1 antibody comprises: (i) a VH comprising a VH-CDR1 comprising the amino acid sequence set forth in SEQ ID NO:272, a VH-CDR2 comprising the amino acid sequence set forth in SEQ ID NO:273, and a VH-CDR3 comprising the amino acid sequence set forth in SEQ ID NO:274; and (ii) a VL comprising a VL-CDR1 comprising the amino acid sequence set forth in SEQ ID NO:275, a VL-CDR2 comprising the amino acid sequence set forth in SEQ ID NO:276, and a VL-CDR3 comprising the amino acid sequence set forth in SEQ ID NO:277. In certain embodiments, the anti-LAIR-1 antibody comprises: (i) a VH comprising a VH-CDR1 comprising the amino acid sequence set forth in SEQ ID NO:288, a VH-CDR2 comprising the amino acid sequence set forth in SEQ ID NO:289, and a VH-CDR3 comprising the amino acid sequence set forth in SEQ ID NO:290; and (ii) a VL comprising a VL-CDR1 comprising the amino acid sequence set forth in SEQ ID NO:291, a VL-CDR2 comprising the amino acid sequence set forth in SEQ ID NO:292, and a VL-CDR3 comprising the amino acid sequence set forth in SEQ ID NO:293. In certain embodiments, the anti-LAIR-1 antibody comprises: (i) a VH comprising a VH-CDR1 comprising the amino acid sequence set forth in SEQ ID NO:251, a VH-CDR2 comprising the amino acid sequence set forth in SEQ ID NO:300, and a VH-CDR3 comprising the amino acid sequence set forth in SEQ ID NO:301; and (ii) a VL comprising a VL-CDR1 comprising the amino acid sequence set forth in SEQ ID NO:302, a VL-CDR2 comprising the amino acid sequence set forth in SEQ ID NO:255, and a VL-CDR3 comprising the amino acid sequence set forth in SEQ ID NO:303. In certain embodiments, the anti-LAIR-1 antibody comprises: (i) a VH comprising a VH-CDR1 comprising the amino acid sequence set forth in SEQ ID NO:251, a VH-CDR2 comprising the amino acid sequence set forth in SEQ ID NO:310, and a VH-CDR3 comprising the amino acid sequence set forth in SEQ ID NO:311; and (ii) a VL comprising a VL-CDR1 comprising the amino acid sequence set forth in SEQ ID NO:312, a VL-CDR2 comprising the amino acid sequence set forth in SEQ ID NO:313, and a VL-CDR3 comprising the amino acid sequence set forth in SEQ ID NO:314. In certain embodiments, the anti-LAIR-1 antibody comprises: (i) a VH comprising a VH-CDR1 comprising the amino acid sequence set forth in SEQ ID NO:325, a VH-CDR2 comprising the amino acid sequence set forth in SEQ ID NO:326, and a VH-CDR3 comprising the amino acid sequence set forth in SEQ ID NO:327; and (ii) a VL comprising a VL-CDR1 comprising the amino acid sequence set forth in SEQ ID NO:328, a VL-CDR2 comprising the amino acid sequence set forth in SEQ ID NO:329, and a VL-CDR3 comprising the amino acid sequence set forth in SEQ ID NO:330.

In certain embodiments, the anti-LAIR-1 antibody comprises a humanized framework region (FR) sequence, e.g., as described herein.

In certain embodiments, the anti-LAIR-1 antibody comprises a VH comprising the VH-CDR1, the VH-CDR2, and the VH-CDR3 of antibody 47A1 (or a humanized version thereof), and wherein the VH comprises an amino acid sequence having at least 75%, at least 80%, at least 85%, at least 90%, at least 95%, at least 96%, at least 97%, at least 98%, or at least 99% identity to the amino acid sequence set forth in SEQ ID NO:175.

In certain embodiments, the anti-LAIR-1 antibody comprises a VL comprising the VL-CDR1, the VL-CDR2, and the VL-CDR3 of antibody 47A1 (or a humanized version thereof), and wherein the VL comprises an amino acid sequence having at least 75%, at least 80%, at least 85%, at least 90%, at least 95%, at least 96%, at least 97%, at least 98%, or at least 99% identity to the amino acid sequence set forth in SEQ ID NO:176.

In certain embodiments, the anti-LAIR-1 antibody comprises a VH comprising the VH-CDR1, the VH-CDR2, and the VH-CDR3 of antibody 47H1 (or a humanized version thereof), and wherein the VH comprises an amino acid sequence having at least 75%, at least 80%, at least 85%, at least 90%, at least 95%, at least 96%, at least 97%, at least 98%, or at least 99% identity to the amino acid sequence set forth in SEQ ID NO:177.

In certain embodiments, the anti-LAIR-1 antibody comprises a VL comprising the VL-CDR1, the VL-CDR2, and the VL-CDR3 of antibody 47H1 (or a humanized version thereof), and wherein the VL comprises an amino acid sequence having at least 75%, at least 80%, at least 85%, at least 90%, at least 95%, at least 96%, at least 97%, at least 98%, or at least 99% identity to the amino acid sequence set forth in SEQ ID NO:178.

In certain embodiments, the anti-LAIR-1 antibody comprises a VH comprising the VH-CDR1, the VH-CDR2, and the VH-CDR3 of antibody Hz47H1.v4, and wherein the VH comprises an amino acid sequence having at least 75%, at least 80%, at least 85%, at least 90%, at least 95%, at least 96%, at least 97%, at least 98%, or at least 99% identity to the amino acid sequence set forth in SEQ ID NO:179.

In certain embodiments, the anti-LAIR-1 antibody comprises a VL comprising the VL-CDR1, the VL-CDR2, and the VL-CDR3 of antibody Hz47H1.v4, and wherein the VL comprises an amino acid sequence having at least 75%, at least 80%, at least 85%, at least 90%, at least 95%, at least 96%, at least 97%, at least 98%, or at least 99% identity to the amino acid sequence set forth in SEQ ID NO:180.

In certain embodiments, the anti-LAIR-1 antibody comprises a VH comprising the VH-CDR1, the VH-CDR2, and the VH-CDR3 of antibody 57D12 (or a humanized version thereof), and wherein the VH comprises an amino acid sequence having at least 75%, at least 80%, at least 85%, at least 90%, at least 95%, at least 96%, at least 97%, at least 98%, or at least 99% identity to the amino acid sequence set forth in SEQ ID NO:181.

In certain embodiments, the anti-LAIR-1 antibody comprises a VL comprising the VL-CDR1, the VL-CDR2, and the VL-CDR3 of antibody 57D12 (or a humanized version thereof), and wherein the VL comprises an amino acid sequence having at least 75%, at least 80%, at least 85%, at least 90%, at least 95%, at least 96%, at least 97%, at least 98%, or at least 99% identity to the amino acid sequence set forth in SEQ ID NO:182.

In certain embodiments, the anti-LAIR-1 antibody comprises a VH comprising the VH-CDR1, the VH-CDR2, and the VH-CDR3 of antibody 61H4 (or a humanized version thereof), and wherein the VH comprises an amino acid sequence having at least 75%, at least 80%, at least 85%, at least 90%, at least 95%, at least 96%, at least 97%, at least 98%, or at least 99% identity to the amino acid sequence set forth in SEQ ID NO:183.

In certain embodiments, the anti-LAIR-1 antibody comprises a VL comprising the VL-CDR1, the VL-CDR2, and the VL-CDR3 of antibody 61H4 (or a humanized version thereof), and wherein the VL comprises an amino acid sequence having at least 75%, at least 80%, at least 85%, at least 90%, at least 95%, at least 96%, at least 97%, at least 98%, or at least 99% identity to the amino acid sequence set forth in SEQ ID NO:184.

In certain embodiments, the anti-LAIR-1 antibody comprises a VH comprising the VH-CDR1, the VH-CDR2, and the VH-CDR3 of antibody 62G10 (or a humanized version thereof), and wherein the VH comprises an amino acid sequence having at least 75%, at least 80%, at least 85%, at least 90%, at least 95%, at least 96%, at least 97%, at least 98%, or at least 99% identity to the amino acid sequence set forth in SEQ ID NO:185.

In certain embodiments, the anti-LAIR-1 antibody comprises a VL comprising the VL-CDR1, the VL-CDR2, and the VL-CDR3 of antibody 62G10 (or a humanized version thereof), and wherein the VL comprises an amino acid sequence having at least 75%, at least 80%, at least 85%, at least 90%, at least 95%, at least 96%, at least 97%, at least 98%, or at least 99% identity to the amino acid sequence set forth in SEQ ID NO:186.

In certain embodiments, the anti-LAIR-1 antibody comprises a VH comprising the VH-CDR1, the VH-CDR2, and the VH-CDR3 of antibody Hz62G10.v1, and wherein the VH comprises an amino acid sequence having at least 75%, at least 80%, at least 85%, at least 90%, at least 95%, at least 96%, at least 97%, at least 98%, or at least 99% identity to the amino acid sequence set forth in SEQ ID NO:187.

In certain embodiments, the anti-LAIR-1 antibody comprises a VL comprising the VL-CDR1, the VL-CDR2, and the VL-CDR3 of antibody Hz62G10.v1, and wherein the VL comprises an amino acid sequence having at least 75%, at least 80%, at least 85%, at least 90%, at least 95%, at least 96%, at least 97%, at least 98%, or at least 99% identity to the amino acid sequence set forth in SEQ ID NO:188.

In certain embodiments, the anti-LAIR-1 antibody comprises a VH comprising the VH-CDR1, the VH-CDR2, and the VH-CDR3 of antibody 108D10 (or a humanized version thereof), and wherein the VH comprises an amino acid sequence having at least 75%, at least 80%, at least 85%, at least 90%, at least 95%, at least 96%, at least 97%, at least 98%, or at least 99% identity to the amino acid sequence set forth in SEQ ID NO:189.

In certain embodiments, the anti-LAIR-1 antibody comprises a VL comprising the VL-CDR1, the VL-CDR2, and the VL-CDR3 of antibody 108D10 (or a humanized version thereof), and wherein the VL comprises an amino acid sequence having at least 75%, at least 80%, at least 85%, at least 90%, at least 95%, at least 96%, at least 97%, at least 98%, or at least 99% identity to the amino acid sequence set forth in SEQ ID NO:190.

In certain embodiments, the anti-LAIR-1 antibody comprises: (i) a VH comprising the VH-CDR1, the VH-CDR2, and the VH-CDR3 of antibody 47A1 (or a humanized version thereof), and wherein the VH comprises an amino acid sequence having at least 75%, at least 80%, at least 85%, at least 90%, at least 95%, at least 96%, at least 97%, at least 98%, or at least 99% identity to the amino acid sequence set forth in SEQ ID NO:175; and (ii) a VL comprising the VL-CDR1, the VL-CDR2, and the VL-CDR3 of antibody 47A1 (or a humanized version thereof), and wherein the VL comprises an amino acid sequence having at least 75%, at least 80%, at least 85%, at least 90%, at least 95%, at least 96%, at least 97%, at least 98%, or at least 99% identity to the amino acid sequence set forth in SEQ ID NO:176.

In certain embodiments, the anti-LAIR-1 antibody comprises: (i) a VH comprising the VH-CDR1, the VH-CDR2, and the VH-CDR3 of antibody 47H1 (or a humanized version thereof), and wherein the VH comprises an amino acid sequence having at least 75%, at least 80%, at least 85%, at least 90%, at least 95%, at least 96%, at least 97%, at least 98%, or at least 99% identity to the amino acid sequence set forth in SEQ ID NO:177; and (ii) a VL comprising the VL-CDR1, the VL-CDR2, and the VL-CDR3 of antibody 47H1 (or a humanized version thereof), and wherein the VL comprises an amino acid sequence having at least 75%, at least 80%, at least 85%, at least 90%, at least 95%, at least 96%, at least 97%, at least 98%, or at least 99% identity to the amino acid sequence set forth in SEQ ID NO:178.

In certain embodiments, the anti-LAIR-1 antibody comprises: (i) a VH comprising the VH-CDR1, the VH-CDR2, and the VH-CDR3 of antibody Hz47H1.v4, and wherein the VH comprises an amino acid sequence having at least 75%, at least 80%, at least 85%, at least 90%, at least 95%, at least 96%, at least 97%, at least 98%, or at least 99% identity to the amino acid sequence set forth in SEQ ID NO:179; and (ii) a VL comprising the VL-CDR1, the VL-CDR2, and the VL-CDR3 of antibody Hz47H1.v4, and wherein the VL comprises an amino acid sequence having at least 75%, at least 80%, at least 85%, at least 90%, at least 95%, at least 96%, at least 97%, at least 98%, or at least 99% identity to the amino acid sequence set forth in SEQ ID NO:180.

In certain embodiments, the anti-LAIR-1 antibody comprises: (i) a VH comprising the VH-CDR1, the VH-CDR2, and the VH-CDR3 of antibody 57D12 (or a humanized version thereof), and wherein the VH comprises an amino acid sequence having at least 75%, at least 80%, at least 85%, at least 90%, at least 95%, at least 96%, at least 97%, at least 98%, or at least 99% identity to the amino acid sequence set forth in SEQ ID NO:181; and (ii) a VL comprising the VL-CDR1, the VL-CDR2, and the VL-CDR3 of antibody 57D12 (or a humanized version thereof), and wherein the VL comprises an amino acid sequence having at least 75%, at least 80%, at least 85%, at least 90%, at least 95%, at least 96%, at least 97%, at least 98%, or at least 99% identity to the amino acid sequence set forth in SEQ ID NO:182.

In certain embodiments, the anti-LAIR-1 antibody comprises: (i) a VH comprising the VH-CDR1, the VH-CDR2, and the VH-CDR3 of antibody 61H4 (or a humanized version thereof), and wherein the VH comprises an amino acid sequence having at least 75%, at least 80%, at least 85%, at least 90%, at least 95%, at least 96%, at least 97%, at least 98%, or at least 99% identity to the amino acid sequence set forth in SEQ ID NO:183; and (ii) a VL comprising the VL-CDR1, the VL-CDR2, and the VL-CDR3 of antibody 61H4 (or a humanized version thereof), and wherein the VL comprises an amino acid sequence having at least 75%, at least 80%, at least 85%, at least 90%, at least 95%, at least 96%, at least 97%, at least 98%, or at least 99% identity to the amino acid sequence set forth in SEQ ID NO:184.

In certain embodiments, the anti-LAIR-1 antibody comprises: (i) a VH comprising the VH-CDR1, the VH-CDR2, and the VH-CDR3 of antibody 62G10 (or a humanized version thereof), and wherein the VH comprises an amino acid sequence having at least 75%, at least 80%, at least 85%, at least 90%, at least 95%, at least 96%, at least 97%, at least 98%, or at least 99% identity to the amino acid sequence set forth in SEQ ID NO:185; and (ii) a VL comprising the VL-CDR1, the VL-CDR2, and the VL-CDR3 of antibody 62G10 (or a humanized version thereof), and wherein the VL comprises an amino acid sequence having at least 75%, at least 80%, at least 85%, at least 90%, at least 95%, at least 96%, at least 97%, at least 98%, or at least 99% identity to the amino acid sequence set forth in SEQ ID NO:186.

In certain embodiments, the anti-LAIR-1 antibody comprises: (i) a VH comprising the VH-CDR1, the VH-CDR2, and the VH-CDR3 of antibody Hz62G10.v1, and wherein the VH comprises an amino acid sequence having at least 75%, at least 80%, at least 85%, at least 90%, at least 95%, at least 96%, at least 97%, at least 98%, or at least 99% identity to the amino acid sequence set forth in SEQ ID NO:187; and (ii) a VL comprising the VL-CDR1, the VL-CDR2, and the VL-CDR3 of antibody Hz62G10.v1, and wherein the VL comprises an amino acid sequence having at least 75%, at least 80%, at least 85%, at least 90%, at least 95%, at least 96%, at least 97%, at least 98%, or at least 99% identity to the amino acid sequence set forth in SEQ ID NO:188.

In certain embodiments, the anti-LAIR-1 antibody comprises: (i) a VH comprising the VH-CDR1, the VH-CDR2, and the VH-CDR3 of antibody 108D10 (or a humanized version thereof), and wherein the VH comprises an amino acid sequence having at least 75%, at least 80%, at least 85%, at least 90%, at least 95%, at least 96%, at least 97%, at least 98%, or at least 99% identity to the amino acid sequence set forth in SEQ ID NO:189; and (ii) a VL comprising the VL-CDR1, the VL-CDR2, and the VL-CDR3 of antibody 108D10 (or a humanized version thereof), and wherein the VL comprises an amino acid sequence having at least 75%, at least 80%, at least 85%, at least 90%, at least 95%, at least 96%, at least 97%, at least 98%, or at least 99% identity to the amino acid sequence set forth in SEQ ID NO:190.

In certain embodiments, the anti-LAIR-1 antibody comprises: (i) a VH comprising the VH-CDR1, the VH-CDR2, and the VH-CDR3 of antibody 43H2 (or a humanized version thereof), and wherein the VH comprises an amino acid sequence having at least 75%, at least 80%, at least 85%, at least 90%, at least 95%, at least 96%, at least 97%, at least 98%, or at least 99% identity to the amino acid sequence set forth in SEQ ID NO:191; and (ii) a VL comprising the VL-CDR1, the VL-CDR2, and the VL-CDR3 of antibody 43H2 (or a humanized version thereof), and wherein the VL comprises an amino acid sequence having at least 75%, at least 80%, at least 85%, at least 90%, at least 95%, at least 96%, at least 97%, at least 98%, or at least 99% identity to the amino acid sequence set forth in SEQ ID NO:192.

In certain embodiments, the anti-LAIR-1 antibody comprises a VH comprising the amino acid sequence set forth in SEQ ID NO:175 (or a humanized version thereof).

In certain embodiments, the anti-LAIR-1 antibody comprises a VH comprising the amino acid sequence set forth in SEQ ID NO:177 (or a humanized version thereof).

In certain embodiments, the anti-LAIR-1 antibody comprises a VH comprising the amino acid sequence set forth in SEQ ID NO:179.

In certain embodiments, the anti-LAIR-1 antibody comprises a VH comprising the amino acid sequence set forth in SEQ ID NO:181 (or a humanized version thereof).

In certain embodiments, the anti-LAIR-1 antibody comprises a VH comprising the amino acid sequence set forth in SEQ ID NO:183 (or a humanized version thereof).

In certain embodiments, the anti-LAIR-1 antibody comprises a VH comprising the amino acid sequence set forth in SEQ ID NO:185 (or a humanized version thereof).

In certain embodiments, the anti-LAIR-1 antibody comprises a VH comprising the amino acid sequence set forth in SEQ ID NO:187.

In certain embodiments, the anti-LAIR-1 antibody comprises a VH comprising the amino acid sequence set forth in SEQ ID NO:189 (or a humanized version thereof).

In certain embodiments, the anti-LAIR-1 antibody comprises a VH comprising the amino acid sequence set forth in SEQ ID NO:191 (or a humanized version thereof).

In certain embodiments, the anti-LAIR-1 antibody comprises a VL comprising the amino acid sequence set forth in SEQ ID NO:176 (or a humanized version thereof).

In certain embodiments, the anti-LAIR-1 antibody comprises a VL comprising the amino acid sequence set forth in SEQ ID NO:178 (or a humanized version thereof).

In certain embodiments, the anti-LAIR-1 antibody comprises a VL comprising the amino acid sequence set forth in SEQ ID NO:180.

In certain embodiments, the anti-LAIR-1 antibody comprises a VL comprising the amino acid sequence set forth in SEQ ID NO:182 (or a humanized version thereof).

In certain embodiments, the anti-LAIR-1 antibody comprises a VL comprising the amino acid sequence set forth in SEQ ID NO:184 (or a humanized version thereof).

In certain embodiments, the anti-LAIR-1 antibody comprises a VL comprising the amino acid sequence set forth in SEQ ID NO:186 (or a humanized version thereof).

In certain embodiments, the anti-LAIR-1 antibody comprises a VL comprising the amino acid sequence set forth in SEQ ID NO:188.

In certain embodiments, the anti-LAIR-1 antibody comprises a VL comprising the amino acid sequence set forth in SEQ ID NO:190 (or a humanized version thereof).

In certain embodiments, the anti-LAIR-1 antibody comprises a VL comprising the amino acid sequence set forth in SEQ ID NO:192 (or a humanized version thereof).

In certain embodiments, the anti-LAIR-1 antibody comprises: (i) a VH comprising the amino acid sequence set forth in SEQ ID NO:175 (or a humanized version thereof); and (ii) a VL comprising the amino acid sequence set forth in SEQ ID NO:176 (or a humanized version thereof).

In certain embodiments, the anti-LAIR-1 antibody comprises: (i) a VH comprising the amino acid sequence set forth in SEQ ID NO:177 (or a humanized version thereof); and (ii) a VL comprising the amino acid sequence set forth in SEQ ID NO:178 (or a humanized version thereof).

In certain embodiments, the anti-LAIR-1 antibody comprises: (i) a VH comprising the amino acid sequence set forth in SEQ ID NO:179; and (ii) a VL comprising the amino acid sequence set forth in SEQ ID NO:180.

In certain embodiments, the anti-LAIR-1 antibody comprises: (i) a VH comprising the amino acid sequence set forth in SEQ ID NO:181 (or a humanized version thereof); and (ii) a VL comprising the amino acid sequence set forth in SEQ ID NO:182 (or a humanized version thereof).

In certain embodiments, the anti-LAIR-1 antibody comprises: (i) a VH comprising the amino acid sequence set forth in SEQ ID NO:183 (or a humanized version thereof); and (ii) a VL comprising the amino acid sequence set forth in SEQ ID NO:184 (or a humanized version thereof).

In certain embodiments, the anti-LAIR-1 antibody comprises: (i) a VH comprising the amino acid sequence set forth in SEQ ID NO:185 (or a humanized version thereof); and (ii) a VL comprising the amino acid sequence set forth in SEQ ID NO:186 (or a humanized version thereof).

In certain embodiments, the anti-LAIR-1 antibody comprises: (i) a VH comprising the amino acid sequence set forth in SEQ ID NO:187; and (ii) a VL comprising the amino acid sequence set forth in SEQ ID NO:188.

In certain embodiments, the anti-LAIR-1 antibody comprises: (i) a VH comprising the amino acid sequence set forth in SEQ ID NO:189 (or a humanized version thereof); and (ii) a VL comprising the amino acid sequence set forth in SEQ ID NO:190 (or a humanized version thereof).

In certain embodiments, the anti-LAIR-1 antibody comprises: (i) a VH comprising the amino acid sequence set forth in SEQ ID NO:191 (or a humanized version thereof); and (ii) a VL comprising the amino acid sequence set forth in SEQ ID NO:192 (or a humanized version thereof).

In certain embodiments, the anti-LAIR-1 antibody comprises a heavy chain comprising a VH comprising the VH-CDR1, the VH-CDR2, and the VH-CDR3 from the amino acid sequence set forth in SEQ ID NO:179, and wherein the heavy chain comprises an amino acid sequence having at least 75%, at least 80%, at least 85%, at least 90%, at least 95%, at least 96%, at least 97%, at least 98%, at least 99%, or 100% identity to the amino acid sequence set forth in SEQ ID NO:194.

In certain embodiments, the anti-LAIR-1 antibody comprises a light chain comprising a VL comprising the VL-CDR1, the VL-CDR2, and the VL-CDR3 from the amino acid sequence set forth in SEQ ID NO:180, and wherein the light chain comprises an amino acid sequence having at least 75%, at least 80%, at least 85%, at least 90%, at least 95%, at least 96%, at least 97%, at least 98%, at least 99%, or 100% identity to the amino acid sequence set forth in SEQ ID NO:196.

In certain embodiments, the anti-LAIR-1 antibody comprises a heavy chain comprising a VH comprising the VH-CDR1, the VH-CDR2, and the VH-CDR3 from the amino acid sequence set forth in SEQ ID NO:187, and wherein the heavy chain comprises an amino acid sequence having at least 75%, at least 80%, at least 85%, at least 90%, at least 95%, at least 96%, at least 97%, at least 98%, at least 99%, or 100% identity to the amino acid sequence set forth in SEQ ID NO:198.

In certain embodiments, the anti-LAIR-1 antibody comprises a light chain comprising a VL comprising the VL-CDR1, the VL-CDR2, and the VL-CDR3 from the amino acid sequence set forth in SEQ ID NO:188, and wherein the light chain comprises an amino acid sequence having at least 75%, at least 80%, at least 85%, at least 90%, at least 95%, at least 96%, at least 97%, at least 98%, at least 99%, or 100% identity to the amino acid sequence set forth in SEQ ID NO:200.

In certain embodiments, the anti-LAIR-1 antibody comprises: (i) a heavy chain comprising a VH comprising the VH-CDR1, the VH-CDR2, and the VH-CDR3 from the amino acid sequence set forth in SEQ ID NO:179, and wherein the heavy chain comprises an amino acid sequence having at least 75%, at least 80%, at least 85%, at least 90%, at least 95%, at least 96%, at least 97%, at least 98%, at least 99%, or 100% identity to the amino acid sequence set forth in SEQ ID NO:194; and (ii) a light chain comprising a VL comprising the VL-CDR1, the VL-CDR2, and the VL-CDR3 from the amino acid sequence set forth in SEQ ID NO:180, and wherein the light chain comprises an amino acid sequence having at least 75%, at least 80%, at least 85%, at least 90%, at least 95%, at least 96%, at least 97%, at least 98%, at least 99%, or 100% identity to the amino acid sequence set forth in SEQ ID NO:196.

In certain embodiments, the anti-LAIR-1 antibody comprises: (i) a heavy chain comprising a VH comprising the VH-CDR1, the VH-CDR2, and the VH-CDR3 from the amino acid sequence set forth in SEQ ID NO:187, and wherein the heavy chain comprises an amino acid sequence having at least 75%, at least 80%, at least 85%, at least 90%, at least 95%, at least 96%, at least 97%, at least 98%, at least 99%, or 100% identity to the amino acid sequence set forth in SEQ ID NO:198; and (ii) a light chain comprising a VL comprising the VL-CDR1, the VL-CDR2, and the VL-CDR3 from the amino acid sequence set forth in SEQ ID NO:188, and wherein the light chain comprises an amino acid sequence having at least 75%, at least 80%, at least 85%, at least 90%, at least 95%, at least 96%, at least 97%, at least 98%, at least 99%, or 100% identity to the amino acid sequence set forth in SEQ ID NO:200.

In certain embodiments, the anti-LAIR-1 antibody comprises a heavy chain comprising the amino acid sequence set forth in SEQ ID NO:194. In certain embodiments, the anti-LAIR-1 antibody comprises a heavy chain consisting of the amino acid sequence set forth in SEQ ID NO:194.

In certain embodiments, the anti-LAIR-1 antibody comprises a light chain comprising the amino acid sequence set forth in SEQ ID NO:196. In certain embodiments, the anti-LAIR-1 antibody comprises a light chain consisting of the amino acid sequence set forth in SEQ ID NO:196.

In certain embodiments, the anti-LAIR-1 antibody comprises: (i) a heavy chain comprising the amino acid sequence set forth in SEQ ID NO:194; and (ii) a light chain comprising the amino acid sequence set forth in SEQ ID NO:196. In certain embodiments, the anti-LAIR-1 antibody comprises: (i) a heavy chain consisting of the amino acid sequence set forth in SEQ ID NO:194; and (ii) a light chain consisting of the amino acid sequence set forth in SEQ ID NO:196.

In certain embodiments, the anti-LAIR-1 antibody comprises a heavy chain comprising the amino acid sequence set forth in SEQ ID NO:198. In certain embodiments, the anti-LAIR-1 antibody comprises a heavy chain consisting of the amino acid sequence set forth in SEQ ID NO:198.

In certain embodiments, the anti-LAIR-1 antibody comprises a light chain comprising the amino acid sequence set forth in SEQ ID NO:200. In certain embodiments, the anti-LAIR-1 antibody comprises a light chain consisting of the amino acid sequence set forth in SEQ ID NO:200.

In certain embodiments, the anti-LAIR-1 antibody comprises: (i) a heavy chain comprising the amino acid sequence set forth in SEQ ID NO:198; and (ii) a light chain comprising the amino acid sequence set forth in SEQ ID NO:200. In certain embodiments, the anti-LAIR-1 antibody comprises: (i) a heavy chain consisting of the amino acid sequence set forth in SEQ ID NO:198; and (ii) a light chain consisting of the amino acid sequence set forth in SEQ ID NO:200.

In certain embodiments, the anti-LAIR-1 antibody is an antibody described in any of the following publications US20190338026A1 and WO2018027039A1, which are all incorporated herein by reference in their entireties.

In certain embodiments, the anti-LAIR-1 antibody binds to the same epitope as an antibody having the VH and VL of any one of Tables 9-15. In certain embodiments, the anti-LAIR-1 antibody binds to the same epitope as antibody Hz47H1.v4 (see Table 10B). In certain embodiments, the anti-LAIR-1 antibody binds to the same epitope as antibody Hz62G10.v1 (see Table 13).

In certain embodiments, the anti-LAIR-1 antibody binds LAIR-1 or a fragment of LAIR-1. In certain embodiments, the fragment of LAIR-1 comprises the extracellular domain. In certain embodiments, the fragment of LAIR-1 comprises the Ig-like C2 type domain (D1). In certain embodiments, the fragment of LAIR-1 comprises the Ig-like C2 type domain and the stem region (D1-stem). In certain embodiments, the anti-LAIR-1 antibody binds human LAIR-1 or a fragment of human LAIR-1. In certain embodiments, the fragment of human LAIR-1 comprises the extracellular domain of human LAIR-1. In certain embodiments, the extracellular domain of human LAIR-1 comprises amino acids (aa) 22-165 of SEQ ID NO:167. In certain embodiments, D1 of human LAIR-1 comprises amino acids 29-117 of SEQ ID NO:167. In certain embodiments, D1-stem of human LAIR-1 comprises amino acids 29-165 of SEQ ID NO:167. In certain embodiments, the fragment of human LAIR-1 comprises the amino acid sequence of SEQ ID NO:169. In certain embodiments, the fragment of human LAIR-1 comprises the amino acid sequence of SEQ ID NO:170. In certain embodiments, the anti-LAIR-1 antibody binds cyno LAIR-1 or a fragment of cyno LAIR-1. In certain embodiments, the fragment of cyno LAIR-1 comprises the extracellular domain of cyno LAIR-1. In certain embodiments, the extracellular domain of cyno LAIR-1 comprises amino acids 22-165 of SEQ ID NO:171. In certain embodiments, D1 of cyno LAIR-1 comprises amino acids 29-117 of SEQ ID NO:171. In certain embodiments, D1-stem of cyno LAIR-1 comprises amino acids 29-165 of SEQ ID NO:171. In certain embodiments, a fragment of cyno LAIR-1 comprises the amino acid sequence of SEQ ID NO:173. In certain embodiments, the fragment of cyno LAIR-1 comprises the amino acid sequence of SEQ ID NO:174

In certain embodiments, the anti-LAIR-1 antibody binds human LAIR-1. In certain embodiments, the anti-LAIR-1 antibody binds cyno LAIR-1. In certain embodiments, the anti-LAIR-1 antibody binds mouse LAIR-1. In certain embodiments, the anti-LAIR-1 antibody binds human LAIR-1 and cyno LAIR-1. In certain embodiments, the anti-LAIR-1 antibody binds human LAIR-1 and cyno LAIR-1, but does not bind mouse LAIR-1. In certain embodiments, the anti-LAIR-1 antibody binds SEQ ID NO:167. In certain embodiments, the anti-LAIR-1 antibody binds SEQ ID NO:168. In certain embodiments, the anti-LAIR-1 antibody binds within amino acids 22-165 of SEQ ID NO:167. In certain embodiments, the anti-LAIR-1 antibody binds within amino acids 29-117 of SEQ ID NO:167. In certain embodiments, the anti-LAIR-1 antibody binds within amino acids 29-165 of SEQ ID NO:167. In certain embodiments, the anti-LAIR-1 antibody binds SEQ ID NO:169. In certain embodiments, the anti-LAIR-1 antibody binds SEQ ID NO:170. In certain embodiments, the anti-LAIR-1 antibody binds SEQ ID NO:171. In certain embodiments, the anti-LAIR-1 antibody binds SEQ ID NO:172. In certain embodiments, the anti-LAIR-1 antibody binds within amino acids 22-165 of SEQ ID NO:171. In certain embodiments, the anti-LAIR-1 antibody binds within amino acids 29-117 of SEQ ID NO:171. In certain embodiments, the anti-LAIR-1 antibody binds within amino acids 29-165 of SEQ ID NO:171. In certain embodiments, the anti-LAIR-1 antibody binds SEQ ID NO:173. In certain embodiments, the anti-LAIR-1 antibody binds SEQ ID NO:174. In certain embodiments, the anti-LAIR-1 antibody binds SEQ ID NO:201. In certain embodiments, the anti-LAIR-1 antibody binds SEQ ID NO:202. In certain embodiments, the anti-LAIR-1 antibody binds within amino acids 22-144 of SEQ ID NO:201. In certain embodiments, the anti-LAIR-1 antibody binds within amino acids 27-114 of SEQ ID NO:201. In certain embodiments, the anti-LAIR-1 antibody binds within amino acids 27-144 of SEQ ID NO:201. In certain embodiments, the anti-LAIR-1 antibody binds SEQ ID NO:203. In certain embodiments, the anti-LAIR-1 antibody binds SEQ ID NO:204.

In certain embodiments, the anti-LAIR-1 antibody binds an epitope comprising amino acids within SEQ ID NO:168. In certain embodiments, the anti-LAIR-1 antibody binds an epitope comprising amino acids within SEQ ID NO:169. In certain embodiments, the anti-LAIR-1 antibody binds an epitope comprising amino acids within SEQ ID NO:170. In certain embodiments, the anti-LAIR-1 antibody binds an epitope comprising at least one amino acid within amino acids 70-80 of SEQ ID NO:167. In certain embodiments, the anti-LAIR-1 antibody binds an epitope comprising at least one amino acid within amino acids 61-80 of SEQ ID NO:167. In certain embodiments, the anti-LAIR-1 antibody binds an epitope comprising amino acids within SEQ ID NO:172. In certain embodiments, the anti-LAIR-1 antibody binds an epitope comprising amino acids within SEQ ID NO:173. In certain embodiments, the anti-LAIR-1 antibody binds an epitope comprising amino acids within SEQ ID NO:174. In certain embodiments, the anti-LAIR-1 antibody binds an epitope comprising amino acids within SEQ ID NO:202. In certain embodiments, the anti-LAIR-1 antibody binds an epitope comprising amino acids within SEQ ID NO:203. In certain embodiments, the anti-LAIR-1 antibody binds an epitope comprising amino acids within SEQ ID NO:204.

In certain embodiments, the anti-LAIR-1 antibody is a humanized antibody.

In certain embodiments, the anti-LAIR-1 antibody is a human IgG1 antibody. In certain embodiments, the anti-LAIR-1 antibody is a human IgG2 antibody. In certain embodiments, the anti-LAIR-1 antibody is a human IgG3 antibody. In certain embodiments, the anti-LAIR-1 antibody is a human IgG4 antibody. In certain embodiments, the anti-LAIR-1 antibody comprises a human kappa light chain constant region. In certain embodiments, the anti-LAIR-1 antibody comprises a human lambda light chain constant region.

In certain embodiments, the anti-LAIR-1 antibody comprises a partial constant region sequence of a human IgG1 antibody. In certain embodiments, the anti-LAIR-1 antibody comprises a partial constant region sequence of a human IgG2 antibody. In certain embodiments, the anti-LAIR-1 antibody comprises a partial constant region sequence of a human IgG3 antibody. In certain embodiments, the anti-LAIR-1 antibody comprises a partial constant region sequence of a human IgG4 antibody.

In certain embodiments, the anti-LAIR-1 antibody is an antibody fragment comprising at least one antigen-binding site. In certain embodiments, the anti-LAIR-1 antibody is a Fab, a Fab′, a F(ab)₂, a Fv, an scFv, an (scFv)₂, a single chain antibody, a dual variable region antibody, a single variable region antibody, a linear antibody, a diabody, a nanobody, or a V region antibody. The anti-LAIR-1 antibodies described herein can be produced by any suitable method known in the art. Such methods range from direct protein synthesis methods to constructing a DNA sequence encoding polypeptide sequences and expressing those sequences in a suitable host.

### 5.2. Combinations, Compositions, and Pharmaceutical Compositions Comprising the Antibodies Disclosed Herein

In one aspect, provided herein is a combination comprising an anti-ILT3 antibody described herein (e.g., anti-ILT3 antibodies described in Section 5.1.2), and an anti-LAIR-1 antibody described herein (e.g., anti-LAIR-1 antibodies described in Section 5.1.3). In certain embodiments, the anti-LAIR-1 antibody and the anti-ILT3 antibody are formulated separately. In certain embodiments, the anti-LAIR-1 antibody and the anti-ILT3 antibody are co-formulated. In certain embodiments, the anti-LAIR-1 antibody and the anti-ILT3 antibody are formulated as a pharmaceutical composition in the combination. In certain embodiments, the anti-LAIR-1 antibody and the anti-ILT3 antibody are each separately formulated as a pharmaceutical composition in the combination.

In another aspect, this disclosure provides a pharmaceutical composition comprising an anti-ILT3 antibody described herein and a suitable pharmaceutical carrier, and a separate pharmaceutical composition comprising an anti-LAIR-1 antibody described herein and a suitable pharmaceutical carrier.

In another aspect, this disclosure provides a composition comprising an anti-ILT3 antibody and an anti-LAIR-1 antibody described herein. In certain embodiments, the composition is a pharmaceutical composition comprising an anti-ILT3 antibody and an anti-LAIR-1 antibody described herein, and a suitable pharmaceutical carrier. A suitable carrier includes any materials that when combined with the therapeutic composition retains the therapeutic function of the therapeutic composition and is generally non-reactive with the patient's immune system. Examples include, but are not limited to, any of a number of standard pharmaceutical carriers such as sterile phosphate buffered saline solutions, bacteriostatic water, and the like (see, generally, Remington's Pharmaceutical Sciences 16^{th} Edition, A. Osal., Ed., 1980). Accordingly, an anti-ILT3 antibody and an anti-LAIR-1 antibody are formulated separately for use in any one of the methods and applications described herein.

Any one of the anti-ILT3 antibodies and the anti-LAIR-1 antibodies, as described herein, may be separately formulated (i.e., included in two separate compositions or pharmaceutical compositions) or co-formulated (i.e., included in a composition or a pharmaceutical composition) for use in any of the methods and uses described herein.

In certain embodiments, the anti-ILT3 antibody comprised in a composition or a pharmaceutical composition comprises a VH comprising a VH-CDR1, a VH-CDR2, a VH-CDR3, and a VL comprising a VL-CDR1, a VL-CDR2, and a VL-CDR3, wherein the VH-CDR1, VH-CDR2, VH-CDR3, VL-CDR1, VL-CDR2 and VL-CDR3 are from any one of the VH and VL sequences of the antibodies described herein (e.g., 3A3, 5A7, 12A12, 16C5, 45G10, 48A6, 53F10, or Hz5A7.v5), such as the amino acid sequences depicted in Tables 1, 3-8, and 2, respectively. In certain embodiments, the anti-LAIR-1 antibody comprised in a composition or a pharmaceutical composition comprises a VH comprising a VH-CDR1, a VH-CDR2, a VH-CDR3, and a VL comprising a VL-CDR1, a VL-CDR2, and a VL-CDR3, wherein the VH-CDR1, VH-CDR2, VH-CDR3, VL-CDR1, VL-CDR2 and VL-CDR3 are from any one of the VH and VL sequences of the antibodies described herein (e.g., 47A1, 47H1, Hz47H1.v4, 57D12, 61H4, 62G10, Hz62G10.v1, 108D10, 43H2), such as the amino acid sequences depicted in Tables 9-15, respectively.

In certain embodiments, the anti-ILT3 antibody comprised in a composition or a pharmaceutical composition comprises a VH comprising the VH-CDR1, the VH-CDR2, and the VH-CDR3 of the antibody 5A7 or Hz5A7.v5 (see Table 2 and Table 8) and a VL comprising the VL-CDR1, the VL-CDR2, and the VL-CDR3 of the antibody 5A7 or Hz5A7.v5 (see Table 2 and Table 8). The anti-LAIR-1 antibody comprised in a composition or a pharmaceutical composition comprises a VH comprising the VH-CDR1, the VH-CDR2, and the VH-CDR3 of the antibody 47H1, Hz47H1.v4, 62G10, or Hz62G10.v1 (see Table 10A, Table 10B and Table 13) and a VL comprising the VL-CDR1, the VL-CDR2, and the VL-CDR3 of the antibody 47H1, Hz47H1.v4, 62G10, or Hz62G10.v1 (see Table 10A, Table 10B and Table 13). In certain embodiments, the anti-ILT3 antibody comprised in a composition or a pharmaceutical composition comprises a VH comprising the amino acid sequence set forth in SEQ ID NO:123 and a VL comprising the amino acid sequence set forth in SEQ ID NO:124. The anti-LAIR-1 antibody comprised in a composition or a pharmaceutical composition comprises a VH comprising the amino acid sequence set forth in SEQ ID NO:179 and a VL comprising the amino acid sequence set forth in SEQ ID NO:180. In certain embodiments, the anti-ILT3 antibody comprised in a composition or a pharmaceutical composition comprises a heavy chain comprising the amino acid sequence set forth in SEQ ID NO:126 and a light comprises the amino acid sequence set forth in SEQ ID NO:128. The anti-LAIR-1 antibody comprised in a composition or a pharmaceutical composition comprises a heavy chain comprising the amino acid sequence set forth in SEQ ID NO:194 and a light comprises the amino acid sequence set forth in SEQ ID NO:196.

In certain embodiments, an anti-ILT3 antibody comprised in a composition or a pharmaceutical composition is an anti-ILT3 antibody as described in WO 2021127200A1, US20190153093A1, WO2020056077A1, WO2021183839A2, US20200031926A1, US20210221887A1, US20150110714A1, US20200031926A1, US20190241655A1, WO2020180789A1, or WO2020056077A1. In certain embodiments, an anti-LAIR-1 comprised in a composition or a pharmaceutical composition is an anti-LAIR-1 antibody as described in US20190338026A1 and WO2018027039A1.

### 5.3. Methods and Uses of the Antibodies, Combinations, Compositions, and Pharmaceutical Compositions Disclosed Herein

### 5.3.1. Activating an Immune Cell

In one aspect, the present disclosure provides a method of activating an immune cell, wherein the method comprises contacting the immune cell with an anti-ILT3 antibody and an anti-LAIR-1 antibody. In certain embodiments, the immune cell is associated with a tumor. In certain embodiments, the immune cell resides in a tumor microenvironment. In certain embodiments, the immune cell is associated with but does not reside in a tumor microenvironment. In certain embodiments, the immune cell is not associated with a tumor or a tumor microenvironment, but the immune cell is capable of being recruited to a tumor or a tumor environment. In certain embodiments, the method is an in vitro method. In certain embodiments, the method is an ex vivo method. In certain embodiments, the anti-ILT3 antibody and the anti-LAIR-1 antibody are formulated into two separate pharmaceutical compositions. In certain embodiments, the anti-ILT3 antibody and the anti-LAIR-1 antibody are formulated into a pharmaceutical composition.

In another aspect, this disclosure provides an anti-ILT3 antibody and an anti-LAIR-1 antibody, for use in activating an immune cell, wherein the use comprises contacting the immune cell with an effective amount of the anti-ILT3 antibody and an effective amount of the anti-LAIR-1 antibody. In certain embodiments, the immune cell is associated with a tumor. In certain embodiments, the immune cell resides in a tumor microenvironment. In certain embodiments, the immune cell is associated with but does not reside in a tumor microenvironment. In certain embodiments, the immune cell is not associated with a tumor or a tumor microenvironment, but the immune cell is capable of being recruited to a tumor or a tumor environment. In certain embodiments, the use is an ex vivo use. In certain embodiments, the method is an in vitro use. In certain embodiments, the anti-ILT3 antibody and the anti-LAIR-1 antibody are formulated into two separate pharmaceutical compositions. In certain embodiments, the anti-ILT3 antibody and the anti-LAIR-1 antibody are formulated into a pharmaceutical composition.

In another aspect, this disclosure provides an in vivo method of activating an immune cell in a subject, wherein the method comprises administering a therapeutically effective amount of an anti-ILT3 antibody and a therapeutically effective amount of an anti-LAIR-1 antibody, or pharmaceutical composition(s) thereof, to the subject, thereby activating the immune cell in the subject. In certain embodiments, the immune cell is associated with a tumor. In certain embodiments, the immune cell resides in a tumor microenvironment. In certain embodiments, the immune cell is associated with but does not reside in a tumor microenvironment. In certain embodiments, the immune cell is not associated with a tumor or a tumor microenvironment, but the immune cell is capable of being recruited to a tumor or tumor environment. In certain embodiments, the subject is a mammal. In certain embodiments, the subject is a human. In certain embodiments, the subject is pre-diagnosed with cancer before receiving the combination of an anti-ILT3 antibody and an anti-LAIR-1 antibody, as described herein. In certain embodiments, the subject is presently undergoing a cancer therapy. In certain embodiments, the subject has relapsed from a prior cancer treatment before receiving the combination of an anti-ILT3 antibody and an anti-LAIR-1 antibody, as described herein. In certain embodiments, the subject has been diagnosed for the first time as having cancer by a doctor prior to receiving the combination of an anti-ILT3 antibody and an anti-LAIR-1 antibody, as described herein. In certain embodiments, the anti-ILT3 antibody and the anti-LAIR-1 antibody are formulated into two separate pharmaceutical compositions. In certain embodiments, the anti-ILT3 antibody and the anti-LAIR-1 antibody are formulated into a pharmaceutical composition.

“Activating” or “activate(s)” an immune cell used herein includes or results in one or more of the following: stimulation of an immune cell, reactivation of an immune cell, increase of immune cell activity, increase of immune cell proliferation, increased expression of a cytokine and/or a chemokine from an immune cell, and recruitment of an immune cell into a tumor or tumor microenvironment. The immune cell can be from a myeloid lineage or from a lymphoid lineage, including, but not limited to, a myeloid cell, a T cell, and an NK cell. In certain embodiments, the immune cell is already in an active or partially active state, and the methods and uses described herein further activate the immune cell. In certain embodiments, the methods and uses further activate the immune cell by increasing the immune cell activity, increasing the immune cell proliferation, and/or increasing a cytokine and/or a chemokine expression from the immune cell. In certain embodiments, the methods and uses described herein recruit the immune cell into a tumor or a tumor microenvironment. In certain embodiments, the immune cell is in a suppressed or partially suppressed state, and the methods and uses described herein reactivate the immune cell or restore the activity of the immune cell. In certain embodiments, the methods and uses inhibit a signaling pathway that suppresses the immune cell. In certain embodiments, the methods and uses reactivate the immune cell by increasing the immune cell activity, increasing the immune cell proliferation, increasing a cytokine and/or a chemokine expression from the immune cell, and/or increasing recruitment of the immune cell into a tumor or a tumor microenvironment.

In certain embodiments, the immune cell is a myeloid cell. In certain embodiments, the myeloid cell is associated with a tumor. In certain embodiments, the myeloid cell resides in a tumor microenvironment. In certain embodiments, the myeloid cell is associated with but does not reside in a tumor microenvironment. In certain embodiments, the methods and uses described herein stimulate or activate the myeloid cell. In certain embodiments, the methods and uses described herein reactivate or restore the activity of the myeloid cell. In certain embodiments, the methods and uses increase or restore a cytokine and/or a chemokine expression in the myeloid cell. In certain embodiments, the methods and uses described herein increase recruitment of the myeloid cell into a tumor or a tumor microenvironment. In certain embodiments, the myeloid cell is a monocyte. In certain embodiments, the myeloid cell is a macrophage. In certain embodiments, the myeloid cell is a dendritic cell. In certain embodiments, the myeloid cell is an antigen presenting cell (APC).

In certain embodiments, the immune cell is a T cell. In certain embodiments, the T cell is associated with a tumor. In certain embodiments, the T cell resides in a tumor microenvironment. In certain embodiments, the T cell is associated with but does not reside in a tumor microenvironment. In certain embodiments, the T cell is not associated with a tumor or a tumor microenvironment, but the T cell is capable of being recruited to a tumor or tumor environment. In certain embodiments, the methods and uses described herein stimulate or activate the T cell. In certain embodiments, the methods and uses reactivate the T cell or restores activity of the T cell. In certain embodiments, the methods and uses described herein increase or restore a cytokine and/or a chemokine production in the T cell. In certain embodiments, the methods and uses described herein increase the T cell proliferation. In certain embodiments, the T cell resides in a distance site from a tumor, and the methods and uses described herein increase recruitment of the T cell into the tumor or the tumor microenvironment. In certain embodiments, the T cell is an effector T cell. In certain embodiments, the T cell is a cytotoxic T-cell. In certain embodiments, the T cell is a CD8⁺ T cell.

In certain embodiments, the immune cell is an NK cell. In certain embodiments, the NK cell is associated with a tumor. In certain embodiments, the NK cell resides in a tumor microenvironment. In certain embodiments, the NK cell is associated with but does not reside in a tumor microenvironment. In certain embodiments, the NK cell is not associated with a tumor or a tumor microenvironment, but the NK cell is capable of being recruited to a tumor or tumor environment. In certain embodiments, the methods and uses described herein stimulate or activate the NK cell. In certain embodiments, the methods and uses reactivate the NK cell or restores activity of the NK cell. In certain embodiments, the methods and uses described herein increase or restore a cytokine and/or a chemokine production in the NK cell. In certain embodiments, the methods and uses described herein increase the NK cell proliferation. In certain embodiments, the NK cell does not reside in a tumor or tumor microenvironment, and the methods and uses described herein increase recruitment of the NK cell into the tumor or the tumor microenvironment.

### 5.3.2. Reprogramming an Immune Suppressor Cell

The present disclosure provides a method of reducing or inhibiting immune suppressive activity of an immune cell, wherein the method comprises contacting the immune cell with an anti-ILT3 antibody and an anti-LAIR-1 antibody. In certain embodiments, the immune cell is associated with a tumor. In certain embodiments, the immune cell resides in a tumor microenvironment. In certain embodiments, the immune cell is associated with but does not reside in a tumor microenvironment. In certain embodiments, the method is an in vitro method. In certain embodiments, the method is an ex vivo method. In certain embodiments, the anti-ILT3 antibody and the anti-LAIR-1 antibody are formulated into two separate pharmaceutical compositions. In certain embodiments, the anti-ILT3 antibody and the anti-LAIR-1 antibody are formulated into a pharmaceutical composition.

The present disclosure also provides an anti-ILT3 antibody and an anti-LAIR-1 antibody for use in reducing or inhibiting immune suppressive activity of an immune cell, wherein the use comprises contacting the immune cell with an effective amount of the anti-ILT3 antibody and an effective amount of the anti-LAIR-1 antibody. In certain embodiments, the immune cell is associated with a tumor. In certain embodiments, the immune cell resides in a tumor microenvironment. In certain embodiments, the immune cell is associated with but does not reside in a tumor microenvironment. In certain embodiments, the use is an ex vivo use. In certain embodiments, the method is an in vitro use. In certain embodiments, the anti-ILT3 antibody and the anti-LAIR-1 antibody are formulated into two separate pharmaceutical compositions. In certain embodiments, the anti-ILT3 antibody and the anti-LAIR-1 antibody are formulated into a pharmaceutical composition.

In another aspect, the present disclosure provides an in vivo method of reducing or inhibiting immune suppressive activity of an immune cell in a subject, wherein the method comprises administering a therapeutically effective amount of an anti-ILT3 antibody and a therapeutically effective amount of an anti-LAIR-1 antibody, or pharmaceutical composition(s) thereof, to the subject, thereby reducing or inhibiting immune suppressive activity of the immune cell in the subject. In certain embodiments, the immune cell is associated with a tumor. In certain embodiments, the immune cell resides in a tumor microenvironment. In certain embodiments, the immune cell is associated with but does not reside in a tumor microenvironment. In certain embodiments, the subject is a mammal. In certain embodiments, the subject is a human. In certain embodiments, the subject is pre-diagnosed with cancer before receiving the combination of an anti-ILT3 antibody and an anti-LAIR-1 antibody, as described herein. In certain embodiments, the subject is presently undergoing a cancer therapy. In certain embodiments, the subject has relapsed from a prior cancer treatment before receiving the combination of an anti-ILT3 antibody and an anti-LAIR-1 antibody, as described herein. In certain embodiments, the subject has been diagnosed for the first time as having cancer by a doctor prior to receiving the combination of an anti-ILT3 antibody and an anti-LAIR-1 antibody, as described herein. In certain embodiments, the anti-ILT3 antibody and the anti-LAIR-1 antibody are formulated into two separate pharmaceutical compositions. In certain embodiments, the anti-ILT3 antibody and the anti-LAIR-1 antibody are formulated into a pharmaceutical composition.

In certain embodiments, the methods and uses described herein decrease or inhibit the immune suppressive activity of an immune cell. In certain embodiments, the methods and uses described herein reprogram an immune suppressor cell. In certain embodiments, the immune cell is an immune suppressor cell or an immune cell exhibiting immune suppressive activity. Exemplary immune cells with immune suppressive activity include, but are not limited to, regulatory T-cells (Tregs), tolerogenic dendritic cells (tolDCs), and myeloid-derived suppressor cell (MDSCs).

### 5.3.3. Reversing Stromal-Mediated Immunosuppression

A limitation in the successful development and clinical use of immunotherapies is the ability of tumors to evade and suppress the natural immune response against the tumor cells, by establishing an immunosuppressive tumor microenvironment. Stromal cells play an important role in the immunosuppressive tumor microenvironment by interacting with and suppressing various immune cells within the tumor microenvironment. This phenomenon is known as stromal-mediated immunosuppression.

Provided herein is a method of reversing stromal-mediated immunosuppression in a subject diagnosed with cancer, wherein the method comprises administering to the subject a therapeutically effective amount of an anti-ILT3 antibody and a therapeutically effective amount of an anti-LAIR-1 antibody, or pharmaceutical composition(s) thereof. In certain embodiments, the subject is a mammal. In certain embodiments, the subject is a human. In certain embodiments, the subject is pre-diagnosed with cancer before receiving the combination of an anti-ILT3 antibody and an anti-LAIR-1 antibody, as described herein. In certain embodiments, the subject is presently undergoing a cancer therapy. In certain embodiments, the subject has relapsed from a prior cancer treatment prior to receiving the combination of an anti-ILT3 antibody and an anti-LAIR-1 antibody, as described herein. In certain embodiments, the subject has been diagnosed for the first time as having cancer by a doctor prior to receiving the combination of an anti-ILT3 antibody and an anti-LAIR-1 antibody, as described herein. In certain embodiments, the anti-ILT3 antibody and the anti-LAIR-1 antibody are formulated into two separate pharmaceutical compositions. In certain embodiments, the anti-ILT3 antibody and the anti-LAIR-1 antibody are formulated into a pharmaceutical composition.

Also provided herein are uses of an anti-ILT3 antibody and an anti-LAIR-1 antibody, as described herein, for reversing stromal-mediated immunosuppression in a subject diagnosed with cancer, wherein the use comprises administering to the subject a therapeutically effective amount of the anti-ILT3 antibody and a therapeutically effective amount of the anti-LAIR-1 antibody. In certain embodiments, the subject is a mammal. In certain embodiments, the subject is a human. In certain embodiments, the subject is pre-diagnosed with cancer before receiving the composition or pharmaceutical composition. In certain embodiments, the subject is presently undergoing a cancer therapy. In certain embodiments, the subject has relapsed from a prior cancer treatment prior to receiving the composition or pharmaceutical composition. In certain embodiments, the subject has been diagnosed for the first time as having cancer by a doctor prior to receiving the composition or pharmaceutical composition. In certain embodiments, the anti-ILT3 antibody and the anti-LAIR-1 antibody are formulated into two separate pharmaceutical compositions for use. In certain embodiments, the anti-ILT3 antibody and the anti-LAIR-1 antibody are formulated into a pharmaceutical composition for use.

In certain embodiments, the reversal of stromal-mediated immunosuppression is achieved by the combination of an anti-ILT3 antibody and an anti-LAIR-1 antibody, which blocks the interaction between ILT3 and fibronectin, APOE or CNTFR, and the interaction between LAIR-1 and collagen, MARCO or COLEC12, thereby reversing stromal cell-mediated inhibition of immune cells associated with a tumor or tumor microenvironment. In certain embodiments, the immune cells are myeloid cells (e.g., macrophages, dendritic cells, monocytes, and/or APCs). As a result, the myeloid cells are activated or reactivated. They express pro-inflammatory cytokines and/or chemokines, recruiting effector T cells to the tumor or the tumor microenvironment, and thereby activating an immune response against the cancer.

In certain embodiments, the combination of an anti-ILT3 antibody and an anti-LAIR-1 antibody reverses stromal cell-mediated inhibition of other immune cells, such as T cells and/or NK cells, thereby activating an immune response against the cancer. In certain embodiments, the combination of an anti-ILT3 antibody and an anti-LAIR-1 antibody reverses stromal-mediated immunosuppression by inhibiting or reducing immunosuppressive activity of some other immune cells (e.g., tolerogenic dendritic cells, MDSCs and/or Tregs). As a result, these cells are reprogramed and the immunosuppression is relieved.

In certain embodiments, the reversal of stromal-mediated immunosuppression is a “full” reversal (i.e., the tumor microenvironment has no immunosuppressive properties or there is no stromal-mediated immunosuppression). In certain embodiments, the reversal of stromal-mediated immunosuppression is a “partial” reversal (i.e., less than a full reversal such as 90%, 80%, 70%, 60%, 50%, 40%, 30%, 20%, or 10% less than the full reversal).

### 5.3.4. Treating Cancer

Also provided herein are methods of treating cancer in a subject, wherein the method comprises administering to the subject a therapeutically effective amount of an anti-ILT3 antibody and a therapeutically effective amount of an anti-LAIR-1 antibody, or pharmaceutical composition(s) thereof. Also provided herein are uses of an anti-ILT3 antibody and an anti-LAIR-1 antibody in treating cancer in a subject, wherein the use comprises administering to the subject a therapeutically effective amount of the anti-ILT3 antibody and a therapeutically effective amount of the anti-LAIR-1 antibody. In certain embodiments, the anti-ILT3 antibody and the anti-LAIR-1 antibody are formulated into two separate pharmaceutical compositions for use. In certain embodiments, the anti-ILT3 antibody and the anti-LAIR-1 antibody are formulated into a pharmaceutical composition for use.

In certain embodiments, the subject is a mammal. In certain embodiments, the subject is a human. In certain embodiments, the subject is pre-diagnosed with cancer before receiving the combination of an anti-ILT3 antibody and an anti-LAIR-1 antibody, as described herein. In certain embodiments, the subject is presently undergoing a cancer therapy. In certain embodiments, the subject has relapsed from a prior cancer treatment prior to receiving the combination of an anti-ILT3 antibody and an anti-LAIR-1 antibody, as described herein. In certain embodiments, the subject has been diagnosed for the first time as having cancer by a doctor prior to receiving the combination of an anti-ILT3 antibody and an anti-LAIR-1 antibody, as described herein.

The term “treat” or “treatment” or “treating” or “to treat” as used herein refers to therapeutic measures that aim to relieve, slow down progression of, lessen symptoms of, and/or halt progression of a pathologic condition or disorder. Thus, those in need of treatment include those already with the disorder. In certain embodiments, treating cancer means stabilizing progression of the cancer. In certain embodiments, treating cancer means slowing down progression of the cancer. In certain embodiments, treating cancer means halting progression of the cancer. In certain embodiments, treating cancer means shrinking the cancer size. In certain embodiments, treating cancer means increasing the overall survival of the subject diagnosed with the cancer. Methods of assessing the progression of cancer are known in the art and include, for example, evaluation of target lesions using imaging (e.g., X-ray, computerized tomography scan, magnetic resonance imaging, caliper measurement, or positron emission tomography scan), cytology or histology, or expression of tumor marker(s) (see, e.g., Eisenhauer et al., 2009, European Journal of Cancer 45:228-247 and Schwartz et al., 2016, European Journal of Cancer 62:132-137; each of which is incorporated by reference herein in its entirety).

### 5.3.5. Enhancing an Immune Response Towards Cancer

Also described herein are methods of enhancing an immune response to a cancer cell or cancer cells in a subject diagnosed with the cancer, wherein the method comprises administering to the subject a therapeutically effective amount of an anti-ILT3 antibody and a therapeutically effective amount of an anti-LAIR-1 antibody, or pharmaceutical composition (s) thereof. Also described herein are uses of an anti-ILT3 antibody and an anti-LAIR-1 antibody in enhancing an immune response to a cancer cell or cancer cells in a subject diagnosed with the cancer, wherein the use comprises administering to the subject a therapeutically effective amount of the anti-ILT3 antibody and a therapeutically effective amount of an anti-LAIR-1 antibody. In certain embodiments, the anti-ILT3 antibody and the anti-LAIR-1 antibody are formulated into two separate pharmaceutical compositions for use. In certain embodiments, the anti-ILT3 antibody and the anti-LAIR-1 antibody are formulated into a pharmaceutical composition for use.

In certain embodiments, the subject is a mammal. In certain embodiments, the subject is a human. In certain embodiments, the subject is pre-diagnosed with the cancer before receiving the combination of an anti-ILT3 antibody and an anti-LAIR-1 antibody, as described herein. In certain embodiments, the subject is presently undergoing a cancer therapy. In certain embodiments, the subject has relapsed from a prior cancer treatment prior to receiving the combination of an anti-ILT3 antibody and an anti-LAIR-1 antibody, as described herein. In certain embodiments, the subject has been diagnosed for the first time as having the cancer by a doctor prior to receiving the combination of an anti-ILT3 antibody and an anti-LAIR-1 antibody, as described herein.

In certain embodiments, the enhancement of an immune response towards cancer is achieved through activation of an immune cell, as described herein. In certain embodiments, the enhancement of an immune response towards cancer is achieved through reduction or inhibition of the immune suppressive activity of an immune cell, as described herein. In certain embodiments, the enhancement of an immune response towards cancer is achieved through enhancement of cell-mediated immunity, for example, activation or reactivation of an effector T cell such as a cytotoxic T-cell.

The methods and uses for enhancing an immune response towards cancer can lead to a short-term, long-term, or an immediate enhancement of an immune response to a cancer or cancer cells. Short-term includes any period of days, weeks, or months. For example, short-term includes less than one year, 12 months, 11 months, 10 months, 9 months, 8 months, 7 months, 6 months, 5 months, 4 months, 3 months, 2 months, 1 month, 4 weeks, 3 weeks, 2 weeks, 1 week, 6 days, 5 days, 4 days, 3 days, 2 days, and 1 day. Long-term includes any period of more than one year. For example, long-term includes 1 year, 2 years, 3 years, 4 years, 5 years, 6 years, 7 years, 8 years, 9 years, 10 years, 10-15 years, 15-20 years.

### 5.3.6. Applicable to All Methods and Uses Described Herein

The words “cancer” and “tumor” are used interchangeably in this disclosure. In certain embodiments of all the methods and uses described herein, the cancer is a hematologic cancer. In certain embodiments, the cancer is a solid tumor. In certain embodiments, the cancer is a pancreatic cancer, breast cancer, mesothelioma, gastric cancer, (e.g., non-small cell lung cancer (NSCLC)), cervical cancer, endocervical cancer, biliary duct cancer, head and neck cancer (e.g., squamous cell carcinoma of the head and neck (SCCHN)), bladder cancer, urothelial cancer, colorectal cancer (CRC), esophageal cancer, ovarian cancer, kidney cancer (e.g., renal cell carcinoma (RCC)), prostate cancer or melanoma. In certain embodiments, the pancreatic cancer is pancreatic ductal adenocarcinoma.

In certain embodiments, the cancer is a tumor mutational burden-high (TMB-H) cancer (>10 mutations/megabase). Tumor mutational burden (TMB) is a measure of the total number of mutations per coding area of a tumor genome. Levels are measured by the number of non-inherited mutations occurring per megabase (1 million DNA base pairs) of the tumor genome. TMB can be measured with both tissue and blood-based comprehensive genomic tests.

In certain embodiments, the cancer is a microsatellite instability-high (MSI-H) cancer. Microsatellite testing that shows mutations in 30% or more microsatellites is called microsatellite instability-high. Microsatellite instability-high (MSI-H) can be found in many types of cancer, including colorectal cancer, endometrial cancer, biliary cancer, bladder cancer, breast cancer, esophageal cancer, gastric or gastroesophageal junction cancer, pancreatic cancer, prostate cancer, renal cell cancer, retroperitoneal adenocarcinoma, sarcoma, small cell lung, small intestinal cancer and thyroid cancer.

In certain embodiments of all the methods or uses described herein, an anti-ILT3 antibody and an anti-LAIR-1 antibody described herein are used in combination with one or more additional therapeutic agents. In certain embodiments, the use of one or more additional therapeutic agents in combination of an anti-ILT3 antibody and an anti-LAIR-1 antibody results in additive or synergistic effects compared to the combination of the anti-ILT3 antibody and the anti-LAIR-1 antibody alone. In certain embodiments, the use of one or more additional therapeutic agents in combination of an anti-ILT3 antibody and an anti-LAIR-1 antibody results in an increase in the therapeutic index of the anti-ILT3 antibody and the anti-LAIR-1 antibody. In certain embodiments, the use of one or more additional therapeutic agents in combination of an anti-ILT3 antibody and an anti-LAIR-1 antibody results in an increase in the therapeutic index of the additional therapeutic agent(s). In certain embodiments, the use of one or more additional therapeutic agents in combination of an anti-ILT3 antibody and an anti-LAIR-1 antibody results in a decrease in the toxicity and/or side effects of the anti-ILT3 antibody and the anti-LAIR-1 antibody. In certain embodiments, the use of one or more additional therapeutic agents in combination of an anti-ILT3 antibody and an anti-LAIR-1 antibody results in a decrease in the toxicity and/or side effects of the additional therapeutic agent(s).

In certain embodiments, the one or more therapeutic agents include, but are not limited to, anti-tubulin agents, auristatins, DNA minor groove binders, DNA replication inhibitors, alkylating agents (e.g., platinum complexes such as cisplatin, mono(platinum), bis(platinum) and tri-nuclear platinum complexes and carboplatin), anthracyclines, antibiotics, anti-folates, anti-metabolites, chemotherapy sensitizers, duocarmycins, etoposides, fhiorinated pyrimidines, ionophores, lexitropsins, nitrosoureas, platinols, purine antimetabolites, puromycins, radiation sensitizers, steroids, taxanes, topoisomerase inhibitors, vinca alkaloids, or the like.

In certain embodiments, the one or more additional therapeutic agents are selected from an alkylating agent, an antimetabolite, an antimitotic, a topoisomerase inhibitor and an angiogenesis inhibitor. In certain embodiments, the one or more additional therapeutic agents are chemotherapeutic agents, including, but are not limited to, alkylating agents such as thiotepa and cyclophosphamide (CYTOXAN); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamime; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, caminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytosine arabinoside, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenishers such as folinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK; razoxane; sizofuran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (Ara-C); taxoids, e.g. paclitaxel (TAXOL) and docetaxel (TAXOTERE); chlorambucil; gemcitabine; 6-thioguanine; mercaptopurine; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; ibandronate; CPT 11; topoisomerase inhibitor RFS 2000; difluoromethylornithine (DMFO); retinoic acid; esperamicins; capecitabine (XELODA); and pharmaceutically acceptable salts, acids or derivatives of any of the above.

In certain embodiments, the one or more additional therapeutic agents include anti-hormonal agents that act to regulate or inhibit hormone action on tumors, such as anti-estrogens including for example tamoxifen, raloxifene, aromatase inhibiting 4(5)-imidazoles, 4-hydroxytamoxifen, trioxifene, keoxifene, LY117018, onapristone, and toremifene (FARESTON); and anti-androgens including for example flutamide, nilutamide, bicalutamide, leuprolide, and goserelin; and pharmaceutically acceptable salts, acids or derivatives of any of the above.

In certain embodiments, the one or more additional therapeutic agents include a topoisomerase inhibitor. A topoisomerase inhibitor is chemotherapy agent that interferes with the action of a topoisomerase enzyme (e.g., topoisomerase I or II). A topoisomerase inhibitor that can be in this invention includes, but is not limited to, doxorubicin HCl, daunorubicin citrate, mitoxantrone HCl, actinomycin D, etoposide, topotecan HCl, teniposide (VM-26), and irinotecan, as well as a pharmaceutically acceptable salt, acid, or derivative thereof.

In certain embodiments, the one or more additional therapeutic agents include an anti-metabolite. An anti-metabolite is a chemical with a structure that is similar to a metabolite required for normal biochemical reactions, yet different enough to interfere with one or more normal functions of cells, such as cell division. An anti-metabolite that can be used in this invention includes, but is not limited to, gemcitabine, fluorouracil, capecitabine, methotrexate sodium, ralitrexed, pemetrexed, tegafur, cytosine arabinoside, thioguanine, 5-azacytidine, 6-mercaptopurine, azathioprine, 6-thioguanine, pentostatin, fludarabine phosphate, and cladribine, as well as a pharmaceutically acceptable salt, acid, or derivative thereof. In certain embodiments, the additional therapeutic agent is gemcitabine.

In certain embodiments, the one or more additional therapeutic agents includes an antimitotic agent, including, but not limited to, an agent that binds tubulin. In certain embodiments, the antimitotic agent is taxane. In certain embodiments, the antimitotic agent is paclitaxel or docetaxel, or a pharmaceutically acceptable salt, acid, or derivative of paclitaxel or docetaxel. In certain embodiments, the antimitotic agent is paclitaxel (TAXOL), docetaxel (TAXOTERE), albumin-bound paclitaxel (nab-paclitaxel; ABRAXANE), DHA-paclitaxel, or PG-paclitaxel. In certain embodiments, the antimitotic agent is a vinca alkaloid, such as vincristine, vinblastine, vinorelbine, or vindesine, or a pharmaceutically acceptable salt, acid, or derivative thereof. In certain embodiments, the antimitotic agent is an inhibitor of kinesin Eg5 or an inhibitor of a mitotic kinase such as Aurora A or Plk1. In certain embodiments, the additional therapeutic agent is paclitaxel. In certain embodiments, the additional therapeutic agent is nab-paclitaxel.

In certain embodiments, the one or more additional therapeutic agents include a small molecule, which acts as an inhibitor of a tumor-associated antigen, including, but not limited to, EGFR, HER2 (ErbB2), and VEGF. In certain embodiments, the one or more additional therapeutic agents include a protein kinase inhibitor including, but not limited to, gefitinib, erlotinib, sunitinib, lapatanib, vandetanib, AEE788, CI-1033, cediranib, sorafenib, and pazopanib. In certain embodiments, the one or more additional therapeutic agents includes an mTOR inhibitor.

In certain embodiments, the one or more additional therapeutic agents include a biological molecule, for example, an antibody binding to a tumor-associated antigen (e.g., an antibody that binds EGFR, HER2/ErbB2, or VEGF, including bevacizumab, ramucirumab, trastuzumab, pertuzumab, panitumumab, nimotuzumab, zalutumumab, or cetuximab). In certain embodiments, the one or more additional therapeutic agents include an antibody that is an angiogenesis inhibitor (e.g., an anti-VEGF or VEGF receptor antibody). In certain embodiments, the one or more additional therapeutic agents include a cytokine (e.g., a lymphokine, an interleukin, or a tumor necrosis factor).

In certain embodiments, the one or more additional therapeutic agents include a growth factor, for example, adrenomedullin (AM), angiopoietin (Ang), BMPs, BDNF, EGF, erythropoietin (EPO), FGF, GDNF, G-CSF, GM-CSF, GDF9, HGF, HDGF, IGF, migration-stimulating factor, myostatin (GDF-8), NGF, neurotrophins, PDGF, thrombopoietin, TGF-a, TGF-β, TNF-a, VEGF, PIGF, IL-1, IL-2, IL-3, IL-4, IL-5, IL-6, IL-7, IL-12, IL-15, or IL-18.

In certain embodiments, the one or more additional therapeutic agents include an agent that modulates an immune cell or immune response (an immunomodulatory agent). In certain embodiments, the agent is selected from the group consisting of granulocyte-macrophage colony stimulating factor (GM-CSF), macrophage colony stimulating factor (M-CSF), granulocyte colony stimulating factor (G-CSF), interleukin 3 (IL-3), interleukin 12 (IL-12), interleukin 1 (IL-1), interleukin 2 (IL-2), B7-1 (CD80), B7-2 (CD86), 4-1BB ligand, anti-CD3 antibody, anti-CTLA-4 antibody, anti-TIGIT antibody, anti-PD-1 antibody, anti-PD-L1 antibody, anti-LAG-3 antibody, and anti-TIM-3 antibody. In certain embodiments, an agent is selected from the group consisting of a modulator of PD-1 activity, a modulator of PD-L1 activity, a modulator of PD-L2 activity, a modulator of CTLA-4 activity, a modulator of CD28 activity, a modulator of CD80 activity, a modulator of CD86 activity, a modulator of 4-1BB activity, an modulator of OX40 activity, a modulator of KIR activity, a modulator of Tim-3 activity, a modulator of LAG3 activity, a modulator of CD27 activity, a modulator of CD40 activity, a modulator of GITR activity, a modulator of TIGIT activity, a modulator of CD20 activity, a modulator of CD96 activity, a modulator of IDO1 activity, a cytokine, a chemokine, an interferon, an interleukin, a lymphokine, a member of the tumor necrosis factor (TNF) family, and an immunostimulatory oligonucleotide. In certain embodiments, the agent is selected from the group consisting of a PD-1 antagonist, a PD-L1 antagonist, a PD-L2 antagonist, a CTLA-4 antagonist, a CD80 antagonist, a CD86 antagonist, a KIR antagonist, a Tim-3 antagonist, a LAG3 antagonist, a TIGIT antagonist, a CD20 antagonist, a CD96 antagonist, and an IDO1 antagonist.

In certain embodiments, the PD-1 antagonist is an antibody that specifically binds PD-1. In certain embodiments, the antibody that binds PD-1 is pembrolizumab, pidilizumab, nivolumab, MEDI0680, REGN2810, BGB-A317, PDR-001, or STI-A1110. In certain embodiments, the antibody that binds PD-1 is described in WO 2014179664A1, for example, an antibody identified as APE2058, APE1922, APE1923, APE1924, APE 1950, or APE1963, or an antibody containing the CDR regions of any of these antibodies. In other embodiments, the PD-1 antagonist is a fusion protein that includes PD-L2, for example, AMP-224. In other embodiments, the PD-1 antagonist is a peptide inhibitor, for example, AU P-12.

In certain embodiments, the PD-L1 antagonist is an antibody that specifically binds PD-L1. In certain embodiments, the antibody that binds PD-L1 is atezolizumab, MEDI4736, BMS-936559 (MDX-1105), avelumab, durvalumab, KD033, the antibody portion of KD033, or STI-A1014. In certain embodiments, the antibody that binds PD-L1 is described in WO 2014055897A1, for example, Ab-14, Ab-16, Ab-30, Ab-31, Ab-42, Ab-50, Ab-52, or Ab-55, or an antibody that contains the CDR regions of any of these antibodies.

In certain embodiments, the CTLA-4 antagonist is an antibody that specifically binds CTLA-4. In certain embodiments, the antibody that binds CTLA-4 is ipilimumab or tremelimumab. In certain embodiments, the CTLA-4 antagonist a CTLA-4 fusion protein, for example, KAHR-102.

In certain embodiments, the LAG3 antagonist is an antibody that specifically binds LAG3. In certain embodiments, the antibody that binds LAG3 is IMP701, IMP731, BMS-986016, LAG525, and GSK2831781. In certain embodiments, the LAG3 antagonist includes a soluble LAG3 receptor, for example, IMP321.

In certain embodiments, the KIR antagonist is an antibody that specifically binds KIR. In certain embodiments, the antibody that binds KIR is lirilumab.

In certain embodiments, the immunomodulatory agent includes an agent selected from the group consisting of a CD28 agonist, a 4-1BB agonist, an OX40 agonist, a CD27 agonist, a CD80 agonist, a CD86 agonist, a CD40 agonist, and a GITR agonist.

In certain embodiments, the OX40 agonist includes OX40 ligand, or an OX40-binding portion thereof. For example, the OX40 agonist may be MEDI6383. In certain embodiments, the OX40 agonist is an antibody that specifically binds OX40. In certain embodiments, the antibody that binds OX40 is MEDI6469, MEDI0562, or MOXR0916 (RG7888). In certain embodiments, the OX40 agonist is a vector (e.g., an expression vector or virus, such as an adenovirus) capable of expressing OX40 ligand. In certain embodiments the OX40-expressing vector is Delta −24-RGDOX or DNX2401.

In certain embodiments, the 4-1BB (CD137) agonist is a binding molecule, such as an anticalin. In certain embodiments, the anticalin is PRS-343. In certain embodiments, the 4-1BB agonist is an antibody that specifically binds 4-1BB. In certain embodiments, antibody that binds 4-1BB is PF-2566 (PF-05082566) or urelumab.

In certain embodiments, the CD27 agonist is an antibody that specifically binds CD27. In certain embodiments, the antibody that binds CD27 is varlilumab.

In certain embodiments, the GITR agonist includes a GITR ligand or a GITR-binding portion thereof. In certain embodiments, the GITR agonist is an antibody that specifically binds GITR. In certain embodiments, the antibody that binds GITR is TRX518, MK-4166, or INBRX-110.

In certain embodiments, the immunomodulatory agent includes an anti-PD-1 antibody, an anti-PD-L1 antibody, an anti-PD-L2 antibody, an anti-CTLA-4 antibody, an anti-CD28 antibody, an anti-CD80 antibody, an anti-CD86 antibody, an anti-4-1BB antibody, an anti-OX40 antibody, an anti-KIR antibody, an anti-Tim-3 antibody, an anti-LAG3 antibody, an anti-CD27 antibody, an anti-CD40 antibody, an anti-GITR antibody, an anti-TIGIT antibody, an anti-CD20 antibody, an anti-CD96 antibody, or an anti-IDO1 antibody.

In any one of the methods and uses described herein, an anti-ILT3 antibody and an anti-LAIR-1 antibody may be co-formulated or formulated separately. In any one of the methods and uses described herein, an anti-ILT3 antibody and an anti-LAIR-1 antibody (individually formulated or co-formulated) may be administered simultaneously, in any order, at different times, or in different frequencies.

In any one of the methods and uses described herein, an anti-ILT3 antibody, an anti-LAIR-1 antibody (individually formulated or co-formulated), and the one or more additional therapeutic agents may be administered simultaneously, in any order, at different times, or in different frequencies.

### 5.4. Kits

Also provided herein is a kit that comprises a first container, a second container and a package insert, wherein the first container includes at least one dose of a medicament comprising an anti-ILT3 antibody, the second container comprises at least one dose of a medicament comprising an anti-LAIR-1 antibody, and the package insert comprises instructions for treating a subject diagnosed with a cancer using the medicaments. In certain embodiments, the subject is a mammal. In certain embodiments, the subject is a human. In certain embodiments, the subject is pre-diagnosed with a cancer. In certain embodiments, the subject is presently undergoing a cancer therapy. In certain embodiments, the subject has relapsed from a prior cancer treatment before receiving the combination of an anti-ILT3 antibody and an anti-LAIR-1 antibody, as described herein. In certain embodiments, the subject has been diagnosed for the first time as having cancer by a doctor prior to receiving the combination of an anti-ILT3 antibody and an anti-LAIR-1 antibody, as described herein.

In certain embodiments, the kit comprises an anti-ILT3 antibody as described in WO 2021127200A1, US20190153093A1, WO2020056077A1, WO2021183839A2, US20200031926A1, US20210221887A1, US20150110714A1, US20200031926A1, US20190241655A1, WO2020180789A1, or WO2020056077A1. In certain embodiments, the anti-ILT3 antibody comprises a VH comprising a VH-CDR1, a VH-CDR2, and a VH-CDR3, and a VL comprising a VL-CDR1, a VL-CDR2, and a VL-CDR3, wherein the VH-CDR1, VH-CDR2, VH-CDR3, VL-CDR1, VL-CDR2 and VL-CDR3 are from any one of the VH and VL sequences of the antibodies described herein (e.g., 3A3, 5A7, 12A12, 16C5, 45G10, 48A6, 53F10, or Hz5A7.v5), such as the amino acid sequences depicted in Tables 1, 3-8, and 2 respectively). In certain embodiments, the anti-ILT3 antibody comprises a VH comprising the VH-CDR1, the VH-CDR2, and the VH-CDR3 of the antibody 5A7 or Hz5A7.v5 (see Table 2 and Table 8) and a VL comprising the VL-CDR1, the VL-CDR2, and the VL-CDR3 of the antibody 5A7 or Hz5A7.v5 (see Table 2 and Table 8). In certain embodiments, the anti-ILT3 antibody comprises a VH comprising the amino acid sequence set forth in SEQ ID NO:123 and a VL comprising the amino acid sequence set forth in SEQ ID NO:124. In certain embodiments, the anti-ILT3 antibody comprising a heavy chain comprising the amino acid sequence set forth in SEQ ID NO:126 and a light chain comprising the amino acid sequence set forth in SEQ ID NO:128.

In certain embodiments, the kit comprises an anti-LAIR-1 antibody as described in US20190338026A1 and WO2018027039A1. In certain embodiments, the anti-LAIR-1 antibody comprises a VH comprising a VH-CDR1, a VH-CDR2, a VH-CDR3, and a VL comprising a VL-CDR1, a VL-CDR2, and a VL-CDR3, wherein the VH-CDR1, VH-CDR2, VH-CDR3, VL-CDR1, VL-CDR2 and VL-CDR3 are from any one of the VH and VL sequences of the antibodies described herein (e.g., 47A1, 47H1, Hz47H1.v4, 57D12, 61H4, 62G10, Hz62G10.v1, 108D10, 43H2), such as the amino acid sequences depicted in Tables 9-15. In certain embodiments, the anti-LAIR-1 antibody comprises a VH comprising the VH-CDR1, VH-CDR2, and VH-CDR3 of the antibody 47H1, Hz47H1.v4, 62G10, or Hz62G10.v1 (see Table 10A, Table 10B and Table 13) and a VL comprising the VL-CDR1, VL-CDR2, and VL-CDR3 of the antibody 47H1, Hz47H1.v4, 62G10, or Hz62G10.v1 (see Table 10A, Table 10B and Table 13). In certain embodiments, the anti-LAIR-1 antibody comprises a VH comprising the amino acid sequence set forth in SEQ ID NO:179 and a VL comprising the amino acid sequence set forth in SEQ ID NO:180. In certain embodiments, the anti-LAIR-1 antibody comprises a heavy chain comprising the amino acid sequence set forth in SEQ ID NO:194 and a light chain comprising the amino acid sequence set forth in SEQ ID NO:196.

### 6. EMBODIMENTS

This invention provides the following non-limiting embodiments:

1. A method of activating an immune cell associated with a tumor or a tumor microenvironment, wherein the method comprises contacting the immune cell with an effective amount of an anti-ILT3 antibody and an effective amount of an anti-LAIR-1 antibody.
2. The method of embodiment 1, wherein the immune cell is a myeloid cell.
3. The method of embodiment 2, wherein the myeloid cell is a monocyte, a macrophage, a dendritic cell, or an APC.
4. The method of embodiment 1, wherein the immune cell is a T cell.
5. The method of embodiment 4, wherein the T cell is a cytotoxic T-cell (CTL).
6. The method of embodiment 1, wherein the immune cell is a natural killer cell.
7. A method of reducing or inhibiting immune suppressive activity of an immune cell associated with a tumor or a tumor microenvironment, wherein the method comprises contacting the immune cell with an effective amount of an anti-ILT3 antibody and an effective amount of an anti-LAIR-1 antibody.
8. The method of embodiment 7, wherein the immune cell is a regulatory T-cell (Treg).
9. The method of embodiment 7, wherein the immune cell is a tolerogenic dendritic cell.
10. The method of embodiment 7, wherein the immune cell is a myeloid-derived suppressor cell (MDSC).
11. The method of any one of embodiments 1-10, further comprising contacting the immune cell with an additional therapeutic agent.
12. The method of embodiment 11, wherein the additional therapeutic agent is an immune-checkpoint inhibitor.
13. The method of embodiment 12, wherein the immune-checkpoint inhibitor is a PD-1 inhibitor or a PD-L1 inhibitor.
14. A method of activating an immune cell in a subject diagnosed with cancer, wherein the method comprises administering to the subject a therapeutically effective amount of an anti-ILT3 antibody and a therapeutically effective amount of an anti-LAIR-1 antibody.
15. The method of embodiment 14, wherein the immune cell is a myeloid cell.
16. The method of embodiment 15, wherein the myeloid cell is a monocyte, a macrophage, a dendritic cell, or an APC.
17. The method of embodiment 14, wherein the immune cell is a T cell.
18. The method of embodiment 17, wherein the T cell is a cytotoxic T-cell (CTL).
19. The method of embodiment 14, wherein the immune cell is a natural killer cell.
20. A method of reducing or inhibiting immune suppressive activity of in an immune cell in a subject diagnosed with cancer, wherein the method comprises administering to the subject a therapeutically effective amount of an anti-ILT3 antibody and a therapeutically effective amount of an anti-LAIR-1 antibody.
21. The method of embodiment 20, wherein the immune cell is a regulatory T-cell (Treg).
22. The method of embodiment 20, wherein the immune cell is a tolerogenic dendritic cell.
23. The method of embodiment 20, wherein the immune cell is a myeloid-derived suppressor cell (MDSC).
24. A method of reversing stromal-mediated immunosuppression in a subject diagnosed with cancer, wherein the method comprises administering to the subject a therapeutically effective amount of an anti-ILT3 antibody and a therapeutically effective amount of an anti-LAIR-1 antibody.
25. A method of treating cancer in a subject, wherein the method comprises administering to the subject a therapeutically effective amount of an anti-ILT3 antibody and a therapeutically effective amount of an anti-LAIR-1 antibody.
26. A method of enhancing an immune response to cancer in a subject diagnosed with the cancer, wherein the method comprises administering to the subject a therapeutically effective amount of an anti-ILT3 antibody and a therapeutically effective amount of an anti-LAIR-1 antibody.
27. The method of any one of embodiments 14-26, wherein the cancer is a hematologic cancer.
28. The method of any one of embodiments 14-26, wherein the cancer is a solid tumor.
29. The method of embodiment 28, wherein the cancer is a pancreatic cancer, a breast cancer, a mesothelioma, a gastric cancer, an NSCLC, a cervical cancer, an endocervical cancer, a biliary duct cancer, a SCCHN, a bladder cancer, an urothelial cancer, a CRC, an esophageal cancer, an ovarian cancer, an RCC, a prostate cancer, or a melanoma.
30. The method of embodiment 29, wherein the pancreatic cancer is pancreatic ductal adenocarcinoma.
31. The method of any one of embodiments 14-26, wherein the cancer is a tumor mutational burden-high (TMB-H) cancer.
32. The method of any one of embodiments 14-26, wherein the cancer is a microsatellite instability-high (MSI-H) cancer.
33. The method of any one of embodiments 14-32, further comprising administering to the subject an additional therapeutic agent.
34. The method of embodiment 33, wherein the additional therapeutic agent is an immune-checkpoint inhibitor.
35. The method of embodiment 34, wherein the immune-checkpoint inhibitor is a PD-1 inhibitor or a PD-L1 inhibitor.
36. The method of any one of embodiments 14-35, wherein the subject is a human.
37. The method of any one of embodiments 1-36, wherein the anti-ILT3 antibody inhibits binding of ILT3 to fibronectin.
38. The method of any one of embodiments 1-37, wherein the anti-ILT3 antibody inhibits binding of ILT3 to APOE.
39. The method of any one of embodiments 1-38, wherein the anti-ILT3 antibody inhibits binding of ILT3 to CNTFR.
40. The method of any one of embodiments 1-39, wherein the anti-ILT3 antibody inhibits ILT3 activity.
41. The method of any one of embodiments 1-40, wherein the anti-LAIR-1 antibody inhibits LAIR-1 activity.
42. The method of any one of embodiments 1-41, wherein the anti-LAIR-1 antibody inhibits binding of LAIR-1 to collagen.
43. The method of any one of embodiments 1-42, wherein the anti-LAIR-1 antibody inhibits binding of LAIR-1 to MARCO.
44. The method of any one of embodiments 1-43, wherein the anti-LAIR-1 antibody inhibits binding of LAIR-1 to COLEC12.
45. The method of any one of embodiments 1-44, wherein the anti-ILT3 antibody comprises:

(a) a heavy chain variable region (VH) comprising a VH-complementarity determining region (CDR)1, a VH-CDR2, and a VH-CDR3 from the amino acid sequence of SEQ ID NO:111; and a light chain variable region (VL) comprising a VL-CDR1, a VL-CDR2, and a VL-CDR3 from the amino acid sequence of SEQ ID NO:112; or

(b) a VH comprising a VH-CDR1, a VH-CDR2, and a VH-CDR3 from the amino acid sequence of SEQ ID NO:123; and a VL comprising a VL-CDR1, a VL-CDR2, and a VL-CDR3 from the amino acid sequence of SEQ ID NO:124.

46. The method of embodiment 45, wherein:

(a) the VH comprises a VH-CDR1 comprising the amino acid sequence GFTFSSYGMS (SEQ ID NO:27), a VH-CDR2 comprising the amino acid sequence TISGGGSYTNYPDSVKG (SEQ ID NO:28), and a VH-CDR3 comprising the amino acid sequence REWRYTLYAMDY (SEQ ID NO:105); and the VL comprises a VL-CDR1 comprising the amino acid sequence RASESVESYGSSFMH (SEQ ID NO:106), a VL-CDR2 comprising the amino acid sequence LTSNLES (SEQ ID NO:31), and a VL-CDR3 comprising the amino acid sequence QQNNEDPFT (SEQ ID NO:32);

(b) the VH comprises a VH-CDR1 comprising the amino acid sequence GFTFSSY (SEQ ID NO:33), a VH-CDR2 comprising the amino acid sequence SGGGSY (SEQ ID NO:34), and a VH-CDR3 comprising the amino acid sequence REWRYTLYAMDY (SEQ ID NO:105); and the VL comprises a VL-CDR1 comprising the amino acid sequence RASESVESYGSSFMH (SEQ ID NO:106), a VL-CDR2 comprising the amino acid sequence LTSNLES (SEQ ID NO:31), and a VL-CDR3 comprising the amino acid sequence QQNNEDPFT (SEQ ID NO:32);

(c) the VH comprises a VH-CDR1 comprising the amino acid sequence GFTFSSYGMS (SEQ ID NO:27), a VH-CDR2 comprising the amino acid sequence TISGGGSYTN (SEQ ID NO:35), and a VH-CDR3 comprising the amino acid sequence REWRYTLYAMDY (SEQ ID NO:105); and the VL comprises a VL-CDR1 comprising the amino acid sequence RASESVESYGSSFMH (SEQ ID NO:106), a VL-CDR2 comprising the amino acid sequence LTSNLES (SEQ ID NO:31), and a VL-CDR3 comprising the amino acid sequence QQNNEDPFT (SEQ ID NO:32);

(d) the VH comprises a VH-CDR1 comprising the amino acid sequence SYGMS (SEQ ID NO:36), a VH-CDR2 comprising the amino acid sequence TISGGGSYTNYPDSVKG (SEQ ID NO:28), and a VH-CDR3 comprising the amino acid sequence REWRYTLYAMDY (SEQ ID NO:105); and the VL comprises a VL-CDR1 comprising the amino acid sequence RASESVESYGSSFMH (SEQ ID NO:106), a VL-CDR2 comprising the amino acid sequence LTSNLES (SEQ ID NO:31), and a VL-CDR3 comprising the amino acid sequence QQNNEDPFT (SEQ ID NO:32); or

(e) the VH comprises a VH-CDR1 comprising the amino acid sequence SSYGMS (SEQ ID NO:37), a VH-CDR2 comprising the amino acid sequence WVATISGGGSYTN (SEQ ID NO:38), and a VH-CDR3 comprising the amino acid sequence ARREWRYTLYAMD (SEQ ID NO:107), and the VL comprises a VL-CDR1 comprising the amino acid sequence ESYGSSFMHWY (SEQ ID NO:108), a VL-CDR2 comprising the amino acid sequence LLIYLTSNLE (SEQ ID NO:41), and a VL-CDR3 comprising the amino acid sequence QQNNEDPF (SEQ ID NO:42).

47. The method of embodiment 46, wherein:

(a) the VH comprises an amino acid sequence having at least 80% sequence identity to the amino acid sequence of SEQ ID NO:123;

(b) the VL comprises an amino acid sequence having at least 80% sequence identity to the amino acid sequence of SEQ ID NO:124; or

(c) the VH comprises an amino acid sequence having at least 80% sequence identity to the amino acid sequence of SEQ ID NO:123 and the VL comprises an amino acid sequence having at least 80% sequence identity to the amino acid sequence of SEQ ID NO:124.

48. The method of embodiment 47, wherein the VH comprises the amino acid sequence of SEQ ID NO:123 and the VL comprises the amino acid sequence of SEQ ID NO:124.
49. The method of any one of embodiments 1-48, wherein the anti-ILT3 antibody comprises:

(a) a heavy chain comprising an amino acid sequence having at least 80% sequence identity to the amino acid sequence of SEQ ID NO:126;

(b) a light chain comprising an amino acid sequence having at least 80% sequence identity to the amino acid sequence of SEQ ID NO:128; or

(c) a heavy chain comprising an amino acid sequence having at least 80% sequence identity to the amino acid sequence of SEQ ID NO:126, and a light chain comprising an amino acid sequence having at least 80% sequence identity to the amino acid sequence of SEQ ID NO:128.

50. The method of embodiment 49, wherein the heavy chain comprises the amino acid sequence of SEQ ID NO:126 and the light chain comprises the amino acid sequence of SEQ ID NO:128.
51. The method of any one of embodiments 1-50, wherein the anti-LAIR-1 antibody comprises a heavy chain variable region (VH) comprising a VH-complementarity determining region (CDR)1, a VH-CDR2 and a VH-CDR3 from SEQ ID NO:179, and a light chain variable region (VL) comprising a VL-CDR1, a VL-CDR2 and a VL-CDR3 from SEQ ID NO:180.
52. The method of embodiment 51, wherein:

(a) the VH comprises the VH-CDR1 comprising the amino acid sequence of SEQ ID NO:242, the VH-CDR2 comprising the amino acid sequence of SEQ ID NO:257, and the VH-CDR3 comprising the amino acid sequence of SEQ ID NO:244; and the VL comprises the VL-CDR1 comprising the amino acid sequence of SEQ ID NO:245, the VL-CDR2 comprising the amino acid sequence of SEQ ID NO:258, and the VL-CDR3 comprising the amino acid sequence of SEQ ID NO:247;

(b) the VH comprises the VH-CDR1 comprising the amino acid sequence of SEQ ID NO:248, the VH-CDR2 comprising the amino acid sequence of SEQ ID NO:259, and the VH-CDR3 comprising the amino acid sequence of SEQ ID NO:244; and the VL comprises the VL-CDR1 comprising the amino acid sequence of SEQ ID NO:245, the VL-CDR2 comprising the amino acid sequence of SEQ ID NO:258, and the VL-CDR3 comprising the amino acid sequence of SEQ ID NO:247;

(c) the VH comprises the VH-CDR1 comprising the amino acid sequence of SEQ ID NO:242, the VH-CDR2 comprising the amino acid sequence of SEQ ID NO:260, and the VH-CDR3 comprising the amino acid sequence of SEQ ID NO:244; and the VL comprises the VL-CDR1 comprising the amino acid sequence of SEQ ID NO:245, the VL-CDR2 comprising the amino acid sequence of SEQ ID NO:258, and the VL-CDR3 comprising the amino acid sequence of SEQ ID NO:247;

(d) the VH comprises the VH-CDR1 comprising the amino acid sequence of SEQ ID NO:250, the VH-CDR2 comprising the amino acid sequence of SEQ ID NO:257, and the VH-CDR3 comprising the amino acid sequence of SEQ ID NO:244; and the VL comprises the VL-CDR1 comprising the amino acid sequence of SEQ ID NO:245, the VL-CDR2 comprising the amino acid sequence of SEQ ID NO:258, and the VL-CDR3 comprising the amino acid sequence of SEQ ID NO:247; or

(e) the VH comprises the VH-CDR1 comprising the amino acid sequence of SEQ ID NO:251, the VH-CDR2 comprising the amino acid sequence of SEQ ID NO:261, and the VH-CDR3 comprising the amino acid sequence of SEQ ID NO:253; and the VL comprises the VL-CDR1 comprising the amino acid sequence of SEQ ID NO:254, the VL-CDR2 comprising the amino acid sequence of SEQ ID NO:255, and the VL-CDR3 comprising the amino acid sequence of SEQ ID NO:256.

53. The method of embodiment 51 or 52, wherein the VH has at least 80% sequence identity to the amino acid sequence of SEQ ID NO:179, and/or the VL has at least 80% sequence identity to the amino acid sequence of SEQ ID NO:180.
54. The method of embodiment 53, wherein the VH comprises the amino acid sequence of SEQ ID NO:179, and/or the VL comprises the amino acid sequence of SEQ ID NO:180.
55. The method of any one of embodiments 1-54, wherein the anti-LAIR-1 antibody comprises: a heavy chain comprising an amino acid sequence having 80% identity to the amino acid sequence of SEQ ID NO:194, and/or a light chain comprising an amino acid sequence having 80% identity to the amino acid sequence of SEQ ID NO:196.
56. The method of embodiment 55, wherein the heavy chain comprises the amino acid sequence of SEQ ID NO:194, and/or the light chain comprises the amino acid sequence of SEQ ID NO:196.
57. The method of any one of embodiments 1-56, wherein the anti-ILT3 antibody and the anti-LAIR-1 antibody are formulated into two separate pharmaceutical compositions, where a first pharmaceutical composition comprises the anti-ILT3 antibody and a pharmaceutically acceptable carrier; and a second pharmaceutical composition comprises the anti-LAIR-1 antibody and a pharmaceutically acceptable carrier.
58. The method of any one of embodiments 1-56, wherein the anti-ILT3 antibody and the anti-LAIR-1 antibody are formulated into a pharmaceutical composition, wherein the pharmaceutical composition comprises the anti-ILT3 antibody, the anti-LAIR-1 antibody, and a pharmaceutically acceptable carrier.
59. Use of an anti-ILT3 antibody and an anti-LAIR-1 antibody for activating an immune cell associated with a tumor or a tumor microenvironment, wherein the use comprises contacting the immune cell with an effective amount of the anti-ILT3 antibody and an effective amount of the anti-LAIR-1 antibody.
60. The use of embodiment 59, wherein the immune cell is a myeloid cell.
61. The use of embodiment 60, wherein the myeloid cell is a monocyte, a macrophage, a dendritic cell, or an APC.
62. The use of embodiment 60, wherein the immune cell is a T cell.
63. The use of embodiment 62, wherein the T cell is a cytotoxic T-cell (CTL).
64. The use of embodiment 60, wherein the immune cell is a natural killer cell.
65. Use of an anti-ILT3 antibody and an anti-LAIR-1 antibody for reducing or inhibiting immune suppressive activity of an immune cell associated with a tumor or a tumor microenvironment, wherein the use comprises contacting the immune cell with an effective amount of the anti-ILT3 antibody and an effective amount of the anti-LAIR-1 antibody.
66. The use of embodiment 65, wherein the immune cell is a regulatory T-cell (Treg).
67. The use of embodiment 65, wherein the immune cell is a tolerogenic dendritic cell. 68. The use of embodiment 65, wherein the immune cell is a myeloid-derived suppressor cell (MDSC).
69. The use of any one of embodiments 59-68, further comprising contacting the immune cell with an additional therapeutic agent.
70. The use of embodiment 69, wherein the additional therapeutic agent is an immune checkpoint inhibitor.
71. The use of embodiment 70, wherein the immune-checkpoint inhibitor is a PD-1 inhibitor or a PD-L1 inhibitor.
72. Use of an anti-ILT3 antibody and an anti-LAIR-1 antibody for activating an immune cell in a subject diagnosed with a cancer, wherein the use comprises administering to the subject a therapeutically effective amount of the anti-ILT3 antibody and a therapeutically effective amount of the anti-LAIR-1 antibody.
73. The use of embodiment 72, wherein the immune cell is a myeloid cell.
74. The use of embodiment 73, wherein the myeloid cell is a monocyte, a macrophage, a dendritic cell, or an APC.
75. The use of embodiment 72, wherein the immune cell is a T cell.
76. The use of embodiment 75, wherein the T cell is a cytotoxic T-cell (CTL).
77. The use of embodiment 72, wherein the immune cell is a natural killer cell.
78. Use of an anti-ILT3 antibody and an anti-LAIR-1 antibody for reducing or inhibiting immune suppressive activity of an immune cell in a subject diagnosed with cancer, wherein the use comprises administering to the subject a therapeutically effective amount of the anti-ILT3 antibody and a therapeutically effective amount of the anti-LAIR-1 antibody.
79. The use of embodiment 78, wherein the immune cell is a tolerogenic dendritic cell.
80. The use of embodiment 78, wherein the immune cell is a regulatory T-cell (Treg).
81. The use of embodiment 78, wherein the immune cell is a myeloid-derived suppressor cell (MDSC).
82. Use of an anti-ILT3 antibody and an anti-LAIR-1 antibody for reversing stromal-mediated immunosuppression in a subject diagnosed with cancer, wherein the use comprises administering to the subject a therapeutically effective amount of the anti-ILT3 antibody and a therapeutically effective amount of the anti-LAIR-1 antibody.
83. Use of an anti-ILT3 antibody and an anti-LAIR-1 antibody for treating cancer in a subject, wherein the use comprises administering to the subject a therapeutically effective amount of the anti-ILT3 antibody and a therapeutically effective amount of the anti-LAIR-1 antibody.
84. Use of an anti-ILT3 antibody and an anti-LAIR-1 antibody for enhancing an immune response to cancer in a subject diagnosed with the cancer, wherein the use comprises administering to the subject a therapeutically effective amount of the anti-ILT3 antibody and a therapeutically effective amount of the anti-LAIR-1 antibody.
85. The use of any one of embodiments 59-84, wherein the tumor or cancer is a hematologic cancer.
86. The use of any one of embodiments 59-84, wherein the tumor or cancer is a solid tumor.
87. The use of embodiment 86, wherein the tumor or cancer is a pancreatic cancer, a breast cancer, a mesothelioma, a gastric cancer, an NSCLC, a cervical cancer, an endocervical cancer, a biliary duct cancer, a SCCHN, a bladder cancer, an urothelial cancer, a CRC, an esophageal cancer, an ovarian cancer, a RCC, a prostate cancer, or a melanoma.
88. The use of embodiment 87, wherein the pancreatic cancer is pancreatic ductal adenocarcinoma.
89. The use of any of embodiments 59-84, wherein the tumor or cancer is a tumor mutational burden-high (TMB-H) cancer.
90. The use of any of embodiments 59-84, wherein the tumor or cancer is a microsatellite instability-high (MSI-H) cancer.
91. The use of any of embodiments 72-90, further comprising administering to the subject an additional therapeutic agent.
92. The use of embodiment 91, wherein the additional therapeutic agent is an immune-checkpoint inhibitor.
93. The use of embodiment 92, wherein the immune-checkpoint inhibitor is a PD-1 inhibitor or a PD-L1 inhibitor.
94. The use of any one of embodiments 72-93, wherein the subject is a human.
95. The use of any one of embodiments 59-94, wherein the anti-ILT3 antibody inhibits binding of ILT3 to fibronectin.
96. The use of any one of embodiments 59-95, wherein the anti-ILT3 antibody inhibits binding of ILT3 to APOE.
97. The use of any one of embodiments 59-96, wherein the anti-ILT3 antibody inhibits binding of ILT3 to CNTFR.
98. The use of any one of embodiments 59-97, wherein the anti-ILT3 antibody inhibits ILT3 activity.
99. The use of any one of embodiments 59-98, wherein the anti-LAIR-1 antibody inhibits LAIR-1 activity.
100. The use of any one of embodiments 59-99, wherein the anti-LAIR-1 antibody inhibits binding of LAIR-1 to collagen.
101. The use of any of embodiments 59-100, wherein the anti-LAIR-1 antibody inhibits binding of LAIR-1 to MARCO.
102. The use of any of embodiments 59-101, wherein the anti-LAIR-1 antibody inhibits binding of LAIR-1 to COLEC12.
103. The use of any one of embodiments 59-102, wherein the anti-ILT3 antibody comprises:

(a) a heavy chain variable region (VH) comprising a VH-complementarity determining region (CDR)1, a VH-CDR2, and a VH-CDR3 from the amino acid sequence of SEQ ID NO:111; and a light chain variable region (VL) comprising a VL-CDR1, a VL-CDR2, and a VL-CDR3 from the amino acid sequence of SEQ ID NO:112; or

(b) a VH comprising a VH-CDR1, a VH-CDR2, and a VH-CDR3 from the amino acid sequence of SEQ ID NO:123; and a VL comprising a VL-CDR1, a VL-CDR2, and a VL-CDR3 from the amino acid sequence of SEQ ID NO:124.

104. The use of embodiment 103, wherein:

(a) the VH comprises a VH-CDR1 comprising the amino acid sequence GFTFSSYGMS (SEQ ID NO:27), a VH-CDR2 comprising the amino acid sequence TISGGGSYTNYPDSVKG (SEQ ID NO:28), and a VH-CDR3 comprising the amino acid sequence REWRYTLYAMDY (SEQ ID NO:105); and the VL comprises a VL-CDR1 comprising the amino acid sequence RASESVESYGSSFMH (SEQ ID NO:106), a VL-CDR2 comprising the amino acid sequence LTSNLES (SEQ ID NO:31), and a VL-CDR3 comprising the amino acid sequence QQNNEDPFT (SEQ ID NO:32);

(b) the VH comprises a VH-CDR1 comprising the amino acid sequence GFTFSSY (SEQ ID NO:33), a VH-CDR2 comprising the amino acid sequence SGGGSY (SEQ ID NO:34), and a VH-CDR3 comprising the amino acid sequence REWRYTLYAMDY (SEQ ID NO:105); and the VL comprises a VL-CDR1 comprising the amino acid sequence RASESVESYGSSFMH (SEQ ID NO:106), a VL-CDR2 comprising the amino acid sequence LTSNLES (SEQ ID NO:31), and a VL-CDR3 comprising the amino acid sequence QQNNEDPFT (SEQ ID NO:32);

(c) the VH comprises a VH-CDR1 comprising the amino acid sequence GFTFSSYGMS (SEQ ID NO:27), a VH-CDR2 comprising the amino acid sequence TISGGGSYTN (SEQ ID NO:35), and a VH-CDR3 comprising the amino acid sequence REWRYTLYAMDY (SEQ ID NO:105); and the VL comprises a VL-CDR1 comprising the amino acid sequence RASESVESYGSSFMH (SEQ ID NO:106), a VL-CDR2 comprising the amino acid sequence LTSNLES (SEQ ID NO:31), and a VL-CDR3 comprising the amino acid sequence QQNNEDPFT (SEQ ID NO:32);

(d) the VH comprises a VH-CDR1 comprising the amino acid sequence SYGMS (SEQ ID NO:36), a VH-CDR2 comprising the amino acid sequence TISGGGSYTNYPDSVKG (SEQ ID NO:28), and a VH-CDR3 comprising the amino acid sequence REWRYTLYAMDY (SEQ ID NO:105); and the VL comprises a VL-CDR1 comprising the amino acid sequence RASESVESYGSSFMH (SEQ ID NO:106), a VL-CDR2 comprising the amino acid sequence LTSNLES (SEQ ID NO:31), and a VL-CDR3 comprising the amino acid sequence QQNNEDPFT (SEQ ID NO:32); or

(e) the VH comprises a VH-CDR1 comprising the amino acid sequence SSYGMS (SEQ ID NO:37), a VH-CDR2 comprising the amino acid sequence WVATISGGGSYTN (SEQ ID NO:38), and a VH-CDR3 comprising the amino acid sequence ARREWRYTLYAMD (SEQ ID NO:107), and the VL comprises a VL-CDR1 comprising the amino acid sequence ESYGSSFMHWY (SEQ ID NO:108), a VL-CDR2 comprising the amino acid sequence LLIYLTSNLE (SEQ ID NO:41), and a VL-CDR3 comprising the amino acid sequence QQNNEDPF (SEQ ID NO:42).

105. The use of embodiment 103 or 104, wherein:

(a) the VH comprises an amino acid sequence having at least 80% sequence identity to the amino acid sequence of SEQ ID NO:123;

(b) the VL comprises an amino acid sequence having at least 80% sequence identity to the amino acid sequence of SEQ ID NO:124; or

(c) the VH comprises an amino acid sequence having at least 80% sequence identity to the amino acid sequence of SEQ ID NO:123 and the VL comprises an amino acid sequence having at least 80% sequence identity to the amino acid sequence of SEQ ID NO:124.

106. The use of embodiment 105, wherein the VH comprises the amino acid sequence of SEQ ID NO:123 and the VL comprises the amino acid sequence of SEQ ID NO:124.
107. The use of any one of embodiments 103-105, wherein the anti-ILT3 antibody comprises:

(a) a heavy chain comprising an amino acid sequence having at least 80% sequence identity to the amino acid sequence of SEQ ID NO:126;

(b) a light chain comprising an amino acid sequence having at least 80% sequence identity to the amino acid sequence of SEQ ID NO:128; or

(c) a heavy chain comprising an amino acid sequence having at least 80% sequence identity to the amino acid sequence of SEQ ID NO:126, and a light chain comprising an amino acid sequence having at least 80% sequence identity to the amino acid sequence of SEQ ID NO:128.

108. The use of embodiment 107, wherein the heavy chain comprises the amino acid sequence of SEQ ID NO:126 and the light chain comprises the amino acid sequence of SEQ ID NO:128.
109. The use of any one of embodiments 59-108, wherein the anti-LAIR-1 antibody comprises: a heavy chain variable region (VH) comprising a VH-complementarity determining region (CDR)1, a VH-CDR2 and a VH-CDR3 from SEQ ID NO:179, and a light chain variable region (VL) comprising a VL-CDR1, a VL-CDR2 and a VL-CDR3 from SEQ ID NO:180.
110. The use of embodiment 109, wherein:

(a) the VH comprises the VH-CDR1 comprising the amino acid sequence of SEQ ID NO:242, the VH-CDR2 comprising the amino acid sequence of SEQ ID NO:257, and the VH-CDR3 comprising the amino acid sequence of SEQ ID NO:244; and the VL comprises the VL-CDR1 comprising the amino acid sequence of SEQ ID NO:245, the VL-CDR2 comprising the amino acid sequence of SEQ ID NO:258, and the VL-CDR3 comprising the amino acid sequence of SEQ ID NO:247;

(b) the VH comprises the VH-CDR1 comprising the amino acid sequence of SEQ ID NO:248, the VH-CDR2 comprising the amino acid sequence of SEQ ID NO:259, and the VH-CDR3 comprising the amino acid sequence of SEQ ID NO:244; and the VL comprises the VL-CDR1 comprising the amino acid sequence of SEQ ID NO:245, the VL-CDR2 comprising the amino acid sequence of SEQ ID NO:258, and the VL-CDR3 comprising the amino acid sequence of SEQ ID NO:247;

(c) the VH comprises the VH-CDR1 comprising the amino acid sequence of SEQ ID NO:242, the VH-CDR2 comprising the amino acid sequence of SEQ ID NO:260, and the VH-CDR3 comprising the amino acid sequence of SEQ ID NO:244; and the VL comprises the VL-CDR1 comprising the amino acid sequence of SEQ ID NO:245, the VL-CDR2 comprising the amino acid sequence of SEQ ID NO:258, and the VL-CDR3 comprising the amino acid sequence of SEQ ID NO:247;

(d) the VH comprises the VH-CDR1 comprising the amino acid sequence of SEQ ID NO:250, the VH-CDR2 comprising the amino acid sequence of SEQ ID NO:257, and the VH-CDR3 comprising the amino acid sequence of SEQ ID NO:244; and the VL comprises the VL-CDR1 comprising the amino acid sequence of SEQ ID NO:245, the VL-CDR2 comprising the amino acid sequence of SEQ ID NO:258, and the VL-CDR3 comprising the amino acid sequence of SEQ ID NO:247; or

(e) the VH comprises the VH-CDR1 comprising the amino acid sequence of SEQ ID NO:251, the VH-CDR2 comprising the amino acid sequence of SEQ ID NO:261, and the VH-CDR3 comprising the amino acid sequence of SEQ ID NO:253; and the VL comprises the VL-CDR1 comprising the amino acid sequence of SEQ ID NO:254, the VL-CDR2 comprising the amino acid sequence of SEQ ID NO:255, and the VL-CDR3 comprising the amino acid sequence of SEQ ID NO:256.

111. The use of any one of embodiments 109 or 110, wherein: the VH has at least 80% sequence identity to the amino acid sequence of SEQ ID NO:179 and/or the VL has at least 80% sequence identity to the amino acid sequence of SEQ ID NO:180.
112. The use of embodiment 111, wherein: the VH comprises the amino acid sequence of SEQ ID NO:179, and/or the VL comprises the amino acid sequence of SEQ ID NO:180.
113. The use of any one of embodiments 59-112, wherein the anti-LAIR-1 antibody comprises: a heavy chain comprising an amino acid sequence with 80% identity to the amino acid sequence of SEQ ID NO:194, and/or a light chain comprising an amino acid sequence with 80% identity to the amino acid sequence of SEQ ID NO:196.
114. The use of embodiment 113, wherein the heavy chain comprises the amino acid sequence of SEQ ID NO:194, and/or the light chain comprises the amino acid sequence of SEQ ID NO:196.
115. The use of any one of embodiments 59-113, wherein the anti-ILT3 antibody and the anti-LAIR-1 antibody are formulated into two separate pharmaceutical compositions, where a first pharmaceutical composition comprises the anti-ILT3 antibody and a pharmaceutically acceptable carrier; and a second pharmaceutical composition comprises the anti-LAIR-1 antibody and a pharmaceutically acceptable carrier.
116. The use of any one of embodiments 59-113, wherein the anti-ILT3 antibody and the anti-LAIR-1 antibody are formulated into a pharmaceutical composition, wherein the pharmaceutical composition comprises the anti-ILT3 antibody, the anti-LAIR-1 antibody, and a pharmaceutically acceptable carrier.
117. A kit comprising a first container, a second container and a package insert, wherein the first container comprises at least one dose of a medicament comprising an anti-ILT3 antibody, the second container comprises at least one dose of a medicament comprising an anti-LAIR-1 antibody, and the package insert comprises instructions for treating a subject diagnosed with cancer using the medicaments.
118. The kit of embodiment 117, wherein the subject is a human.
119. The kit of embodiment 117 or 118, wherein the anti-ILT3 antibody comprises:

(a) a heavy chain variable region (VH) comprising a VH-complementarity determining region (CDR)1, a VH-CDR2, and a VH-CDR3 from the amino acid sequence of SEQ ID NO:111; and a light chain variable region (VL) comprising a VL-CDR1, a VL-CDR2, and a VL-CDR3 from the amino acid sequence of SEQ ID NO:112; or

(b) a VH comprising a VH-CDR1, a VH-CDR2, and a VH-CDR3 from the amino acid sequence of SEQ ID NO:123; and a VL comprising a VL-CDR1, a VL-CDR2, and a VL-CDR3 from the amino acid sequence of SEQ ID NO:124.

120. The kit of embodiment 119, wherein:

(a) the VH comprises a VH-CDR1 comprising the amino acid sequence GFTFSSYGMS (SEQ ID NO:27), a VH-CDR2 comprising the amino acid sequence TISGGGSYTNYPDSVKG (SEQ ID NO:28), and a VH-CDR3 comprising the amino acid sequence REWRYTLYAMDY (SEQ ID NO:105); and the VL comprises a VL-CDR1 comprising the amino acid sequence RASESVESYGSSFMH (SEQ ID NO:106), a VL-CDR2 comprising the amino acid sequence LTSNLES (SEQ ID NO:31), and a VL-CDR3 comprising the amino acid sequence QQNNEDPFT (SEQ ID NO:32);

(b) the VH comprises a VH-CDR1 comprising the amino acid sequence GFTFSSY (SEQ ID NO:33), a VH-CDR2 comprising the amino acid sequence SGGGSY (SEQ ID NO:34), and a VH-CDR3 comprising the amino acid sequence REWRYTLYAMDY (SEQ ID NO:105); and the VL comprises a VL-CDR1 comprising the amino acid sequence RASESVESYGSSFMH (SEQ ID NO:106), a VL-CDR2 comprising the amino acid sequence LTSNLES (SEQ ID NO:31), and a VL-CDR3 comprising the amino acid sequence QQNNEDPFT (SEQ ID NO:32);

(c) the VH comprises a VH-CDR1 comprising the amino acid sequence GFTFSSYGMS (SEQ ID NO:27), a VH-CDR2 comprising the amino acid sequence TISGGGSYTN (SEQ ID NO:35), and a VH-CDR3 comprising the amino acid sequence REWRYTLYAMDY (SEQ ID NO:105); and the VL comprises a VL-CDR1 comprising the amino acid sequence RASESVESYGSSFMH (SEQ ID NO:106), a VL-CDR2 comprising the amino acid sequence LTSNLES (SEQ ID NO:31), and a VL-CDR3 comprising the amino acid sequence QQNNEDPFT (SEQ ID NO:32);

(d) the VH comprises a VH-CDR1 comprising the amino acid sequence SYGMS (SEQ ID NO:36), a VH-CDR2 comprising the amino acid sequence TISGGGSYTNYPDSVKG (SEQ ID NO:28), and a VH-CDR3 comprising the amino acid sequence REWRYTLYAMDY (SEQ ID NO:105); and the VL comprises a VL-CDR1 comprising the amino acid sequence RASESVESYGSSFMH (SEQ ID NO:106), a VL-CDR2 comprising the amino acid sequence LTSNLES (SEQ ID NO:31), and a VL-CDR3 comprising the amino acid sequence QQNNEDPFT (SEQ ID NO:32); or

(e) the VH comprises a VH-CDR1 comprising the amino acid sequence SSYGMS (SEQ ID NO:37), a VH-CDR2 comprising the amino acid sequence WVATISGGGSYTN (SEQ ID NO:38), and a VH-CDR3 comprising the amino acid sequence ARREWRYTLYAMD (SEQ ID NO:107), and the VL comprises a VL-CDR1 comprising the amino acid sequence ESYGSSFMHWY (SEQ ID NO:108), a VL-CDR2 comprising the amino acid sequence LLIYLTSNLE (SEQ ID NO:41), and a VL-CDR3 comprising the amino acid sequence QQNNEDPF (SEQ ID NO:42).

121. The kit of embodiment 119 or 120, wherein:

(a) the VH comprises an amino acid sequence having at least 80% sequence identity to the amino acid sequence of SEQ ID NO:123;

(b) the VL comprises an amino acid sequence having at least 80% sequence identity to the amino acid sequence of SEQ ID NO:124; or

(c) the VH comprises an amino acid sequence having at least 80% sequence identity to the amino acid sequence of SEQ ID NO:123 and the VL comprises an amino acid sequence having at least 80% sequence identity to the amino acid sequence of SEQ ID NO:124.

122. The kit of embodiment 121, wherein the VH comprises the amino acid sequence of SEQ ID NO:123 and the VL comprises the amino acid sequence of SEQ ID NO:124.
123. The kit of any one of embodiments 117-122, wherein the anti-ILT3 antibody comprises:

(a) a heavy chain comprising an amino acid sequence having at least 80% sequence identity to the amino acid sequence of SEQ ID NO:126;

(b) a light chain comprising an amino acid sequence having at least 80% sequence identity to the amino acid sequence of SEQ ID NO:128; or

(c) a heavy chain comprising an amino acid sequence having at least 80% sequence identity to the amino acid sequence of SEQ ID NO:126, and a light chain comprising an amino acid sequence having at least 80% sequence identity to the amino acid sequence of SEQ ID NO:128.

124. The kit of embodiment 123, wherein the heavy chain comprises the amino acid sequence of SEQ ID NO:126 and the light chain comprises the amino acid sequence of SEQ ID NO:128.
125. The kit any one of embodiments 117-124, wherein the anti-LAIR-1 antibody comprises: a heavy chain variable region (VH) comprising a VH-complementarity determining region (CDR)1, a VH-CDR2 and a VH-CDR3 from SEQ ID NO:179, and a light chain variable region (VL) comprising a VL-CDR1, a VL-CDR2 and a VL-CDR3 from SEQ ID NO:180.
126. The kit of embodiment 125, wherein:

(a) the VH comprises the VH-CDR1 comprising the amino acid sequence of SEQ ID NO:242, the VH-CDR2 comprising the amino acid sequence of SEQ ID NO:257, and the VH-CDR3 comprising the amino acid sequence of SEQ ID NO:244; and the VL comprises the VL-CDR1 comprising the amino acid sequence of SEQ ID NO:245, the VL-CDR2 comprising the amino acid sequence of SEQ ID NO:258, and the VL-CDR3 comprising the amino acid sequence of SEQ ID NO:247;

(b) the VH comprises the VH-CDR1 comprising the amino acid sequence of SEQ ID NO:248, the VH-CDR2 comprising the amino acid sequence of SEQ ID NO:259, and the VH-CDR3 comprising the amino acid sequence of SEQ ID NO:244; and the VL comprises the VL-CDR1 comprising the amino acid sequence of SEQ ID NO:245, the VL-CDR2 comprising the amino acid sequence of SEQ ID NO:258, and the VL-CDR3 comprising the amino acid sequence of SEQ ID NO:247;

(c) the VH comprises the VH-CDR1 comprising the amino acid sequence of SEQ ID NO:242, the VH-CDR2 comprising the amino acid sequence of SEQ ID NO:260, and the VH-CDR3 comprising the amino acid sequence of SEQ ID NO:244; and the VL comprises the VL-CDR1 comprising the amino acid sequence of SEQ ID NO:245, the VL-CDR2 comprising the amino acid sequence of SEQ ID NO:258, and the VL-CDR3 comprising the amino acid sequence of SEQ ID NO:247;

(d) the VH comprises the VH-CDR1 comprising the amino acid sequence of SEQ ID NO:250, the VH-CDR2 comprising the amino acid sequence of SEQ ID NO:257, and the VH-CDR3 comprising the amino acid sequence of SEQ ID NO:244; and the VL comprises the VL-CDR1 comprising the amino acid sequence of SEQ ID NO:245, the VL-CDR2 comprising the amino acid sequence of SEQ ID NO:258, and the VL-CDR3 comprising the amino acid sequence of SEQ ID NO:247; or

(e) the VH comprises the VH-CDR1 comprising the amino acid sequence of SEQ ID NO:251, the VH-CDR2 comprising the amino acid sequence of SEQ ID NO:261, and the VH-CDR3 comprising the amino acid sequence of SEQ ID NO:253; and the VL comprises the VL-CDR1 comprising the amino acid sequence of SEQ ID NO:254, the VL-CDR2 comprising the amino acid sequence of SEQ ID NO:255, and the VL-CDR3 comprising the amino acid sequence of SEQ ID NO:256.

127. The kit of embodiment 125 or 126, wherein: the VH has at least 80% sequence identity to the amino acid sequence of SEQ ID NO:179; and/or the VL has at least 80% sequence identity to the amino acid sequence of SEQ ID NO:180.
128. The kit of embodiment 127, wherein: the VH comprises the amino acid sequence of SEQ ID NO:179; and/or the VL comprises the amino acid sequence of SEQ ID NO:180.
129. The kit of any of embodiments 117-128, wherein the anti-LAIR-1 antibody comprises: a heavy chain comprising an amino acid sequence having 80% identity to the amino acid sequence of SEQ ID NO:194, and/or a light chain comprising an amino acid sequence having 80% identity to the amino acid sequence of SEQ ID NO:196.
130. The kit of embodiment 129, wherein the heavy chain comprises the amino acid sequence of SEQ ID NO:194, and/or the light chain comprises the amino acid sequence of SEQ ID NO:196.
131. A combination comprising an anti-LAIR-1 antibody and an anti-ILT3 antibody.
132. The combination of embodiment 131, wherein the anti-LAIR-1 antibody and the anti-ILT3 antibody are formulated separately.
133. The combination of embodiment 131, wherein the anti-LAIR-1 antibody and the anti-ILT3 antibody are co-formulated.
134. The combination of embodiment 131, wherein the anti-LAIR-1 antibody and the anti-ILT3 antibody are co-formulated as a pharmaceutical composition.
135. The combination of embodiment 131, wherein the anti-LAIR-1 antibody and the anti-ILT3 antibody are each separately formulated as a pharmaceutical composition.
136. The combination of embodiment 131, wherein the anti-LAIR-1 antibody and the anti-ILT3 antibody synergistically overcomes stromal-mediated immunosuppression in a tumor microenvironment.
137. The combination of any one of embodiments 131-136, wherein the anti-ILT3 antibody comprises:

(a) a heavy chain variable region (VH) comprising a VH-complementarity determining region (CDR)1, a VH-CDR2, and a VH-CDR3 from the amino acid sequence of SEQ ID NO:111; and a light chain variable region (VL) comprising a VL-CDR1, a VL-CDR2, and a VL-CDR3 from the amino acid sequence of SEQ ID NO:112; or

(b) a VH comprising a VH-CDR1, a VH-CDR2, and a VH-CDR3 from the amino acid sequence of SEQ ID NO:123; and a VL comprising a VL-CDR1, a VL-CDR2, and a VL-CDR3 from the amino acid sequence of SEQ ID NO:124.

138. The combination of embodiment 137, wherein the anti-ILT3 antibody comprises:

(a) the VH comprises a VH-CDR1 comprising the amino acid sequence GFTFSSYGMS (SEQ ID NO:27), a VH-CDR2 comprising the amino acid sequence TISGGGSYTNYPDSVKG (SEQ ID NO:28), and a VH-CDR3 comprising the amino acid sequence REWRYTLYAMDY (SEQ ID NO:105); and the VL comprises a VL-CDR1 comprising the amino acid sequence RASESVESYGSSFMH (SEQ ID NO:106), a VL-CDR2 comprising the amino acid sequence LTSNLES (SEQ ID NO:31), and a VL-CDR3 comprising the amino acid sequence QQNNEDPFT (SEQ ID NO:32);

(b) the VH comprises a VH-CDR1 comprising the amino acid sequence GFTFSSY (SEQ ID NO:33), a VH-CDR2 comprising the amino acid sequence SGGGSY (SEQ ID NO:34), and a VH-CDR3 comprising the amino acid sequence REWRYTLYAMDY (SEQ ID NO:105); and the VL comprises a VL-CDR1 comprising the amino acid sequence RASESVESYGSSFMH (SEQ ID NO:106), a VL-CDR2 comprising the amino acid sequence LTSNLES (SEQ ID NO:31), and a VL-CDR3 comprising the amino acid sequence QQNNEDPFT (SEQ ID NO:32);

(c) the VH comprises a VH-CDR1 comprising the amino acid sequence GFTFSSYGMS (SEQ ID NO:27), a VH-CDR2 comprising the amino acid sequence TISGGGSYTN (SEQ ID NO:35), and a VH-CDR3 comprising the amino acid sequence REWRYTLYAMDY (SEQ ID NO:105); and the VL comprises a VL-CDR1 comprising the amino acid sequence RASESVESYGSSFMH (SEQ ID NO:106), a VL-CDR2 comprising the amino acid sequence LTSNLES (SEQ ID NO:31), and a VL-CDR3 comprising the amino acid sequence QQNNEDPFT (SEQ ID NO:32);

(d) the VH comprises a VH-CDR1 comprising the amino acid sequence SYGMS (SEQ ID NO:36), a VH-CDR2 comprising the amino acid sequence TISGGGSYTNYPDSVKG (SEQ ID NO:28), and a VH-CDR3 comprising the amino acid sequence REWRYTLYAMDY (SEQ ID NO:105); and the VL comprises a VL-CDR1 comprising the amino acid sequence RASESVESYGSSFMH (SEQ ID NO:106), a VL-CDR2 comprising the amino acid sequence LTSNLES (SEQ ID NO:31), and a VL-CDR3 comprising the amino acid sequence QQNNEDPFT (SEQ ID NO:32); or

(e) the VH comprises a VH-CDR1 comprising the amino acid sequence SSYGMS (SEQ ID NO:37), a VH-CDR2 comprising the amino acid sequence WVATISGGGSYTN (SEQ ID NO:38), and a VH-CDR3 comprising the amino acid sequence ARREWRYTLYAMD (SEQ ID NO:107), and the VL comprises a VL-CDR1 comprising the amino acid sequence ESYGSSFMHWY (SEQ ID NO:108), a VL-CDR2 comprising the amino acid sequence LLIYLTSNLE (SEQ ID NO:41), and a VL-CDR3 comprising the amino acid sequence QQNNEDPF (SEQ ID NO:42).

139. The combination of embodiment 137 or 138, wherein:

(a) the VH comprises an amino acid sequence having at least 80% sequence identity to the amino acid sequence of SEQ ID NO:123;

(b) the VL comprises an amino acid sequence having at least 80% sequence identity to the amino acid sequence of SEQ ID NO:124; or

(c) the VH comprises an amino acid sequence having at least 80% sequence identity to the amino acid sequence of SEQ ID NO:123 and the VL comprises an amino acid sequence having at least 80% sequence identity to the amino acid sequence of SEQ ID NO:124.

140. The combination of embodiment 139, wherein the anti-ILT3 antibody comprises: the heavy chain variable region comprising the amino acid sequence of SEQ ID NO:123 and the light chain variable region comprising the amino acid sequence of SEQ ID NO:124.
141. The combination of any one of embodiments 137-139, wherein the anti-ILT3 antibody comprises:

(a) a heavy chain comprising an amino acid sequence having at least 80% sequence identity to the sequence of SEQ ID NO:126;

(b) a light chain comprising an amino acid sequence having at least 80% sequence identity to the sequence of SEQ ID NO:128; or

(c) a heavy chain comprising an amino acid sequence having at least 80% sequence identity to the sequence of SEQ ID NO:126 and a light chain comprising an amino acid sequence having at least 80% sequence identity to the sequence of SEQ ID NO:128.

142. The combination of embodiment 141, wherein the VH comprises the amino acid sequence of SEQ ID NO:123 and the VL comprises the amino acid sequence of SEQ ID NO:124.
143. The combination of any one of embodiments 131-142, wherein the anti-LAIR-1 antibody comprises: a heavy chain variable region (VH) comprising a VH-complementarity determining region (CDR)1, a VH-CDR2 and a VH-CDR3 from SEQ ID NO:179, and a light chain variable region (VL) comprising a VL-CDR1, a VL-CDR2 and a VL-CDR3 from SEQ ID NO:180.
144. The combination of embodiment 143, wherein:

(a) the VH comprises the VH-CDR1 comprising the amino acid sequence of SEQ ID NO:242, the VH-CDR2 comprising the amino acid sequence of SEQ ID NO:257, and the VH-CDR3 comprising the amino acid sequence of SEQ ID NO:244; and the VL comprises the VL-CDR1 comprising the amino acid sequence of SEQ ID NO:245, the VL-CDR2 comprising the amino acid sequence of SEQ ID NO:258, and the VL-CDR3 comprising the amino acid sequence of SEQ ID NO:247;

(b) the VH comprises the VH-CDR1 comprising the amino acid sequence of SEQ ID NO:248, the VH-CDR2 comprising the amino acid sequence of SEQ ID NO:259, and the VH-CDR3 comprising the amino acid sequence of SEQ ID NO:244; and the VL comprises the VL-CDR1 comprising the amino acid sequence of SEQ ID NO:245, the VL-CDR2 comprising the amino acid sequence of SEQ ID NO:258, and the VL-CDR3 comprising the amino acid sequence of SEQ ID NO:247;

(c) the VH comprises the VH-CDR1 comprising the amino acid sequence of SEQ ID NO:242, the VH-CDR2 comprising the amino acid sequence of SEQ ID NO:260, and the VH-CDR3 comprising the amino acid sequence of SEQ ID NO:244; and the VL comprises the VL-CDR1 comprising the amino acid sequence of SEQ ID NO:245, the VL-CDR2 comprising the amino acid sequence of SEQ ID NO:258, and the VL-CDR3 comprising the amino acid sequence of SEQ ID NO:247;

(d) the VH comprises the VH-CDR1 comprising the amino acid sequence of SEQ ID NO:250, the VH-CDR2 comprising the amino acid sequence of SEQ ID NO:257, and the VH-CDR3 comprising the amino acid sequence of SEQ ID NO:244; and the VL comprises the VL-CDR1 comprising the amino acid sequence of SEQ ID NO:245, the VL-CDR2 comprising the amino acid sequence of SEQ ID NO:258, and the VL-CDR3 comprising the amino acid sequence of SEQ ID NO:247; or

(e) the VH comprises the VH-CDR1 comprising the amino acid sequence of SEQ ID NO:251, the VH-CDR2 comprising the amino acid sequence of SEQ ID NO:261, and the VH-CDR3 comprising the amino acid sequence of SEQ ID NO:253; and the VL comprises the VL-CDR1 comprising the amino acid sequence of SEQ ID NO:254, the VL-CDR2 comprising the amino acid sequence of SEQ ID NO:255, and the VL-CDR3 comprising the amino acid sequence of SEQ ID NO:256.

145. The combination of any one of embodiments 143 or 144, wherein the VH has at least 80% sequence identity to the amino acid sequence of SEQ ID NO:179, and/or the VL has at least 80% sequence identity to the amino acid sequence of SEQ ID NO:180.
146. The combination of embodiment 145, wherein: the VH comprises the amino acid sequence of SEQ ID NO:179, and/or the VL comprises the amino acid sequence of SEQ ID NO:180.
147. The combination of any one of embodiments 131-146, wherein the anti-LAIR-1 antibody comprises: a heavy chain comprising an amino acid sequence with 80% identity to the amino acid sequence of SEQ ID NO:194 and/or a light chain comprising an amino acid sequence with 80% identity to the amino acid sequence of SEQ ID NO:196.
148. The combination of embodiment 147, wherein the heavy chain comprises the amino acid sequence of SEQ ID NO:194, and/or the light chain comprises the amino acid sequence of SEQ ID NO:196.

### 7. EXAMPLES

The following is a description of various methods and materials used in the studies, and are put forth so as to provide those of ordinary skill in the art with a complete disclosure and description of how to make and use the present invention, and are not intended to limit the scope of what the inventors regard as their invention nor are they intended to represent that the experiments below were performed and are all of the experiments that may be performed. It is to be understood that exemplary descriptions written in the present tense were not necessarily performed, but rather that the descriptions can be performed to generate the data and the like associated with the teachings of the present invention. Efforts have been made to ensure accuracy with respect to numbers used (e.g., amounts, temperature, etc.), but some experimental errors and deviations should be accounted for.

### Example 1: Materials and Methods Used in Examples 2-9

### General Protocol for Differentiation of Monocyte Derived DCs and Tolerogenic DCs:

Human peripheral blood mononuclear cells (PBMCs) were isolated from freshly drawn leukopacks (AllCells) by centrifugation over a Ficoll density gradient (GE Healthcare), resuspended in CryoStor cell freezing medium (StemCell Technologies), and stored in liquid nitrogen until use. Primary human monocytes were isolated from cryopreserved PBMCs by negative selection using the Miltenyi Monocyte Isolation Kit, according to the manufacturer's instructions. The monocytes were plated at 2×10⁶ cells/mL in X-Vivo 15 media (Lonza) containing 50 ng/mL recombinant human GM-CSF and 50 ng/mL recombinant human IL-4 (both from Peprotech) and cultured for 5-7 days to generate monocyte-derived DCs (moDCs). To generate tolerogenic DCs (tolDCs), monocytes were treated with GM-CSF and IL-4 for 5 days and then treated for 2 additional days with 10 nM dexamethasone and 100 nM vitamin D3 (1α,25-dihydroxyvitamin D3) (both from Sigma-Aldrich).

### General Protocol for Differentiation of Monocyte-Derived Macrophages:

Human peripheral blood mononuclear cells (PBMCs) were isolated from freshly drawn leukopacks (AllCells) by centrifugation over a Ficoll density gradient (GE Healthcare), resuspended in CryoStor cell freezing medium (StemCell Technologies), and stored in liquid nitrogen until use. Primary human monocytes were isolated from cryopreserved peripheral blood mononuclear cells by negative selection using the Miltenyi Monocyte Isolation Kit, according to the manufacturer's instructions. For differentiation of macrophages, monocytes were plated at 2×10⁶ cells/mL in X-Vivo 15 media (Lonza) containing 100 ng/mL recombinant human M-CSF. After 2-3 days, the media was spiked with fresh M-CSF at the same concentration. After another 2-3 days, the media was removed from the unpolarized (M0) macrophages and replaced with fresh media containing 100 ng/mL M-CSF plus 50 ng/mL recombinant human IL-4 to generate M2a macrophages. All recombinant cytokines were from Peprotech.

### Example 2: Anti-ILT3 Antibody and Anti-LAIR-1 Antibody Synergistically Enhanced FcR-Driven Cytokine Production in Myeloid Cells

The effect of the combination of an anti-ILT3 antibody and an anti-LAIR-1 antibody to reactivate myeloid cells in a tumor stromal microenvironment was examined using an in vitro system, where a human IgG1 anti-KLH monoclonal antibody with functional Fc receptor was used, and the myeloid cell responsiveness to FcγR engagement was measured.

Wells of 96-well Maxisorp plates were coated with human fibronectin (Millipore, FC0101, 5 μg/mL in PBS) and human collagen (Millipore, CC050, 1 μg/mL in PBS) along with human IgG1, anti-KLH (5 μg/mL in PBS) at room temperature for 1 hour, then washed in PBS and blocked with X-Vivo 15 media (Lonza) for 30 minutes. MoDCs were generated from primary human monocytes as described above, then harvested, washed, and pre-incubated with an isotype control, an anti-ILT3 antibody hz5A7.v5, an anti-LAIR-1 antibody hz47H1.v4, or the combination of hz5A7.v5 and hz47H1.v4 at room temperature for 20 minutes. The starting concentration for each antibody was 10 μg/mL of each antibody, and a series of 3-fold dilution were performed. MoDCs were then plated onto the coated wells (7×10⁴ cells/well in a 100 μL volume) and incubated overnight. Media was harvested after 24 hours and TNF-α secretion was analyzed by Luminex assay (ProcartaPlex system; Thermo Fisher Scientific) according to the manufacturer's instructions.

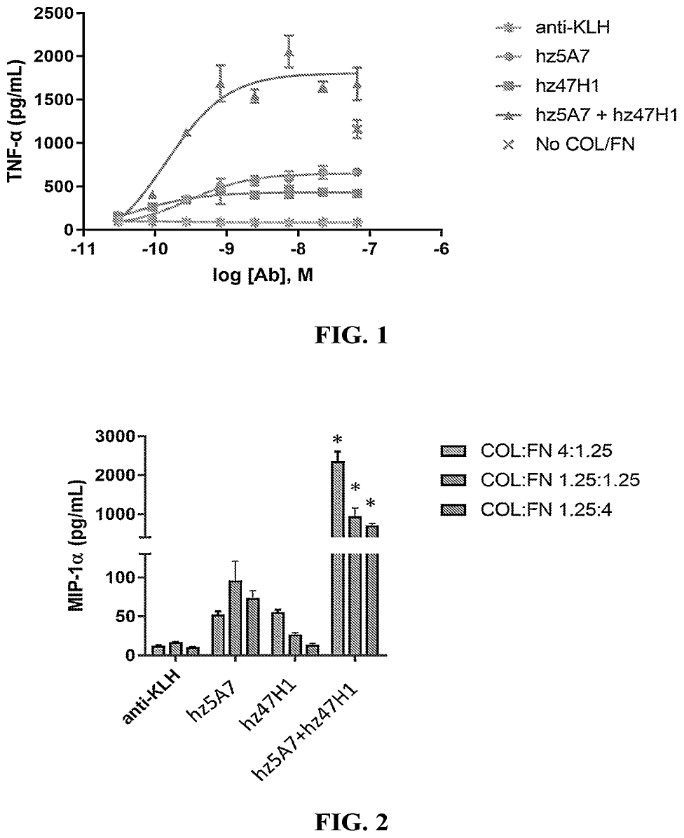

As shown in FIG. 1, anti-KLH monoclonal antibody with functional Fc receptor binding activity activated FcγRs on the moDCs and induced TNF-α production. When co-coated with fibronectin and collagen, anti-KLH antibody failed to induce TNF-α production from the moDCs. After an anti-ILT3 antibody which blocks the fibronectin-ILT3 interaction was introduced, TNF-α production in the moDCs was induced. Similarly, after an anti-LAIR-1 antibody which blocks the collagen-LAIR-1 interaction was introduced, TNF-α production in the moDCs was induced. Co-blockade of both inhibitory fibronectin-ILT3 and collagen-LAIR-1 interactions with the combination of an anti-ILT3 antibody and an anti-LAIR-1 antibody induced significantly more TNF-α production than was induced with either antibody alone.

### Example 3: Anti-ILT3 Antibody and Anti-LAIR-1 Antibody Synergistically Induced Chemokine Production from Myeloid Cells Plated on Collagen and Fibronectin

To evaluate the effect of an anti-ILT3 antibody and an anti-LAIR-1 antibody on chemokine production by myeloid cells, an in vitro assay was used, wherein human monocyte-derived dendritic cells (DCs) were cultured on fibronectin/collagen co-coated wells and MIP-1α secretion from these cells was measured.

Monocyte-derived DCs were generated from primary human monocytes as described above. Wells of 96-well, flat bottom tissue culture plates were co-coated with human fibronectin and human collagen at the indicated ratios at room temperature for 1 hour. The wells were then washed with PBS and blocked with X-Vivo 15 media for 30 minutes. Monocyte-derived DCs were plated at 2×10⁵ cells/well in a 50 μL volume of X-Vivo 15 media containing a 1:50 dilution of human Fc receptor binding inhibitor (eBioscience/Thermo Fisher Scientific) and an isotype control antibody, hz5A7.v5, hz47H1.v4, or the combination of hz5A7.v5 and hz47H1.v4. Each antibody was used at 10 μg/ml. The cells were incubated at 37° C. and media was harvested for measurement of MIP-1α by Luminex assay (ProcartaPlex system; Thermo Fisher Scientific) after 48 hours.

As shown in FIG. 2, in the presence of both collagen and fibronectin, minimal MIP-1α was induced by blockade of either the fibronectin-ILT3 with an anti-ILT3 antibody or the collagen-LAIR-1 interaction with an anti-LAIR-1 antibody. Blockade of both pathways with the combination of an anti-ILT3 antibody and an anti-LAIR-1 antibody synergistically increased MIP-1α release by moDCs compared with either antibody alone. Notably, this synergistic effect was observed across all tested collagen:fibronectin ratios.

### Example 4: Anti-ILT3 Antibody and Anti-LAIR-1 Antibody Synergistically Induced Chemokine Production from Tolerogenic DCs

The effect of an anti-ILT3 antibody and an anti-LAIR-1 antibody on chemokine production by tolerogenic dendritic cells (DCs) was assayed. Tolerogenic DCs were generated from primary human monocytes as described above. Wells of 96-well, flat bottom tissue culture plates were coated with human fibronectin (Millipore) (1.25 μg/mL) and human collagen (Millipore) (4 μg/mL) room temperature for 1 hour, then washed with PBS and blocked with X-Vivo 15 media for 30 minutes. Tolerogenic DCs were plated at 2×10⁵ cells/well in a 50 μL volume of X-Vivo 15 media containing a 1:50 dilution of human Fc receptor binding inhibitor (eBioscience/Thermo Fisher Scientific) and a dose titration of an isotype control antibody, hz5A7.v5, hz47H1.v4, or the combination of hz5A7.v5 and hz47H1.v4 (starting concentration of 10 μg/mL for each antibody; 2-fold dilutions). The cells were incubated at 37° C. and media was harvested for measurement of MIP-1α by Luminex assay (ProcartaPlex system; Thermo Fisher Scientific) after 48 hours.

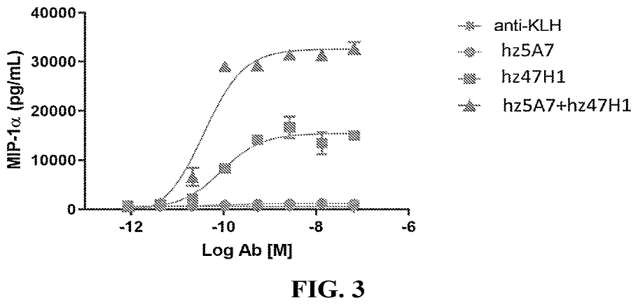

As shown in FIG. 3, blockade of ILT3-fibronectin and LAIR-1-collagen interactions by the combination of an anti-ILT3 antibody and an anti-LAIR-1 antibody synergistically increased MIP-1α release by tolerogenic DCs compared with treatment with either antibody alone.

### Example 5: Anti-ILT3 Antibody and Anti-LAIR-1 Antibody Synergistically Induced Chemokine Production from Tolerogenic DCs

Tolerogenic DCs were generated from primary human monocytes as described above. Wells of 96-well, flat bottom tissue culture plates were coated with human fibronectin (Millipore) (1.25 μg/mL) and human collagen (Millipore) (4 μg/mL) at room temperature for 1 hour, then washed with PBS and blocked with X-Vivo 15 media for 30 minutes. Tolerogenic DCs were plated at 2×10⁵ cells/well in a 50 μL volume of X-Vivo 15 media containing a 1:50 dilution of human Fc receptor binding inhibitor (eBioscience/Thermo Fisher Scientific) and an isotype control antibody, hz5A7.v5, hz47H1.v4, or the combination of hz5A7.v5 and hz47H1.v4 (10 μg/mL of each antibody). The cells were incubated at 37° C. and media was harvested for measurement of chemokines by Luminex assay (ProcartaPlex system; Thermo Fisher Scientific) after 48 hours.

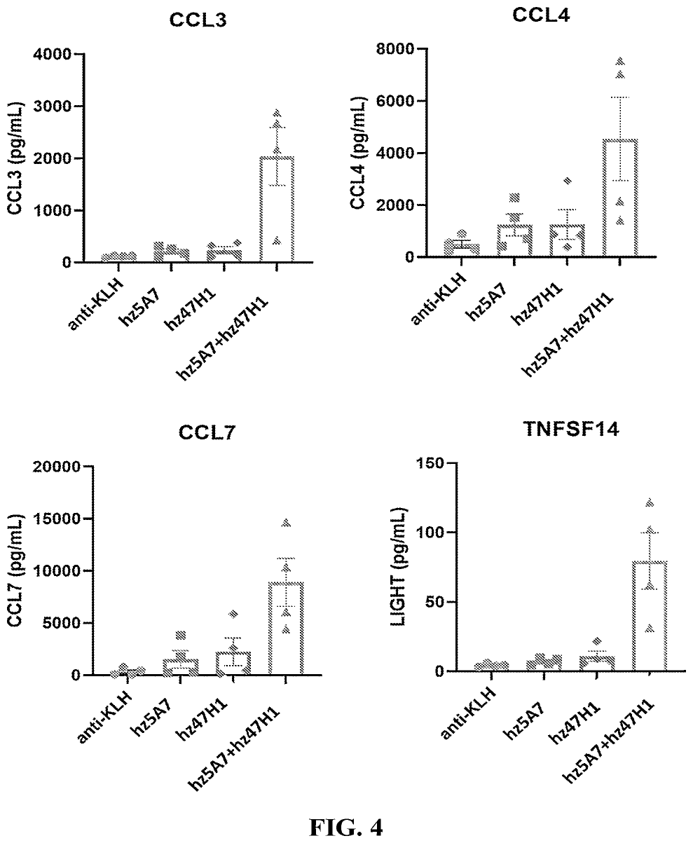

As shown in FIG. 4, the combination of hz5A7.v5 and hz47H1.v4 induced increased secretion of MIP-1α (CCL3), MIP-1β (CCL4), MCP-3 (CCL7), and LIGHT (TNFSF14) from tolerogenic DCs compared with either antibody alone. These chemokines are known to induce the migration of numerous immune cell types, including T cells. These data suggest that combined blockade of the fibronectin-ILT3 and collagen-LAIR-1 interactions can increase immune cell infiltration within the tumor microenvironment by increasing chemokine production from tolerogenic DCs.

### Example 6: Anti-ILT3 Antibody and Anti-LAIR-1 Antibody Synergistically Induced Chemokine Production from M2a Macrophages

To evaluate the effect of an anti-ILT3 antibody and anti-LAIR-1 antibody on chemokine production by macrophages, IL-4-polarized (M2a) macrophages were cultured on fibronectin/collagen co-coated wells and chemokine secretion was measured. M2a macrophages were generated from primary human monocytes as described above. Wells of 96-well, flat bottom tissue culture plates were coated with human fibronectin (Millipore) (1 μg/mL) and human collagen (Millipore) (4 μg/mL) room temperature for 1 hour, then washed with PBS and blocked with X-Vivo 15 media for 30 minutes. M2c macrophages were plated at 2×10⁵ cells/well in a 50 μL volume of X-Vivo 15 media containing a 1:50 dilution of human Fc receptor binding inhibitor (eBioscience/Thermo Fisher Scientific) and an isotype control antibody, hz5A7.v5, hz47H1.v4, or the combination of hz5A7.v5 and hz47H1.v4 (10 μg/mL of each antibody). The cells were incubated at 37° C. and media was harvested for measurement of chemokines by Luminex assay (ProcartaPlex system; Thermo Fisher Scientific) after 48 hours.

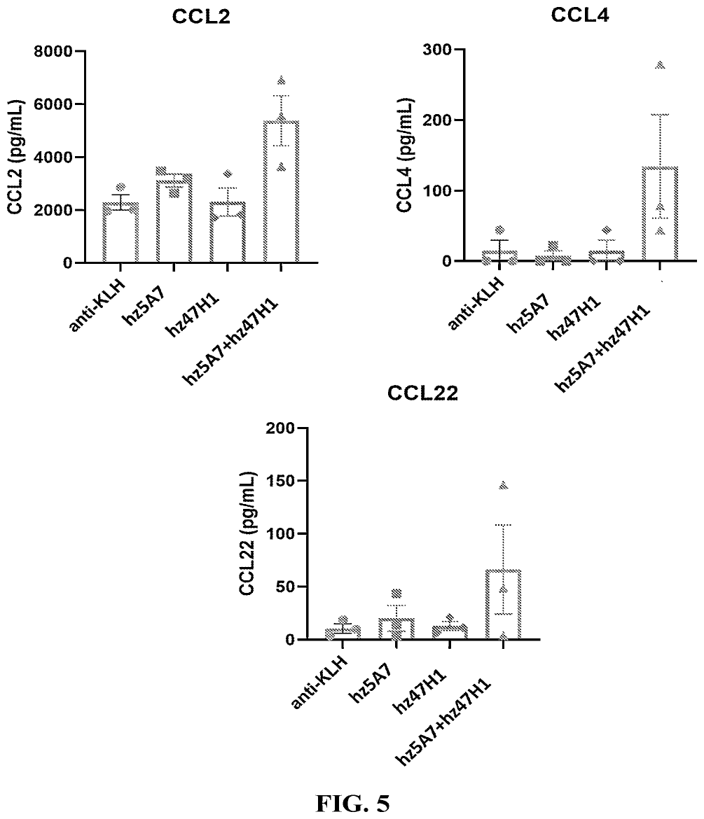

As shown in FIG. 5, the combination of hz5A7.v5 and hz47H1.v4 induced increased secretion of MCP-1 (CCL2), MIP-1β (CCL4), and MDC (CCL22) in the macrophages compared with either antibody alone. These chemokines are known to induce the migration of numerous immune cell types, including T cells. These data suggest that combined blockade of the fibronectin-ILT3 and collagen-LAIR-1 interactions may increase immune cell infiltration within the tumor microenvironment by increasing chemokine production from tumor-associated macrophages.

### Example 7: The Combination of an Anti-ILT3 Antibody and Anti-LAIR-1 Antibody Reprogramed Suppressive Myeloid Cells

To evaluate myeloid cell reprogramming by the combination of an anti-ILT3 antibody and anti-LAIR-1 antibody, RNA sequencing was performed. Briefly, wells of 24-well, flat bottom tissue culture plates were coated with PBS, human fibronectin (Millipore) (1 μg/mL), human collagen (Millipore) (4 μg/mL), or the combination of fibronectin and collagen (same concentrations) at room temperature for 1 hour. The wells were then washed with PBS and blocked with X-Vivo 15 media for 30 minutes. Tolerogenic DCs were generated as described above and plated at 6×10⁵ cells/well in a 200 μL volume of X-Vivo 15 media containing a 1:50 dilution of human Fc receptor binding inhibitor (eBioscience/Thermo Fisher Scientific) and an isotype control antibody, hz5A7.v5, hz47H1.v4, or the combination of hz5A7.v5 and hz47H1.v4 (each at 10 μg/mL). After 48 hrs, cells were harvested for RNA expression profiling. Total RNA was extracted from the cells and prepared for sequencing using the TruSeq Stranded mRNA kit (Illumina) and sequenced on an Illumina NovaSeq 6000 S4 (100 bp single-end reads; an average of 28 million reads were generated for each sample). For sequencing alignment, the raw reads were filtered using Trim Galore to remove low-quality and adaptor bases, and reads shorter than 20 nt were discarded. Filtered reads were mapped to UCSC hg19 genome sequences using STAR (v2.6.0a). Finally, counts of all samples were generated using featureCounts (v1.6.2). EdgeR (v3.22.3) was used to obtain normalized counts and perform differential gene expression analysis. Genes were considered to be differentially expressed if the fold change was >1.5 and the p-value was <0.05.

Of the 1256 genes significantly regulated by hz5A7.v5 on fibronectin-coated wells, only 474 (38%) were also regulated by hz5A7.v5 on collagen/fibronectin co-coated wells. Similarly, of the 1565 genes significantly regulated by hz47H1.v4 on collagen-coated wells, only 342 (22%) were also regulated by hz47H1.v4 on collagen/fibronectin co-coated wells. These data demonstrate that the addition of collagen significantly suppresses the effects of hz5A7.v5, while the addition of fibronectin significantly suppresses the effects of hz47H1.v4.

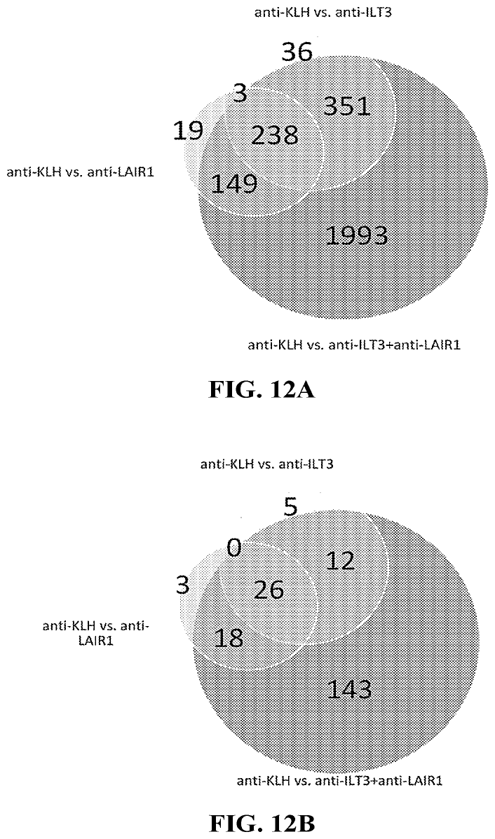

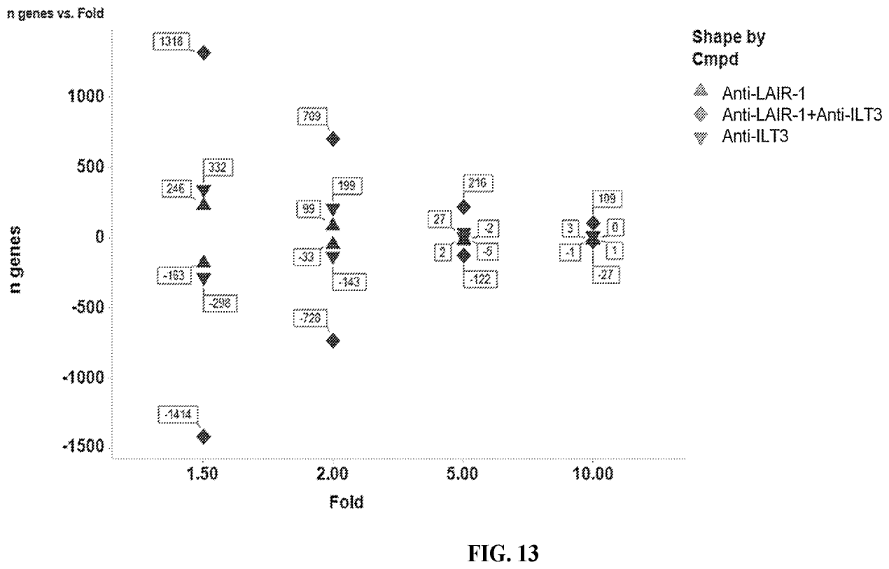

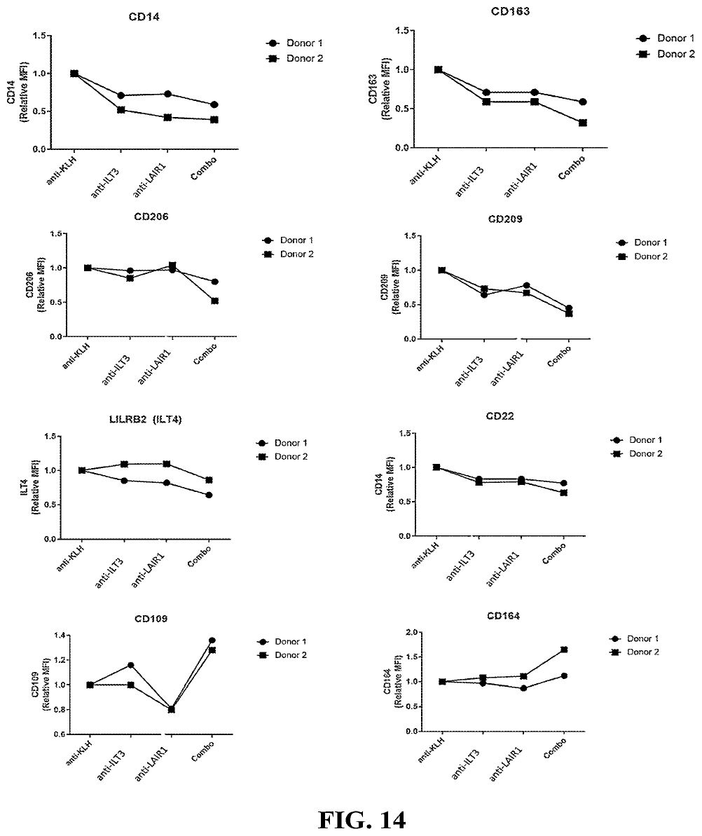

On collagen/fibronectin co-coated wells, blockade of the fibronectin-ILT3 interaction with hz5A7.v5 altered the expression of 628 genes, while blockade of the collagen-LAIR-1 interaction with hz47H1.v4 altered the expression of 409 genes. Of the 628 genes regulated by hz5A7.v5 on collagen/fibronectin co-coated wells, 589 (94%) were also regulated by the combination of hz5A7.v5 and hz47H1.v4. Similarly, of the 409 genes regulated by hz47H1.v4 on collagen/fibronectin co-coated wells, 387 (95%) were also regulated by the combination of hz5A7.v5 and hz47H1.v4. Importantly, the combination of hz47H1.v4 and hz5A7.v5 altered the expression of an additional 1993 genes that were not regulated by either hz5A7.v5 or hz47H1.v4 alone on collagen/fibronectin co-coated wells, as shown in FIG. 12A. The gene expression data was imported into Ingenuity Pathway Analysis (IPA) software, and differentially expressed genes and canonical pathways were evaluated using a cut-off of gene differentially expressed with a log 2 (fold change)>1.5 and p<0.05. Representation of the pathways that were regulated by hz5A7.v5, hz47H1.v4, or the combination thereof on collagen- and fibronectin-coated wells is shown in FIG. 12B. Almost all the pathways regulated by anti-ILT3 and anti-LAIR-1 individually were also regulated by the combination treatment. In addition, the combination of anti-ILT3 and anti-LAIR-1 regulated an additional ˜140 pathways. FIG. 13 shows the number of upregulated and downregulated genes and the expression fold changes by hz5A7.v5, hz47H1.v4, or the combination thereof on collagen- and fibronectin-co-coated wells. Few genes were upregulated or downregulated 10-fold by anti-ILT3 antibody or anti-LAIR-1 antibody alone, but the combination resulted in a 10-fold upregulation of 109 genes and a 10-fold downregulation of 27 genes. These data show that combination of anti-ILT3 antibody and anti-LAIR-1 antibody had a synergistic effect on gene expression (both the number of differentially expressed genes and the magnitude of the gene expression change) in the presence of collagen and fibronectin.

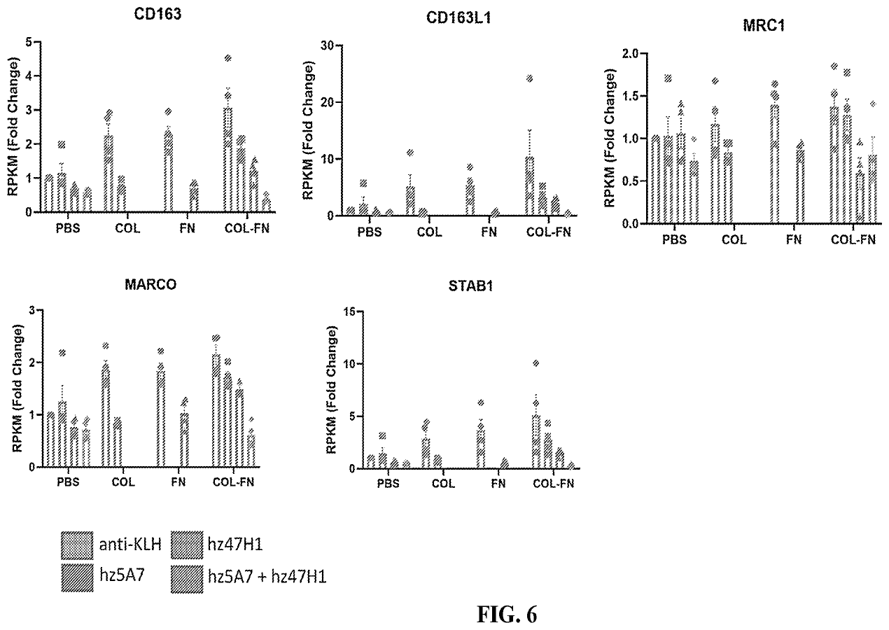

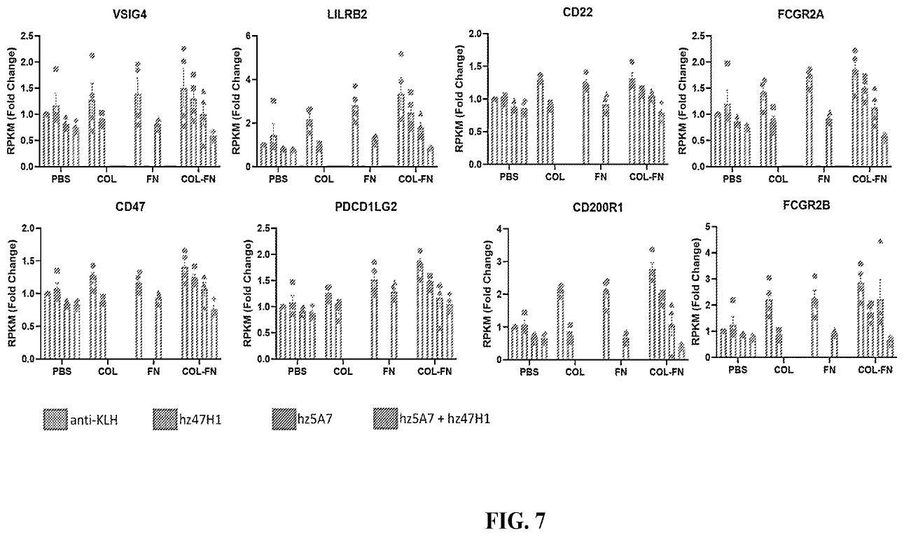

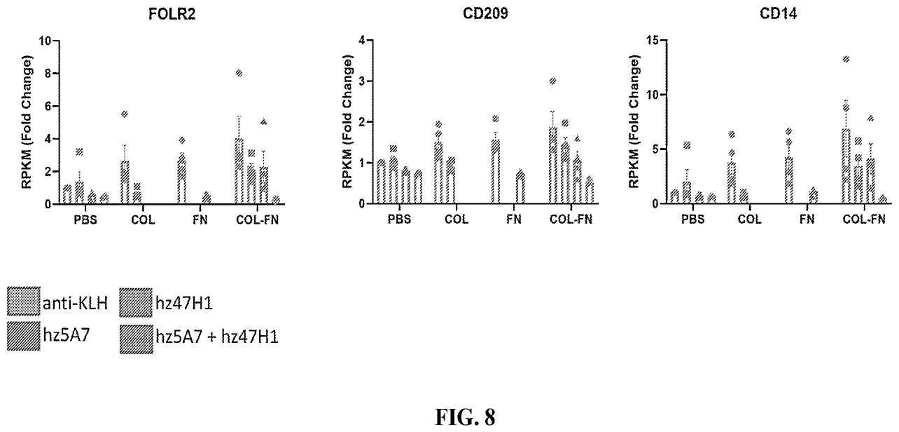

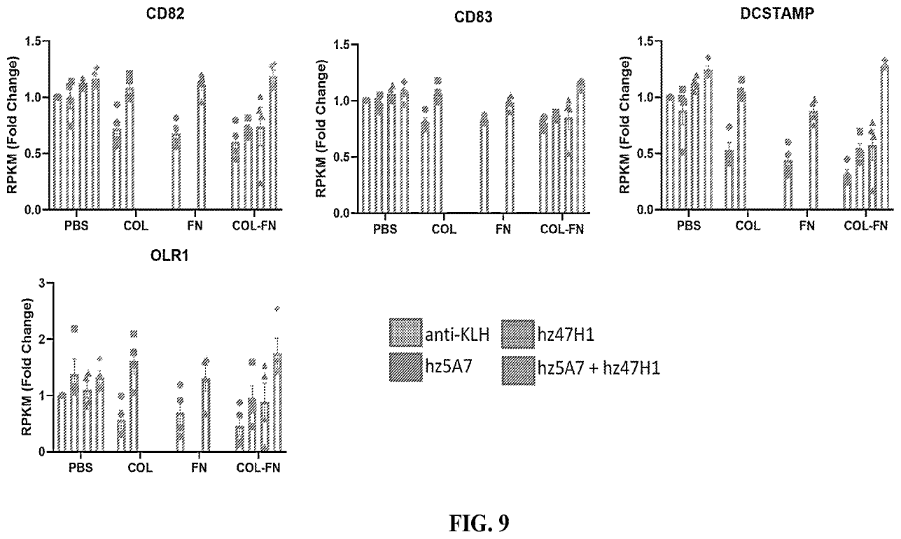

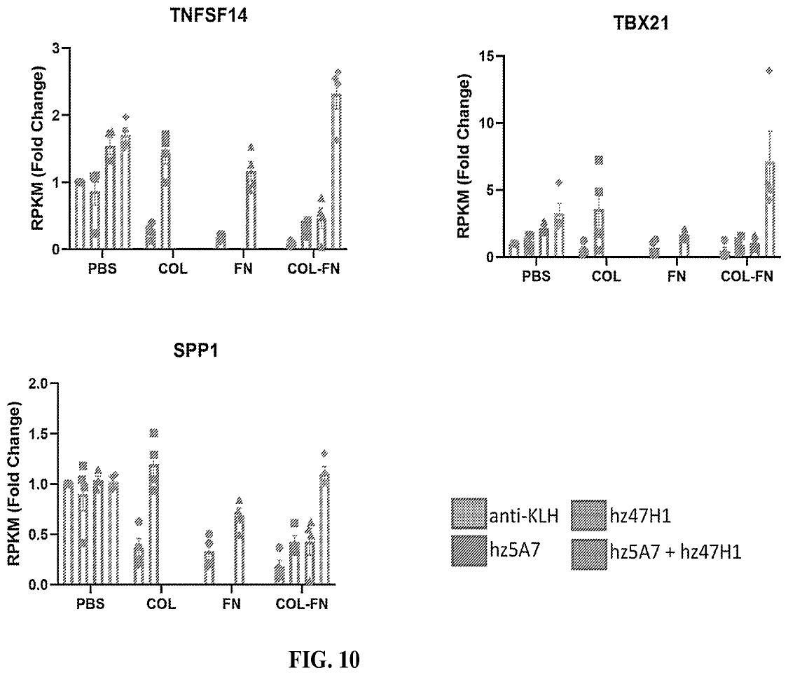

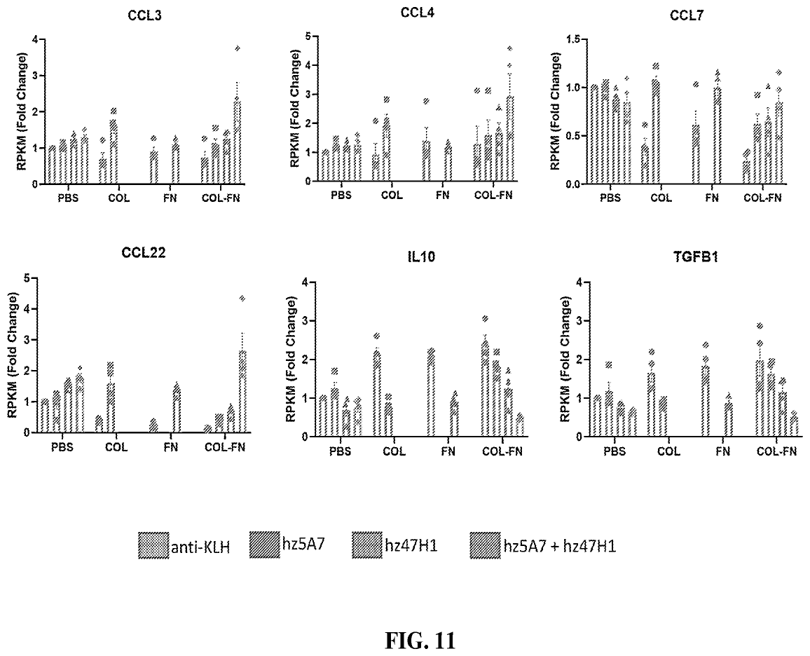

In total, 2731 genes were differentially regulated in fibronectin/collagen treated tolerogenic DCs by blocking ILT3-fibronectin and LAIR-1-collagen interactions via an anti-ILT3 antibody (e.g., hz47H1.v4) and an anti-LAIR-1 antibody (e.g., hz5A7.v5). Among these genes, reduced expressions of scavenger receptors (e.g., CD163, CD163L1, MRC1, MARCO, STAB1), myeloid cell inhibitory receptors (e.g., PDCD1LG2, VSIG4, CD47, LILRB2, CD200R1, CD22, FCGR2A, FCGR2B) and markers of dendritic cell tolerization and immaturity (e.g., FOLR2, CD209, CD14) were observed (FIGS. 6-8). Increased expressions of genes involved in antigen presentation and DC activation (e.g., CD82, CD83, DCSTAMP, OLR1), T cell co-stimulation (e.g., TNFSF14), and TH1 polarization (e.g., TBX21, SPP1) were observed (FIGS. 9-10). In addition, the combination of an anti-ILT3 antibody (e.g., hz47H1.v4) and an anti-LAIR-1 antibody (e.g., hz5A7.v5) increased expression of chemokines known to induce T cell migration (e.g., CCL3, CCL4, CCL5, CCL7, CCL22) and decreased the expression of immunosuppressive cytokines (e.g., IL10, TGFB1) (FIG. 11).

Protein expression levels of the differentially regulated genes were assayed by flow cytometry. Tolerogenic dendritic cells were generated and treated with an isotype control antibody, hz5A7.v5, hz47H1.v4, or the combination on PBS-coated or fibronectin/collagen co-coated plates, as described above. After 48 hrs, the cells were harvested and stained for 30 minutes at 4° C. with fluorophore conjugated antibodies. Signals were acquired on a FACSCalibur flow cytometer and analyzed using FlowJo software. For example, the combination of an anti-ILT3 antibody (e.g., hz47H1.v4) and an anti-LAIR-1 antibody (e.g., hz5A7.v5) further reduced expressions of scavenger receptors (e.g., MRC1), myeloid cell inhibitory receptors (e.g., LILRB2) and markers of dendritic cell tolerization and immaturity (e.g., CD209, CD14) than an anti-ILT3 antibody or an anti-LAIR-1 antibody alone (data not shown).

An array of human immune cell surface markers (Biolegend LEGENDscreen kit) was also utilized to evaluate protein expression levels of the differentially regulated genes. Wells of 24-well, flat-bottom tissue culture plates were coated with human fibronectin (Millipore) (1 μg/mL) and collagen (Millipore) (4 μg/mL) at room temperature for 1 hour, then washed with PBS and blocked with X-Vivo 15 media for 30 minutes. Tolerogenic DCs were plated on fibronectin- and collagen-coated plates at 0.6 million cells/mL, in a 200 μL volume, in X-Vivo 15 media containing an isotype control antibody, hz47H1.v4, hz5A7.v5, or the combination thereof (each at 10 μg/mL). The tolerogenic DCs were either left unlabeled (for hz47H1.v4+hz5A7.v5-treated cells) or were pre-labeled with CellTrace Violet (for hz5A7.v5-treated cells), CellTrace FarRed (for hz47H1.v4-treated cells), or the combination thereof (for control antibody-treated cells) for 30 minutes at 37° C. After 48 hours of incubation, the cells were analyzed using the LEGENDScreen human PE kit (Biolegend). The tolerogenic DCs were harvested and mixed in staining buffer containing human Fc receptor binding inhibitor (eBioscience/Thermo Fisher Scientific) and SytoxGreen at a 1:3000 dilution for 5 minutes, then added to the FACS plates at 75 μL cells/well and incubated at 4° C. for 30 minutes. The cells were then washed and fixed before acquisition on an LSRFortessa flow cytometer (BD Biosciences). Surface marker expression on each of the four treatment groups (control antibody, hz47H1.v4, hz5A7.v5, hz47H1.v4+hz5A7.v5) was analyzed separately based on the CellTrace labeling.

As shown in FIG. 14, the change of cell surface receptor expression level induced by the anti-ILT3 antibody, the anti-LAIR-1 antibody, or the combination was consistent with the differential RNA expression results shown above. The anti-ILT3 antibody and anti-LAIR-1 antibody individually decreased the expression of scavenger receptors and markers of a tolerogenic phenotype (e.g., CD163, MRC1, CD14, CD209) by tolerogenic dendritic cells, and the combination of the anti-ILT3 antibody and anti-LAIR-1 antibody had a great effect on suppression of these markers than each of these two antibodies alone.

These data show that the combination of an anti-ILT3 antibody and an anti-LAIR-1 antibody reprograms collagen- and fibronectin-treated dendritic cells to a more stimulatory phenotype. Further, since these gene expression changes were only observed upon dual blockade of both the ILT3-fibronectin and the LAIR-1-collagen interactions, blockade of both these inhibitory pathways synergistically overcomes stromal-mediated immunosuppression in tumor microenvironment.

### Example 8: Treatment of Mice with an Anti-ILT3 Antibody Increased LAIR-1 Expression in Tumor-Associated Myeloid APCs

Four-week-old NSG-SGM3 mice (Jackson Laboratories) were irradiated at a dose of 1 Grey using an RS2000 X-Ray irradiator (Radsource). The following day, the mice were reconstituted with human immune cells via intraveneous (i.v.) injection of CD34 positive human cord blood stem cells (Allcells; 1×10⁵ cells/mouse). Approximately 3 months later, 50 μL of blood was drawn from each mouse. Human immune cell engraftment was evaluated by flow cytometry. Mice were considered to have efficient engraftment if >15% of live immune cells were human CD45⁺. The humanized mice were then inoculated subcutaneously with human SK-MEL-5 melanoma cells (1×10⁶ cells in 20% Matrigel; 100 μL injection volume). Once the tumors reached a volume of approximately 200 mm³, mice were randomized to receive weekly intraperitoneal (i.p.) injections of a control antibody (anti-KLH) or an anti-ILT3 antibody (e.g., hz5A7.v5) at 20 mg/kg (n=5 mice/group). After 14 days, mice were sacrificed and peripheral blood, spleens, bone marrow, and tumor cells were harvested for analysis of LAIR-1 expression by flow cytometry. For flow cytometry analysis, cells were incubated with mouse Fc block (Miltenyi Biotec) to inhibit non-specific binding and then an antibody cocktail containing a viability dye (eBioscience/Thermo Fisher Scientific), a CD14 antibody (clone MφP9), and a LAIR-1 antibody was added to each sample. Cells were acquired on an LSRFortessa instrument and data were analyzed using FlowJo software (Beckton Dickenson).

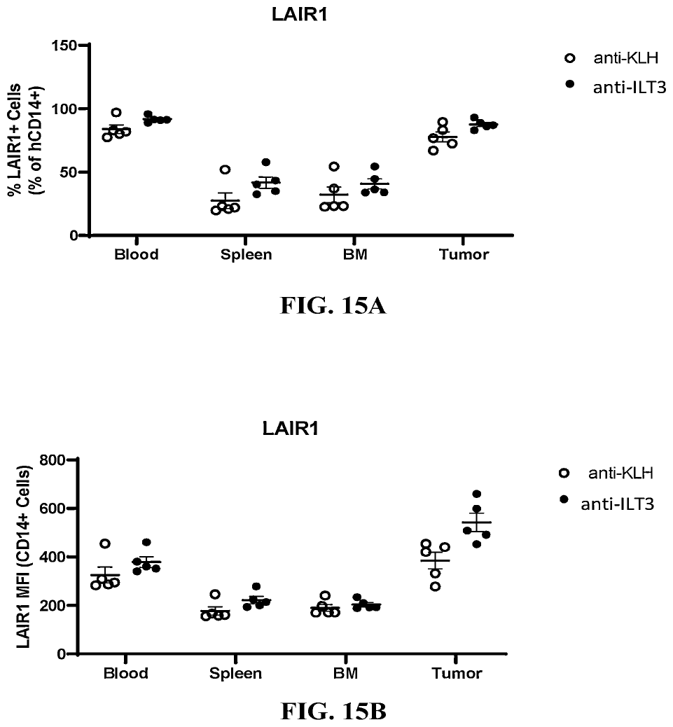

As shown in FIG. 15A and FIG. 15B, treatment of humanized mice with an anti-ILT3 antibody did not increase the number of LAIR-1 positive cells, but increased LAIR-1 expression on tumor-associated myeloid APCs, suggesting that these cells could become more dependent on LAIR-1 signaling when ILT3 activity was inhibited by an anti-ILT3 antibody. These results provide rationale for a combination therapy that targeted both ILT3 and LAIR-1, which more fully reprogramed tumor-associated myeloid cells by blocking both ILT3 and LAIR-1 immunosuppressive pathways.

### Example 9: The Combination of an Anti-ILT3 Antibody and Anti-LAIR-1 Antibody Increased the Migration of Stimulatory Myeloid APCs to the Tumor Microenvironment

The presently disclosed data had shown that dendritic cells stimulated with anti-ILT3 antibody and anti-LAIR-1 antibody secreted increased levels of chemokines such as MIP-1α (CCL3), MIP-1β (CCL4), and MCP-3 (CCL7). Migration assays were thus performed to test whether the increased chemokine production resulted in increased migration of immune cells towards stimulated dendritic cells.

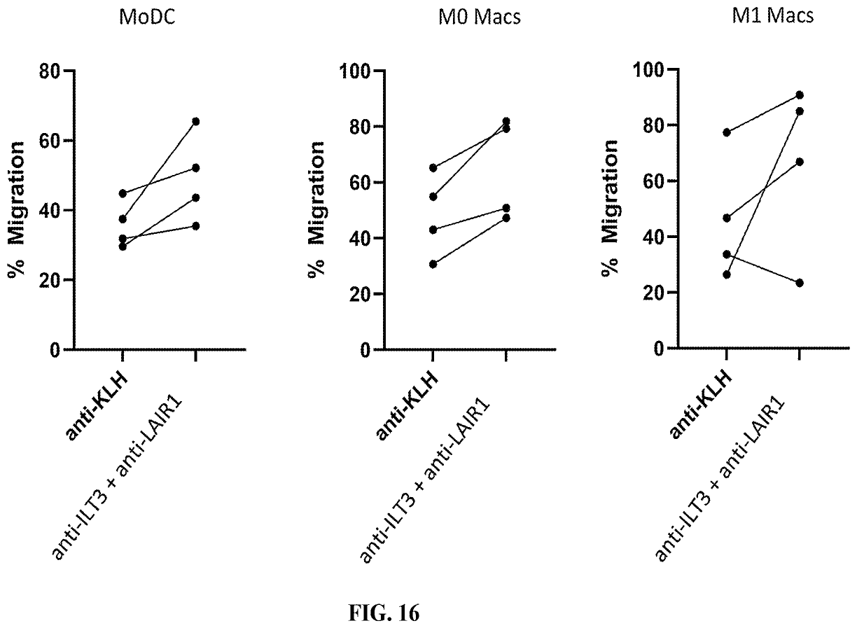

Briefly, the bottom chambers of Incucyte Clearview 96-well chemotaxis plates were co-coated with human fibronectin (Millipore) (1 μg/mL) and collagen (Millipore) (4 μg/mL) at room temperature for 1 hour, washed with PBS, and blocked with X-Vivo 15 media for 30 minutes, then seeded with tolerogenic DCs or macrophages (generated as described above) at 20K cells/well in X-Vivo media containing anti-KLH or anti-ILT3 (e.g., hz5A7.v5) and anti-LAIR-1 (e.g., hz47H1.v4) (5 μg/mL each). The following day, dendritic cells or macrophages were seeded in the top wells of the migration chambers at 8K cells/well. The plates were imaged on an Incucyte Zoom (Sartorius) every 12 hours for 6 days, and cell migration was analyzed using the Incucyte Zoom analysis software (version 2018A). Migration of the cells after 6 days is shown in FIG. 16. These data show that treatment of tolerogenic dendritic cells with anti-ILT3 and anti-LAIR-1 increased the migration of monocyte-derived dendritic cells (MoDCs), unpolarized macrophages (M0 Macs), and M1 macrophages (M1 Macs). These results suggest that reprogramming tumor-associated tolerogenic dendritic cells by the combination treatment of anti-ILT3 antibody and anti-LAIR-1 antibody increased the migration of stimulatory myeloid APCs to the tumor microenvironment.

7. SEQUENCES
Human ILT3 amino acid sequence with predicted
signal sequence underlined
(SEQ ID NO: 1)
MIPTFTALLCLGLSLGPRTHMQAGPLPKPTLWAEPGSVISWGNSV
TIWCQGTLEAREYRLDKEESPAPWDRQNPLEPKNKARFSIPSMTE
DYAGRYRCYYRSPVGWSQPSDPLELVMTGAYSKPTLSALPSPLVT
SGKSVTLLCQSRSPMDTFLLIKERAAHPLLHLRSEHGAQQHQAEF
PMSPVTSVHGGTYRCFSSHGFSHYLLSHPSDPLELIVSGSLEDPR
PSPTRSVSTAAGPEDQPLMPTGSVPHSGLRRHWEVLIGVLVVSIL
LLSLLLFLLLQHWRQGKHRTLAQRQADFQRPPGAAEPEPKDGGLQ
RRSSPAADVQGENFCAAVKNTQPEDGVEMDTRQSPHDEDPQAVTY
AKVKHSRPRREMASPPSPLSGEFLDTKDRQAEEDRQMDTEAAASE
APQDVTYAQLHSFTLRQKATEPPPSQEGASPAEPSVYATLAIH
Human ILT3 amino acid sequence without
predicted signal sequence
(SEQ ID NO: 2)
QAGPLPKPTLWAEPGSVISWGNSVTIWCQGTLEAREYRLDKEESP
APWDRQNPLEPKNKARFSIPSMTEDYAGRYRCYYRSPVGWSQPSD
PLELVMTGAYSKPTLSALPSPLVTSGKSVTLLCQSRSPMDTFLLI
KERAAHPLLHLRSEHGAQQHQAEFPMSPVTSVHGGTYRCFSSHGF
SHYLLSHPSDPLELIVSGSLEDPRPSPTRSVSTAAGPEDQPLMPT
GSVPHSGLRRHWEVLIGVLVVSILLLSLLLFLLLQHWRQGKHRTL
AQRQADFQRPPGAAEPEPKDGGLQRRSSPAADVQGENFCAAVKNT
QPEDGVEMDTRQSPHDEDPQAVTYAKVKHSRPRREMASPPSPLSG
EFLDTKDRQAEEDRQMDTEAAASEAPQDVTYAQLHSFTLRQKATE
PPPSQEGASPAEPSVYATLAIH
Human ILT3 Extracellular domain (aa 22-259)
(SEQ ID NO: 3)
QAGPLPKPTLWAEPGSVISWGNSVTIWCQGTLEAREYRLDKEESP
APWDRQNPLEPKNKARFSIPSMTEDYAGRYRCYYRSPVGWSQPSD
PLELVMTGAYSKPTLSALPSPLVTSGKSVTLLCQSRSPMDTFLLI
KERAAHPLLHLRSEHGAQQHQAEFPMSPVTSVHGGTYRCFSSHGF
SHYLLSHPSDPLELIVSGSLEDPRPSPTRSVSTAAGPEDQPLMPT
GSVPHSGLRRHWE
Human ILT3 Ig-like C2-type Domain 1 amino
acid sequence without
N-terminal domain (aa 27-118)
(SEQ ID NO: 4)
PKPTLWAEPGSVISWGNSVTIWCQGTLEAREYRLDKEESPAPWDR
QNPLEPKNKARFSIPSMTEDYAGRYRCYYRSPVGWSQPSDPLELV
MT
Human ILT3 Ig-like C2-type Domain 2 amino
acid sequence (aa 124-218)
(SEQ ID NO: 5)
PTLSALPSPLVTSGKSVTLLCQSRSPMDTFLLIKERAAHPLLHLR
SEHGAQQHQAEFPMSPVTSVHGGTYRCFSSHGFSHYLLSHPSDPL
ELIVS
Cyno ILT3 amino acid sequence with predicted
signal sequence underlined
(SEQ ID NO: 6)
mtppltvlfclglslgprtcvqagplpkptvwaepgsviswgspv
tiwcqgtldaqeyhldkegspapwdtqnplepmkakfsipsmtqh
yagryrcyyhshpdwsedsdpldlvmtgayskpilsvlpsplvts
gesvtllcqsqspmdtfllfkegaahplprlrsqhgaqlhwaefp
mgpvtsvhggtyrcissrsfshyllsrpsdpveltvlgslespsp
sptrsisaaagpedqslmptgsdpqsglrrhwevligvlvvsill
lslvfflllqhwrqgkhrtsaqrqadfqrppgaaepepkdgglqr
rsrpaadvqgenpnaamkdtqpedgveldsrqrphdedpqavtya
rvkhsgprremasppsplseefldtkdtqaeedrqmdtqaatsea
pqdvtyaqlqsltlrreateppppqkrepsaepsvyatlaiH
Cyno ILT3 amino acid sequence without predicted
signal sequence
(SEQ ID NO: 7)
qagplpkptvwaepgsviswgspvtiwcqgtldaqeyhldkegsp
apwdtqnpleprnkakfsipsmtqhyagryrcyyhshpdwsedsd
pldlvmtgayskpilsvlpsplvtsgesvtllcqsqspmdtfllf
kegaahplprlrsqhgaqlhwaefpmgpvtsvhggtyrcissrsf
shyllsrpsdpveltvlgslespspsptrsisaaagpedqslmpt
gsdpqsglrrhwevligvlvvsilllslvfflllqhwrqgkhrts
aqrqadfqrppgaaepepkdgglqrrsrpaadvqgenpnaamkdt
qpedgveldsrqrphdedpqavtyarvkhsgprremasppsplse
efldtkdtqaeedrqmdtqaatseapqdvtyaqlqsltlrreate
ppppqkrepsaepsvyatlaiH
Cyno ILT3 Extracellular domain (aa 22-259)
(SEQ ID NO: 8)
qagplpkptvwaepgsviswgspvtiwcqgtldaqeyhldkegsp
apwdtqnpleprnkakfsipsmtqhyagryrcyyhshpdwsedsd
pldlvmtgayskpilsvlpsplvtsgesvtllcqsqspmdtfllf
kegaahplprlrsqhgaqlhwaefpmgpvtsvhggtyrcissrsf
shyllsrpsdpveltvlgslespspsptrsisaaagpedqslmpt
gsdpqsglrrhwe
Cyno ILT3 Ig-like C2-type Domain 1 amino acid
sequence without N-terminal domain (aa 27-118)
(SEQ ID NO: 9)
pkptvwaepgsviswgspvtiwcqgtldaqeyhldkegspapwdt
qnpleprnkakfsipsmtqhyagryrcyyhshpdwsedsdpldlv
mt
Cyno ILT3 Ig-like C2-type Domain 2 amino acid
sequence without N-terminal domain (aa 124-218)
(SEQ ID NO: 10)
pilsvlpsplvtsgesvtllcqsqspmdtfllfkegaahplprlr
sqhgaqlhwaefpmgpvtsvhggtyrcissrsfshyllsrpsdpv
eltvl
3A3 Heavy chain variable region amino acid
sequence
(SEQ ID NO: 109)
QVQLKESGPGLVAPSQSLSITCTVSGFSLTSYGVHWVRQPPGKGL
EWLGVIWPGGTINYNSALMSRLSISKDNSKSQVFLKLNSLQTDDT
AMYYCASDKYDGGWFAYWGQGTLVTVSA
3A3 Light chain variable region amino acid
sequence
(SEQ ID NO: 110)
DIVMTQSQKFMSTSVGDRVSITCKASQNVRTAVAWYQQKPGQSPE
ALIYLASNRHTGVPDRFTGSGSGTDFSLSISNVQSEDLADYFCLQ
HLNYPLTFGSGTKLEIK
5A7 Heavy chain variable region amino acid
sequence
(SEQ ID NO: 111)
EVKLVESGGGLVKPGGSLKLSCAASGFTFSSYGMSWVRQTPEKRL
EWVATISGGGSYTNYPDSVKGRLTISRDNAKKNLYLEMSSLRSED
TALYYCARREWRMTLYAMDYWGQGTSVTVSS
5A7 Light chain variable region amino acid
sequence
(SEQ ID NO: 112)
NIVLTQSPASLAVSLGQRATISCRASESVDSYGNSFMHWYQQKPG
QAPKLLIYLTSNLESGVPARFSGSGSRTDFTLTIDPVEADDAATY
YCQQNNEDPFTFGSGTKLEIK
12A12 Heavy chain variable region amino acid
sequence
(SEQ ID NO: 113)
EVQLQQSGPELVKPGASVKISCKASGYTFTDYNMDWVKQSHGKSL
EWIGYIYPNNGGTGYNQKFNSKATLTVDKSSSTAYMELHSLTSED
SAVYYCASSPYYDYVGSYAMDYWGQGTSVTVSS
12A12 Light chain variable region amino acid
sequence
(SEQ ID NO: 114)
QIVLTQSPAIMSASLGERVTMTCTASSSVSSSYLHWYQQKPGSSP
KLWIYSTSNLASGVPARFSGSGSGTSYSLTISSMEAEDAATYYCH
QYHRSPRTFGGGTKLEIK
16C5 Heavy chain variable region amino acid
sequence
(SEQ ID NO: 115)
EVQLQQSGPELVKPGASVKISCKASGYTFTDYNMDWVKQSHGKSL
EWIGYIYPSNGGTGYNQKFKSKATLTVDKSSNTAYMELHSLTSED
SAVYYCARVPYYDYLYYYAMDYWGQGTSVTVSS
16C5 Light chain variable region amino acid
sequence
(SEQ ID NO: 116)
QIVLSQSPAILSASPGEKVTMACRASSSVSFMHWYQQKPGSSPQP
WIYATSNLASGVPARFSGSGSGTSYSLTISRVEAEDAATYYCQQW
STNPYMYTFGGGTKLEIK
45G10 Heavy chain variable region amino acid
sequence
(SEQ ID NO: 117)
EVQLVESGGGLVKPGGSLKLSCAASGFTFSDYGMHWVRQAPEKGL
EWVAYIFSGSSTIYYADTVKGRFTISRDNAKNTLFLQMTSLRSED
TAMYYCARADGRGAMDYWGQGTSVTVSS
45G10 Light chain variable region amino acid
sequence
(SEQ ID NO: 118)
DVQMTQTTSSLSASLGDRVTISCRASQDISKFLNWYQQKPDGTVT
LLIYYTSRLHSGVPSRFSGSGSGTDYSLTISNLDQEDIATYFCQQ
GNTLPWTFGGGTKLEIK
48A6 Heavy chain variable region amino acid
sequence
(SEQ ID NO: 119)
EVQLVESGGDLMKPGGSLKLSCAASGFTFSSYGMSWVRQTPDKRL
EWVATISSGGTYTFYPDSVKGRFTISRDNAKNTLYLQMSSLKSED
TAMYYCARRGWLLHYYAMDYWGQGTSVTVSS
48A6 Light chain variable region amino acid
sequence
(SEQ ID NO: 120)
NIVLTQSPASLAVSLGQRATISCRPSESVDSFGNSFMHWFQQKPG
QPPKLLIYLSSKLESGVPARFSGSGSRTDFTLTIDPVEADDAATY
YCQQHNEDPFTFGSGTKLEIK
53F10 Heavy chain variable region amino acid
sequence
(SEQ ID NO: 121)
EVQVVESGGGLVKPGGSLKLSCAASGFTFSDYGMHWVRQAPEKGL
EWVAYISTGIITVYYADTVKGRFTMSRDNAKNTLFLQMTSLRSED
TAIYYCARADGRGAMDYWGQGTSVIVSS
53F10 Light chain variable region amino acid
sequence
(SEQ ID NO: 122)
DIQMTQTTSSLSASLGDRVTISCRASQDISNFLNWYQQKPDGTVT
LLIYYTSRLHSGVPSRFSGSGSGTDYSLTISNLEQEDFATYFCQQ
GNTLPWTFGGGTKLEIK
Hz5A7.v5 Heavy chain variable region amino
acid sequence
(SEQ ID NO: 123)
EVQLVESGGGLVQPGGSLRLSCAASGFTFSSYGMSWVRQAPGKGL
EWVATISGGGSYTNYPDSVKGRFTISRDNAKNSLYLQMNSLRAED
TAVYYCARREWRYTLYAMDYWGQGTTVTVSS
Hz5A7.v5 Light chain variable region amino
acid sequence
(SEQ ID NO: 124)
DIQLTQSPSFLSASVGDRVTITCRASESVESYGSSFMHWYQQKPG
KAPKLLIYLTSNLESGVPSRFSGSGSGTEFTLTISSLQPEDFATY
YCQQNNEDPFTFGQGTKLEIK
Hz5A7.v5 Heavy chain amino acid sequence with
signal sequence underlined
(SEQ ID NO: 125)
MDMRVPAQLLGLLLLWLRGARCEVQLVESGGGLVQPGGSLRLSCA
ASGFTFSSYGMSWVRQAPGKGLEWVATISGGGSYTNYPDSVKGRF
TISRDNAKNSLYLQMNSLRAEDTAVYYCARREWRYTLYAMDYWGQ
GTTVTVSSASTKGPSVFPLAPSSKSTSGGTAALGCLVKDYFPEPV
TVSWNSGALTSGVHTFPAVLQSSGLYSLSSVVTVPSSSLGTQTYI
CNVNHKPSNTKVDKKVEPKSCDKTHTCPPCPAPELLGGPSVFLFP
PKPKDTLMISRTPEVTCVVVDVSHEDPEVKFNWYVDGVEVHNAKT
KPREEQYGSTYRVVSVLTVLHQDWLNGKEYKCKVSNKALPAPIEK
TISKAKGQPREPQVYTLPPSREEMTKNQVSLTCLVKGFYPSDIAV
EWESNGQPENNYKTTPPVLDSDGSFFLYSKLTVDKSRWQQGNVFS
CSVMHEALHNHYTQKSLSLSPGK
Hz5A7.v5 Heavy chain amino acid sequence
without signal sequence
(SEQ ID NO: 126)
EVQLVESGGGLVQPGGSLRLSCAASGFTFSSYGMSWVRQAPGKGL
EWVATISGGGSYTNYPDSVKGRFTISRDNAKNSLYLQMNSLRAED
TAVYYCARREWRYTLYAMDYWGQGTTVTVSSASTKGPSVFPLAPS
SKSTSGGTAALGCLVKDYFPEPVTVSWNSGALTSGVHTFPAVLQS
SGLYSLSSVVTVPSSSLGTQTYICNVNHKPSNTKVDKKVEPKSCD
KTHTCPPCPAPELLGGPSVFLFPPKPKDTLMISRTPEVTCVVVDV
SHEDPEVKFNWYVDGVEVHNAKTKPREEQYGSTYRVVSVLTVLHQ
DWLNGKEYKCKVSNKALPAPIEKTISKAKGQPREPQVYTLPPSRE
EMTKNQVSLTCLVKGFYPSDIAVEWESNGQPENNYKTTPPVLDSD
GSFFLYSKLTVDKSRWQQGNVFSCSVMHEALHNHYTQKSLSLSPG
K
Hz5A7.v5 Light chain amino acid sequence
with signal sequence underlined
(SEQ ID NO: 127)
MDMRVPAQLLGLLLLWLRGARCDIOLTOSPSFLSASVGDRVTITC
RASESVESYGSSFMHWYOQKPGKAPKLLIYLTSNLESGVPSRFSG
SGSGTEFTLTISSLQPEDFATYYCQQNNEDPFTFGQGTKLEIKRT
VAAPSVFIFPPSDEQLKSGTASVVCLLNNFYPREAKVQWKVDNAL
QSGNSQESVTEQDSKDSTYSLSSTLTLSKADYEKHKVYACEVTHQ
GLSSPVTKSFNRGEC
Hz5A7.v5 Light chain amino acid sequence
without signal sequence
(SEQ ID NO: 128)
DIQLTQSPSFLSASVGDRVTITCRASESVESYGSSFMHWYQQKPG
KAPKLLIYLTSNLESGVPSRFSGSGSGTEFTLTISSLQPEDFATY
YCQQNNEDPFTFGQGTKLEIKRTVAAPSVFIFPPSDEQLKSGTAS
VVCLLNNFYPREAKVQWKVDNALQSGNSQESVTEQDSKDSTYSLS
STLTLSKADYEKHKVYACEVTHQGLSSPVTKSFNRGEC
Human IgG1 constant region
(SEQ ID NO: 129)
ASTKGPSVFPLAPSSKSTSGGTAALGCLVKDYFPEPVTVSWNSGA
LTSGVHTFPAVLQSSGLYSLSSVVTVPSSSLGTQTYICNVNHKPS
NTKVDKKVEPKSCDKTHTCPPCPAPELLGGPSVFLFPPKPKDTLM
ISRTPEVTCVVVDVSHEDPEVKFNWYVDGVEVHNAKTKPREEQYN
STYRVVSVLTVLHQDWLNGKEYKCKVSNKALPAPIEKTISKAKGQ
PREPQVYTLPPSREEMTKNQVSLTCLVKGFYPSDIAVEWESNGQP
ENNYKTTPPVLDSDGSFFLYSKLTVDKSRWQQGNVFSCSVMHEAL
HNHYTQKSLSLSPGK
Human IgG1 constant region E233A/L235A
(SEQ ID NO: 130)
ASTKGPSVFPLAPSSKSTSGGTAALGCLVKDYFPEPVTVSWNSGA
LTSGVHTFPAVLQSSGLYSLSSVVTVPSSSLGTQTYICNVNHKPS
NTKVDKKVEPKSCDKTHTCPPCPAPALAGGPSVFLFPPKPKDTLM
ISRTPEVTCVVVDVSHEDPEVKFNWYVDGVEVHNAKTKPREEQYN
STYRVVSVLTVLHQDWLNGKEYKCKVSNKALPAPIEKTISKAKGQ
PREPQVYTLPPSREEMTKNQVSLTCLVKGFYPSDIAVEWESNGQP
ENNYKTTPPVLDSDGSFFLYSKLTVDKSRWQQGNVFSCSVMHEAL
HNHYTQKSLSLSPGK
Human IgG1 constant region L234A/L235A
(SEQ ID NO: 131)
ASTKGPSVFPLAPSSKSTSGGTAALGCLVKDYFPEPVTVSWNSGA
LTSGVHTFPAVLQSSGLYSLSSVVTVPSSSLGTQTYICNVNHKPS
NTKVDKKVEPKSCDKTHTCPPCPAPEAAGGPSVFLFPPKPKDTLM
ISRTPEVTCVVVDVSHEDPEVKFNWYVDGVEVHNAKTKPREEQYN
STYRVVSVLTVLHQDWLNGKEYKCKVSNKALPAPIEKTISKAKGQ
PREPQVYTLPPSREEMTKNQVSLTCLVKGFYPSDIAVEWESNGQP
ENNYKTTPPVLDSDGSFFLYSKLTVDKSRWQQGNVFSCSVMHEAL
HNHYTQKSLSLSPGK
Human IgG1 constant region L234A/L235A/P329G
(SEQ ID NO: 132)
ASTKGPSVFPLAPSSKSTSGGTAALGCLVKDYFPEPVTVSWNSGA
LTSGVHTFPAVLQSSGLYSLSSVVTVPSSSLGTQTYICNVNHKPS
NTKVDKKVEPKSCDKTHTCPPCPAPEAAGGPSVFLFPPKPKDTLM
ISRTPEVTCVVVDVSHEDPEVKFNWYVDGVEVHNAKTKPREEQYN
STYRVVSVLTVLHQDWLNGKEYKCKVSNKALGAPIEKTISKAKGQ
PREPQVYTLPPSREEMTKNQVSLTCLVKGFYPSDIAVEWESNGQP
ENNYKTTPPVLDSDGSFFLYSKLTVDKSRWQQGNVFSCSVMHEAL
HNHYTQKSLSLSPGK
Human IgG1 constant region N297G
(SEQ ID NO: 133)
ASTKGPSVFPLAPSSKSTSGGTAALGCLVKDYFPEPVTVSWNSGA
LTSGVHTFPAVLQSSGLYSLSSVVTVPSSSLGTQTYICNVNHKPS
NTKVDKKVEPKSCDKTHTCPPCPAPELLGGPSVFLFPPKPKDTLM
ISRTPEVTCVVVDVSHEDPEVKFNWYVDGVEVHNAKTKPREEQYG
STYRVVSVLTVLHQDWLNGKEYKCKVSNKALPAPIEKTISKAKGQ
PREPQVYTLPPSREEMTKNQVSLTCLVKGFYPSDIAVEWESNGQP
ENNYKTTPPVLDSDGSFFLYSKLTVDKSRWQQGNVFSCSVMHEAL
HNHYTQKSLSLSPGK
Human IgG1 constant region N297G/H310A
(SEQ ID NO: 134)
ASTKGPSVFPLAPSSKSTSGGTAALGCLVKDYFPEPVTVSWNSGA
LTSGVHTFPAVLQSSGLYSLSSVVTVPSSSLGTQTYICNVNHKPS
NTKVDKKVEPKSCDKTHTCPPCPAPELLGGPSVFLFPPKPKDTLM
ISRTPEVTCVVVDVSHEDPEVKFNWYVDGVEVHNAKTKPREEQYG
STYRVVSVLTVLAQDWLNGKEYKCKVSNKALPAPIEKTISKAKGQ
PREPQVYTLPPSREEMTKNQVSLTCLVKGFYPSDIAVEWESNGQP
ENNYKTTPPVLDSDGSFFLYSKLTVDKSRWQQGNVFSCSVMHEAL
HNHYTQKSLSLSPGK
Human IgG1 constant region L234F/L235E/P331G
(SEQ ID NO: 135)
ASTKGPSVFPLAPSSKSTSGGTAALGCLVKDYFPEPVTVSWNSGA
LTSGVHTFPAVLQSSGLYSLSSVVTVPSSSLGTQTYICNVNHKPS
NTKVDKKVEPKSCDKTHTCPPCPAPEFEGGPSVFLFPPKPKDTLM
ISRTPEVTCVVVDVSHEDPEVKFNWYVDGVEVHNAKTKPREEQYN
STYRVVSVLTVLHQDWLNGKEYKCKVSNKALPAGIEKTISKAKGQ
PREPQVYTLPPSREEMTKNQVSLTCLVKGFYPSDIAVEWESNGQP
ENNYKTTPPVLDSDGSFFLYSKLTVDKSRWQQGNVFSCSVMHEAL
HNHYTQKSLSLSPGK
Human IgG1 constant region L234A/L235A/P331G
(SEQ ID NO: 136)
ASTKGPSVFPLAPSSKSTSGGTAALGCLVKDYFPEPVTVSWNSGA
LTSGVHTFPAVLQSSGLYSLSSVVTVPSSSLGTQTYICNVNHKPS
NTKVDKKVEPKSCDKTHTCPPCPAPEAAGGPSVFLFPPKPKDTLM
ISRTPEVTCVVVDVSHEDPEVKFNWYVDGVEVHNAKTKPREEQYN
STYRVVSVLTVLHQDWLNGKEYKCKVSNKALPAGIEKTISKAKGQ
PREPQVYTLPPSREEMTKNQVSLTCLVKGFYPSDIAVEWESNGQP
ENNYKTTPPVLDSDGSFFLYSKLTVDKSRWQQGNVFSCSVMHEAL
HNHYTQKSLSLSPGK
Human Kappa light chain constant region
(SEQ ID NO: 137)
RTVAAPSVFIFPPSDEQLKSGTASVVCLLNNFYPREAKVQWKVDN
ALQSGNSQESVTEQDSKDSTYSLSSTLTLSKADYEKHKVYACEVT
HQGLSSPVTKSFNRGEC
Human Lambda light chain constant region
(SEQ ID NO: 138)
GQPKAAPSVTLFPPSSEELQANKATLVCLISDFYPGAVTVAWKAD
SSPVKAGVETTTPSKQSNNKYAASSYLSLTPEQWKSHRSYSCQVT
HEGSTVEKTVAPTECS
Human ILT3 Ig-like C2-type D1 and D2 amino
acid sequence without N-terminal domain
(aa 27-218)
(SEQ ID NO: 139)
QAGPLPKPTLWAEPGSVISWGNSVTIWCQGTLEAREYRLDKEESP
APWDRQNPLEPKNKARFSIPSMTEDYAGRYRCYYRSPVGWSQPSD
PLELVMTGAYSKPTLSALPSPLVTSGKSVTLLCQSRSPMDTFLLI
KERAAHPLLHLRSEHGAQQHQAEFPMSPVTSVHGGTYRCFSSHGF
SHYLLSHPSDPLELIVS
Human Fibronectin with predicted signal
sequence underlined
(SEQ ID NO: 140)
MLRGPGPGLLLLAVQCLGTAVPSTGASKSKRQAQQMVQPQSPVAV
SQSKPGCYDNGKHYQINQQWERTYLGNALVCTCYGGSRGFNCESK
PEAEETCFDKYTGNTYRVGDTYERPKDSMIWDCTCIGAGRGRISC
TIANRCHEGGQSYKIGDTWRRPHETGGYMLECVCLGNGKGEWTCK
PIAEKCFDHAAGTSYVVGETWEKPYQGWMMVDCTCLGEGSGRITC
TSRNRCNDQDTRTSYRIGDTWSKKDNRGNLLQCICTGNGRGEWKC
ERHTSVQTTSSGSGPFTDVRAAVYQPQPHPQPPPYGHCVTDSGVV
YSVGMQWLKTQGNKQMLCTCLGNGVSCQETAVTQTYGGNSNGEPC
VLPFTYNGRTFYSCTTEGRQDGHLWCSTTSNYEQDQKYSFCTDHT
VLVQTRGGNSNGALCHFPFLYNNHNYTDCTSEGRRDNMKWCGTTQ
NYDADQKFGFCPMAAHEEICTTNEGVMYRIGDQWDKQHDMGHMMR
CTCVGNGRGEWTCIAYSQLRDQCIVDDITYNVNDTFHKRHEEGHM
LNCTCFGQGRGRWKCDPVDQCQDSETGTFYQIGDSWEKYVHGVRY
QCYCYGRGIGEWHCQPLQTYPSSSGPVEVFITETPSQPNSHPIQW
NAPQPSHISKYILRWRPKNSVGRWKEATIPGHLNSYTIKGLKPGV
VYEGQLISIQQYGHQEVTRFDFTTTSTSTPVTSNTVTGETTPFSP
LVATSESVTEITASSFVVSWVSASDTVSGFRVEYELSEEGDEPQY
LDLPSTATSVNIPDLLPGRKYIVNVYQISEDGEQSLILSTSQTTA
PDAPPDTTVDQVDDTSIVVRWSRPQAPITGYRIVYSPSVEGSSTE
LNLPETANSVTLSDLQPGVQYNITIYAVEENQESTPVVIQQETTG
TPRSDTVPSPRDLQFVEVTDVKVTIMWTPPESAVTGYRVDVIPVN
LPGEHGQRLPISRNTFAEVTGLSPGVTYYFKVFAVSHGRESKPLT
AQQTTKLDAPTNLQFVNETDSTVLVRWTPPRAQITGYRLTVGLTR
RGQPRQYNVGPSVSKYPLRNLQPASEYTVSLVAIKGNQESPKATG
VFTTLQPGSSIPPYNTEVTETTIVITWTPAPRIGFKLGVRPSQGG
EAPREVTSDSGSIVVSGLTPGVEYVYTIQVLRDGQERDAPIVNKV
VTPLSPPTNLHLEANPDTGVLTVSWERSTTPDITGYRITTTPTNG
QQGNSLEEVVHADQSSCTFDNLSPGLEYNVSVYTVKDDKESVPIS
DTIIPEVPQLTDLSFVDITDSSIGLRWTPLNSSTIIGYRITVVAA
GEGIPIFEDFVDSSVGYYTVTGLEPGIDYDISVITLINGGESAPT
TLTQQTAVPPPTDLRFTNIGPDTMRVTWAPPPSIDLTNFLVRYSP
VKNEEDVAELSISPSDNAVVLTNLLPGTEYVVSVSSVYEQHESTP
LRGRQKTGLDSPTGIDFSDITANSFTVHWIAPRATITGYRIRHHP
EHFSGRPREDRVPHSRNSITLTNLTPGTEYVVSIVALNGREESPL
LIGQQSTVSDVPRDLEVVAATPTSLLISWDAPAVTVRYYRITYGE
TGGNSPVQEFTVPGSKSTATISGLKPGVDYTITVYAVTGRGDSPA
SSKPISINYRTEIDKPSQMQVTDVQDNSISVKWLPSSSPVTGYRV
TTTPKNGPGPTKTKTAGPDQTEMTIEGLQPTVEYVVSVYAQNPSG
ESQPLVQTAVTNIDRPKGLAFTDVDVDSIKIAWESPQGQVSRYRV
TYSSPEDGIHELFPAPDGEEDTAELQGLRPGSEYTVSVVALHDDM
ESQPLIGTQSTAIPAPTDLKFTQVTPTSLSAQWTPPNVQLTGYRV
RVTPKEKTGPMKEINLAPDSSSVVVSGLMVATKYEVSVYALKDTL
TSRPAQGVVTTLENVSPPRRARVTDATETTITISWRTKTETITGF
QVDAVPANGQTPIQRTIKPDVRSYTITGLQPGTDYKIYLYTLNDN
ARSSPVVIDASTAIDAPSNLRFLATTPNSLLVSWQPPRARITGYI
IKYEKPGSPPREVVPRPRPGVTEATITGLEPGTEYTIYVIALKNN
QKSEPLIGRKKTDELPQLVTLPHPNLHGPEILDVPSTVQKTPFVT
HPGYDTGNGIQLPGTSGQQPSVGQQMIFEEHGFRRTTPPTTATPI
RHRPRPYPPNVGEEIQIGHIPREDVDYHLYPHGPGLNPNASTGQE
ALSQTTISWAPFQDTSEYIISCHPVGTDEEPLQFRVPGTSTSATL
TGLTRGATYNVIVEALKDQQRHKVREEVVTVGNSVNEGLNQPTDD
SCFDPYTVSHYAVGDEWERMSESGFKLLCQCLGFGSGHFRCDSSR
WCHDNGVNYKIGEKWDRQGENGQMMSCTCLGNGKGEFKCDPHEAT
CYDDGKTYHVGEQWQKEYLGAICSCTCFGGQRGWRCDNCRRPGGE
PSPEGTTGQSYNQYSQRYHQRTNTNVNCPIECFMPLDVQADREDS
RE
Human Fibronectin without predicted signal
sequence
(SEQ ID NO: 141)
QAQQMVQPQSPVAVSQSKPGCYDNGKHYQINQQWERTYLGNALVC
TCYGGSRGFNCESKPEAEETCFDKYTGNTYRVGDTYERPKDSMIW
DCTCIGAGRGRISCTIANRCHEGGQSYKIGDTWRRPHETGGYMLE
CVCLGNGKGEWTCKPIAEKCFDHAAGTSYVVGETWEKPYQGWMMV
DCTCLGEGSGRITCTSRNRCNDQDTRTSYRIGDTWSKKDNRGNLL
QCICTGNGRGEWKCERHTSVQTTSSGSGPFTDVRAAVYQPQPHPQ
PPPYGHCVTDSGVVYSVGMQWLKTQGNKQMLCTCLGNGVSCQETA
VTQTYGGNSNGEPCVLPFTYNGRTFYSCTTEGRQDGHLWCSTTSN
YEQDQKYSFCTDHTVLVQTRGGNSNGALCHFPFLYNNHNYTDCTS
EGRRDNMKWCGTTQNYDADQKFGFCPMAAHEEICTTNEGVMYRIG
DQWDKQHDMGHMMRCTCVGNGRGEWTCIAYSQLRDQCIVDDITYN
VNDTFHKRHEEGHMLNCTCFGQGRGRWKCDPVDQCQDSETGTFYQ
IGDSWEKYVHGVRYQCYCYGRGIGEWHCQPLQTYPSSSGPVEVFI
TETPSQPNSHPIQWNAPQPSHISKYILRWRPKNSVGRWKEATIPG
HLNSYTIKGLKPGVVYEGQLISIQQYGHQEVTRFDFTTTSTSTPV
TSNTVTGETTPFSPLVATSESVTEITASSFVVSWVSASDTVSGFR
VEYELSEEGDEPQYLDLPSTATSVNIPDLLPGRKYIVNVYQISED
GEQSLILSTSQTTAPDAPPDTTVDQVDDTSIVVRWSRPQAPITGY
RIVYSPSVEGSSTELNLPETANSVTLSDLQPGVQYNITIYAVEEN
QESTPVVIQQETTGTPRSDTVPSPRDLQFVEVTDVKVTIMWTPPE
SAVTGYRVDVIPVNLPGEHGQRLPISRNTFAEVTGLSPGVTYYFK
VFAVSHGRESKPLTAQQTTKLDAPTNLQFVNETDSTVLVRWTPPR
AQITGYRLTVGLTRRGQPRQYNVGPSVSKYPLRNLQPASEYTVSL
VAIKGNQESPKATGVFTTLQPGSSIPPYNTEVTETTIVITWTPAP
RIGFKLGVRPSQGGEAPREVTSDSGSIVVSGLTPGVEYVYTIQVL
RDGQERDAPIVNKVVTPLSPPTNLHLEANPDTGVLTVSWERSTTP
DITGYRITTTPTNGQQGNSLEEVVHADQSSCTFDNLSPGLEYNVS
VYTVKDDKESVPISDTIIPEVPQLTDLSFVDITDSSIGLRWTPLN
SSTIIGYRITVVAAGEGIPIFEDFVDSSVGYYTVTGLEPGIDYDI
SVITLINGGESAPTTLTQQTAVPPPTDLRFTNIGPDTMRVTWAPP
PSIDLTNFLVRYSPVKNEEDVAELSISPSDNAVVLTNLLPGTEYV
VSVSSVYEQHESTPLRGRQKTGLDSPTGIDFSDITANSFTVHWIA
PRATITGYRIRHHPEHFSGRPREDRVPHSRNSITLTNLTPGTEYV
VSIVALNGREESPLLIGQQSTVSDVPRDLEVVAATPTSLLISWDA
PAVTVRYYRITYGETGGNSPVQEFTVPGSKSTATISGLKPGVDYT
ITVYAVTGRGDSPASSKPISINYRTEIDKPSQMQVTDVQDNSISV
KWLPSSSPVTGYRVTTTPKNGPGPTKTKTAGPDQTEMTIEGLQPT
VEYVVSVYAQNPSGESQPLVQTAVTNIDRPKGLAFTDVDVDSIKI
AWESPQGQVSRYRVTYSSPEDGIHELFPAPDGEEDTAELQGLRPG
SEYTVSVVALHDDMESQPLIGTQSTAIPAPTDLKFTQVTPTSLSA
QWTPPNVQLTGYRVRVTPKEKTGPMKEINLAPDSSSVVVSGLMVA
TKYEVSVYALKDTLTSRPAQGVVTTLENVSPPRRARVTDATETTI
TISWRTKTETITGFQVDAVPANGQTPIQRTIKPDVRSYTITGLQP
GTDYKIYLYTLNDNARSSPVVIDASTAIDAPSNLRFLATTPNSLL
VSWQPPRARITGYIIKYEKPGSPPREVVPRPRPGVTEATITGLEP
GTEYTIYVIALKNNQKSEPLIGRKKTDELPQLVTLPHPNLHGPEI
LDVPSTVQKTPFVTHPGYDTGNGIQLPGTSGQQPSVGQQMIFEEH
GFRRTTPPTTATPIRHRPRPYPPNVGEEIQIGHIPREDVDYHLYP
HGPGLNPNASTGQEALSQTTISWAPFQDTSEYIISCHPVG
TDEEPLQFRVPGTSTSATLTGLTRGATYNVIVEALKDQQRHKVRE
EVVTVGNSVNEGLNQPTDDSCFDPYTVSHYAVGDEWERMSESGFK
LLCQCLGFGSGHFRCDSSRWCHDNGVNYKIGEKWDRQGENGQMMS
CTCLGNGKGEFKCDPHEATCYDDGKTYHVGEQWQKEYLGAICSCT
CFGGQRGWRCDNCRRPGGEPSPEGTTGQSYNQYSQRYHQRTNTNV
NCPIECFMPLDVQADREDSRE
Human Fibronectin fragment containing
heparin-binding and collagen-binding
domains (~70 kDa
fragment)
(SEQ ID NO: 142)
QAQQMVQPQSPVAVSQSKPGCYDNGKHYQINQQWERTYLGNALVC
TCYGGSRGFNCESKPEAEETCFDKYTGNTYRVGDTYERPKDSMIW
DCTCIGAGRGRISCTIANRCHEGGQSYKIGDTWRRPHETGGYMLE
CVCLGNGKGEWTCKPIAEKCFDHAAGTSYVVGETWEKPYQGWMMV
DCTCLGEGSGRITCTSRNRCNDQDTRTSYRIGDTWSKKDNRGNLL
QCICTGNGRGEWKCERHTSVQTTSSGSGPFTDVRAAVYQPQPHPQ
PPPYGHCVTDSGVVYSVGMQWLKTQGNKQMLCTCLGNGVSCQETA
VTQTYGGNSNGEPCVLPFTYNGRTFYSCTTEGRQDGHLWCSTTSN
YEQDQKYSFCTDHTVLVQTRGGNSNGALCHFPFLYNNHNYTDCTS
EGRRDNMKWCGTTQNYDADQKFGFCPMAAHEEICTTNEGVMYRIG
DQWDKQHDMGHMMRCTCVGNGRGEWTCIAYSQLRDQCIVDDITYN
VNDTFHKRHEEGHMLNCTCFGQGRGRWKCDPVDQCQDSETGTFYQ
IGDSWEKYVHGVRYQCYCYGRGIGEWHCQPLQTYPSS
Human Fibronectin fragment containing
heparin-binding domain (~30 kDa fragment)
(SEQ ID NO: 143)
QAQQMVQPQSPVAVSQSKPGCYDNGKHYQINQQWERTYLGNALVC
TCYGGSRGFNCESKPEAEETCFDKYTGNTYRVGDTYERPKDSMIW
DCTCIGAGRGRISCTIANRCHEGGQSYKIGDTWRRPHETGGYMLE
CVCLGNGKGEWTCKPIAEKCFDHAAGTSYVVGETWEKPYQGWMMV
DCTCLGEGSGRITCTSRNRCNDQDTRTSYRIGDTWSKKDNRGNLL
QCICTGNGRGEWKCER
Human Fibronectin fragment containing
collagen-binding domain (~45 kDa fragment)
(SEQ ID NO: 144)
HTSVQTTSSGSGPFTDVRAAVYQPQPHPQPPPYGHCVTDSGVVYS
VGMQWLKTQGNKQMLCTCLGNGVSCQETAVTQTYGGNSNGEPCVL
PFTYNGRTFYSCTTEGRQDGHLWCSTTSNYEQDQKYSFCTDHTVL
VQTRGGNSNGALCHFPFLYNNHNYTDCTSEGRRDNMKWCGTTQNY
DADQKFGFCPMAAHEEICTTNEGVMYRIGDQWDKQHDMGHMMRCT
CVGNGRGEWTCIAYSQLRDQCIVDDITYNVNDTFHKRHEEGHMLN
CTCFGQGRGRWKCDPVDQCQDSETGTFYQIGDSWEKYVHGVRYQC
YCYGRGIGEWHCQPLQTYPSS
Human Fibronectin fragment containing
type I repeat 1
(SEQ ID NO: 145)
PGCYDNGKHYQINQQWERTYLGNALVCTCYGGSRGFNCESKPEA
Human Fibronectin fragment containing
type I repeat 2
(SEQ ID NO: 146)
ETCFDKYTGNTYRVGDTYERPKDSMIWDCTCIGAGRGRISCTIA
Human Fibronectin fragment containing
type I repeat 3
(SEQ ID NO: 147)
NRCHEGGQSYKIGDTWRRPHETGGYMLECVCLGNGKGEWTCKPI
Human Fibronectin fragment containing
type I repeat 4
(SEQ ID NO: 148)
AEKCFDHAAGTSYVVGETWEKPYQGWMMVDCTCLGEGSGRITC
TSR
Human Fibronectin fragment containing
type I repeat 5
(SEQ ID NO: 149)
NRCNDQDTRTSYRIGDTWSKKDNRGNLLQCICTGNGRGEWKCERH
Human CNTFR-alpha with predicted signal
sequence underlined
(SEQ ID NO: 150)
MAAPVPWACCAVLAAAAAVVYAQRHSPQEAPHVQYERLGSDVTLP
CGTANWDAAVTWRVNGTDLAPDLLNGSQLVLHGLELGHSGLYACF
HRDSWHLRHQVLLHVGLPPREPVLSCRSNTYPKGFYCSWHLPTPT
YIPNTFNVTVLHGSKIMVCEKDPALKNRCHIRYMHLFSTIKYKVS
ISVSNALGHNATAITFDEFTIVKPDPPENVVARPVPSNPRRLEVT
WQTPSTWPDPESFPLKFFLRYRPLILDQWQHVELSDGTAHTITDA
YAGKEYIIQVAAKDNEIGTWSDWSVAAHATPWTEEPRHLTTEAQA
AETTTSTTSSLAPPPTTKICDPGELGSGGGPSAPFLVSVPITLAL
AAAAATASSLLI
Human CNTFR-alpha without predicted signal
sequence
(SEQ ID NO: 151)
QRHSPQEAPHVQYERLGSDVTLPCGTANWDAAVTWRVNGTDLAPD
LLNGSQLVLHGLELGHSGLYACFHRDSWHLRHQVLLHVGLPPREP
VLSCRSNTYPKGFYCSWHLPTPTYIPNTFNVTVLHGSKIMVCEKD
PALKNRCHIRYMHLFSTIKYKVSISVSNALGHNATAITFDEFTIV
KPDPPENVVARPVPSNPRRLEVTWQTPSTWPDPESFPLKFFLRYR
PLILDQWQHVELSDGTAHTITDAYAGKEYIIQVAAKDNEIGTWSD
WSVAAHATPWTEEPRHLTTEAQAAETTTSTTSSLAPPPTTKICDP
GELGSGGGPSAPFLVSVPITLALAAAAATASSLLI
Human CNTFR-alpha - mature form
(SEQ ID NO: 152)
QRHSPQEAPHVQYERLGSDVTLPCGTANWDAAVTWRVNGTDLAPD
LLNGSQLVLHGLELGHSGLYACFHRDSWHLRHQVLLHVGLPPREP
VLSCRSNTYPKGFYCSWHLPTPTYIPNTFNVTVLHGSKIMVCEKD
PALKNRCHIRYMHLFSTIKYKVSISVSNALGHNATAITFDEFTIV
KPDPPENVVARPVPSNPRRLEVTWQTPSTWPDPESFPLKFFLRYR
PLILDQWQHVELSDGTAHTITDAYAGKEYIIQVAAKDNEIGTWSD
WSVAAHATPWTEEPRHLTTEAQAAETTTSTTSSLAPPPTTKICDP
GELGS
Human CNTFR-alpha fragment containing
Ig-like C2 type domain
(SEQ ID NO: 153)
QRHSPQEAPHVQYERLGSDVTLPCGTANWDAAVTWRVNGTDLAPD
LLNGSQLVLHGLELGHSGLYACFHRDSWHLRHQVLLH
Human CNTFR-alpha fragment containing
FN type III domain 1
(SEQ ID NO: 154)
PPREPVLSCRSNTYPKGFYCSWHLPTPTYIPNTFNVTVLHGSKIM
VCEKDPALKNRCHIRYMHLFSTIKYKVSISVSNALGHNATAITFD
EFTIVKPDPPENVVARPVPSNPRRLEVT
Human CNTFR-alpha fragment containing
FN type III domain 2
(SEQ ID NO: 155)
WQTPSTWPDPESFPLKFFLRYRPLILDQWQHVELSDGTAHTITDA
YAGKEYIIQVAAKDNEIGTWSDWSVAAHATPWTEEP
Hexahistidine peptide tag
(SEQ ID NO: 156)
HHHHHH
Human ILT3 Domain 2 with stem amino acid
sequence (aa 124-259)
(SEQ ID NO: 157)
PTLSALPSPLVTSGKSVTLLCQSRSPMDTFLLIKERAAHPLLHLR
SEHGAQQHQAEFPMSPVTSVHGGTYRCFSSHGFSHYLLSHPSDPL
ELIVSGSLEDPRPSPTRSVSTAAGPEDQPLMPTGSVPHSGLRRHW
E
Hz48A6 heavy chain variable region amino
acid sequences:
HC-BO
(SEQ ID NO: 158)
EVQLVESGGGLVQPGGSLRLSCAASGFTFSSYGMSWVRQAPGKGL
EWVATISSGGTYTFYPDSVKGRFTISRDNAKNSLYLQMNSLRAED
TAVYYCARRGWLLHYYAMDYWGQGTSVTVSS
HC-B1
(SEQ ID NO: 159)
EVQLVESGGGLMQPGGSLRLSCAASGFTFSSYGMSWVRQAPGKGL
EWVATISSGGTYTFYPDSVKGRFTISRDNAKNSLYLQMNSLRAED
TAVYYCARRGWLLHYYAMDYWGQGTSVTVSS
HC-C0
(SEQ ID NO: 160)
EVQLVESGGGLVQPGGSLRLSCAASGFTFSSYGMSWVRQAPGKGL
EWVGTISSGGTYTFYPDSVKGRFTISRDNSKNTLYLQMNSLRAED
TAVYYCARRGWLLHYYAMDYWGQGTLVTVSS
HC-C1
(SEQ ID NO: 161)
EVQLVESGGGLVQPGGSLRLSCAASGFTFSSYGMSWVRQAPGKGL
EWVATISSGGTYTFYPDSVKGRFTISRDNSKNTLYLQMNSLRAED
TAVYYCARRGWLLHYYAMDYWGQGTLVTVSS
HC-C2
(SEQ ID NO: 162)
EVQLVESGGGLMQPGGSLRLSCAASGFTFSSYGMSWVRQAPGKGL
EWVATISSGGTYTFYPDSVKGRFTISRDNSKNTLYLQMNSLRAED
TAVYYCARRGWLLHYYAMDYWGQGTLVTVSS
Hz48A6 light chain variable region amino
acid sequence
LC-C2
(SEQ ID NO: 163)
DIQLTQSPSFLSASVGDRVTITCRPSESVDSFGNSFMHWFQQKPG
KAPKLLIYLSSKLESGVPSRFSGSGSGTEFTLTISSLQPEDFATY
YCQQHNEDPFTFGQGTKVEIK
Hz45G10 heavy chain variable region amino
acid sequences:
HC-A2
(SEQ ID NO: 164)
EVQLVESGGGLVQPGGSLRLSCAASGFTFSDYGMHWVRQAPGKGL
EWVAYIFSGSSTIYYADTVKGRFTISRDNAKNSLYLQMNSLRAED
TAVYYCARADGRGAMDYWGQGTTVTVSS
HC-B1
(SEQ ID NO: 165)
EVQLVESGGGLVKPGGSLRLSCAASGFTFSDYGMHWVRQAPGKGL
EWVSYIFSGSSTIYYADTVKGRFTISRDNAKNSLYLQMNSLRAED
TAVYYCARADGRGAMDYWGQGTLVTVSS
Hz45G10 light chain variable region amino
acid sequence
LC-A4
(SEQ ID NO: 166)
DIQMTQSPSSLSASVGDRVTITCRASQDISKFLNWYQQKPGKAPK
LLIYYTSRLHSGVPSRFSGSGSGTDFTFTISSLQPEDIATYFCQQ
GNTLPWTFGGGTKLEIK
Human LAIR-1 amino acid sequence with
predicted signal sequence underlined
(SEQ ID NO: 167)
MSPHPTALLGLVLCLAQTIHTQEEDLPRPSISAEPGTVIPLGSHV
TFVCRGPVGVOTFRLERESRSTYNDTEDVSQASPSESEARFRIDS
VSEGNAGPYRCIYYKPPKWSEQSDYLELLVKETSGGPDSPDTEPG
SSAGPTQRPSDNSHNEHAPASQGLKAEHLYILIGVSVVFLFCLLL
LVLFCLHRQNQIKQGPPRSKDEEQKPQQRPDLAVDVLERTADKAT
VNGLPEKDRETDTSALAAGSSQEVTYAQLDHWALTQRTARAVSPQ
STKPMAESITYAAVARH
Human LAIR-1 amino acid sequence without
predicted signal sequence
(SEQ ID NO: 168)
QEEDLPRPSISAEPGTVIPLGSHVTFVCRGPVGVQTFRLERESRS
TYNDTEDVSQASPSESEARFRIDSVSEGNAGPYRCIYYKPPKWSE
QSDYLELLVKETSGGPDSPDTEPGSSAGPTQRPSDNSHNEHAPAS
QGLKAEHLYILIGVSVVFLFCLLLLVLFCLHRQNQIKQGPPRSKD
EEQKPQQRPDLAVDVLERTADKATVNGLPEKDRETDTSALAAGSS
QEVTYAQLDHWALTQRTARAVSPQSTKPMAESITYAAVARH
Human LAIR-1 extracellular domain (aa 22-165)
(SEQ ID NO: 169)
QEEDLPRPSISAEPGTVIPLGSHVTFVCRGPVGVQTFRLERESRS
TYNDTEDVSQASPSESEARFRIDSVSEGNAGPYRCIYYKPPKWSE
QSDYLELLVKETSGGPDSPDTEPGSSAGPTQRPSDNSHNEHAPAS
QGLKAEHLY
Human LAIR-1 Ig-like C2-type domain amino
acid sequence (aa 29-117)
(SEQ ID NO: 170)
PSISAEPGTVIPLGSHVTFVCRGPVGVQTFRLERESRSTYNDTED
VSQASPSESEARFRIDSVSEGNAGPYRCIYYKPPKWSEQSDYLE
Cyno LAIR-1 amino acid sequence with
predicted signal sequence underlined
(SEQ ID NO: 171)
MSPHPTALLGLVLCLAQTIHAQEGPLPRPSISAEPGTVIPPGRPV
TIVCRGPVGVDOFRLEREDRSKFNDTKDVSQASPSESEARFRIDS
VSEGNAGHYRCLYVKSTRWSEHSDYLDLVVKETSGDTDSPVTEPD
SSAGPTQRPSDNSHNEHAPASQGLSAEHLYILIGVSVVFLFCLLL
LVLFFLHRQNQMKQGPPRSKDEEQKLQQRPDLAVDVLERTADKAT
VNGLPEKDRETDTSAPAAGSSQEVTYAQLDHWALTWRTAQAVSPQ
STEPMAESSTYAAVARH
Cyno LAIR-1 amino acid sequence without
predicted signal sequence
(SEQ ID NO: 172)
QEGPLPRPSISAEPGTVIPPGRPVTIVCRGPVGVDQFRLEREDRS
KFNDTKDVSQASPSESEARFRIDSVSEGNAGHYRCLYVKSTRWSE
HSDYLDLVVKETSGDTDSPVTEPDSSAGPTQRPSDNSHNEHAPAS
QGLSAEHLYILIGVSVVFLFCLLLLVLFFLHRQNQMKQGPPRSKD
EEQKLQQRPDLAVDVLERTADKATVNGLPEKDRETDTSAPAAGSS
QEVTYAQLDHWALTWRTAQAVSPQSTEPMAESSTYAAVARH
Cyno LAIR-1 extracellular domain (aa 22-165)
(SEQ ID NO: 173)
QEGPLPRPSISAEPGTVIPPGRPVTIVCRGPVGVDQFRLEREDRS
KFNDTKDVSQASPSESEARFRIDSVSEGNAGHYRCLYVKSTRWSE
HSDYLDLVVKETSGDTDSPVTEPDSSAGPTQRPSDNSHNEHAPAS
QGLSAEH
Cyno LAIR-1 Ig-like C2-type domain amino
acid sequence (aa 29-117)
(SEQ ID NO: 174)
EPGTVIPPGRPVTIVCRGPVGVDQFRLEREDRSKFNDTKDVSQAS
PSESEARFRIDSVSEGNAGHYRCLYVKSTRWSEHSDYLDLVVK
47A1 Heavy chain variable region amino
acid sequence
(SEQ ID NO: 175)
EVQLVESGGGLVQPKGSLKLSCAASGFTFNTYAIHWVRQAPGKGL
EWVARIRSKSTNYATYYADSVKDRFTISRDDSQSMVFLQMNNLKT
EDTAMYYCVRENWYYYALDYWGQGTSVTVSS
47A1 Light chain variable region amino
acid sequence
(SEQ ID NO: 176)
DIQMTQSPASLSASVGETVTITCRASGNIHNYLTWYQQKQGKSPQ
VLVYNAKTLEDGVPSRFSGSESGTQYSLKINSLQPEDFGSYYCQH
FWSTPFTFGSGTKLEIK
47H1 Heavy chain variable region amino
acid sequence
(SEQ ID NO: 177)
EVQLVETGGGLVQPKGSLKLSCAASGFTFNINAMNWVRQAPGKGL
EWVARIRTKNNNYATFYADSVKDRFTISRDDSQSMLYLQMNNLKT
DDTAMYYCVRDRAGFFAYWGQGTPVTVSA
47H1 Light chain variable region amino
acid sequence
(SEQ ID NO: 178)
DIQMTQSPASQSASLGESVTITCLASQTIGTWLGWYRQKPGKSPQ
LLIYAATSLADGVPSRFSGSGSGTKFSFKISSLQAEDFVIYYCQQ
LYSTPLTFGSGTKLEIK
Hz47H1.v4 Heavy chain variable region
amino acid sequence
(SEQ ID NO: 179)
EVQLVESGGGLVQPGGSLRLSCAASGFTFNINAMNWVRQAPGKGL
EWVARIRTKNYNYATFYADSVKDRFTISRDDSKNSLYLQMNSLKT
EDTAVYYCVRDRAGFFAYWGQGTTVTVSS
Hz47H1.v4 Light chain variable region
amino acid sequence
(SEQ ID NO: 180)
DIQMTQSPSSLSASVGDRVTITCLASQTIGTWLGWYQQKPGKAPK
LLIYAATSLAEGVPSRFSGSGSGTDFTLTISSLQPEDFATYYCQQ
LYSTPLTFGGGTKVEIK
57D12 Heavy chain variable region amino
acid sequence
(SEQ ID NO: 181)
QVQLQQSGAELARPGASVNLSCRASGYSFTSFGISWVKQRTGQGL
EWIGEIYPRSDNTFYNEKFKGKATLTADKSSSTAYMELRSLTSED
SAVYFCARHFGSSSFDYWGQGTTLTVSS
57D12 Light chain variable region amino
acid sequence
(SEQ ID NO: 182)
ENVLTQSPPIMAASLGQKVTMTCSASSSVSSIYFHWYQQKSGTSP
KPLIHRASNLASGVPARFSGSGSGTSYSLTISSVEAEDDATYYCQ
QWSGYPLTFGGGTKLEIK
61H4 Heavy chain variable region amino
acid sequence
(SEQ ID NO: 183)
EVQLQQSGPEVLKPGASVKISCKASGYTFTDYYYMNWVKQSHGKS
LEWIGYIYPNNGATSYNQKFKGKATLTVDKSSSTAYMELRSLTSE
DSAVYYCARDGYSSNYYTMDYWGQGTSVTVSS
61H4 Light chain variable region amino
acid sequence
(SEQ ID NO: 184)
DVQMIQSPSSLSASLGDIVTMTCQASQGTSINLNWFQQKPGKAPK
LLIYGASNLEDGVPSRFSGSRYGTDFTLTISSLEDEDMATYFCLQ
HTYLPYTFGGGTKLEIK
62G10 Heavy chain variable region amino
acid sequence
(SEQ ID NO: 185)
EVQLVETGGGLVQPKGSLKLSCATSGFTFNINAMNWVRQAPGKGL
EWVARIRTKNNNFATYYADSVKDRFTISRDDSQSMLYLQMNNLKT
EDTAMYYCVRGPYFDYWGQGTTLTVSS
62G10 Light chain variable region amino
acid sequence
(SEQ ID NO: 186)
DIQMTQSPASQSASLGESVTITCLASQTIGTWLAWYQQKPGKSPQ
LLIYAATSLADGVPSRFSGSGSGTKFSFKISNLQAEDFVTYYCQQ
LYSTPYTFGGGTKLEIK
Hz62G10.v1 Heavy chain variable region
amino acid sequence
(SEQ ID NO: 187)
EVQLVESGGGLVKPGGSLRLSCAASGFTFNINAMNWVRQAPGKGL
EWVARIRTKNNNFATYYADSVKDRFTISRDDSKNTLYLQMNSLKT
EDTAVYYCVRGPYFDYWGQGTLVTVSS
Hz62G10.v1 Light chain variable region
amino acid sequence
(SEQ ID NO: 188)
DIQMTQSPSSLSASVGDRVTITCLASQTIGTWLAWYQQKPGKAPK
LLIYAATSLADGVPSRFSGSGSGTDFTLTISSLQPEDFATYYCQQ
LYSTPYTFGGGTKVEIK
108D10 Heavy chain variable region amino
acid sequence
(SEQ ID NO: 189)
EVQLVETGGGLVQPKGSLKLSCAASGFTFNINAMNWVRQAPGKGL
EWVARIRTKNNNYATYYADSVKDRFTISRDDSESMLYLQMNNLKT
EDTAMYYCVRDRYGGAMAYWGQGTSVTVSS
108D10 Light chain variable region amino
acid sequence
(SEQ ID NO: 190)
DIQMTQSSSSFSVSLGDRVTITCKASEDIYNRLAWYQQKPGNVPR
LLISSATSLETGVPSRFSGSGSGKDYTLSLTSLQSEDVATYYCQQ
YWTIPYTFGGGTKLEIK
43H2 Heavy chain variable region amino
acid sequence
(SEQ ID NO: 191)
EVQLVESGGGLVQPGRSLKVSCAASGFTFSNYGIHWIRQAPTKGL
EWVASISPSGRSTYFRDSVKGRFTISRDNAKNTLYLQLDSLRSED
TATYYCATGINYSSFDYWGQGVMVTVSS
43H2 Light chain variable region amino
acid sequence
(SEQ ID NO: 192)
DIVMTQSPTSMSISVGDRVTMNCKASQNVGSHVDWYQQKTGQSPK
LLISTASNRYTGVPDRFTGSGSGTDFTFTINNMQTEDLAVYYCMQ
SNSYPPTFGGGTKLELK
Hz47H1.v4 Heavy chain amino acid sequence
with signal sequence underlined
(SEQ ID NO: 193)
MDMRVPAQLLGLLLLWLRGARCEVQLVESGGGLVQPGGSLRLSCA
ASGFTFNINAMNWVRQAPGKGLEWVARIRTKNYNYATFYADSVKD
RFTISRDDSKNSLYLQMNSLKTEDTAVYYCVRDRAGFFAYWGQGT
TVTVSSASTKGPSVFPLAPSSKSTSGGTAALGCLVKDYFPEPVTV
SWNSGALTSGVHTFPAVLQSSGLYSLSSVVTVPSSSLGTQTYICN
VNHKPSNTKVDKKVEPKSCDKTHTCPPCPAPELLGGPSVFLFPPK
PKDTLMISRTPEVTCVVVDVSHEDPEVKFNWYVDGVEVHNAKTKP
REEQYGSTYRVVSVLTVLHQDWLNGKEYKCKVSNKALPAPIEKTI
SKAKGQPREPQVYTLPPSREEMTKNQVSLTCLVKGFYPSDIAVEW
ESNGQPENNYKTTPPVLDSDGSFFLYSKLTVDKSRWQQGNVFSCS
VMHEALHNHYTQKSLSLSPGK
Hz47H1.v4 Heavy chain amino acid sequence
without signal sequence
(SEQ ID NO: 194)
EVQLVESGGGLVQPGGSLRLSCAASGFTFNINAMNWVRQAPGKGL
EWVARIRTKNYNYATFYADSVKDRFTISRDDSKNSLYLQMNSLKT
EDTAVYYCVRDRAGFFAYWGQGTTVTVSSASTKGPSVFPLAPSSK
STSGGTAALGCLVKDYFPEPVTVSWNSGALTSGVHTFPAVLQSSG
LYSLSSVVTVPSSSLGTQTYICNVNHKPSNTKVDKKVEPKSCDKT
HTCPPCPAPELLGGPSVFLFPPKPKDTLMISRTPEVTCVVVDVSH
EDPEVKFNWYVDGVEVHNAKTKPREEQYGSTYRVVSVLTVLHQDW
LNGKEYKCKVSNKALPAPIEKTISKAKGQPREPQVYTLPPSREEM
TKNQVSLTCLVKGFYPSDIAVEWESNGQPENNYKTTPPVLDSDGS
FFLYSKLTVDKSRWQQGNVFSCSVMHEALHNHYTQKSLSLSPGK
Hz47H1.v4 Light chain amino acid sequence
with signal sequence underlined
(SEQ ID NO: 195)
MDMRVPAQLLGLLLLWLRGARCDIQMTQSPSSLSASVGDRVTITC
LASQTIGTWLGWYQQKPGKAPKLLIYAATSLAEGVPSRFSGSGSG
TDFTLTISSLQPEDFATYYCQQLYSTPLTFGGGTKVEIKRTVAAP
SVFIFPPSDEQLKSGTASVVCLLNNFYPREAKVQWKVDNALQSGN
SQESVTEQDSKDSTYSLSSTLTLSKADYEKHKVYACEVTHQGLSS
PVTKSFNRGEC
Hz47H1.v4 Light chain amino acid sequence
without signal sequence
(SEQ ID NO: 196)
DIQMTQSPSSLSASVGDRVTITCLASQTIGTWLGWYQQKPGKAPK
LLIYAATSLAEGVPSRFSGSGSGTDFTLTISSLQPEDFATYYCQQ
LYSTPLTFGGGTKVEIKRTVAAPSVFIFPPSDEQLKSGTASVVCL
LNNFYPREAKVQWKVDNALQSGNSQESVTEQDSKDSTYSLSSTLT
LSKADYEKHKVYACEVTHQGLSSPVTKSFNRGEC
Hz62G10.v1 Heavy chain amino acid sequence
with signal sequence underlined
(SEQ ID NO: 197)
MDMRVPAQLLGLLLLWLRGARCEVQLVESGGGLVKPGGSLRLSCA
ASGFTFNINAMNWVRQAPGKGLEWVARIRTKNNNFATYYADSVKD
RFTISRDDSKNTLYLQMNSLKTEDTAVYYCVRGPYFDYWGQGTLV
TVSSASTKGPSVFPLAPSSKSTSGGTAALGCLVKDYFPEPVTVSW
NSGALTSGVHTFPAVLQSSGLYSLSSVVTVPSSSLGTQTYICNVN
HKPSNTKVDKKVEPKSCDKTHTCPPCPAPELLGGPSVFLFPPKPK
DTLMISRTPEVTCVVVDVSHEDPEVKFNWYVDGVEVHNAKTKPRE
EQYGSTYRVVSVLTVLHQDWLNGKEYKCKVSNKALPAPIEKTISK
AKGQPREPQVYTLPPSREEMTKNQVSLTCLVKGFYPSDIAVEWES
NGQPENNYKTTPPVLDSDGSFFLYSKLTVDKSRWQQGNVFSCSVM
HEALHNHYTQKSLSLSPGK
Hz62G10.v1 Heavy chain amino acid sequence
without signal sequence
(SEQ ID NO: 198)
EVQLVESGGGLVKPGGSLRLSCAASGFTFNINAMNWVRQAPGKGL
EWVARIRTKNNNFATYYADSVKDRFTISRDDSKNTLYLQMNSLKT
EDTAVYYCVRGPYFDYWGQGTLVTVSSASTKGPSVFPLAPSSKST
SGGTAALGCLVKDYFPEPVTVSWNSGALTSGVHTFPAVLQSSGLY
SLSSVVTVPSSSLGTQTYICNVNHKPSNTKVDKKVEPKSCDKTHT
CPPCPAPELLGGPSVFLFPPKPKDTLMISRTPEVTCVVVDVSHED
PEVKFNWYVDGVEVHNAKTKPREEQYGSTYRVVSVLTVLHQDWLN
GKEYKCKVSNKALPAPIEKTISKAKGQPREPQVYTLPPSREEMTK
NQVSLTCLVKGFYPSDIAVEWESNGQPENNYKTTPPVLDSDGSFF
LYSKLTVDKSRWQQGNVFSCSVMHEALHNHYTQKSLSLSPGK
Hz62G10.v1 Light chain amino acid sequence
with signal sequence underlined
(SEQ ID NO: 199)
MDMRVPAQLLGLLLLWLRGARCDIQMTQSPSSLSASVGDRVTITC
LASQTIGTWLAWYQQKPGKAPKLLIYAATSLADGVPSRFSGSGSG
TDFTLTISSLQPEDFATYYCQQLYSTPYTFGGGTKVEIKRTVAAP
SVFIFPPSDEQLKSGTASVVCLLNNFYPREAKVQWKVDNALQSGN
SQESVTEQDSKDSTYSLSSTLTLSKADYEKHKVYACEVTHQGLSS
PVTKSFNRGEC
Hz62G10.v1 Light chain amino acid sequence
without signal sequence
(SEQ ID NO: 200)
DIQMTQSPSSLSASVGDRVTITCLASQTIGTWLAWYQQKPGKAPK
LLIYAATSLADGVPSRFSGSGSGTDFTLTISSLQPEDFATYYCQQ
LYSTPYTFGGGTKVEIKRTVAAPSVFIFPPSDEQLKSGTASVVCL
LNNFYPREAKVQWKVDNALQSGNSQESVTEQDSKDSTYSLSSTLT
LSKADYEKHKVYACEVTHQGLSSPVTKSFNRGEC
Mouse LAIR-1 amino acid sequence with
predicted signal sequence underlined
(SEQ ID NO: 201)
MSLHPVILLVLVLCLGWKINTQEGSLPDITIFPNSSLMISQGTFV
TVVCSYSDKHDLYNMVRLEKDGSTFMEKSTEPYKTEDEFEIGPVN
ETITGHYSCIYSKGITWSERSKTLELKVIKENVIQTPAPGPTSDT
SWLKTYSIYIFTVVSVIFLLCLSALLFCFLRHRQKKQGLPNNKRQ
QQRPEERLNLATNGLEMTPDIVADDRLPEDRWTETWTPVAGDLQE
VTYIQLDHHSLTQRAVGAVTSQSTDMAESSTYAAIIRH
Mouse LAIR-1 amino acid sequence without
predicted signal sequence
(SEQ ID NO: 202)
QEGSLPDITIFPNSSLMISQGTFVTVVCSYSDKHDLYNMVRLEKD
GSTFMEKSTEPYKTEDEFEIGPVNETITGHYSCIYSKGITWSERS
KTLELKVIKENVIQTPAPGPTSDTSWLKTYSIYIFTVVSVIFLLC
LSALLFCFLRHRQKKQGLPNNKRQQQRPEERLNLATNGLEMTPDI
VADDRLPEDRWTETWTPVAGDLQEVTYIQLDHHSLTQRAVGAVTS
QSTDMAESSTYAAIIRH
Mouse LAIR-1 extracellular domain (aa 22-144)
(SEQ ID NO: 203)
QEGSLPDITIFPNSSLMISQGTFVTVVCSYSDKHDLYNMVRLEKD
GSTFMEKSTEPYKTEDEFEIGPVNETITGHYSCIYSKGITWSERS
KTLELKVIKENVIQTPAPGPTSDTSWLKTYSIY
Mouse LAIR-1 Ig-like C2-type domain amino
acid sequence (aa 27-114)
(SEQ ID NO: 204)
PDITIFPNSSLMISQGTFVTVVCSYSDKHDLYNMVRLEKDGSTFM
EKSTEPYKTEDEFEIGPVNETITGHYSCIYSKGITWSERSKTL
6D8 Heavy chain variable region amino
acid sequence
(SEQ ID NO: 205)
QVQLKESGPGLVAPSQSLSIACTVSGFSLTNYAISWVRQPPGKGL
EWLGVIWTGGGTNYNSTLKSRLSISKDNSKSQVFLKMNSLQTDDT
ARYNCARNSGDWYFDVWGPGTTVTVSS
6D8 Light chain variable region amino
acid sequence
(SEQ ID NO: 206)
DIVMSQSPSSLAVSVGEKVTMSCKSSQSLLYSSNQKNYLAWYQQK
PGQSPHLLIYWASTRESGVPDRFIGSGSGTDFTLTISSVKAEDLA
VYYCQQYYDYPPTFGSGTKLEIK
10G4 Heavy chain variable region amino
acid sequence
(SEQ ID NO: 207)
QVQLKESGPGLVAPSQSLSITCTVSGFSLSSYPISWVRQPPGKGL
EWLGIIWTGGGTNYNSALKSRLSISKDNSKSQVFLKMNSLQTDDT
ARYYCASQNWDVNSALDYWGQGTSVTVSS
10G4 Light chain variable region amino
acid sequence
(SEQ ID NO: 208)
DIQMTQSPASLSVSVGETVTITCRASENIYSNLAWYQQKQGKSPQ
LLVYTATNLADGVPSRFSGSGSGTQYSLRINRLQSEDFGNYYCQH
FWNAPWTFGGGTKLEIK
15A3 Heavy chain variable region amino
acid sequence
(SEQ ID NO: 209)
EIQLQQSGPELVKPGASVKVSCKASGYSFTDYNMYWVKQSHGKSL
EWIGYIDPYNGGTSYNQKFKGKATLTVDKSSSTAFMHLNSLTSED
SAVYYCARLRSPYDYDRGDYVMDYWGQGTSVTVSS
15A3 Light chain variable region amino
acid sequence
(SEQ ID NO: 210)
DIVLTQSPASLAVSLGQRATISCRASKSVSTSGYTYMHWYQQKPG
QPPKLLIYLASNLESGVPARFSGSGSGTDFTLNIHPVEEEDAATY
YCQHSRELPLTFGAGTKLELK
6D8 Heavy chain amino acid sequence with
signal sequence underlined
(SEQ ID NO: 211)
MDMRVPAQLLGLLLLWLRGARCQVQLKESGPGLVAPSQSLSIACT
VSGFSLTNYAISWVRQPPGKGLEWLGVIWTGGGTNYNSTLKSRLS
ISKDNSKSQVFLKMNSLQTDDTARYNCARNSGDWYFDVWGPGTTV
TVSSAKTTAPSVYPLAPVCGDTTGSSVTLGCLVKGYFPEPVTLTW
NSGSLSSGVHTFPAVLQSDLYTLSSSVTVTSSTWPSQSITCNVAH
PASSTKVDKKIEPRGPTIKPCPPCKCPAPNAAGGPSVFIFPPKIK
DVLMISLSPIVTCVVVDVSEDDPDVQISWFVNNVEVHTAQTQTHR
EDYNSTLRVVSALPIQHQDWMSGKEFKCKVNNKDLGAPIERTISK
PKGSVRAPQVYVLPPPEEEMTKKQVTLTCMVTDFMPEDIYVEWTN
NGKTELNYKNTEPVLDSDGSYFMYSKLRVEKKNWVERNSYSCSVV
HEGLHNHHTTKSFSRTPGK
6D8 Heavy chain amino acid sequence without
signal sequence
(SEQ ID NO: 212)
QVQLKESGPGLVAPSQSLSIACTVSGFSLTNYAISWVRQPPGKGL
EWLGVIWTGGGTNYNSTLKSRLSISKDNSKSQVFLKMNSLQTDDT
ARYNCARNSGDWYFDVWGPGTTVTVSSAKTTAPSVYPLAPVCGDT
TGSSVTLGCLVKGYFPEPVTLTWNSGSLSSGVHTFPAVLQSDLYT
LSSSVTVTSSTWPSQSITCNVAHPASSTKVDKKIEPRGPTIKPCP
PCKCPAPNAAGGPSVFIFPPKIKDVLMISLSPIVTCVVVDVSEDD
PDVQISWFVNNVEVHTAQTQTHREDYNSTLRVVSALPIQHQDWMS
GKEFKCKVNNKDLGAPIERTISKPKGSVRAPQVYVLPPPEEEMTK
KQVTLTCMVTDFMPEDIYVEWTNNGKTELNYKNTEPVLDSDGSYF
MYSKLRVEKKNWVERNSYSCSVVHEGLHNHHTTKSFSRTPGK
6D8 Light chain amino acid sequence with
signal sequence underlined
(SEQ ID NO: 213)
MDMRVPAQLLGLLLLWLRGARCDIVMSQSPSSLAVSVGEKVTMSC
KSSQSLLYSSNQKNYLAWYQQKPGQSPHLLIYWASTRESGVPDRF
IGSGSGTDFTLTISSVKAEDLAVYYCQQYYDYPPTFGSGTKLEIK
RADAAPTVSIFPPSSEQLTSGGASVVCFLNNFYPKDINVKWKIDG
SERQNGVLNSWTDQDSKDSTYSMSSTLTLTKDEYERHNSYTCEAT
HKTSTSPIVKSFNRNEC
6D8 Light chain amino acid sequence without
signal sequence
(SEQ ID NO: 214)
DIVMSQSPSSLAVSVGEKVTMSCKSSQSLLYSSNQKNYLAWYQQK
PGQSPHLLIYWASTRESGVPDRFIGSGSGTDFTLTISSVKAEDLA
VYYCQQYYDYPPTFGSGTKLEIKRADAAPTVSIFPPSSEQLTSGG
ASVVCFLNNFYPKDINVKWKIDGSERQNGVLNSWTDQDSKDSTYS
MSSTLTLTKDEYERHNSYTCEATHKTSTSPIVKSFNRNEC
10G4 Heavy chain amino acid sequence with
signal sequence underlined
(SEQ ID NO: 215)
MDMRVPAQLLGLLLLWLRGARCQVQLKESGPGLVAPSQSLSITCT
VSGFSLSSYPISWVRQPPGKGLEWLGIIWTGGGTNYNSALKSRLS
ISKDNSKSQVFLKMNSLQTDDTARYYCASQNWDVNSALDYWGQGT
SVTVSSAKTTAPSVYPLAPVCGDTTGSSVTLGCLVKGYFPEPVTL
TWNSGSLSSGVHTFPAVLQSDLYTLSSSVTVTSSTWPSQSITCNV
AHPASSTKVDKKIEPRGPTIKPCPPCKCPAPNAAGGPSVFIFPPK
IKDVLMISLSPIVTCVVVDVSEDDPDVQISWFVNNVEVHTAQTQT
HREDYNSTLRVVSALPIQHQDWMSGKEFKCKVNNKDLGAPIERTI
SKPKGSVRAPQVYVLPPPEEEMTKKQVTLTCMVTDFMPEDIYVEW
TNNGKTELNYKNTEPVLDSDGSYFMYSKLRVEKKNWVERNSYSCS
VVHEGLHNHHTTKSFSRTPGK
10G4 Heavy chain amino acid sequence without
signal sequence
(SEQ ID NO: 216)
QVQLKESGPGLVAPSQSLSITCTVSGFSLSSYPISWVRQPPGKGL
EWLGIIWTGGGTNYNSALKSRLSISKDNSKSQVFLKMNSLQTDDT
ARYYCASQNWDVNSALDYWGQGTSVTVSSAKTTAPSVYPLAPVCG
DTTGSSVTLGCLVKGYFPEPVTLTWNSGSLSSGVHTFPAVLQSDL
YTLSSSVTVTSSTWPSQSITCNVAHPASSTKVDKKIEPRGPTIKP
CPPCKCPAPNAAGGPSVFIFPPKIKDVLMISLSPIVTCVVVDVSE
DDPDVQISWFVNNVEVHTAQTQTHREDYNSTLRVVSALPIQHQDW
MSGKEFKCKVNNKDLGAPIERTISKPKGSVRAPQVYVLPPPEEEM
TKKQVTLTCMVTDFMPEDIYVEWTNNGKTELNYKNTEPVLDSDGS
YFMYSKLRVEKKNWVERNSYSCSVVHEGLHNHHTTKSFSRTPGK
10G4 Light chain amino acid sequence with
signal sequence underlined
(SEQ ID NO: 217)
MDMRVPAQLLGLLLLWLRGARCDIQMTQSPASLSVSVGETVTITC
RASENIYSNLAWYQQKQGKSPQLLVYTATNLADGVPSRFSGSGSG
TQYSLRINRLQSEDFGNYYCQHFWNAPWTFGGGTKLEIKRADAAP
TVSIFPPSSEQLTSGGASVVCFLNNFYPKDINVKWKIDGSERQNG
VLNSWTDQDSKDSTYSMSSTLTLTKDEYERHNSYTCEATHKTSTS
PIVKSFNRNEC
10G4 Light chain amino acid sequence without
signal sequence
(SEQ ID NO: 218)
DIQMTQSPASLSVSVGETVTITCRASENIYSNLAWYQQKQGKSPQ
LLVYTATNLADGVPSRFSGSGSGTQYSLRINRLQSEDFGNYYCQH
FWNAPWTFGGGTKLEIKRADAAPTVSIFPPSSEQLTSGGASVVCF
LNNFYPKDINVKWKIDGSERQNGVLNSWTDQDSKDSTYSMSSTLT
LTKDEYERHNSYTCEATHKTSTSPIVKSFNRNEC
15A3 Heavy chain amino acid sequence with
signal sequence underlined
(SEQ ID NO: 219)
MDMRVPAQLLGLLLLWLRGARCEIQLQQSGPELVKPGASVKVSCK
ASGYSFTDYNMYWVKQSHGKSLEWIGYIDPYNGGTSYNQKFKGKA
TLTVDKSSSTAFMHLNSLTSEDSAVYYCARLRSPYDYDRGDYVMD
YWGQGTSVTVSSAKTTAPSVYPLAPVCGDTTGSSVTLGCLVKGYF
PEPVTLTWNSGSLSSGVHTFPAVLQSDLYTLSSSVTVTSSTWPSQ
SITCNVAHPASSTKVDKKIEPRGPTIKPCPPCKCPAPNAAGGPSV
FIFPPKIKDVLMISLSPIVTCVVVDVSEDDPDVQISWFVNNVEVH
TAQTQTHREDYNSTLRVVSALPIQHQDWMSGKEFKCKVNNKDLGA
PIERTISKPKGSVRAPQVYVLPPPEEEMTKKQVTLTCMVTDFMPE
DIYVEWTNNGKTELNYKNTEPVLDSDGSYFMYSKLRVEKKNWVER
NSYSCSVVHEGLHNHHTTKSFSRTPGK
15A3 Heavy chain amino acid sequence without
signal sequence
(SEQ ID NO: 220)
EIQLQQSGPELVKPGASVKVSCKASGYSFTDYNMYWVKQSHGKSL
EWIGYIDPYNGGTSYNQKFKGKATLTVDKSSSTAFMHLNSLTSED
SAVYYCARLRSPYDYDRGDYVMDYWGQGTSVTVSSAKTTAPSVYP
LAPVCGDTTGSSVTLGCLVKGYFPEPVTLTWNSGSLSSGVHTFPA
VLQSDLYTLSSSVTVTSSTWPSQSITCNVAHPASSTKVDKKIEPR
GPTIKPCPPCKCPAPNAAGGPSVFIFPPKIKDVLMISLSPIVTCV
VVDVSEDDPDVQISWFVNNVEVHTAQTQTHREDYNSTLRVVSALP
IQHQDWMSGKEFKCKVNNKDLGAPIERTISKPKGSVRAPQVYVLP
PPEEEMTKKQVTLTCMVTDFMPEDIYVEWTNNGKTELNYKNTEPV
LDSDGSYFMYSKLRVEKKNWVERNSYSCSVVHEGLHNHHTTKSFS
RTPGK
15A3 Light chain amino acid sequence with
signal sequence underlined
(SEQ ID NO: 221)
MDMRVPAQLLGLLLLWLRGARCDIVLTQSPASLAVSLGQRATISC
RASKSVSTSGYTYMHWYQQKPGQPPKLLIYLASNLESGVPARFSG
SGSGTDFTLNIHPVEEEDAATYYCQHSRELPLTFGAGTKLELKRA
DAAPTVSIFPPSSEQLTSGGASVVCFLNNFYPKDINVKWKIDGSE
RQNGVLNSWTDQDSKDSTYSMSSTLTLTKDEYERHNSYTCEATHK
TSTSPIVKSFNRNEC
15A3 Light chain amino acid sequence without
signal sequence
(SEQ ID NO: 222)
DIVLTQSPASLAVSLGQRATISCRASKSVSTSGYTYMHWYQQKPG
QPPKLLIYLASNLESGVPARFSGSGSGTDFTLNIHPVEEEDAATY
YCQHSRELPLTFGAGTKLELKRADAAPTVSIFPPSSEQLTSGGAS
VVCFLNNFYPKDINVKWKIDGSERQNGVLNSWTDQDSKDSTYSMS
STLTLTKDEYERHNSYTCEATHKTSTSPIVKSFNRNEC
Human LAIR-2 amino acid sequence with
predicted signal sequence underlined
(SEQ ID NO: 223)
MSPHLTALLGLVLCLAQTIHTQEGALPRPSISAEPGTVISPGSHV
TFMCRGPVGVOTFRLEREDRAKYKDSYNVFRLGPSESEARFHIDS
VSEGNAGLYRCLYYKPPGWSEHSDFLELLVKESSGGPDSPDTEPG
SSAGTVPGTEASGFDAP
Human LAIR-2 amino acid sequence without
predicted signal sequence
(SEQ ID NO: 224)
QEGALPRPSISAEPGTVISPGSHVTFMCRGPVGVQTFRLEREDRA
KYKDSYNVFRLGPSESEARFHIDSVSEGNAGLYRCLYYKPPGWSE
HSDFLELLVKESSGGPDSPDTEPGSSAGTVPGTEASGFDAP
Human LAIR-2 amino acid sequence Ig-like
C2 type domain (aa 29-177)
(SEQ ID NO: 225)
PSISAEPGTVISPGSHVTFMCRGPVGVQTFRLEREDRAKYKDSYN
VFRLGPSESEARFHIDSVSEGNAGLYRCLYYKPPGWSEHSDFLE

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of activating an immune cell associated with a tumor or a tumor microenvironment, wherein the method comprises contacting the immune cell with an effective amount of an anti-ILT3 antibody and an effective amount of an anti-LAIR-1 antibody.

2. The method of claim 1, wherein the immune cell is a myeloid cell.

3. The method of claim 2, wherein the myeloid cell is a monocyte, a macrophage, a dendritic cell, or an APC.

4. The method of claim 1, wherein the immune cell is a T cell.

5. The method of claim 4, wherein the T cell is a cytotoxic T-cell (CTL).

6. The method of claim 1, wherein the immune cell is a natural killer cell.

7. A method of reducing or inhibiting immune suppressive activity of an immune cell associated with a tumor or a tumor microenvironment, wherein the method comprises contacting the immune cell with an effective amount of an anti-ILT3 antibody and an effective amount of an anti-LAIR-1 antibody.

8. The method of claim 7, wherein the immune cell is a regulatory T-cell (Treg).

9. The method of claim 7, wherein the immune cell is a tolerogenic dendritic cell.

10. The method of claim 7, wherein the immune cell is a myeloid-derived suppressor cell (MDSC).

11. The method of any one of claims 1-10, further comprising contacting the immune cell with an additional therapeutic agent.

12. The method of claim 11, wherein the additional therapeutic agent is an immune-checkpoint inhibitor.

13. The method of claim 12, wherein the immune-checkpoint inhibitor is a PD-1 inhibitor or a PD-L1 inhibitor.

14. A method of activating an immune cell in a subject diagnosed with cancer, wherein the method comprises administering to the subject a therapeutically effective amount of an anti-ILT3 antibody and a therapeutically effective amount of an anti-LAIR-1 antibody.

15. The method of claim 14, wherein the immune cell is a myeloid cell.

16. The method of claim 15, wherein the myeloid cell is a monocyte, a macrophage, a dendritic cell, or an APC.

17. The method of claim 14, wherein the immune cell is a T cell.

18. The method of claim 17, wherein the T cell is a cytotoxic T-cell (CTL).

19. The method of claim 14, wherein the immune cell is a natural killer cell.

20. A method of reducing or inhibiting immune suppressive activity of in an immune cell in a subject diagnosed with cancer, wherein the method comprises administering to the subject a therapeutically effective amount of an anti-ILT3 antibody and a therapeutically effective amount of an anti-LAIR-1 antibody.

21. The method of claim 20, wherein the immune cell is a regulatory T-cell (Treg).

22. The method of claim 20, wherein the immune cell is a tolerogenic dendritic cell.

23. The method of claim 20, wherein the immune cell is a myeloid-derived suppressor cell (MDSC).

24. A method of reversing stromal-mediated immunosuppression in a subject diagnosed with cancer, wherein the method comprises administering to the subject a therapeutically effective amount of an anti-ILT3 antibody and a therapeutically effective amount of an anti-LAIR-1 antibody.

25. A method of treating cancer in a subject, wherein the method comprises administering to the subject a therapeutically effective amount of an anti-ILT3 antibody and a therapeutically effective amount of an anti-LAIR-1 antibody.

26. A method of enhancing an immune response to cancer in a subject diagnosed with the cancer, wherein the method comprises administering to the subject a therapeutically effective amount of an anti-ILT3 antibody and a therapeutically effective amount of an anti-LAIR-1 antibody.

27. The method of any one of claims 14-26, wherein the cancer is a hematologic cancer.

28. The method of any one of claims 14-26, wherein the cancer is a solid tumor.

29. The method of claim 28, wherein the cancer is a pancreatic cancer, a breast cancer, a mesothelioma, a gastric cancer, an NSCLC, a cervical cancer, an endocervical cancer, a biliary duct cancer, a SCCHN, a bladder cancer, an urothelial cancer, a CRC, an esophageal cancer, an ovarian cancer, an RCC, a prostate cancer, or a melanoma.

30. The method of claim 29, wherein the pancreatic cancer is pancreatic ductal adenocarcinoma.

31. The method of any one of claims 14-26, wherein the cancer is a tumor mutational burden-high (TMB-H) cancer.

32. The method of any one of claims 14-26, wherein the cancer is a microsatellite instability-high (MSI-H) cancer.

33. The method of any one of claims 14-32, further comprising administering to the subject an additional therapeutic agent.

34. The method of claim 33, wherein the additional therapeutic agent is an immune-checkpoint inhibitor.

35. The method of claim 34, wherein the immune-checkpoint inhibitor is a PD-1 inhibitor or a PD-L1 inhibitor.

36. The method of any one of claims 14-35, wherein the subject is a human.

37. The method of any one of claims 1-36, wherein the anti-ILT3 antibody inhibits binding of ILT3 to fibronectin.

38. The method of any one of claims 1-37, wherein the anti-ILT3 antibody inhibits binding of ILT3 to APOE.

39. The method of any one of claims 1-38, wherein the anti-ILT3 antibody inhibits binding of ILT3 to CNTFR.

40. The method of any one of claims 1-39, wherein the anti-ILT3 antibody inhibits ILT3 activity.

41. The method of any one of claims 1-40, wherein the anti-LAIR-1 antibody inhibits LAIR-1 activity.

42. The method of any one of claims 1-41, wherein the anti-LAIR-1 antibody inhibits binding of LAIR-1 to collagen.

43. The method of any one of claims 1-42, wherein the anti-LAIR-1 antibody inhibits binding of LAIR-1 to MARCO.

44. The method of any one of claims 1-43, wherein the anti-LAIR-1 antibody inhibits binding of LAIR-1 to COLEC12.

45. The method of any one of claims 1-44, wherein the anti-ILT3 antibody comprises:
(a) a heavy chain variable region (VH) comprising a VH-complementarity determining region (CDR)1, a VH-CDR2, and a VH-CDR3 from the amino acid sequence of SEQ ID NO:111; and a light chain variable region (VL) comprising a VL-CDR1, a VL-CDR2, and a VL-CDR3 from the amino acid sequence of SEQ ID NO:112; or
(b) a VH comprising a VH-CDR1, a VH-CDR2, and a VH-CDR3 from the amino acid sequence of SEQ ID NO:123; and a VL comprising a VL-CDR1, a VL-CDR2, and a VL-CDR3 from the amino acid sequence of SEQ ID NO:124.

46. The method of claim 45, wherein:
(a) the VH comprises a VH-CDR1 comprising the amino acid sequence GFTFSSYGMS (SEQ ID NO:27), a VH-CDR2 comprising the amino acid sequence TISGGGSYTNYPDSVKG (SEQ ID NO:28), and a VH-CDR3 comprising the amino acid sequence REWRYTLYAMDY (SEQ ID NO:105); and the VL comprises a VL-CDR1 comprising the amino acid sequence RASESVESYGSSFMH (SEQ ID NO:106), a VL-CDR2 comprising the amino acid sequence LTSNLES (SEQ ID NO:31), and a VL-CDR3 comprising the amino acid sequence QQNNEDPFT (SEQ ID NO:32);
(b) the VH comprises a VH-CDR1 comprising the amino acid sequence GFTFSSY (SEQ ID NO:33), a VH-CDR2 comprising the amino acid sequence SGGGSY (SEQ ID NO:34), and a VH-CDR3 comprising the amino acid sequence REWRYTLYAMDY (SEQ ID NO:105); and the VL comprises a VL-CDR1 comprising the amino acid sequence RASESVESYGSSFMH (SEQ ID NO:106), a VL-CDR2 comprising the amino acid sequence LTSNLES (SEQ ID NO:31), and a VL-CDR3 comprising the amino acid sequence QQNNEDPFT (SEQ ID NO:32);
(c) the VH comprises a VH-CDR1 comprising the amino acid sequence GFTFSSYGMS (SEQ ID NO:27), a VH-CDR2 comprising the amino acid sequence TISGGGSYTN (SEQ ID NO:35), and a VH-CDR3 comprising the amino acid sequence REWRYTLYAMDY (SEQ ID NO:105); and the VL comprises a VL-CDR1 comprising the amino acid sequence RASESVESYGSSFMH (SEQ ID NO:106), a VL-CDR2 comprising the amino acid sequence LTSNLES (SEQ ID NO:31), and a VL-CDR3 comprising the amino acid sequence QQNNEDPFT (SEQ ID NO:32);
(d) the VH comprises a VH-CDR1 comprising the amino acid sequence SYGMS (SEQ ID NO:36), a VH-CDR2 comprising the amino acid sequence TISGGGSYTNYPDSVKG (SEQ ID NO:28), and a VH-CDR3 comprising the amino acid sequence REWRYTLYAMDY (SEQ ID NO:105); and the VL comprises a VL-CDR1 comprising the amino acid sequence RASESVESYGSSFMH (SEQ ID NO:106), a VL-CDR2 comprising the amino acid sequence LTSNLES (SEQ ID NO:31), and a VL-CDR3 comprising the amino acid sequence QQNNEDPFT (SEQ ID NO:32); or
(e) the VH comprises a VH-CDR1 comprising the amino acid sequence SSYGMS (SEQ ID NO:37), a VH-CDR2 comprising the amino acid sequence WVATISGGGSYTN (SEQ ID NO:38), and a VH-CDR3 comprising the amino acid sequence ARREWRYTLYAMD (SEQ ID NO:107), and the VL comprises a VL-CDR1 comprising the amino acid sequence ESYGSSFMHWY (SEQ ID NO:108), a VL-CDR2 comprising the amino acid sequence LLIYLTSNLE (SEQ ID NO:41), and a VL-CDR3 comprising the amino acid sequence QQNNEDPF (SEQ ID NO:42).

47. The method of claim 46, wherein:
(a) the VH comprises an amino acid sequence having at least 80% sequence identity to the amino acid sequence of SEQ ID NO:123;
(b) the VL comprises an amino acid sequence having at least 80% sequence identity to the amino acid sequence of SEQ ID NO:124; or
(c) the VH comprises an amino acid sequence having at least 80% sequence identity to the amino acid sequence of SEQ ID NO:123 and the VL comprises an amino acid sequence having at least 80% sequence identity to the amino acid sequence of SEQ ID NO:124.

48. The method of claim 47, wherein the VH comprises the amino acid sequence of SEQ ID NO:123 and the VL comprises the amino acid sequence of SEQ ID NO:124.

49. The method of any one of claims 1-48, wherein the anti-ILT3 antibody comprises:
(a) a heavy chain comprising an amino acid sequence having at least 80% sequence identity to the amino acid sequence of SEQ ID NO:126;
(b) a light chain comprising an amino acid sequence having at least 80% sequence identity to the amino acid sequence of SEQ ID NO:128; or
(c) a heavy chain comprising an amino acid sequence having at least 80% sequence identity to the amino acid sequence of SEQ ID NO:126, and a light chain comprising an amino acid sequence having at least 80% sequence identity to the amino acid sequence of SEQ ID NO:128.

50. The method of claim 49, wherein the heavy chain comprises the amino acid sequence of SEQ ID NO:126 and the light chain comprises the amino acid sequence of SEQ ID NO:128.

51. The method of any one of claims 1-50, wherein the anti-LAIR-1 antibody comprises a heavy chain variable region (VH) comprising a VH-complementarity determining region (CDR)1, a VH-CDR2 and a VH-CDR3 from SEQ ID NO:179, and a light chain variable region (VL) comprising a VL-CDR1, a VL-CDR2 and a VL-CDR3 from SEQ ID NO:180.

52. The method of claim 51, wherein:
(a) the VH comprises the VH-CDR1 comprising the amino acid sequence of SEQ ID NO:242, the VH-CDR2 comprising the amino acid sequence of SEQ ID NO:257, and the VH-CDR3 comprising the amino acid sequence of SEQ ID NO:244; and the VL comprises the VL-CDR1 comprising the amino acid sequence of SEQ ID NO:245, the VL-CDR2 comprising the amino acid sequence of SEQ ID NO:258, and the VL-CDR3 comprising the amino acid sequence of SEQ ID NO:247;
(b) the VH comprises the VH-CDR1 comprising the amino acid sequence of SEQ ID NO:248, the VH-CDR2 comprising the amino acid sequence of SEQ ID NO:259, and the VH-CDR3 comprising the amino acid sequence of SEQ ID NO:244; and the VL comprises the VL-CDR1 comprising the amino acid sequence of SEQ ID NO:245, the VL-CDR2 comprising the amino acid sequence of SEQ ID NO:258, and the VL-CDR3 comprising the amino acid sequence of SEQ ID NO:247;
(c) the VH comprises the VH-CDR1 comprising the amino acid sequence of SEQ ID NO:242, the VH-CDR2 comprising the amino acid sequence of SEQ ID NO:260, and the VH-CDR3 comprising the amino acid sequence of SEQ ID NO:244; and the VL comprises the VL-CDR1 comprising the amino acid sequence of SEQ ID NO:245, the VL-CDR2 comprising the amino acid sequence of SEQ ID NO:258, and the VL-CDR3 comprising the amino acid sequence of SEQ ID NO:247;
(d) the VH comprises the VH-CDR1 comprising the amino acid sequence of SEQ ID NO:250, the VH-CDR2 comprising the amino acid sequence of SEQ ID NO:257, and the VH-CDR3 comprising the amino acid sequence of SEQ ID NO:244; and the VL comprises the VL-CDR1 comprising the amino acid sequence of SEQ ID NO:245, the VL-CDR2 comprising the amino acid sequence of SEQ ID NO:258, and the VL-CDR3 comprising the amino acid sequence of SEQ ID NO:247; or
(e) the VH comprises the VH-CDR1 comprising the amino acid sequence of SEQ ID NO:251, the VH-CDR2 comprising the amino acid sequence of SEQ ID NO:261, and the VH-CDR3 comprising the amino acid sequence of SEQ ID NO:253; and the VL comprises the VL-CDR1 comprising the amino acid sequence of SEQ ID NO:254, the VL-CDR2 comprising the amino acid sequence of SEQ ID NO:255, and the VL-CDR3 comprising the amino acid sequence of SEQ ID NO:256.

53. The method of claim 51 or 52, wherein the VH has at least 80% sequence identity to the amino acid sequence of SEQ ID NO:179, and/or the VL has at least 80% sequence identity to the amino acid sequence of SEQ ID NO:180.

54. The method of claim 53, wherein the VH comprises the amino acid sequence of SEQ ID NO:179, and/or the VL comprises the amino acid sequence of SEQ ID NO:180.

55. The method of any one of claims 1-54, wherein the anti-LAIR-1 antibody comprises: a heavy chain comprising an amino acid sequence having 80% identity to the amino acid sequence of SEQ ID NO:194, and/or a light chain comprising an amino acid sequence having 80% identity to the amino acid sequence of SEQ ID NO:196.

56. The method of claim 55, wherein the heavy chain comprises the amino acid sequence of SEQ ID NO:194, and/or the light chain comprises the amino acid sequence of SEQ ID NO:196.

57. The method of any one of claims 1-56, wherein the anti-ILT3 antibody and the anti-LAIR-1 antibody are formulated into two separate pharmaceutical compositions, where a first pharmaceutical composition comprises the anti-ILT3 antibody and a pharmaceutically acceptable carrier; and a second pharmaceutical composition comprises the anti-LAIR-1 antibody and a pharmaceutically acceptable carrier.

58. The method of any one of claims 1-56, wherein the anti-ILT3 antibody and the anti-LAIR-1 antibody are formulated into a pharmaceutical composition, wherein the pharmaceutical composition comprises the anti-ILT3 antibody, the anti-LAIR-1 antibody, and a pharmaceutically acceptable carrier.

59. Use of an anti-ILT3 antibody and an anti-LAIR-1 antibody for activating an immune cell associated with a tumor or a tumor microenvironment, wherein the use comprises contacting the immune cell with an effective amount of the anti-ILT3 antibody and an effective amount of the anti-LAIR-1 antibody.

60. The use of claim 59, wherein the immune cell is a myeloid cell.

61. The use of claim 60, wherein the myeloid cell is a monocyte, a macrophage, a dendritic cell, or an APC.

62. The use of claim 60, wherein the immune cell is a T cell.

63. The use of claim 62, wherein the T cell is a cytotoxic T-cell (CTL).

64. The use of claim 60, wherein the immune cell is a natural killer cell.

65. Use of an anti-ILT3 antibody and an anti-LAIR-1 antibody for reducing or inhibiting immune suppressive activity of an immune cell associated with a tumor or a tumor microenvironment, wherein the use comprises contacting the immune cell with an effective amount of the anti-ILT3 antibody and an effective amount of the anti-LAIR-1 antibody.

66. The use of claim 65, wherein the immune cell is a regulatory T-cell (Treg).

67. The use of claim 65, wherein the immune cell is a tolerogenic dendritic cell.

68. The use of claim 65, wherein the immune cell is a myeloid-derived suppressor cell (MDSC).

69. The use of any one of claims 59-68, further comprising contacting the immune cell with an additional therapeutic agent.

70. The use of claim 69, wherein the additional therapeutic agent is an immune checkpoint inhibitor.

71. The use of claim 70, wherein the immune-checkpoint inhibitor is a PD-1 inhibitor or a PD-L1 inhibitor.

72. Use of an anti-ILT3 antibody and an anti-LAIR-1 antibody for activating an immune cell in a subject diagnosed with a cancer, wherein the use comprises administering to the subject a therapeutically effective amount of the anti-ILT3 antibody and a therapeutically effective amount of the anti-LAIR-1 antibody.

73. The use of claim 72, wherein the immune cell is a myeloid cell.

74. The use of claim 73, wherein the myeloid cell is a monocyte, a macrophage, a dendritic cell, or an APC.

75. The use of claim 72, wherein the immune cell is a T cell.

76. The use of claim 75, wherein the T cell is a cytotoxic T-cell (CTL).

77. The use of claim 72, wherein the immune cell is a natural killer cell.

78. Use of an anti-ILT3 antibody and an anti-LAIR-1 antibody for reducing or inhibiting immune suppressive activity of an immune cell in a subject diagnosed with cancer, wherein the use comprises administering to the subject a therapeutically effective amount of the anti-ILT3 antibody and a therapeutically effective amount of the anti-LAIR-1 antibody.

79. The use of claim 78, wherein the immune cell is a tolerogenic dendritic cell.

80. The use of claim 78, wherein the immune cell is a regulatory T-cell (Treg).

81. The use of claim 78, wherein the immune cell is a myeloid-derived suppressor cell (MDSC).

82. Use of an anti-ILT3 antibody and an anti-LAIR-1 antibody for reversing stromal-mediated immunosuppression in a subject diagnosed with cancer, wherein the use comprises administering to the subject a therapeutically effective amount of the anti-ILT3 antibody and a therapeutically effective amount of the anti-LAIR-1 antibody.

83. Use of an anti-ILT3 antibody and an anti-LAIR-1 antibody for treating cancer in a subject, wherein the use comprises administering to the subject a therapeutically effective amount of the anti-ILT3 antibody and a therapeutically effective amount of the anti-LAIR-1 antibody.

84. Use of an anti-ILT3 antibody and an anti-LAIR-1 antibody for enhancing an immune response to cancer in a subject diagnosed with the cancer, wherein the use comprises administering to the subject a therapeutically effective amount of the anti-ILT3 antibody and a therapeutically effective amount of the anti-LAIR-1 antibody.

85. The use of any one of claims 59-84, wherein the tumor or cancer is a hematologic cancer.

86. The use of any one of claims 59-84, wherein the tumor or cancer is a solid tumor.

87. The use of claim 86, wherein the tumor or cancer is a pancreatic cancer, a breast cancer, a mesothelioma, a gastric cancer, an NSCLC, a cervical cancer, an endocervical cancer, a biliary duct cancer, a SCCHN, a bladder cancer, an urothelial cancer, a CRC, an esophageal cancer, an ovarian cancer, a RCC, a prostate cancer, or a melanoma.

88. The use of claim 87, wherein the pancreatic cancer is pancreatic ductal adenocarcinoma.

89. The use of any of claims 59-84, wherein the tumor or cancer is a tumor mutational burden-high (TMB-H) cancer.

90. The use of any of claims 59-84, wherein the tumor or cancer is a microsatellite instability-high (MSI-H) cancer.

91. The use of any of claims 72-90, further comprising administering to the subject an additional therapeutic agent.

92. The use of claim 91, wherein the additional therapeutic agent is an immune-checkpoint inhibitor.

93. The use of claim 92, wherein the immune-checkpoint inhibitor is a PD-1 inhibitor or a PD-L1 inhibitor.

94. The use of any one of claims 72-93, wherein the subject is a human.

95. The use of any one of claims 59-94, wherein the anti-ILT3 antibody inhibits binding of ILT3 to fibronectin.

96. The use of any one of claims 59-95, wherein the anti-ILT3 antibody inhibits binding of ILT3 to APOE.

97. The use of any one of claims 59-96, wherein the anti-ILT3 antibody inhibits binding of ILT3 to CNTFR.

98. The use of any one of claims 59-97, wherein the anti-ILT3 antibody inhibits ILT3 activity.

99. The use of any one of claims 59-98, wherein the anti-LAIR-1 antibody inhibits LAIR-1 activity.

100. The use of any one of claims 59-99, wherein the anti-LAIR-1 antibody inhibits binding of LAIR-1 to collagen.

101. The use of any of claims 59-100, wherein the anti-LAIR-1 antibody inhibits binding of LAIR-1 to MARCO.

102. The use of any of claims 59-101, wherein the anti-LAIR-1 antibody inhibits binding of LAIR-1 to COLEC12.

103. The use of any one of claims 59-102, wherein the anti-ILT3 antibody comprises:
(a) a heavy chain variable region (VH) comprising a VH-complementarity determining region (CDR)1, a VH-CDR2, and a VH-CDR3 from the amino acid sequence of SEQ ID NO:111; and a light chain variable region (VL) comprising a VL-CDR1, a VL-CDR2, and a VL-CDR3 from the amino acid sequence of SEQ ID NO:112; or
(b) a VH comprising a VH-CDR1, a VH-CDR2, and a VH-CDR3 from the amino acid sequence of SEQ ID NO:123; and a VL comprising a VL-CDR1, a VL-CDR2, and a VL-CDR3 from the amino acid sequence of SEQ ID NO:124.

104. The use of claim 103, wherein:
(a) the VH comprises a VH-CDR1 comprising the amino acid sequence GFTFSSYGMS (SEQ ID NO:27), a VH-CDR2 comprising the amino acid sequence TISGGGSYTNYPDSVKG (SEQ ID NO:28), and a VH-CDR3 comprising the amino acid sequence REWRYTLYAMDY (SEQ ID NO:105); and the VL comprises a VL-CDR1 comprising the amino acid sequence RASESVESYGSSFMH (SEQ ID NO:106), a VL-CDR2 comprising the amino acid sequence LTSNLES (SEQ ID NO:31), and a VL-CDR3 comprising the amino acid sequence QQNNEDPFT (SEQ ID NO:32);
(b) the VH comprises a VH-CDR1 comprising the amino acid sequence GFTFSSY (SEQ ID NO:33), a VH-CDR2 comprising the amino acid sequence SGGGSY (SEQ ID NO:34), and a VH-CDR3 comprising the amino acid sequence REWRYTLYAMDY (SEQ ID NO:105); and the VL comprises a VL-CDR1 comprising the amino acid sequence RASESVESYGSSFMH (SEQ ID NO:106), a VL-CDR2 comprising the amino acid sequence LTSNLES (SEQ ID NO:31), and a VL-CDR3 comprising the amino acid sequence QQNNEDPFT (SEQ ID NO:32);
(c) the VH comprises a VH-CDR1 comprising the amino acid sequence GFTFSSYGMS (SEQ ID NO:27), a VH-CDR2 comprising the amino acid sequence TISGGGSYTN (SEQ ID NO:35), and a VH-CDR3 comprising the amino acid sequence REWRYTLYAMDY (SEQ ID NO:105); and the VL comprises a VL-CDR1 comprising the amino acid sequence RASESVESYGSSFMH (SEQ ID NO:106), a VL-CDR2 comprising the amino acid sequence LTSNLES (SEQ ID NO:31), and a VL-CDR3 comprising the amino acid sequence QQNNEDPFT (SEQ ID NO:32);
(d) the VH comprises a VH-CDR1 comprising the amino acid sequence SYGMS (SEQ ID NO:36), a VH-CDR2 comprising the amino acid sequence TISGGGSYTNYPDSVKG (SEQ ID NO:28), and a VH-CDR3 comprising the amino acid sequence REWRYTLYAMDY (SEQ ID NO:105); and the VL comprises a VL-CDR1 comprising the amino acid sequence RASESVESYGSSFMH (SEQ ID NO:106), a VL-CDR2 comprising the amino acid sequence LTSNLES (SEQ ID NO:31), and a VL-CDR3 comprising the amino acid sequence QQNNEDPFT (SEQ ID NO:32); or
(e) the VH comprises a VH-CDR1 comprising the amino acid sequence SSYGMS (SEQ ID NO:37), a VH-CDR2 comprising the amino acid sequence WVATISGGGSYTN (SEQ ID NO:38), and a VH-CDR3 comprising the amino acid sequence ARREWRYTLYAMD (SEQ ID NO:107), and the VL comprises a VL-CDR1 comprising the amino acid sequence ESYGSSFMHWY (SEQ ID NO:108), a VL-CDR2 comprising the amino acid sequence LLIYLTSNLE (SEQ ID NO:41), and a VL-CDR3 comprising the amino acid sequence QQNNEDPF (SEQ ID NO:42).

105. The use of claim 103 or 104, wherein:
(a) the VH comprises an amino acid sequence having at least 80% sequence identity to the amino acid sequence of SEQ ID NO:123;
(b) the VL comprises an amino acid sequence having at least 80% sequence identity to the amino acid sequence of SEQ ID NO:124; or
(c) the VH comprises an amino acid sequence having at least 80% sequence identity to the amino acid sequence of SEQ ID NO:123 and the VL comprises an amino acid sequence having at least 80% sequence identity to the amino acid sequence of SEQ ID NO:124.

106. The use of claim 105, wherein the VH comprises the amino acid sequence of SEQ ID NO:123 and the VL comprises the amino acid sequence of SEQ ID NO:124.

107. The use of any one of claims 103-105, wherein the anti-ILT3 antibody comprises:
(a) a heavy chain comprising an amino acid sequence having at least 80% sequence identity to the amino acid sequence of SEQ ID NO:126;
(b) a light chain comprising an amino acid sequence having at least 80% sequence identity to the amino acid sequence of SEQ ID NO:128; or
(c) a heavy chain comprising an amino acid sequence having at least 80% sequence identity to the amino acid sequence of SEQ ID NO:126, and a light chain comprising an amino acid sequence having at least 80% sequence identity to the amino acid sequence of SEQ ID NO:128.

108. The use of claim 107, wherein the heavy chain comprises the amino acid sequence of SEQ ID NO:126 and the light chain comprises the amino acid sequence of SEQ ID NO:128.

109. The use of any one of claims 59-108, wherein the anti-LAIR-1 antibody comprises: a heavy chain variable region (VH) comprising a VH-complementarity determining region (CDR)1, a VH-CDR2 and a VH-CDR3 from SEQ ID NO:179, and a light chain variable region (VL) comprising a VL-CDR1, a VL-CDR2 and a VL-CDR3 from SEQ ID NO:180.

110. The use of claim 109, wherein:
(a) the VH comprises the VH-CDR1 comprising the amino acid sequence of SEQ ID NO:242, the VH-CDR2 comprising the amino acid sequence of SEQ ID NO:257, and the VH-CDR3 comprising the amino acid sequence of SEQ ID NO:244; and the VL comprises the VL-CDR1 comprising the amino acid sequence of SEQ ID NO:245, the VL-CDR2 comprising the amino acid sequence of SEQ ID NO:258, and the VL-CDR3 comprising the amino acid sequence of SEQ ID NO:247;
(b) the VH comprises the VH-CDR1 comprising the amino acid sequence of SEQ ID NO:248, the VH-CDR2 comprising the amino acid sequence of SEQ ID NO:259, and the VH-CDR3 comprising the amino acid sequence of SEQ ID NO:244; and the VL comprises the VL-CDR1 comprising the amino acid sequence of SEQ ID NO:245, the VL-CDR2 comprising the amino acid sequence of SEQ ID NO:258, and the VL-CDR3 comprising the amino acid sequence of SEQ ID NO:247;
(c) the VH comprises the VH-CDR1 comprising the amino acid sequence of SEQ ID NO:242, the VH-CDR2 comprising the amino acid sequence of SEQ ID NO:260, and the VH-CDR3 comprising the amino acid sequence of SEQ ID NO:244; and the VL comprises the VL-CDR1 comprising the amino acid sequence of SEQ ID NO:245, the VL-CDR2 comprising the amino acid sequence of SEQ ID NO:258, and the VL-CDR3 comprising the amino acid sequence of SEQ ID NO:247;
(d) the VH comprises the VH-CDR1 comprising the amino acid sequence of SEQ ID NO:250, the VH-CDR2 comprising the amino acid sequence of SEQ ID NO:257, and the VH-CDR3 comprising the amino acid sequence of SEQ ID NO:244; and the VL comprises the VL-CDR1 comprising the amino acid sequence of SEQ ID NO:245, the VL-CDR2 comprising the amino acid sequence of SEQ ID NO:258, and the VL-CDR3 comprising the amino acid sequence of SEQ ID NO:247; or
(e) the VH comprises the VH-CDR1 comprising the amino acid sequence of SEQ ID NO:251, the VH-CDR2 comprising the amino acid sequence of SEQ ID NO:261, and the VH-CDR3 comprising the amino acid sequence of SEQ ID NO:253; and the VL comprises the VL-CDR1 comprising the amino acid sequence of SEQ ID NO:254, the VL-CDR2 comprising the amino acid sequence of SEQ ID NO:255, and the VL-CDR3 comprising the amino acid sequence of SEQ ID NO:256.

111. The use of any one of claim 109 or 110, wherein: the VH has at least 80% sequence identity to the amino acid sequence of SEQ ID NO:179 and/or the VL has at least 80% sequence identity to the amino acid sequence of SEQ ID NO:180.

112. The use of claim 111, wherein: the VH comprises the amino acid sequence of SEQ ID NO:179, and/or the VL comprises the amino acid sequence of SEQ ID NO:180.

113. The use of any one of claims 59-112, wherein the anti-LAIR-1 antibody comprises: a heavy chain comprising an amino acid sequence with 80% identity to the amino acid sequence of SEQ ID NO:194, and/or a light chain comprising an amino acid sequence with 80% identity to the amino acid sequence of SEQ ID NO:196.

114. The use of claim 113, wherein the heavy chain comprises the amino acid sequence of SEQ ID NO:194, and/or the light chain comprises the amino acid sequence of SEQ ID NO:196.

115. The use of any one of claims 59-113, wherein the anti-ILT3 antibody and the anti-LAIR-1 antibody are formulated into two separate pharmaceutical compositions, where a first pharmaceutical composition comprises the anti-ILT3 antibody and a pharmaceutically acceptable carrier; and a second pharmaceutical composition comprises the anti-LAIR-1 antibody and a pharmaceutically acceptable carrier.

116. The use of any one of claims 59-113, wherein the anti-ILT3 antibody and the anti-LAIR-1 antibody are formulated into a pharmaceutical composition, wherein the pharmaceutical composition comprises the anti-ILT3 antibody, the anti-LAIR-1 antibody, and a pharmaceutically acceptable carrier.

117. A kit comprising a first container, a second container and a package insert, wherein the first container comprises at least one dose of a medicament comprising an anti-ILT3 antibody, the second container comprises at least one dose of a medicament comprising an anti-LAIR-1 antibody, and the package insert comprises instructions for treating a subject diagnosed with cancer using the medicaments.

118. The kit of claim 117, wherein the subject is a human.

119. The kit of claim 117 or 118, wherein the anti-ILT3 antibody comprises:
(a) a heavy chain variable region (VH) comprising a VH-complementarity determining region (CDR)1, a VH-CDR2, and a VH-CDR3 from the amino acid sequence of SEQ ID NO:111; and a light chain variable region (VL) comprising a VL-CDR1, a VL-CDR2, and a VL-CDR3 from the amino acid sequence of SEQ ID NO:112; or
(b) a VH comprising a VH-CDR1, a VH-CDR2, and a VH-CDR3 from the amino acid sequence of SEQ ID NO:123; and a VL comprising a VL-CDR1, a VL-CDR2, and a VL-CDR3 from the amino acid sequence of SEQ ID NO:124.

120. The kit of claim 119, wherein:
(a) the VH comprises a VH-CDR1 comprising the amino acid sequence GFTFSSYGMS (SEQ ID NO:27), a VH-CDR2 comprising the amino acid sequence TISGGGSYTNYPDSVKG (SEQ ID NO:28), and a VH-CDR3 comprising the amino acid sequence REWRYTLYAMDY (SEQ ID NO:105); and the VL comprises a VL-CDR1 comprising the amino acid sequence RASESVESYGSSFMH (SEQ ID NO:106), a VL-CDR2 comprising the amino acid sequence LTSNLES (SEQ ID NO:31), and a VL-CDR3 comprising the amino acid sequence QQNNEDPFT (SEQ ID NO:32);
(b) the VH comprises a VH-CDR1 comprising the amino acid sequence GFTFSSY (SEQ ID NO:33), a VH-CDR2 comprising the amino acid sequence SGGGSY (SEQ ID NO:34), and a VH-CDR3 comprising the amino acid sequence REWRYTLYAMDY (SEQ ID NO:105); and the VL comprises a VL-CDR1 comprising the amino acid sequence RASESVESYGSSFMH (SEQ ID NO:106), a VL-CDR2 comprising the amino acid sequence LTSNLES (SEQ ID NO:31), and a VL-CDR3 comprising the amino acid sequence QQNNEDPFT (SEQ ID NO:32);
(c) the VH comprises a VH-CDR1 comprising the amino acid sequence GFTFSSYGMS (SEQ ID NO:27), a VH-CDR2 comprising the amino acid sequence TISGGGSYTN (SEQ ID NO:35), and a VH-CDR3 comprising the amino acid sequence REWRYTLYAMDY (SEQ ID NO:105); and the VL comprises a VL-CDR1 comprising the amino acid sequence RASESVESYGSSFMH (SEQ ID NO:106), a VL-CDR2 comprising the amino acid sequence LTSNLES (SEQ ID NO:31), and a VL-CDR3 comprising the amino acid sequence QQNNEDPFT (SEQ ID NO:32);
(d) the VH comprises a VH-CDR1 comprising the amino acid sequence SYGMS (SEQ ID NO:36), a VH-CDR2 comprising the amino acid sequence TISGGGSYTNYPDSVKG (SEQ ID NO:28), and a VH-CDR3 comprising the amino acid sequence REWRYTLYAMDY (SEQ ID NO:105); and the VL comprises a VL-CDR1 comprising the amino acid sequence RASESVESYGSSFMH (SEQ ID NO:106), a VL-CDR2 comprising the amino acid sequence LTSNLES (SEQ ID NO:31), and a VL-CDR3 comprising the amino acid sequence QQNNEDPFT (SEQ ID NO:32); or
(e) the VH comprises a VH-CDR1 comprising the amino acid sequence SSYGMS (SEQ ID NO:37), a VH-CDR2 comprising the amino acid sequence WVATISGGGSYTN (SEQ ID NO:38), and a VH-CDR3 comprising the amino acid sequence ARREWRYTLYAMD (SEQ ID NO:107), and the VL comprises a VL-CDR1 comprising the amino acid sequence ESYGSSFMHWY (SEQ ID NO:108), a VL-CDR2 comprising the amino acid sequence LLIYLTSNLE (SEQ ID NO:41), and a VL-CDR3 comprising the amino acid sequence QQNNEDPF (SEQ ID NO:42).

121. The kit of claim 119 or 120, wherein:
(a) the VH comprises an amino acid sequence having at least 80% sequence identity to the amino acid sequence of SEQ ID NO:123;
(b) the VL comprises an amino acid sequence having at least 80% sequence identity to the amino acid sequence of SEQ ID NO:124; or
(c) the VH comprises an amino acid sequence having at least 80% sequence identity to the amino acid sequence of SEQ ID NO:123 and the VL comprises an amino acid sequence having at least 80% sequence identity to the amino acid sequence of SEQ ID NO:124.

122. The kit of claim 121, wherein the VH comprises the amino acid sequence of SEQ ID NO:123 and the VL comprises the amino acid sequence of SEQ ID NO:124.

123. The kit of any one of claims 117-122, wherein the anti-ILT3 antibody comprises:
(a) a heavy chain comprising an amino acid sequence having at least 80% sequence identity to the amino acid sequence of SEQ ID NO:126;
(b) a light chain comprising an amino acid sequence having at least 80% sequence identity to the amino acid sequence of SEQ ID NO:128; or
(c) a heavy chain comprising an amino acid sequence having at least 80% sequence identity to the amino acid sequence of SEQ ID NO:126, and a light chain comprising an amino acid sequence having at least 80% sequence identity to the amino acid sequence of SEQ ID NO:128.

124. The kit of claim 123, wherein the heavy chain comprises the amino acid sequence of SEQ ID NO:126 and the light chain comprises the amino acid sequence of SEQ ID NO:128.

125. The kit any one of claims 117-124, wherein the anti-LAIR-1 antibody comprises: a heavy chain variable region (VH) comprising a VH-complementarity determining region (CDR)1, a VH-CDR2 and a VH-CDR3 from SEQ ID NO:179, and a light chain variable region (VL) comprising a VL-CDR1, a VL-CDR2 and a VL-CDR3 from SEQ ID NO:180.

126. The kit of claim 125, wherein:
(a) the VH comprises the VH-CDR1 comprising the amino acid sequence of SEQ ID NO:242, the VH-CDR2 comprising the amino acid sequence of SEQ ID NO:257, and the VH-CDR3 comprising the amino acid sequence of SEQ ID NO:244; and the VL comprises the VL-CDR1 comprising the amino acid sequence of SEQ ID NO:245, the VL-CDR2 comprising the amino acid sequence of SEQ ID NO:258, and the VL-CDR3 comprising the amino acid sequence of SEQ ID NO:247;
(b) the VH comprises the VH-CDR1 comprising the amino acid sequence of SEQ ID NO:248, the VH-CDR2 comprising the amino acid sequence of SEQ ID NO:259, and the VH-CDR3 comprising the amino acid sequence of SEQ ID NO:244; and the VL comprises the VL-CDR1 comprising the amino acid sequence of SEQ ID NO:245, the VL-CDR2 comprising the amino acid sequence of SEQ ID NO:258, and the VL-CDR3 comprising the amino acid sequence of SEQ ID NO:247;
(c) the VH comprises the VH-CDR1 comprising the amino acid sequence of SEQ ID NO:242, the VH-CDR2 comprising the amino acid sequence of SEQ ID NO:260, and the VH-CDR3 comprising the amino acid sequence of SEQ ID NO:244; and the VL comprises the VL-CDR1 comprising the amino acid sequence of SEQ ID NO:245, the VL-CDR2 comprising the amino acid sequence of SEQ ID NO:258, and the VL-CDR3 comprising the amino acid sequence of SEQ ID NO:247;
(d) the VH comprises the VH-CDR1 comprising the amino acid sequence of SEQ ID NO:250, the VH-CDR2 comprising the amino acid sequence of SEQ ID NO:257, and the VH-CDR3 comprising the amino acid sequence of SEQ ID NO:244; and the VL comprises the VL-CDR1 comprising the amino acid sequence of SEQ ID NO:245, the VL-CDR2 comprising the amino acid sequence of SEQ ID NO:258, and the VL-CDR3 comprising the amino acid sequence of SEQ ID NO:247; or
(e) the VH comprises the VH-CDR1 comprising the amino acid sequence of SEQ ID NO:251, the VH-CDR2 comprising the amino acid sequence of SEQ ID NO:261, and the VH-CDR3 comprising the amino acid sequence of SEQ ID NO:253; and the VL comprises the VL-CDR1 comprising the amino acid sequence of SEQ ID NO:254, the VL-CDR2 comprising the amino acid sequence of SEQ ID NO:255, and the VL-CDR3 comprising the amino acid sequence of SEQ ID NO:256.

127. The kit of claim 125 or 126, wherein: the VH has at least 80% sequence identity to the amino acid sequence of SEQ ID NO:179; and/or the VL has at least 80% sequence identity to the amino acid sequence of SEQ ID NO:180.

128. The kit of claim 127, wherein: the VH comprises the amino acid sequence of SEQ ID NO:179; and/or the VL comprises the amino acid sequence of SEQ ID NO:180.

129. The kit of any of claims 117-128, wherein the anti-LAIR-1 antibody comprises: a heavy chain comprising an amino acid sequence having 80% identity to the amino acid sequence of SEQ ID NO:194, and/or a light chain comprising an amino acid sequence having 80% identity to the amino acid sequence of SEQ ID NO:196.

130. The kit of claim 129, wherein the heavy chain comprises the amino acid sequence of SEQ ID NO:194, and/or the light chain comprises the amino acid sequence of SEQ ID NO:196.

131. A combination comprising an anti-LAIR-1 antibody and an anti-ILT3 antibody.

132. The combination of claim 131, wherein the anti-LAIR-1 antibody and the anti-ILT3 antibody are formulated separately.

133. The combination of claim 131, wherein the anti-LAIR-1 antibody and the anti-ILT3 antibody are co-formulated.

134. The combination of claim 131, wherein the anti-LAIR-1 antibody and the anti-ILT3 antibody are co-formulated as a pharmaceutical composition.

135. The combination of claim 131, wherein the anti-LAIR-1 antibody and the anti-ILT3 antibody are each separately formulated as a pharmaceutical composition.

136. The combination of claim 131, wherein the anti-LAIR-1 antibody and the anti-ILT3 antibody synergistically overcomes stromal-mediated immunosuppression in a tumor microenvironment.

137. The combination of any one of claims 131-136, wherein the anti-ILT3 antibody comprises:
(a) a heavy chain variable region (VH) comprising a VH-complementarity determining region (CDR)1, a VH-CDR2, and a VH-CDR3 from the amino acid sequence of SEQ ID NO:111; and a light chain variable region (VL) comprising a VL-CDR1, a VL-CDR2, and a VL-CDR3 from the amino acid sequence of SEQ ID NO:112; or
(b) a VH comprising a VH-CDR1, a VH-CDR2, and a VH-CDR3 from the amino acid sequence of SEQ ID NO:123; and a VL comprising a VL-CDR1, a VL-CDR2, and a VL-CDR3 from the amino acid sequence of SEQ ID NO:124.

138. The combination of claim 137, wherein the anti-ILT3 antibody comprises:
(a) the VH comprises a VH-CDR1 comprising the amino acid sequence GFTFSSYGMS (SEQ ID NO:27), a VH-CDR2 comprising the amino acid sequence TISGGGSYTNYPDSVKG (SEQ ID NO:28), and a VH-CDR3 comprising the amino acid sequence REWRYTLYAMDY (SEQ ID NO:105); and the VL comprises a VL-CDR1 comprising the amino acid sequence RASESVESYGSSFMH (SEQ ID NO:106), a VL-CDR2 comprising the amino acid sequence LTSNLES (SEQ ID NO:31), and a VL-CDR3 comprising the amino acid sequence QQNNEDPFT (SEQ ID NO:32);
(b) the VH comprises a VH-CDR1 comprising the amino acid sequence GFTFSSY (SEQ ID NO:33), a VH-CDR2 comprising the amino acid sequence SGGGSY (SEQ ID NO:34), and a VH-CDR3 comprising the amino acid sequence REWRYTLYAMDY (SEQ ID NO:105); and the VL comprises a VL-CDR1 comprising the amino acid sequence RASESVESYGSSFMH (SEQ ID NO:106), a VL-CDR2 comprising the amino acid sequence LTSNLES (SEQ ID NO:31), and a VL-CDR3 comprising the amino acid sequence QQNNEDPFT (SEQ ID NO:32);
(c) the VH comprises a VH-CDR1 comprising the amino acid sequence GFTFSSYGMS (SEQ ID NO:27), a VH-CDR2 comprising the amino acid sequence TISGGGSYTN (SEQ ID NO:35), and a VH-CDR3 comprising the amino acid sequence REWRYTLYAMDY (SEQ ID NO:105); and the VL comprises a VL-CDR1 comprising the amino acid sequence RASESVESYGSSFMH (SEQ ID NO:106), a VL-CDR2 comprising the amino acid sequence LTSNLES (SEQ ID NO:31), and a VL-CDR3 comprising the amino acid sequence QQNNEDPFT (SEQ ID NO:32);
(d) the VH comprises a VH-CDR1 comprising the amino acid sequence SYGMS (SEQ ID NO:36), a VH-CDR2 comprising the amino acid sequence TISGGGSYTNYPDSVKG (SEQ ID NO:28), and a VH-CDR3 comprising the amino acid sequence REWRYTLYAMDY (SEQ ID NO:105); and the VL comprises a VL-CDR1 comprising the amino acid sequence RASESVESYGSSFMH (SEQ ID NO:106), a VL-CDR2 comprising the amino acid sequence LTSNLES (SEQ ID NO:31), and a VL-CDR3 comprising the amino acid sequence QQNNEDPFT (SEQ ID NO:32); or
(e) the VH comprises a VH-CDR1 comprising the amino acid sequence SSYGMS (SEQ ID NO:37), a VH-CDR2 comprising the amino acid sequence WVATISGGGSYTN (SEQ ID NO:38), and a VH-CDR3 comprising the amino acid sequence ARREWRYTLYAMD (SEQ ID NO:107), and the VL comprises a VL-CDR1 comprising the amino acid sequence ESYGSSFMHWY (SEQ ID NO:108), a VL-CDR2 comprising the amino acid sequence LLIYLTSNLE (SEQ ID NO:41), and a VL-CDR3 comprising the amino acid sequence QQNNEDPF (SEQ ID NO:42).

139. The combination of claim 137 or 138, wherein:
(a) the VH comprises an amino acid sequence having at least 80% sequence identity to the amino acid sequence of SEQ ID NO:123;
(b) the VL comprises an amino acid sequence having at least 80% sequence identity to the amino acid sequence of SEQ ID NO:124; or
(c) the VH comprises an amino acid sequence having at least 80% sequence identity to the amino acid sequence of SEQ ID NO:123 and the VL comprises an amino acid sequence having at least 80% sequence identity to the amino acid sequence of SEQ ID NO:124.

140. The combination of claim 139, wherein the anti-ILT3 antibody comprises: the heavy chain variable region comprising the amino acid sequence of SEQ ID NO:123 and the light chain variable region comprising the amino acid sequence of SEQ ID NO:124.

141. The combination of any one of claims 137-139, wherein the anti-ILT3 antibody comprises:
(a) a heavy chain comprising an amino acid sequence having at least 80% sequence identity to the sequence of SEQ ID NO:126;
(b) a light chain comprising an amino acid sequence having at least 80% sequence identity to the sequence of SEQ ID NO:128; or
(c) a heavy chain comprising an amino acid sequence having at least 80% sequence identity to the sequence of SEQ ID NO:126 and a light chain comprising an amino acid sequence having at least 80% sequence identity to the sequence of SEQ ID NO:128.

142. The combination of claim 141, wherein the VH comprises the amino acid sequence of SEQ ID NO:123 and the VL comprises the amino acid sequence of SEQ ID NO:124.

143. The combination of any one of claims 131-142, wherein the anti-LAIR-1 antibody comprises: a heavy chain variable region (VH) comprising a VH-complementarity determining region (CDR)1, a VH-CDR2 and a VH-CDR3 from SEQ ID NO:179, and a light chain variable region (VL) comprising a VL-CDR1, a VL-CDR2 and a VL-CDR3 from SEQ ID NO:180.

144. The combination of claim 143, wherein:
(a) the VH comprises the VH-CDR1 comprising the amino acid sequence of SEQ ID NO:242, the VH-CDR2 comprising the amino acid sequence of SEQ ID NO:257, and the VH-CDR3 comprising the amino acid sequence of SEQ ID NO:244; and the VL comprises the VL-CDR1 comprising the amino acid sequence of SEQ ID NO:245, the VL-CDR2 comprising the amino acid sequence of SEQ ID NO:258, and the VL-CDR3 comprising the amino acid sequence of SEQ ID NO:247;
(b) the VH comprises the VH-CDR1 comprising the amino acid sequence of SEQ ID NO:248, the VH-CDR2 comprising the amino acid sequence of SEQ ID NO:259, and the VH-CDR3 comprising the amino acid sequence of SEQ ID NO:244; and the VL comprises the VL-CDR1 comprising the amino acid sequence of SEQ ID NO:245, the VL-CDR2 comprising the amino acid sequence of SEQ ID NO:258, and the VL-CDR3 comprising the amino acid sequence of SEQ ID NO:247;
(c) the VH comprises the VH-CDR1 comprising the amino acid sequence of SEQ ID NO:242, the VH-CDR2 comprising the amino acid sequence of SEQ ID NO:260, and the VH-CDR3 comprising the amino acid sequence of SEQ ID NO:244; and the VL comprises the VL-CDR1 comprising the amino acid sequence of SEQ ID NO:245, the VL-CDR2 comprising the amino acid sequence of SEQ ID NO:258, and the VL-CDR3 comprising the amino acid sequence of SEQ ID NO:247;
(d) the VH comprises the VH-CDR1 comprising the amino acid sequence of SEQ ID NO:250, the VH-CDR2 comprising the amino acid sequence of SEQ ID NO:257, and the VH-CDR3 comprising the amino acid sequence of SEQ ID NO:244; and the VL comprises the VL-CDR1 comprising the amino acid sequence of SEQ ID NO:245, the VL-CDR2 comprising the amino acid sequence of SEQ ID NO:258, and the VL-CDR3 comprising the amino acid sequence of SEQ ID NO:247; or
(e) the VH comprises the VH-CDR1 comprising the amino acid sequence of SEQ ID NO:251, the VH-CDR2 comprising the amino acid sequence of SEQ ID NO:261, and the VH-CDR3 comprising the amino acid sequence of SEQ ID NO:253; and the VL comprises the VL-CDR1 comprising the amino acid sequence of SEQ ID NO:254, the VL-CDR2 comprising the amino acid sequence of SEQ ID NO:255, and the VL-CDR3 comprising the amino acid sequence of SEQ ID NO:256.

145. The combination of any one of claim 143 or 144, wherein the VH has at least 80% sequence identity to the amino acid sequence of SEQ ID NO:179, and/or the VL has at least 80% sequence identity to the amino acid sequence of SEQ ID NO:180.

146. The combination of claim 145, wherein: the VH comprises the amino acid sequence of SEQ ID NO:179, and/or the VL comprises the amino acid sequence of SEQ ID NO:180.

147. The combination of any one of claims 131-146, wherein the anti-LAIR-1 antibody comprises: a heavy chain comprising an amino acid sequence with 80% identity to the amino acid sequence of SEQ ID NO:194 and/or a light chain comprising an amino acid sequence with 80% identity to the amino acid sequence of SEQ ID NO:196.

148. The combination of claim 147, wherein the heavy chain comprises the amino acid sequence of SEQ ID NO:194, and/or the light chain comprises the amino acid sequence of SEQ ID NO:196.
